(19) United States
(12) Patent Application Publication
(10) Pub. No.: US 2024/0360451 A1
(43) Pub. Date: Oct. 31, 2024

(54) MODULATION OF GENE TRANSCRIPTION USING ANTISENSE OLIGONUCLEOTIDES TARGETING REGULATORY RNAS

(71) Applicant: Camp4 Therapeutics Corporation, Cambridge, MA (US)

(72) Inventors: Alfica Sehgal, Belmont, MA (US); Bryan J. Matthews, Somerville, MA (US); Cynthia M. Smith, Boston, MA (US); David A. Bumcrot, Belmont, MA (US); Justin A. Caravella, Cambridge, MA (US); Mario Esteban Contreras Gamboa, Cambridge, MA (US); Rachana S. Kelkar, Malden, MA (US); Yun Joon Jung, Lexington, MA (US); Yuting Liu, Lexington, MA (US); Subhadeep Roy, Lafayette, CO (US); Brynn Nicole-Yoshiko Akerberg, Brookline, MA (US); Rutuja Sudhakar Pai, Charlestown, MA (US)

(21) Appl. No.: 18/688,004

(22) PCT Filed: Sep. 2, 2022
(86) PCT No.: PCT/US2022/075934

§ 371 Date: Feb. 29, 2024

Related U.S. Application Data
(60) Provisional application No. 63/292,792, filed on Dec. 22, 2021.
(60) Provisional application No. 63/240,838, filed on Sep. 3, 2021.

(51) Int. Cl. C12N 15/113; A61P 3/00

(52) U.S. Cl. CPC C12N 15/113; A61P 3/00; C12N 2310/11; C12N 2310/321; C12N 2310/3231; C12N 2310/3341; C12N 2310/351

(57) ABSTRACT

Described herein are methods of modulating OTC gene transcription using antisense oligonucleotides (ASOs) targeting regulatory RNAs, such as promoter-associated RNAs and enhancer RNAs. These methods are useful for increasing expression of Ornithine transcarbamylase (OTC), thereby treating diseases associated with aberrant gene expression.

### CROSS REFERENCE TO RELATED APPLICATIONS

This application claims the benefit of U.S. Provisional Application No. 63/240,838, filed Sep. 3, 2021 and U.S. Provisional Application No. 63/292,792, filed Dec. 22, 2021; each of which are hereby incorporated in its entirety by reference.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### FIELD OF THE INVENTION

The invention relates to methods of upregulating or downregulating OTC gene transcription using antisense oligonucleotides (ASOs) targeting OTC regulatory RNAs, such as promoter-associated RNAs and enhancer RNAs.

### BACKGROUND

Transcription factors bind specific sequences in promoter and enhancer DNA elements to regulate gene transcription. It was recently reported that active promoters and enhancer elements are themselves transcribed, generating noncoding regulatory RNAs (regRNAs) such as promoter-associated RNAs (paRNAs) and enhancer RNAs (eRNAs) (see Sartorelli and Lauberth, Nat. Struct. Mol. Biol. (2020) 27, 521-28). Unlike coding RNAs, regRNAs are transcribed bi-directionally. Various models have been proposed for the functions of regRNAs, including nucleosome remodeling (see Mousavi et al., Mol. Cell (2013) 51(5):606-17), modulation of enhancer-promoter looping (see Lai et al., Nature (2013) 494(7438):497-501), and direct interaction with transcription regulators (see Sigova et al., Science (2015) 350, 978-81).

Gene expression has been generally known as an undruggable biological process. Despite on-going efforts into understanding the biology of gene transcription and regRNAs, clinically suitable methods of modulating gene expression are limited. There remains a need for new and useful methods for treating diseases associated with aberrant gene expression.

### SUMMARY

The present invention provides antisense oligonucleotides (ASOs) targeting regulatory RNAs, such as promoter-associated RNAs and enhancer RNAs, and methods using these ASOs to regulate gene expression. These methods are useful for modulating the levels of gene products, for example, modulating expression levels of disease-causing genes such as Ornithine transcarbamylase (OTC), thereby to treat diseases associated with aberrant gene expression such as urea cycle disorders.

In one aspect, provided herein is an antisense oligonucleotide (ASO) complementary to at least 8 contiguous nucleotides of a regulatory RNA of human Ornithine Transcarbamylase (OTC), wherein the regulatory RNA has a nucleotide sequence selected from the group consisting of SEQ ID NOs: 1-4 or 1077.

In some embodiments, the ASO is complementary to a sequence in the regRNA that is no more than 200 nucleotides from the 3′ end of the regRNA.

In some embodiments, the ASO is complementary to a sequence in the regRNA that is no more than 200 nucleotides from the 5′ end of the regRNA.

In some embodiments, the regRNA is not a polyadenylated RNA.

In some embodiments, the ASO does not induce RNAse H-mediated degradation of the regRNA.

In some embodiments, the regulatory RNA has a nucleotide sequence of SEQ ID NO: 1, and the ASO comprises a nucleotide sequence selected from the group consisting of SEQ ID NOs: 6-14, 18-35, 39, 41, 75, 76, 77, 78, 87-124, or 143-892.

In some embodiments, the regulatory RNA has a nucleotide sequence of SEQ ID NO: 2, and the ASO comprises the nucleotide sequence of SEQ ID NO: 15-17, 36-38, 64-74, 125-142, or 893-1029.

In some embodiments, the regulatory RNA has a nucleotide sequence of SEQ ID NO: 2, and the ASO comprises the nucleotide sequence of SEQ ID NO: 17.

In some embodiments, the ASO is no more than 50, 40, 30, or 25 nucleotides in length.

In some embodiments, the ASO comprises a RNA polynucleotide comprising one or more chemical modifications.

In some embodiments, at least 3, 4, or 5 nucleotides at the 5′ end and at least 3, 4, or 5 nucleotides at the 3′ end of the ASO comprise ribonucleotides with one or more chemical modifications.

In some embodiments, the one or more chemical modifications comprise a nucleotide sugar modification comprising one or more of 2′-O—C1-4alkyl such as 2′-O-methyl (2′-OMe), 2′-deoxy (2′-H), 2′-O—C1-3alkyl-O—C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

In some embodiments, the one or more chemical modifications comprise an internucleotide linkage modification comprising one or more of phosphorothioate (“PS” or (P(S))), phosphoramidate (P(NR1R2) such as dimethylaminophosphoramidate (P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate P(CH3), boranophosphonate (P(BH3)), or phosphorodithioate (P(S)2).

In some embodiments, the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

In some embodiments, the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

In some embodiments, the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

In some embodiments, the ASO comprises the nucleotide sequence of SEQ ID NOs: 18-39 or 67-74.

In some embodiments, the ASO does not comprise 10 or more contiguous nucleotides of unmodified DNA.

In some embodiments, the ASO does not comprise a deoxyribonucleotide.

In some embodiments, the ASO does not comprise an unmodified ribonucleotide.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the ASO further comprises a GalNAc moiety.

In some embodiments, the ASO comprises the nucleotide sequence of SEQ ID NO: 142.

In some embodiments, the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

In some embodiments, the ASO comprises the nucleotide sequence of SEQ ID NO: 21.

In some embodiments, the ASO further comprises a GalNAc moiety.

The ASO of claim 22, wherein the ASO comprises the nucleotide sequence of SEQ ID NO: 122.

In some embodiments, each ribonucleotide of the ASO is modified by 2′-O-methoxyethyl.

In some embodiments, the ASO comprises the nucleotide sequence of SEQ ID NO: 25.

In some embodiments, each nucleotide of the ASO is a ribonucleotide modified by 2′-O-methoxyethyl.

In some embodiments, the ASO comprises the nucleotide sequence of SEQ ID NO: 36.

In some embodiments, the ASO comprises 10 or more contiguous nucleotides of unmodified DNA flanked by at least 3 nucleotides of modified ribonucleotides at each of the 5′ end and the 3′ end.

In some embodiments, the ASO comprises the nucleotide sequence of SEQ ID NO: 18.

In some embodiments, each cytidine in the ASO is modified by 5-methyl.

In some embodiments, the regRNA is an eRNA.

In one aspect, provided herein is pharmaceutical composition comprising the ASo described herein and a pharmaceutically acceptable carrier or excipient carrier.

In one aspect, provided herein is method of increasing transcription of OTC in a human cell, the method comprising contacting the cell with the ASO described herein or the pharmaceutical composition described herein.

In some embodiments, the cell is a hepatocyte.

In some embodiments, the ASO increases the amount of the regulatory RNA in the cell.

In some embodiments, the ASO increases the stability of the regulatory RNA in the cell.

In one aspect, provided herein is method of treating urea cycle disorder, the method comprising administering to a subject in need thereof an effective amount of the ASO described herein or the pharmaceutical composition described herein.

In some embodiments, the ASO increases the amount of the regulatory RNA in a cell of the subject.

In some embodiments, the ASO increases the stability of the regulatory RNA in a cell of the subject.

In some embodiments, the cell is a hepatocyte.

### BRIEF DESCRIPTION OF THE DRAWINGS

FIG. 1 shows an illustrative schematic of eRNA, paRNA, mRNA, and natural antisense transcript (NAT) of a gene on the chromosome. The eRNA, paRNA, and NAT are all non-coding RNAs. The eRNA is transcribed bidirectionally from an enhancer of the gene. The paRNA is transcribed from the promoter of the gene, same as the mRNA, but in the antisense direction. The NAT is transcribed from a downstream promoter of its own in the antisense direction, such that the transcript overlaps at least partially with the mRNA. Generally, eRNAs and paRNAs upregulate gene expression whereas NATs downregulates gene expression.

FIG. 2A-D shows that treatment with the indicated ASO resulted in OTC mRNA upregulation in a dose-dependent manner. FIG. 2A shows OTC mRNA after treatment with hOTC-ASOe1-11. FIG. 2B shows OTC mRNA after treatment with hOTC-ASOe1-8. FIG. 2C shows OTC mRNA after treatment with hOTC-ASOe2-1. FIG. 2D shows OTC mRNA after treatment with hOTC-ASOe1-1.

FIG. 3A shows that OTC mRNA increased in cells derived from an OTC-deficient donor after treatment with ASO hOTC-ASOe1-10 and hOTC-ASOe1-2c. FIG. 3B shows that ureagenesis increased in cells derived from an OTC-deficient donor after treatment with ASO hOTC-ASOe1-10 and hOTC-ASOe1-2c. FIG. 3C shows OTC mRNA increased in WT cells after treatment with ASO hOTC-ASOe1-2a. FIG. 3D shows that ureagenesis increased in WT cells after treatment with ASO hOTC-ASOe1-2a.

FIG. 4 shows that the indicated mouse ASOs increased Otc mRNA levels in primary mouse liver cells from wild type mice. One-way ANOVA * p 0.05-0.005; **: p<0.005.

FIG. 5 shows that the indicated mouse ASOs increased Otc mRNA levels in spf^{ash} primary mouse liver cells. One-way ANOVA **: p<0.005.

FIG. 6A shows upregulation of Serping1 after treatment with IFNy. FIG. 6B shows downregulation of Serping1 after treatment with JAK inhibitor tofacitinib.

FIG. 7 shows the correlation of Serping1 mRNA to protein secretion after IFNy induction.

FIG. 8 shows induction of Serping1 mRNA and regRNA in mouse livers treated with IFNy.

FIG. 9A shows Serping1 mRNA and regRNA levels in mouse hepatocytes in a time course study after treatment with IFNy. FIG. 9B shows Serping1 mRNA and regRNA levels in mouse hepatocytes in a time course study after treatment with IFNy.

FIG. 10 shows Serping1 enhancer 2 RNA and promoter 2 RNA levels after treatment with IFNg or PBS (control).

FIG. 11A shows a schematic of the Serping1 chromosomal neighborhood. FIG. 11B shows mRNA levels of Serping1, Irf1, Ube216, and NTC-3_S after treatment with the indicated ASOs.

FIG. 12 shows mRNA levels of Serping1 after treatment with the indicated ASO.

FIG. 13 shows mRNA levels of Serping1 at 24, 48, and 72 hours after treatment with the indicated ASO.

FIG. 14A shows a diagram of an in vivo mouse study timeline. FIG. 14B shows Serping1 mRNA expression increased in vivo after treatment with ASO-2.

FIG. 15A shows the additive effect of IFNy plus the indicated ASO on Serping1 mRNA expression, normalized to untreated cells. FIG. 15B shows the additive effect of IFNy plus the indicated ASOs on Serping1 mRNA expression, normalized to untreated cells.

FIG. 16 shows Serping1 mRNA expression after JAK1 inhibitor tofacitinib or JAK1 inhibitor tofacitinib plus ASO-2 treatment, normalized to untreated cells.

FIG. 17 shows that the indicated ASO treatment in a Serping1 knockdown system using a JAK1 inhibitor tofacitinib increased Serping1 mRNA expression.

FIGS. 18A and 18B shows schematics of various human OTC ASOs with chemical modifications. Light gray indicates a 2′-O-(2-Methoxyethyl) (2′-MOE) modification. Dark gray indicates a locked nucleic acid (LNA) modification. Line brackets indicate a phosphodiester (PO) linkage. *C indicates a 5-methyl on the cytidine. \hat{ } indicates a FANA nucleoside. Unique sequence identifiers are assigned to nucleotide sequences having the specific chemical modifications shown in this figure. FIG. 18C shows a schematic of various mouse OTC ASOs with chemical modifications. Light gray indicates a 2′-O-(2-Methoxyethyl) (2′-MOE) modification. *C indicates a 5-methyl on the cytidine. Unique sequence identifiers are assigned to nucleotide sequences having the specific chemical modifications shown in this figure.

FIG. 19 shows a schematic of various Serping1 ASOs with chemical modifications. Light gray indicates a 2′-O-(2-Methoxyethyl) (2′-MOE) modification. *C indicates a 5-methyl on the cytidine. Unique sequence identifiers are assigned to nucleotide sequences having the specific chemical modifications shown in this figure.

FIG. 20A shows that treatment with the indicated ASO resulted in human OTC mRNA upregulation in a dose-dependent manner. FIG. 20B shows that treatment with the indicated ASO resulted in OTC mRNA upregulation in a dose-dependent manner.

FIG. 21 shows that treatment with the indicated ASO resulted in human OTC mRNA upregulation in a dose-dependent manner.

FIG. 22 shows that treatment with the indicated ASO resulted in human OTC mRNA upregulation in a dose-dependent manner.

FIG. 23 shows that hOTC-ASOe1-1a did not induce IL6, TNFa, IFNa, or IFNb cytokine release by PBMCs.

FIG. 24A shows that treatment with the indicated ASO resulted in mouse OTC mRNA upregulation in a dose-dependent manner. FIG. 24B shows the Otc regRNA-targeting ASO CO-4474 did not increase mouse Otc mRNA in Otc^{def} mice. FIG. 24C shows that CO-4474 decreased ammonia to WT levels.

FIG. 25A shows upregulation of OTC gene expression after treatment with hOTC-ASOe1-10. FIG. 25B shows the alignments and peaks of of paired-end sequenced ChIP-seq libraries to the human hg38 genome. FIG. 25C shows that differential peaks at the OTC enhancer, OTC promoter and control regions (GAPDH, RPGR, TSPAN7) were identified after treatment with hOTC-ASOe1-10.

FIG. 26 shows accessible chromatin regions at the OTC promoter and enhancer and neighboring RPGR promoter (denoted by boxed regions).

FIG. 27A shows the relative expression levels for the minus strand regRNA (RR1) transcribed from the OTC enhancer over time post ASO treatment. FIG. 27B shows the relative expression levels for the plus strand regRNA (RR2) transcribed from the OTC enhancer over time post ASO treatment. FIG. 27C shows the OTC mRNA effects over time post hOTC-ASOe1-10 treatment. FIG. 27D shows the H3K27ac ChIP-qPCR results post hOTC-ASOe1-10 treatment. FIG. 27E provides a temporal model of the transcriptional and chromatin response to OTC ASO.

FIG. 28A shows the relative loss of binding for the indicated negative regulators after treatment with hOTC-ASOe1-10, as compared to NTC ASO. FIG. 28B shows that HDAC5 and NCOR1 binding is not reduced at the OTC enhancer in hepatocytes after RNase treatment.

FIG. 29A shows that treatment with siHDAC5 or siNCOR1 resulted in at least a 50% reduction in target mRNA levels. FIG. 29B shows that siRNA knockdown of HDAC5 or NCOR1 knockdown lead to increased OTC mRNA expression in hepatocytes. FIG. 29C shows the OTC mRNA fold change after treatment with hOTC-ASOe1-10 in untreated hepatocytes, as well as hepatocytes treated with siHDAC5 or siNCOR1.

FIG. 30 provides a model of the OTC gene expression after treatment with an OTC regRNA-targeting ASO.

FIG. 31 shows the ammonia and urea levels in NHPs after treatment with the indicated ASOs.

FIG. 32 shows the relative OTC, NAGS, CPS1, ASS1, ASL, or ARG1 mRNA expression after treatment with the indicated ASO in a humanize mouse model.

FIG. 33 shows that CO-5318 and CO-5319 treatment in humanized mice showed a decrease in ammonia and a corresponding increase in urea over time

FIG. 34A shows that ASOs CO-3265, CO-3279, CO-2043, and CO-2051 increased Serping1 mRNA expression in a dose dependent manner. FIG. 34B shows ASOs CO-2043, CO-2051, CO-3265, CO-3419, CO-4069, and CO-3279 increased Serping1 gene expression in C1NH+/− hepatocytes in a dose dependent manner.

FIG. 35 shows that the indicated ASOs increased Serping1 mRNA in the mice.

FIG. 36 shows that CO-2051 decreased the amount of dye extravasation in both the ears and colons of CINH+/− mice.

FIG. 37A shows that CO-2051 increased Serping1 protein expression in WT mice. FIG. 37B shows that CO-2051 increased Serping1 protein expression in C1NH+/− mice. FIG. 37C shows that CO-2051-GalNAc increased Serping1 protein expression in C1NH+/− mice. FIG. 37D shows quantification of dye extravasation after treatment with CO-2051-GalNAc.

### DETAILED DESCRIPTION

The present invention provides antisense oligonucleotides (ASOs) targeting regulatory RNAs, such as promoter-associated RNAs and enhancer RNAs, and methods using these ASOs to regulate gene expression. These methods are useful for modulating the levels of gene products, for example, modulating expression levels of disease-causing genes such as Ornithine transcarbamylase (OTC), thereby to treat diseases associated with aberrant gene expression such as the urea cycle disease.

Various aspects of the multi-specific binding proteins described in the present application are set forth below in sections.

### Definitions

To facilitate an understanding of the present application, a number of terms and phrases are defined below.

The terms “a” and “an” as used herein mean “one or more” and include the plural unless the context is inappropriate.

As used herein, the term “Ornithine transcarbamylase” or “OTC” refers to the protein of UniProt Accession No. P00480 and related isoforms and orthologs.

As used herein, the terms “regulatory RNA” and “regRNA” are used interchangeably to refer to a noncoding RNA transcribed from a regulatory element of a gene (e.g., a protein-coding gene), wherein the gene is not the noncoding RNA itself. Exemplary regulatory elements include but are not limited to promoters, enhancers, and super-enhancers. A noncoding RNA transcribed from a promoter, in the antisense direction, is also called “promoter RNA” or “paRNA.” A noncoding RNA transcribed from an enhancer or super-enhancer, in either the sense direction or the anti-sense direction, is also called “enhancer RNA” or “eRNA.” It is understood that a natural antisense transcript (NAT) complementary with at least a portion of the transcript of the gene is not a regulatory RNA as used herein.

As used herein, the term “nascent RNA” refers to an RNA that is still being transcribed or has just been transcribed by RNA polymerase and remains tethered to the DNA from which it is transcribed. An RNA that has dissociated from the DNA from which it is transcribed is also called an “untethered RNA.”

As used herein, the term “antisense oligonucleotide” or “ASO” refers to a single-stranded oligonucleotide having a nucleotide sequence that hybridizes with a target nucleic acid under suitable conditions or a conjugate comprising such single-stranded oligonucleotide.

As used herein, the stability of a regRNA is reversely correlated with the degradation rate of the regRNA. Where an ASO increases the stability of a regRNA, it reduces the degradation rate of the regRNA. Where an ASO decreases the stability of a regRNA, it increases the degradation rate of the regRNA. The degradation rate of a regRNA can be measured by blocking synthesis of new regRNA and assessing the half-life of the existing regRNA.

As used herein, the terms “subject” and “patient” refer to an organism to be treated by the methods and compositions described herein. Such organisms preferably include, but are not limited to, mammals (e.g., rodents, primates, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### Antisense Oligonucleotides

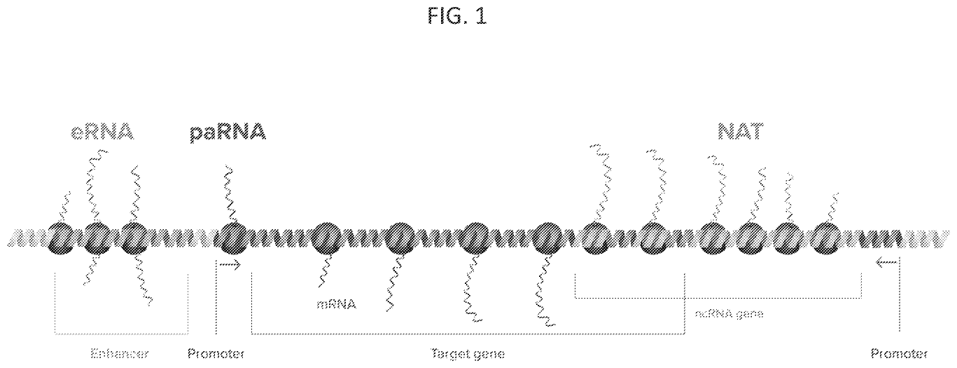

The antisense oligonucleotide (ASO) disclosed herein hybridizes with a regRNA transcribed from a regulatory element of a target gene. It is understood that both eRNAs and paRNAs are regRNAs facilitating or upregulating gene expression (FIG. 1). In certain embodiments, the target regRNA is an eRNA. In certain embodiments, the target regRNA is a paRNA. In certain embodiments, the target regRNA is not a polyadenylated RNA. eRNAs can be identified using methods known in the art, such as Assay for Transposase-Accessible Chromatin using sequencing (ATAC-seq), global run-on sequencing, precision run-on sequencing, cap analysis gene expression, and histone modification analysis (see, e.g., Sartorelli & Lauberth, Nat. Struct. Mol. Biol. (2020) 27:521-28; PCT Application Publication No. WO2013/177248). paRNAs are RNAs transcribed from promoters of target genes in the antisense direction (transcripts in the sense direction are mRNAs of the target genes). They can be identified by similar methods, taking into account of their specific location and orientation. In human OTC, eRNAs have been identified to transcribe from the same enhancer region. In mouse SERPING1, a paRNA has been identified to transcribe from the SERPING1 promoter but in an opposite direction as the SERPING1 mRNA. The nucleotide sequences of exemplary regRNAs are provided in Table 1 below. Any of these regRNAs are contemplated as a target regRNA of an ASO disclosed herein.

TABLE 1
Exemplary regRNAs

| regRNA | Nucleotide Sequence |
|---|---|
| Human OTC | CACAGAGCAAACAGGGAAATCACAGAGGTTCAAAGTTCACCAGTGTCTCAACAAT |
| eRNA-1A | CAGCCCTAATGCTCCCTGTGACTACCAGACACTCCCAGGACCTGAGTGATGGGGAT |
| (minus strand) | CCCATGAGATCATTTTCATTGCTTTCTACTGACCAGGGTTTGGTCTAGGAGCACTGT |
| SEQ ID NO: 1 | CCCAGTAATAATTTTCATGGCAATATTCTCCCCTTGAGCCCAGGAAACATGTCTTGG |
| | ATGGCTTCAAAAGTCACTGCTTGGTGAATGCCTTCTCTGCCCATTTCTACTTTTTGG |
| | TGAAACTTGAAACCATCTTTGTAGTTGGTGCCTCTCTTCAGACCCTACTTGGGAGGT |
| | GCTCTTGACCTGCTATTGATTGCTTTATTGGGCTATATCTACTAAGCAGGGGCTCTG |
| | CCCTCACCTTAAGCTAATGATTAAACACAGCCTTCTTCTCTCAAGGCTGCTCCACTG |
| | GTAACAACTCTGTGGCCTGTAAAGATGGGACCTATTTAGGGTCTGGAAGATAGACC |
| | ATGGGAATCCTGTCTTCAAGATTCAAGAGAAACAAGCCCTTTTCATGGGGCTTTGT |
| | TGAGTGTTTGGAGCCTAGGTCATAGGTGCTACATATTCACCATTATTGATTTATTCC |
| | TCCAGAATTTTTCAACTGGAGTTCACCATTTCTTCCAGGGAACCAAGGAGTTCATG |
| | GATTTCCAGGTCACCTTCATTGTTATGCAATGTATTACCATTAAGAAGTAAGCGAA |
| | TCATCTGGCAAGCAGTACTAGCAGCTCCTACTCATAGCTTTGTTGTGAGTATGAAA |
| | TGTAATAATGAATAGAGAGTACTGTAGCACAGTACCTAGCTCAGTGTTCAATAAAT |
| | GTTAGCTTTTGTTAACTACTACCATTGGCACATGTGGTGAGAGGCCCCATCCCTGGC |
| | TCAGTTCTTGGCTTATTCTAATCACTTTCCTACAAATAAAAGTGTTGAGGTGTCCGT |
| | CTTTCTTTCATACCCCCACCCCACTCCAGAGCTGTATTAAA |
| Human OTC | CACAGAGCAAACAGGGAAATCACAGAGGTTCAAAGTTCACCAGTGTCTCAACAAT |
| eRNA-1B | CAGCCCTAATGCTCCCTGTGACTACCAGACACTCCCAGGACCTGAGTGATGGGGAT |
| (minus strand, | CCCATGAGATCATTTTCATTGCTTTCTACTGACCAGGGTTTGGTCTAGGAGCACTGT |
| extended) | CCCAGTAATAATTTTCATGGCAATATTCTCCCCTTGAGCCCAGGAAACATGTCTTGG |
| SEQ ID NO: 2 | ATGGCTTCAAAAGTCACTGCTTGGTGAATGCCTTCTCTGCCCATTTCTACTTTTTGG |
| | TGAAACTTGAAACCATCTTTGTAGTTGGTGCCTCTCTTCAGACCCTACTTGGGAGGT |
| | GCTCTTGACCTGCTATTGATTGCTTTATTGGGCTATATCTACTAAGCAGGGGCTCTG |
| | CCCTCACCTTAAGCTAATGATTAAACACAGCCTTCTTCTCTCAAGGCTGCTCCACTG |
| | GTAACAACTCTGTGGCCTGTAAAGATGGGACCTATTTAGGGTCTGGAAGATAGACC |
| | ATGGGAATCCTGTCTTCAAGATTCAAGAGAAACAAGCCCTTTTCATGGGGCTTTGT |
| | TGAGTGTTTGGAGCCTAGGTCATAGGTGCTACATATTCACCATTATTGATTTATTCC |
| | TCCAGAATTTTTCAACTGGAGTTCACCATTTCTTCCAGGGAACCAAGGAGTTCATG |
| | GATTTCCAGGTCACCTTCATTGTTATGCAATGTATTACCATTAAGAAGTAAGCGAA |
| | TCATCTGGCAAGCAGTACTAGCAGCTCCTACTCATAGCTTTGTTGTGAGTATGAAA |
| | TGTAATAATGAATAGAGAGTACTGTAGCACAGTACCTAGCTCAGTGTTCAATAAAT |
| | GTTAGCTTTTGTTAACTACTACCATTGGCACATGTGGTGAGAGGCCCCATCCCTGGC |
| | TCAGTTCTTGGCTTATTCTAATCACTTTCCTACAAATAAAAGTGTTGAGGTGTCCGT |
| | CTTTCTTTCATACCCCCACCCCACTCCAGAGCTGTATTAAAAGTGAAATTCAGGCTG |
| | GGCATGGTGGCTCACGCCTGTAATCCCAGCACTTTGAGGCGGGCGGATCACGAGGT |
| | CAGGAGTTCGAGACTAGCCTGACCAACGTGGTGAAACCCCGGCTCTACTAAAAAT |
| | ACAAAAATTAGCCAGGCATGGTGGCGGACACCTGTAATCCCAGCTATGCATCGAG |
| | AGGCTGAGGCAGGAGAATTGCTTGAACCCGGGAGGCGGAGGTTGCAGTGAGCCGA |
| | GATAGTGCCACTGCTCTCTAGCCTGGGCGACAGAGCGAGACACCATCTCCATTAAA |
| | AAAAAAAAAAAAAAAAAAAGTGAAATTTAAGAAAATAATAAATTTAAATAAAAT |
| | AAAATAAAGAATAAAAAAAGACAAAAAACAACAAACAAAGAAATCACACTCTTG |
| | TCGTCTAAACTAGTGGAAAATAAAAAGGAAACAGTGCTTCTTACTGAAGACCTATG |
| | ATGTGTTTTCTTCTTTCTTCTTTTCTCTTTTTGCCTAAATAATGTGAAAGCCAGGAAC |
| | TTTGTTCTCCCTCAGTACAACTGTCTAACGTTACAAGTTTCTAATCTTTTATTGTCCT |
| | TCTGTGACCCTGTTATGCTAACCAACCTATCCCATTTAAGATGAAAATAAGATAAA |
| | ACAATAAATGACTGTTAAGTACTTCAAAAATACAAAAGACTCTAAATGCCAAGAG |
| | TGATTATTGCCTTCCGGTGTAATGAAGATGCATAATAAAAATGGATCATAAAATCC |
| | CTTCGCTACCATGGCCACTACTCTTTTTGCCAGTTAAAATTTACAATTACAGTCTGT |
| | TTATTCAAAGCAACAGGGGACATGGAAACAACTCAGTCATTTTCTAAAGTAGGTTC |
| | CCCCAATCACTTGAACACAGAATGACTCTAGAGACATCAGTGATACATTTTTCAAA |
| | ATCCATATGAAAATAACACATCATCTTCAGTGTCTAATAATGTCACTGAAACCTCC |
| | CCACCCCCATATTCTTTTTATCCAATGTATATGTT |
| Human OTC | CGTGCTTTGCATCTCAACTGCCTCTGACACAAGGTTGGGTCATTAACCATATTTATT |
| eRNA-2A | TACCCCTTTCTTACTTCATAAATTGTGTTGTGCATTTCCTAATCTTGTCTTCTGTGCA |
| (plus strand) | GAAATTTACCAGATCACAAAATAAAAAGAACCAGGTATGGAAACTTGAGACTTTT |
| SEQ ID NO: 3 | GTCCATATCTAAGCTCAAAAAATTCTGGATTCTGCTTAAAATCAATAACTTCCAGG |
| | GCAAATGATTAACAGAATAATGATTTCAATGGAAATGTGGTCTATATGACAAGAG |
| | AGAAGCGAGATTATACTTGAATTTGATGATTGGCATTTCACACTCTGTTCAGAATTT |
| | TAGCCTTTATGAAGGACCCTGTCTACCTTCTTCAAATGCCTCAATTGAAAGGGTATG |
| | GCAGGAAAAGGGATGGAGAGGGGGTCAGAGATAATCCTCACTGATTTCACAATTT |
| | CCCCTAGGGCCCACCTGTTTGAATAAATACCTAGTGGTGATGTAGCAAGAAAACAC |
| | CCCTGAATCAATATTTTTAGACAAACTACCATTACCATTACCATCTGTTAATTAGAA |
| | AGTAACTCTCAGATCGCTTGAACCCAGGAGGCAGAGGTTGCCATGAGCCGAGGTT |
| | GTGCCACTGCATTCCAGCCTGGGTGACAGAGCGAGACTCTGTCTCAAACAAACAAA |
| | CAAACAAAAAACATAAAAAATAAAAAATAAAGTAATTCTCAGATTCTCTGTAAAT |
| | CAGGAGCTCTTTTGGTGATATATATATATACACACACACACACACACACACACACA |
| | CACACACACACGTGGAGAGAAAAAAAGGAGCCAGCTAATGGGTGATTTCCTTTAC |
| | TATAATAATTCTAATTTATTTCTTATTGCCAGTTTCCACACAGGTCCAAGCCGCAAA |
| | ACAATACCGTCTCACAACAGAGCAGAGCTTTACAGCTTATAAAACATTCTGGGAAT |
| | ATTAGAATATTAACACATGAAGATTTTTGGGTCCTTCAGCACGTTAGATATGACCT |
| | GGGACATAGAGGACTCTTAAGAAAAGAGAAATTGGGCATCCACATGTGGAAAAAA |
| | AAACTCAATCTCAACCTCAAGCCTTAGACAAAACTCACAGTGGATCACAGATCTGA |
| | ATGGAAAATGTAAGGCTACAACACTTTCAGAAGACATAGGGAGAGAATCTTTGTT |
| | ACCCTGGGTTATGCAAAGAGTTCAACAGCATGATCCAGAGAAAGGAAACAAATGG |
| | TAAATTGAACTTGATCAAAATTTAAAAATTTTGCTTTGTGAAAAACATAGTCAGGA |
| | GACTGAAAAGACAAGCAACAGACTGGGAAAACACATTTGCAAGTTACATATCCAA |
| | TAAAAGTGTTGTATCCAGAATATATATGTAGAACATTCAAAACTAAACATTAAGAA |
| | AATAAACAATTCAAGTAAAAAAAAAAAAAGAAGAAGAATAGGGGTCAGGCAAGC |
| | TCAGTGGCTCACGCTTGTAATCCCAGTGCTTTGGGAGTCTAAGGCTGGAGGACTGT |
| | TCAAGACCAGGAATGTGAGACAAGCCTGGGCAACATAGTGAGACTCTATACCTAC |
| | CAAAAAATTTAGTTTGGTGTGGTGGCACACACCTATACTCCTAGCTATTTGGGAGG |
| | CTGAGGTTGGAGGATCGCTTGAGCCCAGGAGGTCAAGGTTACAGTGATCTATGATC |
| | ACACTATTGTGAAACCGCCTTTGCAAAATTATGACAGACAGTGAAAGAAATCTAAC |
| | TTAACCGAATCCATCTTGCTTCTAACCTTCAAGCTGTCCTTGTTCATTGCTGGGCAT |
| | AGGCTGAACTAATTTTGGGAGAAACTTAGTTTATAGTTTATGGTTTAAACAAAGAC |
| | GGTAACACCCCTTTCCCAAAGTGACCTCCTTCCTGCCTGGGGACTAGACTTCCTTTG |
| | TAGGACTAACATTAGCCACAAGATCAGATGAGCCAGTTTATTGATCTGGTGGTGCC |
| | AGCTGATCCATCAAGTGCAGTACTGATACCCCAAGCACTGATTTT |
| Human OTC | AATCAATAGCAGGTCAAGAGCACCTCCCAAGTAGGGTCTGAAGAGAGGCACCAAC |
| eRNA-2B | TACAAAGATGGTTTCAAGTTTCACCAAAAAGTAGAAATGGGCAGAGAAGGCATTC |
| (plus strand, | ACCAAGCAGTGACTTTTGAAGCCATCCAAGACATGTTTCCTGGGCTCAAGGGGAGA |
| extended) | ATATTGCCATGAAAATTATTACTGGGACAGTGCTCCTAGACCAAACCCTGGTCAGT |
| SEQ ID NO: 4 | AGAAAGCAATGAAAATGATCTCATGGGATCCCCATCACTCAGGTCCTGGGAGTGTC |
| | TGGTAGTCACAGGGAGCATTAGGGCTGATTGTTGAGACACTGGTGAACTTTGAACC |
| | TCTGTGATTTCCCTGTTTGCTCTGTGCCTGATAGCTTTCAGTCTGCTAACAAATCTCC |
| | TTTATGCAGTTTAACCTCTGTACTTCCAATGGGGAGGAATTGGAATCAGCCTATGG |
| | GAGAAGAGATAGCTCTAGGATTCTGTGTGGGAACTTGAAGAGATAAAAGGGCATC |
| | CTTGAAAGAGAACCTCGTGCTTTGCATCTCAACTGCCTCTGACACAAGGTTGGGTC |
| | ATTAACCATATTTATTTACCCCTTTCTTACTTCATAAATTGTGTTGTGCATTTCCTAA |
| | TCTTGTCTTCTGTGCAGAAATTTACCAGATCACAAAATAAAAAGAACCAGGTATGG |
| | AAACTTGAGACTTTTGTCCATATCTAAGCTCAAAAAATTCTGGATTCTGCTTAAAAT |
| | CAATAACTTCCAGGGCAAATGATTAACAGAATAATGATTTCAATGGAAATGTGGTC |
| | TATATGACAAGAGAGAAGCGAGATTATACTTGAATTTGATGATTGGCATTTCACAC |
| | TCTGTTCAGAATTTTAGCCTTTATGAAGGACCCTGTCTACCTTCTTCAAATGCCTCA |
| | ATTGAAAGGGTATGGCAGGAAAAGGGATGGAGAGGGGGTCAGAGATAATCCTCAC |
| | TGATTTCACAATTTCCCCTAGGGCCCACCTGTTTGAATAAATACCTAGTGGTGATGT |
| | AGCAAGAAAACACCCCTGAATCAATATTTTTAGACAAACTACCATTACCATTACCA |
| | TCTGTTAATTAGAAAGTAACTCTCAGATCGCTTGAACCCAGGAGGCAGAGGTTGCC |
| | ATGAGCCGAGGTTGTGCCACTGCATTCCAGCCTGGGTGACAGAGCGAGACTCTGTC |
| | TCAAACAAACAAACAAACAAAAAACATAAAAAAAAAAAATAAAGTAATTCTCAG |
| | ATTCTCTGTAAATCAGGAGCTCTTTTGGTGATATATATATATACACACACACACAC |
| | ACACACACACACACACACACACACGTGGAGAGAAAAAAAGGAGCCAGCTAATGG |
| | GTGATTTCCTTTACTATAATAATTCTAATTTATTTCTTATTGCCAGTTTCCACACAGG |
| | TCCAAGCCGCAAAACAATACCGTCTCACAACAGAGCAGAGCTTTACAGCTTATAAA |
| | ACATTCTGGGAATATTAGAATATTAACACATGAAGATTTTTGGGTCCTTCAGCACG |
| | TTAGATATGACCTGGGACATAGAGGACTCTTAAGAAAAGAGAAATTGGGCATCCA |
| | CATGTGGAAAAAAAAACTCAATCTCAACCTCAAGCCTTAGACAAAACTCACAGTG |
| | GATCACAGATCTGAATGGAAAATGTAAGGCTACAACACTTTCAGAAGACATAGGG |
| | AGAGAATCTTTGTTACCCTGGGTTATGCAAAGAGTTCAACAGCATGATCCAGAGAA |
| | AGGAAACAAATGGTAAATTGAACTTGATCAAAATTTAAAAATTTTGCTTTGTGAAA |
| | AACATAGTCAGGAGACTGAAAAGACAAGCAACAGACTGGGAAAACACATTTGCAA |
| | GTTACATATCCAATAAAAGTGTTGTATCCAGAATATATATGTAGAACATTCAAAAC |
| | TAAACATTAAGAAAATAAACAATTCAAGTAAAAAAAAAAAAAGAAGAAGAATAG |
| | GGGTCAGGCAAGCTCAGTGGCTCACGCTTGTAATCCCAGTGCTTTGGGAGTCTAAG |
| | GCTGGAGGACTGTTCAAGACCAGGAATGTGAGACAAGCCTGGGCAACATAGTGAG |
| | ACTCTATACCTACCAAAAAATTTAGTTTGGTGTGGTGGCACACACCTATACTCCTA |
| | GCTATTTGGGAGGCTGAGGTTGGAGGATCGCTTGAGCCCAGGAGGTCAAGGTTAC |
| | AGTGATCTATGATCACACTATTGTGAAACCGCCTTTGCAAAATTATGACAGACAGT |
| | GAAAGAAATCTAACTTAACCGAATCCATCTTGCTTCTAACCTTCAAGCTGTCCTTGT |
| | TCATTGCTGGGCATAGGCTGAACTAATTTTGGGAGAAACTTAGTTTATAGTTTATG |
| | GTTTAAACAAAGACGGTAACACCCCTTTCCCAAAGTGACCTCCTTCCTGCCTGGGG |
| | ACTAGACTTCCTTTGTAGGACTAACATTAGCCACAAGATCAGATGAGCCAGTTTAT |
| | TGATCTGGTGGTGCCAGCTGATCCATCAAGTGCAGTACTGATACCCCAAGCACTGA |
| | TTTTAGGAGCAGTTTAGGGAGGGTCAGAATTTTGTAGCCTCCAGCTGCATAACTCC |
| | TAAACCATATTTTTTTTTTTTTTGAG |
| Mouse | CATTCCAGGCCTCGACCCTTCCAGGGTGTGTTCTCTCCTCCTCCCTCTGGGGCAACC |
| SERPING1 | TTCTCTCTGTCTCCGCCCTCTGTTTTGTTTTCCCCAAACCGCTTCGCTTTCCCGGCAC |
| paRNA | CGGCTGGCCCTTCTCTCCCTTCACGCCCAGGTCTCGGCCCATCTGTTCAATCTCCCT |
| SEQ ID NO: 5 | GCTCCGCTTAACAGAACAATAAGCCAGAGGCTGCAGGAGGAGGCCAGGGCGAGCT |
| | GGGCAGCACAGCTCACAGCTTTGAGGGAGGAGGAGGGGGAGGGGGGAGGAGGAG |
| | GAGGAGGAGGAGGAGGAGGAGGAGGAGGAGGAGGAGGAGGAGGAGGAGGAGGA |
| | GGAGGAGGAAAGCTCAAGTAGAATCAAGTGTATGGAAACTACAGAAATAACAAAC |
| | GGGATAAGGAGGCTGGAGATCTCCCTGCCAAGCCCTCTTTAACTTTGTGTTCCTCCC |
| | AAAGCCCCCTATTACCAAACCTGGTCCAAGTTCTTGGTCACTAGGCAGCTTCAGGA |
| | CAGACTGAGCAGACAGTCGCAGAGAATCAGTGTGTGGGGGGGGGGCACTCTCAGC |
| | TATTGGCTGGTTTAGGACACAGCCCAGTTAAAAATTAAGGATGATTGATTGCTAGT |
| | GTGAGTTCCTGTAAATCCGGATGGGACGGATGGGACGAGTCTCGAACTGTGGGCC |
| | AGTTAGCCCGGAGTTCCTCTTGGAATGGGGTAGGATGTGACCGGTTGGGATAAGGA |
| | AGCTTGAGTGACGCTCTTAGTGATCTTAGTGTTTTGTGGATCAAGATAAAGGCAGA |
| | AGAAGCACACAACGTGCCTAAGGTTTGACAATGGATGAGCTTTTTGAGATTAATGG |
| | GTAAAGCAAGCCTTTAGAAATAGGCCAAGCCTTAGAAGTAGTTAAATGGCCTTCCA |
| | TTGCTCTCATTTGGAGATATGCCAGCCTTGGGCCCGAATCTCCTAAAAACCTAATG |
| | GCTGCTGGATCTTCCCCATTCCTACACGTAATCACCGGCTCCAGTTTCCAGCCGAGC |
| | CCTGGCTTCAACCCC |
| Mouse | GCCATGTGATTTCTCAACAGCATGTTCCAAAATGTGCACAAGAGTTTATGTAAGCC |
| SERPING1 | AGCAAGGTCAAATCTCTGAACATAGTCCACACTGGAACAGAAGGAAAGGCATGTA |
| eRNA | GACAGCTCTTGGACAGTGAAACATGGTGGGAAGCAGGGATCTTTCTACAGTCTTAG |
| SEQ ID NO: | GACAAGTTGGGGTGACTTGTATGGCTCTTGGTCTTCACTGGAAGACAAGCTACAAC |
| 1078 | AAAGAATTGGCCCAACATTCTGAGGCTTTGCTTGGTTTTGCCTAGCCTGCATGAGG |
| | AGTCAGAAACTGAAGGGAAGCCCGAGGACCAGAACAGAGCCATAGCAGGTGAAT |
| | ATCAAGGAGAAGGAGAAGCAATGACTGTTGCTCCATCTCGTTCCCTCTCCCACTAA |
| | GGCTGCTTTCACTTCAAGCAGAACAGACAAACCAGAAAATTGCAGGAATCAAAGT |
| | TCCTTTCAGGAATAAATCTCTTGGCCACTGTGATGTTTTGAATGAGAATGGCCTCCA |
| | GATGCCCTTTTGTTTGAATAATTAGTCCCTGGTTAGTAGAGCTGTTTGGGGAGGATT |
| | AGGATGTGTGGCCTTGCTGGAAAAGGTGTGTCACCGGGGTTGGCTTTGAGGTTTCA |
| | AAAGCCTGTACTATTGCCAGCCGGTTCTCTCTCTCTCTCTCTCTCTCTCTCTCTCTCT |
| | CTCTCTCTCTCTCTCTCTCTCTTCCCTC |
| Mouse OTC | AGAGATAGAGTAGGGCAGGGTGCAGGGGCACTTGAACTTTAAGCCTAACTCCAGG |
| paRNA-1 | GTTGTTTTTGAAAGAAAGAATCCTAACTCTTTCAGAGCCCAGCCTCTTCGTTTTTCA |
| (minus strand) | TAATGTATTTATGCTGTCACTTGCTTGGATAAAAATCAGTTGACAAGTACCCATTGA |
| SEQ ID NO: | GCATGTAATATATGCAAGGCACTGCCATGCTGGGGCTGCTGTGAAGGTACACTGAA |
| 1073 | ATAACTCATTCAGAAAATGGAAATTGACAGTACAAAACCCCAGATGGAGCTATGA |
| | CAGCCCTGTGTATTCCTGTTGATTTCACTTTCCTACTTAGCACTCCCATCCATTCCAC |
| | ACCAAGAAAAAAAATAATAATAAATAAATAAATAATGGGATGGAT |
| Mouse OTC | TCCCACCCCTTAGAAGTTCAGAGGTCAACCTGTGCATAAAGGAGATTTATTAGCAC |
| eRNA-1 | ACCAAAGGCTATCAGGCATTAAGCAAACAAGGAAATCACAAAGGTTTAAAGTTCA |
| (minus strand) | TCTAACAGCTAAGCCACTAACAGACACTTCCATGGCCAGGAGATTGGAGGCCCTGT |
| SEQ ID NO: | GAGAACTTTGGCGTTGTCTCCTGTTGACAAGTGCTTAGTATAAAAGCACGGTCTGT |
| 1074 | ATAAGAATTTCACTCTTGGAGGGCAGGCAGGTCACATGTCCTTGAACACCCTCAAA |
| | GGCCATTGCTTGGTGAATGACCCCTTTCCCAATTCTAGTTCTTGACAATCCTGTAGT |
| | CATCTTTCTGGTTGACACTAACACCTACTTGTGAACTCTTTTTCCTCTGCTCTGGGTT |
| | GTCTCGGATCAATATCTGCCACACAACAAAGGTGTTTCCTTGCTTTAAGCTATTGGT |
| | TTGGTGCATCTTTTCTTAAGGTTGTTCCACTGCTAACTACTGTGCTGTGTGTAAAAT |
| | GGGACCTAGACACAGCTTGAAGATAGATTTTATGAAAACTTTTTGTATCCAATATG |
| | AAAGTTTGCATAGAATTTGGTCAAGGCTTGCTTATTGGATTTAGATATTCACCATCA |
| | CTGATTTTAATCCTTCACAGTTCTTCAGTGGAGCTCAGCTGGAAAGTTTCTTCGAGG |
| | GAATCAAGTAGTTCATGGATATTTAGTCACCCTGGATTTTTACACAGCGTACTGCC |
| | ATAAAGGAACATTCAGGTCAGGTCACATGGGAATCACTATAAAGAGAACTGACTC |
| | AAAGGTGTTGGAGCCAGTT |
| Mouse OTC | CTGGCCATGGAAGTGTCTGTTAGTGGCTTAGCTGTTAGATGAACTTTAAACCTTTGT |
| eRNA-2 (plus | GATTTCCTTGTTTGCTTAATGCCTGATAGCCTTTGGTGTGCTAATAAATCTCCTTTAT |
| strand) | GCACAGGTTGACCTCTGAACTTCTAAGGGGTGGGATAGGAACCAGCCTATGGGAA |
| SEQ ID | AGGAGATGGCTCTAGAATTCTCAGTGGGATCATGAACAAACAAAAGGTATCCTGG |
| NO: 1075 | AAGAGAATTGGCCCTTTTCATCTCGCCTGCCTCAGATACAAACTTGGATCACTGAC |
| | CGTACTTATTTGCCTTTTTCCAATCTCATTAATTGTGCCGTGCATTTCATAATCTTAT |
| | CTGCTTTACCAGATCACAAAATAAAAAGCATCTGGAAACAGTTGAGATTTTTCTAT |
| | CACTTTCTAAACACAGAAAAATTCATAGCTATGCTCAAAATCAAGAACTGGCAGGG |
| | TAAGTGATAAAGGGAGATTGACTTCAGTGGTAATGTGCTCTACTAGAAATATTAGA |
| | ATTAAATCCCTTGATCAGCATTTCACATTGTTCAGAATTTTGGTCTTCCAATGTTTC |
| | AGTTGAAAGCGTATGGTGAGAAAGGGGGTGGAAAAAAATCTTTACTGATTTCACA |
| | GTTTTCCCTAAGGCTTGCCTATTTAAATAAATATTTTGCAATGACGTAGAAAGAAA |
| | ACATCCCTGAGTCAATATTTTTAGTTAAAGTCCCATTACCATCTGTTAATTGAACAG |
| | TAATTTTCAGATTTTCTATAAATCATGAGCTACTTGGATAACATATTTATACATGAA |
| | AAGAAAAATATAGCCAGATGATAGGTAATTTGGTTTTTCACTATCTTGATTCAATTT |
| | CATTTCCTATTGCCAGTTCTCATGAACATTCAAGCTCCATTCCAGCTGCTTACGAAG |
| | CACTGCCAGATACTAAGAAGTACTAAATTCTCCAGCATGTGGTTTTGACCTAGGAC |
| | ACAGAATTTGAACAAATTACATTTATTTAT |
| Mouse OTC | CTAAAAATATTGACTCAGGGATGTTTTCTTTCTACGTCATTGCAAAATATTTATTTA |
| eRNA-3 | AATAGGCAAGCCTTAGGGAAAACTGTGAAATCAGTAAAGATTTTTTTCCACCCCCT |
| (minus strand) | TTCTCACCATACGCTTTCAACTGAAACATTGGAAGACCAAAATTCTGAACAATGTG |
| SEQ ID | AAATGCTGATCAAGGGATTTAATTCTAATATTTCTAGTAGAGCACATTACCACTGA |
| NO: 1076 | AGTCAATCTCCCTTTATCACTTACCCTGCCAGTTCTTGATTTTGAGCATAGCTATGA |
| | ATTTTTCTGTGTTTAGAAAGTGATAGAAAAATCTCAACTGTTTCCAGATGCTTTTTA |
| | TTTTGTGATCTGGTAAAGCAGATAAGATTATGAAATGCACGGCACAATTAATGAGA |
| | TTGGAAAAAGGCAAATAAGTACGGTCAGTGATCCAAGTTTGTATCTGAGGCAGGC |
| | GAGATGAAAAGGGCCAATTCTCTTCCAGGATACCTTTTGTTTGTTCATGATCCCACT |
| | GAGAATTCTAGAGCCATCTCCTTTCCCATAGGCTGGTTCCTATCCCACCCCTTAGAA |
| | GTTCAGAGGTCAACCTGTGCATAAAGGAGA |
| Human OTC | CACGGTGAAATCTGATTACAGAAGGACTGAGAAATGAAAGCTTTGCTGGAATTAG |
| paRNA-1 | AGGCAGCTGTCTCAAATAAGAGTAAAGCAGAGCGTGGCACTACATTCTGCAGAAA |
| plus strand | GAGGCCTTACTGCAGAATATAGTTTTATGCATCACCATGATTCCTAAATCAAACCC |
| SEO ID | AAGTCTCTGACCATCACTTACCGAAAATTTCGAACCATGAAGTTGTGACCATTTCT |
| NO: [WM1]1077 | AAAAGCTGCATTGTTTAACAGGATCCTCAGATTAAACAGCATCTTCTTTTAATTGTG |
| | TAAAGGACGATTCTATGCCCTTGAAAACTCCACAGCAAGTTAGCCAGCGGGGGCC |
| | ACCTAAAAACTAAGAAATGTGTTCAGTTGCAGTGAGGGAGCAAAGGTAATATCAC |
| | CCTTCAGCTTCCTTATCAAAGTCCCCTGGTTAGAGATACTGCAGGGCAGGGTGTAG |
| | GAGCATTTGAACTTTATGCCAAGCTCCACCGCTGTGTATTTTTTTTGGAAGAAATCC |
| | TGACTCTTTTATTGCCTGGCCTCCTCATTTTTCATAATATATTTATACTGTCACACGC |
| | ATGTACAAAATCAATTGACAAGTACTCATTGAGGATATAATATATGCAAGGCACTG |
| | CGGTACCGGCTGCTGTGAAGGTGAGTAGAAGTAACTCACTCAGAAAATGCAAATT |
| | GGCACTGCAAAAACACAGATGTAGATGTGACAGTCCTGAGTAGTACTATTTCCATT |
| | TATTTCACTTTTCTACCTCTAATTGCCAGTCTTTCCACAAAAACAAATAGAAAGAAG |
| | TGCCCTGGAAGCTATTTTGGGGGTGCAAGATGATAGAGAAGGTATAGTAAAAATA |
| | TCTCAGTCCTGGAGCTCCCTTTGTGCCTTGCACATAAGTCTTCAATAAAGAAATAAT |
| | TGCTGGCCGGGCACGGTGGCTCACGCCTGTAATCCCAGCACTTTGGGAGGCTGAGG |
| | CGGGCGGATCACTTGAGCCCAGGAGTTCGAGACCAGCCTGGCCAACATGGTGAAT |
| | CCCCGTCTCCACTAAAAATACAAAAATTAGCTAGACGTGGTGGTGCACACCTGTAA |
| | TCCCAGCTATTTGGGAGGCTGAGGCATGAGAGTCGCTTGAACCCGGGAGACAGAG |
| | GTTGCAGTGAGCCCAGATTGCACCACTGTACTCCAGCCTGGGTGACAGAGAGAGA |
| | CACTGTCTCAAAAAAAAGAAAGAAAGAAAGAAAAAGAAAAGAAATAGTTGCCTA |
| | TTGATTCTAACAGCACCTGACAGTCAGTACAAGTGGGAAGTGGGGATACCATATGC |
| | TCTTCCCCTAGCAGCCTATTCAGATCACTAAGGGGCCATCTGCAACTTCACATTTAT |
| | TCATTTATCCTCAGTTAGAAATCAGGTGATTCAAAGTCAATTCTGTATCAGATACCG |
| | AGCTGCACTGAAGTTTCAGAGAGGGAAGATGACATCATCTTTGCAGAGTTCACAGT |
| | TCAGTAAAGGTGGTATGATTTATAAACAAATATCCATAATACAAGCCCATAGGAGA |
| | GGTCCAGACAAAGTTGTCTGGCTTCGAAGAAGCGGGAGCCCTCTAAGGTAGGAAG |
| | GGTAGGAGGAAGGAGAGGAGGGGCAGGGGTAGGGCTGCCTCTTCTGGTCAATTTA |
| | T |

The present invention describes ASOs that increase the amount or stability of the target regRNA, thereby to increase expression of the target gene. This is different from the ASOs previously described that were designed to inhibit eRNAs (see, e.g., PCT Application Publication No. WO2013/177248 and PCT Application Publication No. WO2017/075406). Without wishing to be bound by theory, it is hypothesized that the ASOs' ability to upregulate regRNAs is attributable to the selection of a target sequence in the regRNA and/or the chemical modifications of the ASOs.

In some embodiments, the regulatory RNA has a nucleotide sequence of SEQ ID NO: 1. In some embodiments, the regulatory RNA has a nucleotide sequence of SEQ ID NO: 2. In some embodiments, the regulatory RNA has a nucleotide sequence of SEQ ID NO: 3. In some embodiments, the regulatory RNA has a nucleotide sequence of SEQ ID NO: 4. In some embodiments, the regulatory RNA has a nucleotide sequence of SEQ ID NO: 5. In some embodiments, the regulatory RNA has a nucleotide sequence of SEQ ID NO: 1073. In some embodiments, the regulatory RNA has a nucleotide sequence of SEQ ID NO: 1074. In some embodiments, the regulatory RNA has a nucleotide sequence of SEQ ID NO: 1075. In some embodiments, the regulatory RNA has a nucleotide sequence of SEQ ID NO: 1076. In some embodiments, the regulatory RNA has a nucleotide sequence of SEQ ID NO: 1077. In some embodiments, the regulatory RNA has a nucleotide sequence of SEQ ID NO: 1078.

### Sequences of ASOs

As disclosed herein, ASOs that bind a sequence closer to the 5′ or 3′ end of the OTC target regRNA are more likely to upregulate the regRNA. Without wishing to be bound by theory, it is hypothesized that such ASO hybridizes to a terminal portion of the OTC regRNA and prevents or slows 5′→3′ and/or 3′→5′ RNA degradation without blocking the functional region of the regRNA. In certain embodiments, the ASO disclosed herein is complementary to a sequence in the target regRNA that is no more than 300, 250, 200, 150, 100, 50, 40, 30, 20, or 10 nucleotides from the 5′ or 3′ end of the target regRNA. In certain embodiments, the ASO disclosed herein is complementary to a sequence in the target regRNA that is no more than 300, 250, 200, 150, 100, 50, 40, 30, 20, or 10 nucleotides from the 5′ end of the target regRNA (i.e., the 5′ most nucleotide of the regRNA sequence forming a duplex with the ASO is no more than 300, 250, 200, 150, 100, 50, 40, 30, 20, or 10 nucleotides from the 5′ end of the target regRNA). In certain embodiments, the ASO disclosed herein is complementary to a sequence in the target regRNA that is no more than 300, 250, 200, 150, 100, 50, 40, 30, 20, or 10 nucleotides from the 3′ end of the target regRNA (i.e., the 3′ most nucleotide of the regRNA sequence forming a duplex with the ASO is no more than 300, 250, 200, 150, 100, 50, 40, 30, 20, or 10 nucleotides from the 3′ end of the target regRNA).

In certain embodiments, the ASO is no more than 8, 10, 15, 20, 25, 30, 40, 50, 60, 70, 80, 90, or 100 nucleotides in length. In certain embodiments, the ASO is at least 8, 10, 15, 20, 25, 30, 40, 50, 60, 70, 80, 90, or 100 nucleotides in length. In certain embodiments, the ASO is at least 8, 9, 10, 11, 12, 13, 14, 15, 16, 17, 18, 19, or 20 nucleotides in length.

In certain embodiments, the ASO is designed to lack a stable secondary structure formed within itself or between each other, thereby increasing the amount of the ASO in a single-stranded form ready to hybridize with the target regRNA. Methods to predict secondary structures are known in the art (see, e.g., Seetin and Mathews, Methods Mol. Biol. (2012) 905:99-122; Zhao et al., PLoS Comput. Biol. (2021) 17(8):e1009291) and web-based programs (e.g., RNAfold) are available to public users.

For example, ASOs have been designed to target a human OTC eRNA or a mouse SERPING1 paRNA. The nucleotide sequences of these ASOs are provided in Table 2 below.

TABLE 2
Exemplary ASO sequences targeting regRNAs

| | ASO | | SEQ ID |
|---|---|---|---|
| Target regRNA | Name | Sequence | NO |
| Human OTC eRNA-1 | hOTC-ASOe1-1 | TTAATACAGCTCTGGAGTGGGGT | 6 |
| | hOTC-ASOe1-2 | AATACAGCTCTGGAGTGGGGT | 7 |
| | hOTC-ASOe1-3 | TACAGCTCTGGAGTGGGGTG | 8 |
| | hOTC-ASOe1-4 | ATACAGCTCTGGAGTGGGGT | 9 |
| | hOTC-ASOe1-5 | AATACAGCTCTGGAGTGGG | 10 |
| | hOTC-ASOe1-6 | TACAGCTCTGGAGTGGGGT | 11 |
| | hOTC-ASOe1-7 | ACAGCTCTGGAGTGGG | 12 |
| | hOTC-ASOe1-8 | TTTTAATACAGCTCTGGAGTGGGGT | 13 |
| | hOTC-ASOe1-9 | TTTTAATACAGCTCTGGAGTGGGGTGG | 14 |
| | hOTC-ASOe1-10 | TTAATACAGCTCTGGAGTGG | 39 |
| | hOTC-ASOe1-11 | TTAATACAGCTCTGGAGTGGG | 75 |
| | hOTC-ASOe1-12 | TTAATACAGCTCTGGAGTGG | 77 |
| Human OTC eRNA-2 | hOTC-ASOe2-1 | GCTTGGGGTATCAGTACTGC | 15 |
| | hOTC-ASOe2-2 | TATGAAGTAAGAAAGGGGTA | 16 |
| | hOTC-ASOe2-3 | GGGTATCAGTACTGCACTTG | 17 |
| | hOTC-ASOe2-4 | TTATGAAGTAAGAAAGGGGTAA | 64 |
| | hOTC-ASOe2-5 | TTTATGAAGTAAGAAAGGGGTAAA | 65 |
| | hOTC-ASOe2-6 | TGAAGTAAGAAAGGGGTAAA | 66 |
| Mouse OTC paRNA | mOTC-ASOpa-1 | GGGTTTTGTACTGTCAATTT | 40 |
| | mOTC-ASOpa-2 | GGATGGGAGTGCTAAGTAGG | 41 |
| | mOTC-ASOpa-3 | TTCAAAAACAACCCTGGAGT | 42 |
| | mOTC-ASOpa-4 | GGATGGGAGTGCTAAGTAGG | 43 |
| Mouse OTC eRNA | mOTC-ASOe-1 | TGCTTAATGCCTGATAGCCT | 44 |
| | mOTC-ASOe-2 | GATGAACTTTAAACCTTTGT | 45 |
| | mOTC-ASOe-3 | TAACAGACACTTCCATGGCC | 79 |
| | mOTC-ASOe-4 | CAAGCCTTAGGGAAAACTGT | 80 |
| | mOTC-ASOe-5 | GCTTTACCAGATCACAAAAT | 81 |
| | mOTC-ASOe-6 | GGAGATGGCTCTAGAATTCT | 82 |
| Mouse SERPING1 | mSERPING1-ASOpa- | AACAGAGGGCGGAGACAGAG | 46 |
| paRNA | 1 | | |
| | mSERPING1-ASOpa- | CGGAGCAGGGAGATTGAACA | 47 |
| | 2 | | |
| | mSERPING1-ASOpa- | TTGGGAGGAACACAAAGTTA | 48 |
| | 3 | | |
| | mSERPING1-ASOpa- | AAAACAGAGGGCGGAGACAG | 49 |
| | 4 | | |
| | mSERPING1-ASOpa- | TTTGGGAGGAACACAAAGTTAA | 50 |
| | 5 | | |
| | mSERPING1-ASOpa- | AAAGTGGTTGATACCCTGGG | 51 |
| | 6 | | |

Tables 3 and 4 provide additional chemical modifications of hOTC-ASOe1-1 and hOTC-ASOe2-2

TABLE 3
Additional Chemical Modifications of hOTC-ASOe1-1
Sequence TTAATACAGCTCTGGAGTGGGGT

| SEQ | | | |
|---|---|---|---|
| ID | | | |
| NO | Name | Name | Chemical modification |
| 18 | hOTC- | CO-3172 | MT=MT=MA=MA=MT=dA=dC=dA=dG=dC=dT=dC=dT=dG=dG=dA= |
| | ASOe1-1a | | dG=dT=MG=MG=MG=MG=MT |
| 87 | hOTC- | CO-3721 | MT=MT=MA=MA=MT=dA=d5C=dA=dG=d5C=dT=d5C=dT=dG=dG=dA |
| | ASOe1-1i | | =dG=dT=MG=MG=MG=MG=MT |
| 88 | hOTC- | CO-3722 | MT=MT=MA=MA=MT=dA=d5C=dA=dG=d5C=dT=d5C=dT=dG=dG=dA |
| | ASOe1-1j | | =dG=dT=MG=MG=MG=MG=MT-tg |
| 89 | hOTC- | CO-3723 | MT=MT=MA=MA=MT=dA=d5C=dA=dG=d5C=dT=d5C=dT=dG=dG=dA |
| | ASOe1-1k | | =dG=dT=MG=MG=MG=MG=MT-ag |
| 90 | hOTC- | CO-3737 | MT=MT=MA=MA=MT=MA=dC=dA=dG=dC=dT=dC=dT=dG=dG=dA= |
| | ASOe1-1l | | dG=MT=MG=MG=MG=MG=MT |
| 91 | hOTC- | CO-3738 | MT=MT=MA=MA=MT=MA=M5C=dA=dG=dC=dT=dC=dT=dG=dG=dA |
| | ASOe1-1m | | =MG=MT=MG=MG=MG=MG=MT |
| 92 | hOTC- | CO-3739 | MT=MT=MA=MA=MT=MA=M5C=MA=dG=dC=dT=dC=dT=dG=dG= |
| | ASOe1-1n | | MA=MG=MT=MG=MG=MG=MG=MT |
| 24 | hOTC- | CO-3740 | MT=MT=MA=MA=MT=MA=M5C=MA=MG=M5C=MT=M5C=MT=MG |
| | ASOe1-1g | | =MG=MA=MG=MT=MG=MG=MG=MG=MT |
| 25 | hOTC- | CO-3741 | MT=MT=MA=MA=MT=MA=dC=dA=dG=M5C=dT=dC=dT=MG=dG= |
| | ASOe1-1h | | dA=dG=MT=MG=MG=MG=MG=MT |
| 19 | hOTC- | CO-3777 | MT=MT=MA=MA=MT=MA=dC=dA=dG=L5C=dT=dC=dT=LG=dG=dA |
| | ASOe1-1b | | =dG=MT=MG=MG=MG=MG=MT |
| 93 | hOTC- | CO-3778 | MT=MT=MA=MA=MT=LA=dC=dA=dG=L5C=dT=dC=dT=LG=dG=dA= |
| | ASOe1-1o | | dG=LT=MG=MG=MG=MG=MT |
| 94 | hOTC- | CO-3779 | MT=LT=MA=MA=MT=LA=dC=dA=dG=L5C=dT=dC=dT=LG=dG=dA= |
| | ASOe1-1p | | dG=LT=MG=MG=MG=LG=MT |
| 95 | hOTC- | CO-3780 | MT=MT=LA=MA=MT=LA=dC=dA=LG=dC=dT=L5C=dT=dG=LG=dA= |
| | ASOe1-1q | | dG=LT=MG=MG=LG=MG=MT |
| 20 | hOTC- | CO-3781 | MT=LT=MA=LA=MT=LA=dC=LA=dG=L5C=dT=L5C=dT=LG=dG=LA |
| | ASOe1-1c | | =dG=LT=MG=LG=MG=LG=MT |
| 96 | hOTC- | CO-3782 | MT=LT=MA=MA=MT=LA=M5C=MA=MG=L5C=MT=M5C=MT=LG= |
| | ASOe1-1r | | MG=MA=MG=LT=MG=MG=MG=LG=MT |
| 21 | hOTC- | CO-3783 | MT=MT=LA=MA=MT=LA=M5C=MA=LG=M5C=MT=L5C=MT=MG= |
| | ASOe1-1d | | LG=MA=MG=LT=MG=MG=LG=MG=MT |
| 22 | hOTC- | CO-3784 | MT=LT=MA=LA=MT=LA=M5C=LA=MG=L5C=MT=L5C=MT=LG= |
| | ASOe1-1e | | MG=LA=MG=LT=MG=LG=MG=LG=MT |
| 97 | hOTC- | CO-3796 | MT=MT=MA=MA-MT- |
| | ASOe1-1s | | MA=M5C=MA=MG=M5C=MT=M5C=MT=MG=MG=MA=MG=MT= |
| | | | MG-MG-MG=MG=MT |
| 98 | hOTC- | CO-3797 | MT=MT=MA=MA-MT=MA-M5C=MA-MG=M5C-MT=M5C=MT- |
| | ASOe1-1t | | MG=MG-MA=MG-MT=MG-MG=MG=MG=MT |
| 99 | hOTC- | CO-3798 | MT=MT=MA=MA=MT-MA-M5C-MA-MG-M5C-MT-M5C-MT-MG-MG- |
| | ASOe1-1u | | MA-MG-MT-MG=MG=MG=MG=MT |
| 100 | hOTC- | CO-3799 | MT-MT-MA-MA-MT-MA-M5C-MA-MG-M5C-MT-M5C-MT-MG-MG- |
| | ASOe1-1v | | MA-MG-MT-MG-MG-MG-MG-MT |
| 23 | hOTC- | CO-3800 | MT=MT=MA=MA-MT- |
| | ASOe1-1f | | MA=dC=dA=dG=M5C=dT=dC=dT=MG=dG=dA=dG=MT=MG-MG- |
| | | | MG=MG=MT |
| 101 | hOTC- | CO-4334 | MT=MT=LA=MA=MT=LA=M5C=dA=LG=dC=MT=L5C=MT=dG=LG= |
| | ASOe1-1x | | dA=MG=LT=MG=MG=LG=MG=MT |
| 102 | hOTC- | CO-4335 | MT=MT=LA=MA=MT=LA=M5C=dA=MG=dC=MT=M5C=MT=dG=MG |
| | ASOe1-1y | | =dA=MG=LT=MG=MG=LG=MG=MT |
| 103 | hOTC- | CO-4336 | MT=MT=LA=MA=MT=MA=M5C=MA=LG=M5C=MT=M5C=MT=MG= |
| | ASOe1-1z | | LG=MA=MG=MT=MG=MG=LG=MG=MT |
| 104 | hOTC- | CO-4337 | MT=MT=LA=MA=MT=dA=dC=dA=LG=dC=dT=dC=dT=dG=LG=dA= |
| | ASOe1-1aa | | dG=dT=MG=MG=LG=MG=MT |
| 105 | hOTC- | CO-4338 | MT=MT=MA=LA=MT=MA=M5C=LA=MG=M5C=MT=L5C=MT=MG= |
| | ASOe1-1ab | | MG=LA=MG=MT=MG=LG=MG=MG=MT |
| 106 | hOTC- | CO-4339 | MT=MT=MA=LA=MT=dA=dC=LA=dG=dC=dT=L5C=dT=dG=dG=LA= |
| | ASOe1-1ac | | dG=dT=MG=LG=MG=MG=MT |
| 107 | hOTC- | CO-4340 | MT=MT=LA=MA=MT=MA=L5C=MA=MG=M5C=LT=M5C=MT=MG= |
| | ASOe1-1ad | | LG=MA=MG=MT=LG=MG=MG=MG=MT |
| 108 | hOTC- | CO-4341 | MT=MT=MA=MA=LT=MA=M5C=MA=LG=M5C=MT=M5C=LT=MG= |
| | ASOe1-1ae | | MG=MA=LG=MT=MG=MG=LG=MG=MT |
| 109 | hOTC- | CO-4342 | MT=MT=LA=MA-MT- |
| | ASOe1-1af | | LA=M5C=MA=LG=M5C=MT=L5C=MT=MG=LG=MA=MG=LT-MG- |
| | | | MG=LG=MG=MT |
| 110 | hOTC- | CO-5049 | MT=MT=MA-MA- |
| | ASOe1-1ag | | MT=dA=d5C=dA=dG=d5C=dT=d5C=dT=dG=dG=dA=dG=dT=MG-MG- |
| | | | MG=MG=MT |
| 111 | hOTC- | CO-5050 | MT=MT=LA-MA- |
| | ASOe1-1ah | | MT=LA=M5C=MA=LG=M5C=MT=L5C=MT=MG=LG=MA=MG=LT= |
| | | | MG-MG-LG=MG=MT |
| 112 | hOTC- | CO-5055 | MT=MT=MA=MA- |
| | ASOe1-1ai | | MT=dA=d5C=dA=dG=d5C=dT=d5C=dT=dG=dG=dA=dG=dT=MG- |
| | | | MG=MG=MG=MT |
| 113 | hOTC- | CO-5056 | MT=MT-MA-MA- |
| | ASOe1-1aj | | MT=dA=d5C=dA=dG=d5C=dT=d5C=dT=dG=dG=dA=dG=dT=MG-MG- |
| | | | MG-MG=MT |
| 114 | hOTC- | CO-5057 | MT-MT-MA-MA- |
| | ASOe1-1ak | | MT=dA=d5C=dA=dG=d5C=dT=d5C=dT=dG=dG=dA=dG=dT=MG-MG- |
| | | | MG-MG-MT |
| 115 | hOTC- | CO-5058 | MT=MT=LA=MA- |
| | ASOe1-1al | | MT=LA=M5C=MA=LG=M5C=MT=L5C=MT=MG=LG=MA=MG=LT= |
| | | | MG-MG=LG=MG=MT |
| 116 | hOTC- | CO-5059 | MT=MT=LA=MA=MT=LA=M5C=MA- |
| | ASOe1-1am | | LG=M5C=MT=L5C=MT=MG=LG- |
| | | | MA=MG=LT=MG=MG=LG=MG=MT |
| 117 | hOTC- | CO-5060 | MT=MT=LA=MA=MT-LA=M5C=MA=LG-M5C=MT=L5C=MT=MG- |
| | ASOe1-1an | | LG=MA=MG=LT-MG=MG=LG=MG=MT |
| 118 | hOTC- | CO-5061 | MT=MT-LA-MA- |
| | ASOe1-1ao | | MT=LA=M5C=MA=LG=M5C=MT=L5C=MT=MG=LG=MA=MG=LT= |
| | | | MG-MG-LG-MG=MT |
| 119 | hOTC- | CO-5062 | MT=MT=LA-MA=MT=LA=M5C-MA=LG=M5C-MT=L5C=MT- |
| | ASOe1-1ap | | MG=LG=MA-MG=LT=MG=MG-LG=MG=MT |
| 120 | hOTC- | CO-5063 | MT-MT-LA-MA- |
| | ASOe1-1aq | | MT=LA=M5C=MA=LG=M5C=MT=L5C=MT=MG=LG=MA=MG=LT= |
| | | | MG-MG-LG-MG-MT |
| 121 | hOTC- | CO-5064 | MT=MT=LA-MA=MT-LA=M5C=MA-LG=M5C-MT=L5C=MT-MG=LG- |
| | ASOe1-1ar | | MA=MG=LT-MG=MG-LG=MG=MT |
| 122 | hOTC- | CO-5318 | MT=MT=LA=MA=MT=LA=M5C=MA=LG=M5C=MT=L5C=MT=MG=L |
| | ASOe1-1as | | G=MA=MG=LT=MG=MG=LG=MG=MT-tg |
| 123 | hOTC- | CO-6541 | MT=MT=LA=MA=MT=LA=M5C=MA=LG=M5C=MT=L5C=MT=MG= |
| | ASOe1-1at | | LG=MA=MG=LT=MG=MG=LG=MG=MT-ag |
| 124 | hOTC- | CO-6692 | MT=MT=LA=MA=MT=LA=M5C=MA=LG=M5C=MT=L5C=MT=MG= |
| | ASOe1-1au | | LG=MA=MG=LT=MG=MG=LG=MG-MT-tg |

Key:
MOE (M); DNA (d); LNA (L); PS (=); PO(-); 5-MethylCytosine (5C); GalNAc (ag); Teg-GalNAc (tg)

TABLE 4
Additional Chemical Modifications of hOTC-ASOe2-2
Sequence TATGAAGTAAGAAAGGGGTA

| SEQ | | | |
|---|---|---|---|
| ID NO | Name | Name | Chemical modification |
| 37 | hOTC- | CO-3392 | MT=MA=MT=MG=MA=dA=dG=dT=dA=dA=dG=dA=dA=dA=dG=MG= |
| | ASOe2-2a | | MG=MG=MT=MA |
| 67 | hOTC- | CO-4256 | MT=MA=MT=MG=MA=MA=MG=MT=MA=MA=MG=MA=MA=MA= |
| | ASOe2-2b | | MG=MG=MG=MG=MT=MA |
| 125 | hOTC- | CO-4257 | MT=MA=MT=MG=MA=dA=dG=dT=MA=dA=dG=MA=dA=dA=dG=MG |
| | ASOe2-2f | | =MG=MG=MT=MA |
| 126 | hOTC- | CO-4258 | MT=MA=MT=MG=MA=LA=dG=dT=LA=dA=dG=LA=dA=dA=LG=MG |
| | ASOe2-2g | | =MG=MG=MT=MA |
| 68 | hOTC- | CO-4259 | MT=MA=MT=LG=MA=dA=dG=LT=dA=dA=dG=LA=dA=dA=dG=LG= |
| | ASOe2-2c | | MG=MG=MT=MA |
| 127 | hOTC- | CO-4260 | MT=MA=MT=MG=LA=dA=dG=dT=LA=dA=dG=dA=LA=dA=dG=MG= |
| | ASOe2-2h | | LG=MG=MT=MA |
| 128 | hOTC- | CO-4261 | MT=MA=MT=MG=MA=LA=MG=MT=LA=MA=MG=LA=MA=MA=LG |
| | ASOe2-2i | | =MG=MG=MG=MT=MA |
| 129 | hOTC- | CO-4262 | MT=MA=MT=LG=MA=LA=MG=LT=MA=LA=MG=LA=MA=LA=MG= |
| | ASOe2-2j | | LG=MG=LG=MT=MA |
| 130 | hOTC- | CO-4263 | MT=MA=LT=MG=LA=MA=LG=MT=LA=MA=LG=MA=LA=MA=LG= |
| | ASOe2-2k | | MG=LG=MG=MT=MA |
| 131 | hOTC- | CO-4264 | MT=MA=LT=MG=MA=LA=MG=MT=LA=MA=MG=LA=MA=MA=LG |
| | ASOe2-2l | | =MG=MG=LG=MT=MA |
| 132 | hOTC- | CO-4265 | MT=MA=MT=MG=LA=MA=MG=MT=LA=MA=MG=MA=LA=MA=M |
| | ASOe2-2m | | G=MG=LG=MG=MT=MA |
| 69 | hOTC- | CO-4266 | MT=MA=MT=LG=MA=MA=MG=LT=MA=MA=MG=LA=MA=MA=M |
| | ASOe2-2d | | G=LG=MG=MG=MT=MA |
| 70 | hOTC- | CO-4267 | MT=MA=MT=MG=MA=LA=MG=MT=MA=MA=LG=MA=MA=MA=M |
| | ASOe2-2e | | G=LG=MG=MG=MT=MA |
| 133 | hOTC- | CO-4268 | MT=MA=MT=MG=LA=MA=MG=MT=MA=LA=MG=MA=MA=MA=L |
| | ASOe2-2n | | G=MG=MG=MG=MT=MA |
| 134 | hOTC- | CO-5048 | MT=MA=MT-MG- |
| | ASOe2-2o | | MA=LA=MG=MT=MA=MA=LG=MA=MA=MA=MG=LG-MG- |
| | | | MG=MT=MA |
| 135 | hOTC- | CO-5065 | MT=MA=MT=MG- |
| | ASOe2-2p | | MA=LA=MG=MT=MA=MA=LG=MA=MA=MA=MG=LG- |
| | | | MG=MG=MT=MA |
| 136 | hOTC- | CO-5066 | MT=MA=MT=MG=MA=LA=MG-MT=MA=MA=LG=MA=MA- |
| | ASOe2-2q | | MA=MG=LG=MG=MG=MT=MA |
| 137 | hOTC- | CO-5067 | MT=MA=MT=MG-MA=LA=MG=MT-MA=MA=LG=MA- |
| | ASOe2-2r | | MA=MA=MG=LG-MG=MG=MT=MA |
| 138 | hOTC- | CO-5068 | MT=MA-MT-MG- |
| | ASOe2-2s | | MA=LA=MG=MT=MA=MA=LG=MA=MA=MA=MG=LG-MG-MG- |
| | | | MT=MA |
| 139 | hOTC- | CO-5069 | MT=MA=MT-MG=MA=LA-MG=MT=MA-MA=LG-MA=MA=MA- |
| | ASOe2-2t | | MG=LG=MG-MG=MT=MA |
| 140 | hOTC- | CO-5070 | MT-MA-MT-MG- |
| | ASOe2-2u | | MA=LA=MG=MT=MA=MA=LG=MA=MA=MA=MG=LG-MG-MG-MT- |
| | | | MA |
| 141 | hOTC- | CO-5071 | MT=MA=MT-MG=MA-LA=MG-MT=MA-MA=LG-MA=MA-MA=MG- |
| | ASOe2-2v | | LG=MG-MG=MT=MA |
| 142 | hOTC- | CO-5319 | MT=MA=MT=MG=MA=LA=MG=MT=MA=MA=LG=MA=MA=MA=M |
| | ASOe2-2w | | G=LG=MG=MG=MT=MA-tg |

Key:
MOE (M); DNA (d); LNA (L); PS (=); PO(-); 5-MethylCytosine (5C); GalNAc (ag); Teg-GalNAc (tg)

hOTC-ASOe1-1 (SEQ ID NO: 6) is complementary to a sequence 1 nucleotide away from the 3′ end of human OTC eRNA-1A. SEQ ID NOs: 7-14, which are at least partially overlapping with SEQ ID NO: 6, are also complementary to sequences close to the 3′ end of human OTC eRNA-1A. hOTC-ASOe2-1 (SEQ ID NO: 15) is complementary to a sequence 9 nucleotides away from the 3′ end of human OTC eRNA-2A and 87 nucleotides away from the 3′ end of human OTC eRNA-2B. SEQ ID NO: 17, which is partially overlapping with SEQ ID NO: 16, is also complementary to a sequence close to the 3′ end of human OTC eRNA-2A and human OTC eRNA-2B. hOTC-ASOe2-2 (SEQ ID NO: 16) is complementary to a sequence 57 nucleotides away from the 5′ end of human OTC eRNA-2A.

### Hybridization and ΔG

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ, is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the free energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem, Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Aced Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal/mol for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal/mol, such as below −15 kcal/mol, such as below −20 kcal/mol and such as below −25 kcal/mol for oligonucleotides that are 8-30 nucleotides in length. In some embodiments the oligonucleotides hybridize to a target nucleic acid with an estimated ΔG° value of −10 to −60 kcal/mol, such as −12 to −40 kcal/mol, −15 to −30 kcal/mol, −16 to −27 kcal/mol, or −18 to −25 kcal/mol.

### Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 70% or greater complementarity. For example, a mismatch in a duplex region consisting of 19 base pairs results in 94.7% complementarity, rendering the duplex region substantially complementary. Duplex regions can be formed by two separate oligonucleotide strands, as well as by single oligonucleotide strands that can form hairpin structures comprising a duplex region.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an OTC or Serping1 regRNA, such as an eRNA or paRNA. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides. Generally, the duplex structure is between 15 and 50 base pairs in length, e.g., between, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 base pairs in length. Ranges and lengths intermediate to the above recited ranges and lengths are also contemplated to be part of the invention.

Similarly, the region of complementarity to the target sequence can be between 15 and 50 nucleotides in length, e.g., between 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 nucleotides in length. Ranges and lengths intermediate to the above recited ranges and lengths are also contemplated to be part of the invention.

### Chemical Modifications of ASOs

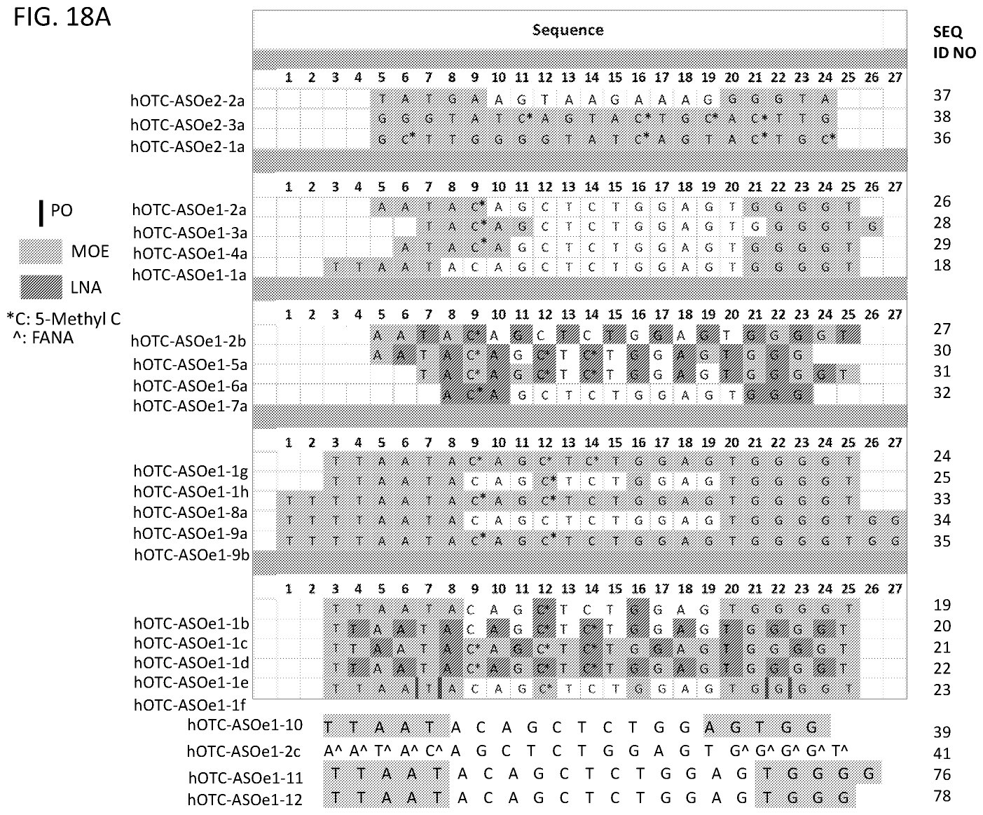

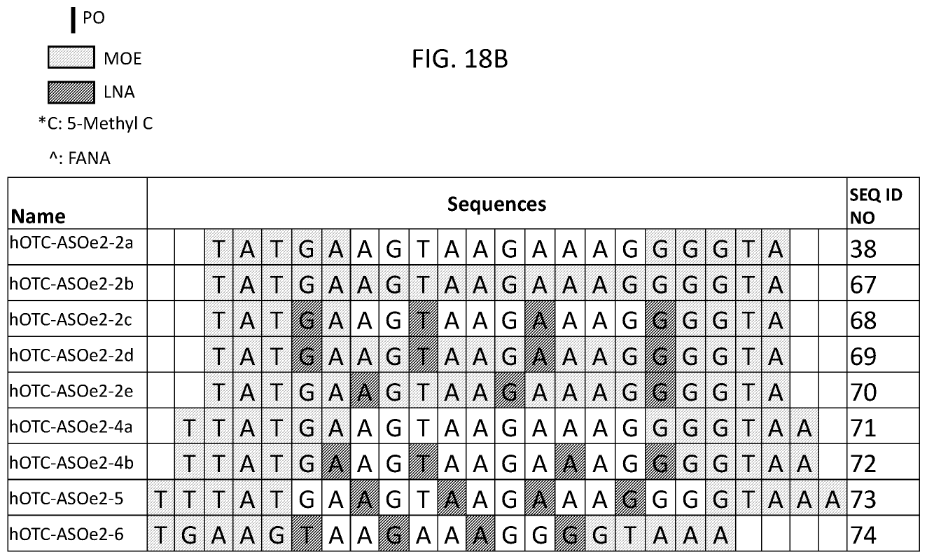

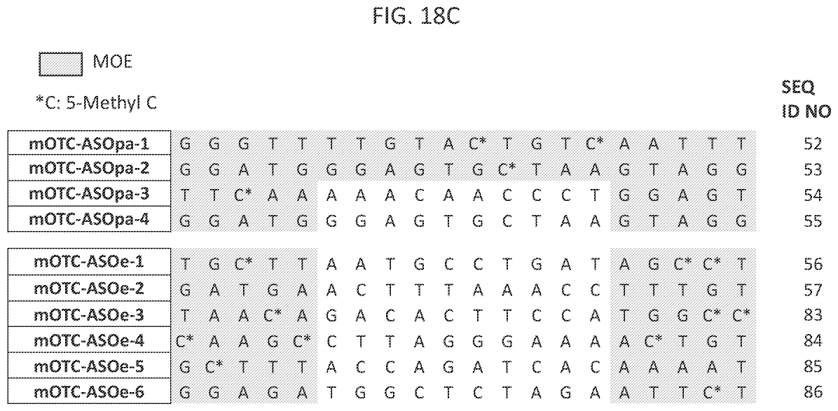

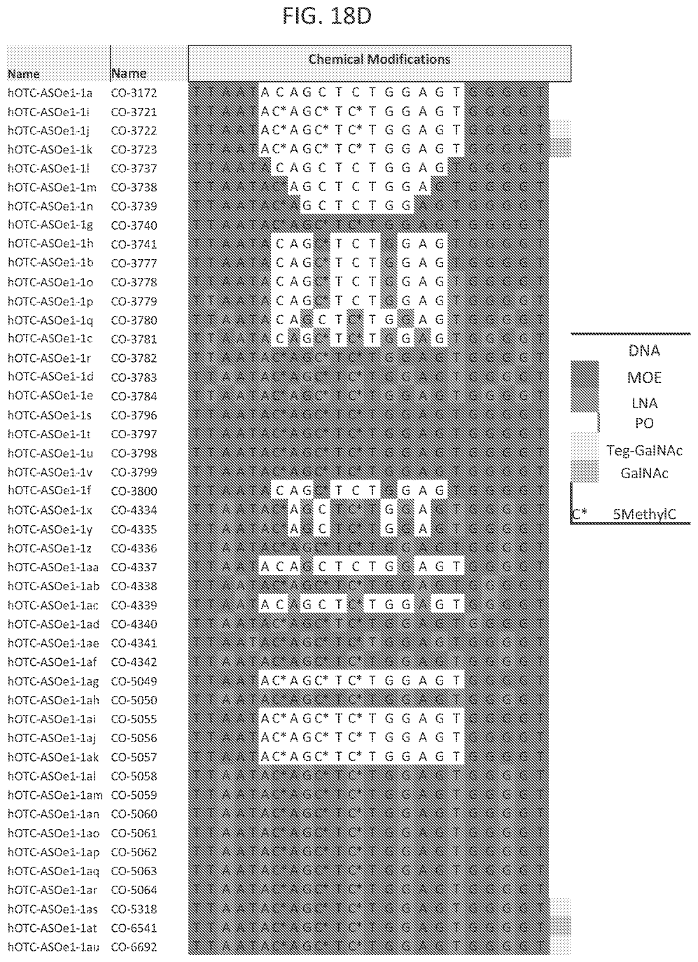

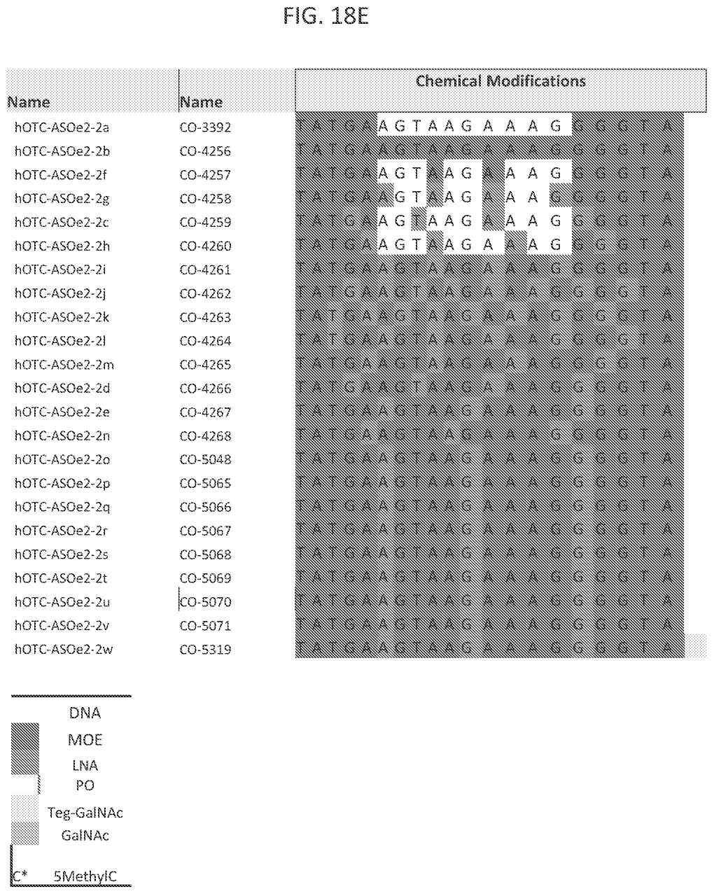

In certain embodiments, the ASO does not consist of only DNA. In certain embodiments, the ASO comprises at least one chemical modification relative to a natural nucleotide (e.g., ribonucleotide). Various chemical modifications can be included in the ASOs of the present disclosure. The modifications can include one or more modifications in a ribose group, one or more modifications in a phosphate group, one or more modifications in a nucleobase, one or more terminal modifications, or a combination thereof. In some embodiments, an exemplary ASO sequence targeting a regRNA as shown in Table 2 is chemically modified. For example, hOTC-ASOe1-1 may be chemically modified to comprise the modifications of any one of hOTC-ASOe1-1a to hOTC-ASOe1-1h as shown in FIG. 18A. Such modifications can be, but are not limited to, 2′-O-(2-Methoxyethyl) (2′-MOE), locked nucleic acid (LNA), 5-methyl on the cytidine, constrained ethyl (cET), phosphorothioate (PS) linkage, and/or a phosphodiester (PO) linkage, or any combination thereof. Chemical modifications of RNA are known in the art and described in, for example, PCT Application Publication No. WO2013/177248. In certain embodiments, each cytidine in the ASO is modified by 5-methyl.

Various chemical modifications for use with ASOs of the present disclosure include, but are not limited to: 3′-terminal deoxy-thymine (dT) nucleotides, 2′-O-methyl modified nucleotides, 2′-fluoro modified nucleotides, 2′-deoxy-modified nucleotides, locked nucleotides, unlocked nucleotides, conformationally restricted nucleotides, constrained ethyl nucleotides, abasic nucleotides, 2′-amino-modified nucleotides, 2′-O-allyl-modified nucleotides, 2′-C-alkyl-modified nucleotides, 2′-hydroxyl-modified nucleotides, 2′-methoxyethyl modified nucleotides, 2′-O-alkyl-modified nucleotides, morpholino nucleotides, phosphoramidates, non-natural base comprising nucleotides, tetrahydropyran modified nucleotides, 1,5-anhydrohexitol modified nucleotides, cyclohexenyl modified nucleotides, nucleotides comprising a phosphorothioate group, nucleotides comprising a methylphosphonate group, nucleotides comprising a 5′-phosphate, and nucleotides comprising a 5′-phosphate mimic.

In certain embodiments, the ASO comprises an RNA polynucleotide chemically modified to be resistant to one or more nuclear RNases (e.g., the exosome complex or RNaseH). In some embodiments, all nucleotide bases are modified in the ASO. In certain embodiments, the chemical modifications comprises β-D-ribonucleosides, 2′-modified nucleosides (e.g., 2′-O-(2-Methoxyethyl) (2′-MOE), 2′-O—CH₃, or 2′-fluoro-arabino (FANA)), bicyclic sugar modified nucleosides (e.g., having a constrained ethyl or locked nucleic acid (LNA)), and/or one or more modified internucleotide bonds (e.g., phosphorothioate internucleotide linkage). In certain embodiments, the chemical modification comprises 2′-MOE and a phosphorothioate internucleotide bond. In certain embodiments, at least 8, 9, 10, 11, 12, 13, 14, 15, 16, 17, 18, 19, 20, or more consecutive nucleotides of the ASO are modified by 2′-MOE. In certain embodiments, each nucleotide of the ASO is modified by 2′-MOE. In certain embodiments, at least 7, 8, 9, 10, 11, 12, 13, 14, 15, 16, 17, 18, 19, 20, or more consecutive internucleotide bonds of the ASO are phosphorothioate internucleotide bonds. In certain embodiments, each internucleotide bond of the ASO is a phosphorothioate internucleotide bond.

Internucleotide linkage modifications that can be used with the ASOs of the present disclosure include, but are not limited to, phosphorothioate “PS” (P(S)), phosphoramidate (P(NR1R2) such as dimethylaminophosphoramidate (P(N(CH3)2)), phosphonocarboxylate (P(CH2)nCOOR) such as phosphoinoacetate “PACE” (P(CH2COO—)), thiophosphonocarboxylate ((S)P(CH12)nCOOR) such as thiophosphonoacetate “thioPACE” ((S)P(CH2COO—), alkylphosphonate (P(C1-3alkyl) such as methylphosphonate —P(CH3), boranophosphonate (P(BH3)), and phosphorodithioate (P(S)2).

In certain embodiments, the ASO comprises one or more chemical modifications at the 5′ end, the 3′ end, or both. Without wishing to be bound by theory, chemical modifications at one or both termini of a polynucleotide (e.g., polyribonucleotide) may stabilize the polynucleotide. In certain embodiments, the ASO comprises one or more chemical modifications in at least 1, 2, 3, 4, or 5 nucleotides at the 5′ end of the ASO. In certain embodiments, the ASO comprises one or more chemical modifications in at least 1, 2, 3, 4, or 5 nucleotides at the 3′ end of the ASO. In certain embodiments, the ASO comprises one or more chemical modifications in at least 1, 2, 3, 4, or 5 nucleotides at the 5′ end of the ASO and one or more chemical modifications in at least 1, 2, 3, 4, or 5 nucleotides at the 3′ end of the ASO.

The chemical structures can also be described in writing. In such cases, ‘M’ indicates MOE; ‘d’ indicates DNA, ‘L’ indicates LNA, ‘=’ indicates a phosphorothioate (PS) linkage, ‘-’ indicates a phosphodiester (PO) linkage; ‘5C’ indicates 5-MethylCytosine, ‘ag’ indicates GalNAc, ‘tg’ indicates Teg-GalNAc, and ‘^{A}’ indicates FANA.

To avoid ambiguity, this LNA has the formula:

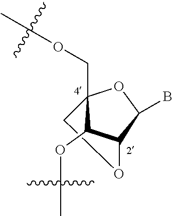

wherein B is the particular designated base.

Exemplary written descriptions of selected ASOs are provided in Table 3 and Table 4, with corresponding FIGS. 18D and 18E providing visual representation of the modifications.

In some embodiments, the ASO comprises a sequence and/or chemical modification selected from the group consisting of SEQ ID NOs: 6-14, 18-35, 39, 41, 75, 76, 77, 78, 87-124, or 143-892. In some embodiments, the ASO comprises a sequence and/or chemical modification selected from the group consisting of SEQ ID NOs: 15-17, 36-38, 64-74, 125-142, or 893-1029. In some embodiments, the ASO comprises a sequence and chemical modification selected from the group consisting of SEQ ID NOs: 87-124. In some embodiments, the ASO comprises a sequence and chemical modification selected from the group consisting of SEQ ID NOs: 125-142. In some embodiments, the ASO comprises a sequence and chemical modification selected from the group consisting of SEQ ID NOs: 1030-1072.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tm). A high affinity modified nucleoside of the present invention preferably result in an increase in melting temperature between +0.5 to +12° C., such as between +1.5 to +10° C. or +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213), each of which are hereby incorporated by reference.

### Sugar Modifications

The ASOs described herein may comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798), both of which are hereby incorporated by reference. Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

In some embodiments, oligonucleotides comprise modified sugar moieties, such as any one of a 2′-O-methyl (2′OMe) moeity, a 2′-O-methoxyethyl moeity, a bicyclic sugar moeity, PNA (e.g., an oligonucleotide comprising one or more N-(2-aminoethyl)-glycine units linked by amide bonds or carbonyl methylene linkage as repeating units in place of a sugar-phosphate backbone), locked nucleoside (LNA) (e.g., an oligonucleotide comprising one or more locked ribose, and can be a mixture of 2′-deoxy nucleotides or 2′OMe nucleotides), c-ET (e.g., an oligonucleotide comprising one or more cET sugar), cMOE (e.g., an oligonucleotide comprising one or more cMOE sugar), morpholino oligomer (e.g., an oligonucleotide comprising a backbone comprising one or more phosphorodiamidate morpholiono oligomers), 2′-deoxy-2′-fluoro nucleoside (e.g., an oligonucleotide comprising one or more 2′-fluoro-β-D-arabinonucleoside), tcDNA (e.g., an oligonucleotide comprising one or more tcDNA modified sugar), constrained ethyl 2′-4′-bridged nucleic acid (cEt), S-cEt, ethylene bridged nucleic acid (ENA) (e.g., an oligonucleotide comprising one or more ENA modified sugar), hexitol nucleic acids (HNA) (e.g., an oligonucleotide comprising one or more HNA modified sugar), or tricyclic analog (tcDNA) (e.g., an oligonucleotide comprising one or more tcDNA modified sugar).

In some embodiments, oligonucleotides comprise nucleobase modifications selected from the group consisting of 2-thiouracil (“2-thioU”), 2-thiocytosine (“2-thiC”), 4-thiouracil (“4-thioU”), 6-thioguanine (“6-thioG”), 2-aminoadenine (“2-aminoA”), 2-aminopurine, pseudouracil, hypoxanthine, 7-deazaguanine. 7-deaza-8-azaguanine, 7-deazaadenine, 7-deaza-8-azaadenine, 5-methylcytosine “5-methylC”), 5-methyluracil (“5-methylU”). 5-hydroxymethylcytosine, 5-hydroxymethyluracil, 5,6-dihydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), and isocytosine (“isoC”), glycerol nucleic acid (GNA), thiomorpholino (C4H9NS) or thiophosphoramidate morpholinos (TMOs). Synthesis of glycerol nucleic acid (GNA) (also known as glycol nucleic acids) is described in Zhang et al, Current Protocols in Nucleic Acid Chemistry 4.40.1-4.40.18, September 2010, hereby incorporated by reference. Synthesis of thiophosphoramidate Morpholino Oligonucleotides is described in Langer et al, J. Am. Chem. Soc. 2020, 142, 38, 16240-16253

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Without wishing to be bound by theory, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937, each of which are hereby incorporated by reference.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. In other words, a locked nucleoside is a nucleoside comprising a bicyclic sugar moiety comprising a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). These nucleosides are also sometimes termed bridged nucleic acid or bicyclic nucleic acid (BNA). The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex. Exemplary LNA nucleosides include beta-D-oxy-LNA, 6′-methyl-beta-D-oxy LNA such as (S)-6′-methyl-beta-D-oxy-LNA (ScET) and ENA.

Examples of bicyclic nucleosides for use in the polynucleotides of the invention include without limitation nucleosides comprising a bridge between the 4′ and the 2′ ribosyl ring atoms. In certain embodiments, the polynucleotide agents of the invention include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see International Publication No. WO 99/14226, contents of which are incorporated by reference herein).

An oligonucleotide of the invention can also be modified to include one or more constrained ethyl nucleosides. As used herein, a “constrained ethyl nucleoside” or “cEt” is a locked nucleoside comprising a bicyclic sugar moiety comprising a 4′-CH(CH₃)—O-2′ bridge. In one embodiment, a constrained ethyl nucleoside is in the S conformation referred to herein as “S-cEt.”

An oligonucleotide of the invention may also include one or more “conformationally restricted nucleosides” (“CRN”). CRN are nucleoside analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide of the invention comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

Potentially stabilizing modifications to the ends of nucleoside molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Additional non-limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667, each of which are hereby incorporated by reference.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the nucleotide sugar modification is 2′-O—C1-4alkyl such as 2′-methyl (2′-OMe), 2′-deoxy (2′H), 2′-C1-3alkyl-O—C1-3alkyl such as 2′-methoxyethyl (“2′-MOE”), 2′-fluoro (“2-F”), 2′-amino (“2′-NH2”), 2′-arabinosyl (“2′-arabino”) nucleotide. 2′-F-arabinosyl (“2′F-arabino”) nucleotide. 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), or 4′-thioribosyl nucleotide.

### Mixmers and Gapmers

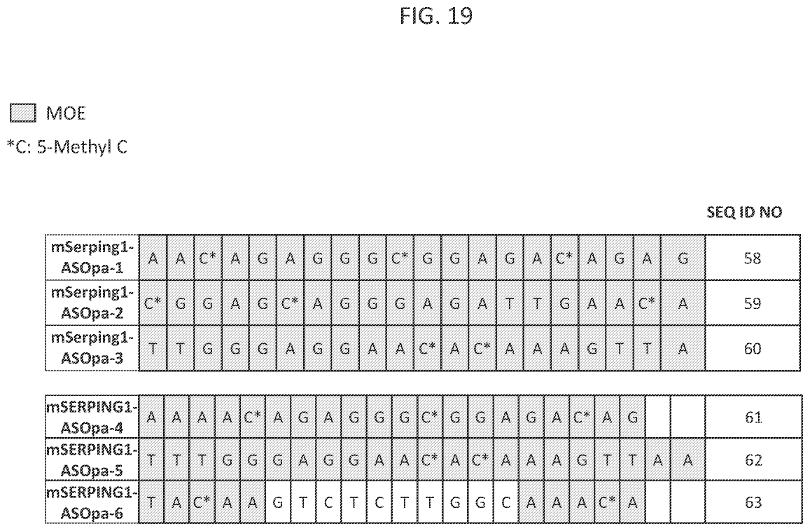

The ASO can have a mixmer and/or gapmer structure, for example, in a pattern disclosed by the ASOs in FIG. 18A, FIG. 18B, FIG. 18C, or FIG. 19.

In certain embodiments, the ASO is a mixmer. As used herein, the term “mixmer” refers to an oligonucleotide comprising an alternating composition of DNA monomers and nucleoside analogue monomers across at least a portion of the oligonucleotide sequence. In certain embodiments, the ASO is a mixmer based on the gapmer structure, comprising a mixture of DNA nucleotides and 2′-MOE nucleotides in the gap, flanked by RNA sequences in the wings. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) mononmers, 2′-fluoro-ANA monomers, HNA monomers, INA monomers, 2′-MOE-RNA (2′-O-methoxyethyl-RNA), 2′Fluoro-DNA, and LNA. In some embodiments, the mixmer is incapable of recruiting RNase H. In some embodiments, the mixmer comprises one type of affinity enhancing nucleotide analogue together with DNA and/or RNA.

Multiple different modifications can be interspaced in a mixmer. For example, the ASO can comprise LNA modification in a plurality of nucleotides and a different modification in some or all of the rest of the nucleotides. In some embodiments, any two adjacent LNA-modified nucleotides are separated by at least 1, 2, 3, 4, or 5 nucleotides. Throughout the ASO, the distance between adjacent LNA-modified nucleotides can either be constant (e.g., any two adjacent LNA-modified nucleotides are separated by 1, 2, 3, 4, or 5 nucleotides) or variable. In some embodiments, the length of the ASO is 3×n, 3×n−1, or 3×n−2 nucleotides (n is an integer of 6 or greater), wherein (a) (i) the nucleotides at positions 3×m−2 (m is an integer from 1 to n) are ribonucleotides comprising a first modification (e.g., LNA), (ii) the nucleotides at positions 3×m−1 (m is an integer from 1 to n) are ribonucleotides comprising a first modification (e.g., LNA), or (iii) the nucleotides at positions 3×m (m is an integer from 1 to n) are ribonucleotides comprising a first modification (e.g., LNA); and (b) the nucleotides at the remaining positions comprise a second, different modification (e.g., 2′-O-methoxyethyl). The ASO called hOTC-ASOe1-1d herein has such a structure. In some embodiments, the length of the ASO is 2×n or 2×n−1 nucleotides (n is an integer of 9 or greater), wherein (a) (i) the nucleotides at positions 2×m 1 (m is an integer from 1 to n) are ribonucleotides comprising a first modification (e.g., LNA), or (ii) the nucleotides at positions 2×m (m is an integer from 1 to n) are ribonucleotides comprising a first modification (e.g., LNA); and (b) the nucleotides at the remaining positions comprise a second, different modification (e.g., 2′-O-methoxyethyl). The ASO called hOTC-ASOe1-1e herein has such a structure. Similar modification patterns, for example, where the first modification is repeated very 4, 5, or more nucleotides, are also contemplated. In some embodiments, the length of the ASO is 4×n, 4×n−1, or 4×n−2 nucleotides (n is an integer of 6 or greater), wherein (a) (i) the nucleotides at positions 4×m−2 (m is an integer from 1 to n) are ribonucleotides comprising a first modification (e.g., LNA), (ii) the nucleotides at positions 4×m−1 (m is an integer from 1 to n) are ribonucleotides comprising a first modification (e.g., LNA), or (iii) the nucleotides at positions 3×m (m is an integer from 1 to n) are ribonucleotides comprising a first modification (e.g., LNA); and (b) the nucleotides at the remaining positions comprise a second, different modification (e.g., 2′-O-methoxyethyl). In some embodiments, the length of the ASO is 5×n, 5×n−1, or 5×n−2 nucleotides (n is an integer of 6 or greater), wherein (a) (i) the nucleotides at positions 5×m−2 (m is an integer from 1 to n) are ribonucleotides comprising a first modification (e.g., LNA), (ii) the nucleotides at positions 5×m−1 (m is an integer from 1 to n) are ribonucleotides comprising a first modification (e.g., LNA), or (iii) the nucleotides at positions 5×m (m is an integer from 1 to n) are ribonucleotides comprising a first modification (e.g., LNA); and (b) the nucleotides at the remaining positions comprise a second, different modification (e.g., 2′-O-methoxyethyl).

In some embodiments, the ASO further comprises a GalNAc or Teg-GalNAc moiety at the 5′ or 3′ end of the ASO.

In certain embodiments, the ASO comprises a DNA sequence (e.g., having at least 8, 9, 10, 11, 12, 13, 14, or 15 contiguous nucleotides of unmodified DNA) flanked by RNA sequences. Such structure is known as “gapmer,” in which the internal DNA region is referred to as the “gap” and the external RNA regions is referred to as the “wings” (see, e.g., PCT Application Publication No. WO2013/177248). Gapmers were known to facilitate degradation of the target RNA by recruiting nuclear RNAses (e.g., RNase H). Surprisingly, in the present disclosure, it has been discovered that a gapmer binding a regRNA (e.g., hOTC-ASOe1-1a), like regRNAs having the same sequence but having different chemical modifications (e.g., hOTC-ASOe1-1d and hOTC-ASOe1-1h), can also increase target gene expression. In certain embodiments, the ASO comprises a DNA sequence flanked by RNA sequences and does not induce RNAse- or RNAse H-mediated degradation.

In certain embodiments, the gapmer is about 20, 21, 22, 23, 24, 25, 26, 27, 28, 29, 30, or more nucleotides in length. In certain embodiments, the gap is about 7, 8, 9, 10, 11, 12, 13, 14, 15, or more nucleotides in length. In certain embodiments, one or both wings are about 2, 3, 4, 5, 6, 7, 8, 9, 10, or more nucleotides in length. In certain embodiments, one or both wings comprises RNA modifications, for example, β-D-ribonucleosides, 2′-modified nucleosides (e.g., 2′-O-(2-Methoxyethyl) (2′-MOE), 2′-O—CH₃, or 2′-fluoro-arabino (FANA)), and bicyclic sugar modified nucleosides (e.g., having a constrained ethyl or locked nucleic acid (LNA)). In certain embodiments, each ribonucleotide in the gapmer is modified by 2′-MOE. In certain embodiments, the gapmer comprises one or more modified internucleotide bonds, e.g., phosphorothioate (PS) internucleotide linkage. In certain embodiments, each two adjacent nucleotides in the gapmer are linked by a phosphorothioate internucleotide bond.

In certain embodiments, the ASO does not comprise 7 or more, 8 or more, 9 or more, 10 or more, 10 or more, 11 or more, 12 or more, 13 or more, 14 or more, or 15 or more contiguous nucleotides of unmodified DNA. In some embodiments, such a DNA sequence is disrupted by modified (e.g., 2′-MOE modified) ribonucleotides every 2, 3, 4, 5, or more nucleotides. The ASO called hOTC-ASOe1-if herein has such a structure. In some embodiments, the ASO comprises only ribonucleotides and no deoxyribonucleotides.

The structural features of mixmer and gapmer can be combined. In certain embodiments, the ASO has a structure similar to that of a mixmer disclosed herein (e.g., one having interspaced modifications), except that the second modification in the gap is changed to a third modification (e.g., deoxyribonucleotide). The ASOs called hOTC-ASOe1-1c, hOTC-ASOe1-2b, hOTC-ASOe1-5a, and hOTC-ASOe1-6a herein have such structures. In certain embodiments, the ASO has a structure similar to that of a gapmer disclosed herein, except that in the gap the nucleotides are modified in a mixmer pattern. The ASO called hOTC-ASOe1-1b herein has such a structure.

In certain embodiments, the ASO further comprises a ligand moiety, e.g., a ligand moiety that specifically targets a tissue or organ in a subject. For example, N-Acetylgalactosamine (GalNAc) specifically targets liver. In certain embodiments, the ligand moiety comprises GalNAc. In certain embodiments, the ligand moiety comprises a three-cluster GalNAc moiety, commonly denoted GAlNAc3. Other types of GalNAc moieties are one-cluster, two cluster or four cluster GAlNAc, denoted as GAlNAc1, GAlNAc2, or GAlNAc4. In certain embodiments, the ligand moiety comprises GalNAc1, GALNAc2, GAlNAc3, or GalNAc4.

### Pharmaceutical Compositions

In certain embodiments, the ASOs disclosed herein can be present in pharmaceutical compositions. The pharmaceutical composition can be formulated for use in a variety of drug delivery systems. One or more pharmaceutically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Exemplary carriers and pharmaceutical formulations suitable for delivering nucleic acids are described in Durymanov and Reineke (2018) Front. Pharmacol. 9:971; Barba et al. (2019) Pharmaceutics 11(8): 360; Ni et al. (2019) Life (Basel) 9(3): 59. It is understood that the presence of a ligand moiety conjugated to the ASO may circumvent the need for a carrier for delivery to a tissue or organ targeted by the ligand moiety.

The delivery of an oligonucleotide of the invention to a cell e.g., a cell within a subject, such as a human subject e.g., a subject in need thereof, such as a subject having an OTC related disorder can be achieved in a number of different ways. For example, delivery may be performed by contacting a cell with an oligonucleotide of the invention either in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the invention (see e.g., Akhtar S. and Julian R L., (1992) Trends Cell. Biol. 2(5):139-144 and WO 94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the invention. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the invention are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

In some embodiments, the compounds described herein may be administered in combination with additional therapeutics. Examples of additional therapeutics include standard of care anti-epilepsy medications such as quinidine and/or sodium channel blockers. Additionally, the compounds described herein may be administered in combination with recommended lifestyle changes such as a ketogenic diet.

### Membranous Molecular Assembly Delivery Methods

Oligonucleotides of the invention can also be delivered using a variety of membranous molecular assembly delivery methods including polymeric, biodegradable microparticle, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S. T. P. Pharma. Sci., 4(6):466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M}1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M}1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2, 405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic. Lipid Nanoparticle-Based Delivery Methods

Oligonucleotides of in the invention may be fully encapsulated in a lipid formulation, e.g., a lipid nanoparticle (LNP), or other nucleic acid-lipid particle. LNPs are useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

Non-limiting examples of cationic lipids include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-−3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (Cis). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

The ASO may also be deliver in a lipidoid. The synthesis of lipidoids has been extensively described and formulations containing these compounds are particularly suited for delivery of modified nucleic acid molecules or ASOs (see Mahon et al, Bioconjug Chem. 2010 21: 1448-1454; Schroeder et al, J Intern Med. 2010 267:9-21; Akinc et al, Nat Biotechnol. 2008 26:561-569; Love et al, Proc Natl Acad Sci USA. 2010 107: 1864-1869; Siegwart et al, Proc Natl Acad Sci USA. 2011 108: 12996-3001; all of which are incorporated herein in their entireties).

Lipid compositions for RNA delivery are disclosed in WO2012170930A1, WO2013149141A1, and WO2014152211A1, each of which are hereby incorporated by reference.

### Therapeutic Applications

The present invention provides methods for treating diseases and disorders associated with decreased gene expression (e.g., decreased OTC gene expression). The method employs an ASO that hybridizes with a regulatory RNA transcribed from a regulatory element of the target gene (e.g., OTC) or a pharmaceutical composition comprising the ASO. The oligonucleotide compositions described herein are useful in the methods of the invention and, while not bound by theory, are believed to exert their desirable effects through their ability to modulate the level, status, and/or activity of OTC, e.g., by increasing the level of the OTC protein in a cell in a subject (e.g., a mammal, a primate, or a human).

An aspect of the present invention relates to methods of treating disorders (e.g., urea cycle disorders) related to OTC in a subject in need thereof. Another aspect of the invention includes increasing the level of OTC in a cell of a subject identified as having a OTC related disorder. Still another aspect includes a method of increasing expression of OTC in a cell in a subject. The methods may include contacting a cell with an oligonucleotide or ASO, in an amount effective to increase expression of OTC in the cell, thereby increasing expression of OTC in the cell.

Based on the above methods, further aspects of the present invention include an oligonucleotide of the invention, or a composition comprising such an oligonucleotide, for use in therapy, or for use as a medicament, or for use in treating OTC or urea cycle related disorders in a subject in need thereof, or for use in increasing the level of OTC in a cell of a subject identified as having a OTC related disorder, or for use in increasing expression of OTC in a cell in a subject. The uses include the contacting of a cell with the oligonucleotide, in an amount effective to increase expression of OTC in the cell, thereby increasing expression of OTC in the cell. Embodiments described below in relation to the methods of the invention are also applicable to these further aspects.

Contacting of a cell with an oligonucleotide may be done in vitro, ex vivo, or in vivo. Contacting a cell in vivo with the oligonucleotide includes contacting a cell or group of cells within a subject, e.g., a human subject, with the oligonucleotide. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest. The cell can be a liver cell (e.g., a hepatocyte).

Administration of the ASO or pharmaceutical composition disclosed herein could be intravenous, intraarterial, intraperitoneal, intramuscular, subcutaneous, intrapleural, intrathecal, intracavitary, by perfusion through a catheter or by direct intralesional injection. In certain embodiments, the ASO or pharmaceutical composition is administered systemically. In certain embodiments, the ASO or pharmaceutical composition is administered by a parenteral route. For example, in certain embodiments, the ASO or pharmaceutical composition is administered by intravenously (e.g., by intravenous infusion), for example, with a prefilled bag, a prefilled pen, or a prefilled syringe. In other embodiments, the ASO or pharmaceutical composition is administered locally to an organ or tissue in which an increase in the target gene expression is desirable (e.g., liver).

In some embodiments, the oligonucleotide is administered to a subject such that the oligonucleotide is delivered to a specific site within the subject. Such targeted delivery can be achieved by either systemic administration or local administration. The increase of expression of OTC may be assessed using measurements of the level or change in the level of OTC mRNA or OTC protein in a sample derived from a specific site within the subject. In certain embodiments, the methods include a clinically relevant increase of expression of OTC, e.g., as demonstrated by a clinically relevant outcome after treatment of a subject with an agent to reduce the expression of OTC.

In other embodiments, the oligonucleotide is administered in an amount and for a time effective to result in reduction (e.g., by 10%, 20%, 30%, 40%, 50%, 60%, 70%, 80%, 90%, or 100%) of one or more symptoms of a OTC disorder, such as high ammonia level in the blood.

### Increase of OTC Expression Level

A therapeutic method disclosed herein, using an ASO that targets OTC, is designed to increase OTC expression level in a subject. Increasing expression of a OTC gene includes any level of increasing of a OTC gene, e.g., at least partial increase of the expression of a OTC gene. Increase may be assessed by an increase in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control). In certain embodiments, the method causes a clinically relevant increase of expression of OTC, e.g. as demonstrated by a clinically relevant outcome after treatment of a subject with an agent to increase the expression of OTC.

In certain embodiments, the method disclosed herein increases OTC gene expression by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose baseline level. In certain embodiments, the method disclosed herein increases OTC gene expression by at least 2 fold, at least 3 fold, at least 4 fold, at least 5 fold, at least 6 fold, at least 7 fold, at least 8 fold, at least 9 fold, or at least 10 fold relative to the pre-dose baseline level. In certain embodiments, the subject has a deficiency in OTC expression, and the method disclosed herein restores the OTC expression level or activity to at least 20%, at least 30%, at least 40%, at least 50%, at least 60%, at least 70%, at least 80%, at least 90%, or at least 100% of the average OTC expression level or activity in subjects of the species of like age and gender.

The expression of a OTC gene may be assessed based on the level of any variable associated with OTC gene expression, e.g., OTC mRNA level or OTC protein level. It is understood that OTC is a X-chromosome gene in certain mammals (e.g., human and mouse) and female subjects exhibit mosaic patterns of X-chromosome inactivation. In certain embodiments, the expression level or activity of OTC herein refers to the average expression level or activity in the liver.

In certain embodiments, surrogate markers can be used to detect an increase of OTC expression level. For example, effective treatment of a OTC related disorder, as demonstrated by acceptable diagnostic and monitoring criteria with an agent to increase OTC expression can be understood to demonstrate a clinically relevant increase in OTC.

Increase of the expression of a OTC gene may be manifested by an increase of the amount of mRNA expressed by a first cell or group of cells (such cells may be present, for example, in a sample derived from a subject) in which a OTC gene is transcribed and which has or have been treated (e.g., by contacting the cell or cells with an oligonucleotide of the invention, or by administering an oligonucleotide of the invention to a subject in which the cells are or were present) such that the expression of a OTC gene is increased, as compared to a second cell or group of cells substantially identical to the first cell or group of cells but which has not or have not been so treated (control cell(s) not treated with an oligonucleotide or not treated with an oligonucleotide targeted to the gene of interest).

In other embodiments, increase of the expression of a OTC gene may be assessed in terms of an increase of a parameter that is functionally linked to OTC gene expression, e.g., OTC protein expression or OTC activity. OTC increase may be determined in any cell expressing OTC, either endogenous or heterologous from an expression construct, and by any assay known in the art.

An increase of OTC expression may be manifested by an increase in the level of the OTC protein that is expressed by a cell or group of cells (e.g., the level of protein expressed in a sample derived from a subject), relative to a control cell or a control group of cells. An increase of OTC expression may also be manifested by an increase in the level of the OTC mRNA level in a treated cell or group of cells, relative to a control cell or a control group of cells.

A control cell or group of cells that may be used to assess the increase of the expression of a OTC gene includes a cell or group of cells that has not yet been contacted with an oligonucleotide of the invention. For example, the control cell or group of cells may be derived from an individual subject (e.g., a human or animal subject) prior to treatment of the subject with an oligonucleotide.

The level of OTC mRNA that is expressed by a cell or group of cells may be determined using any method known in the art for assessing mRNA expression. In one embodiment, the level of expression of OTC in a sample is determined by detecting a transcribed polynucleotide, or portion thereof, e.g., mRNA of the OTC gene. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating OTC mRNA may be detected using methods the described in PCT Publication WO 2012/177906, the entire contents of which are hereby incorporated herein by reference. In some embodiments, the level of expression of OTC is determined using a nucleic acid probe. The term “probe,” as used herein, refers to any molecule that is capable of selectively binding to a specific OTC sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to OTC mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of OTC mRNA.

An alternative method for determining the level of expression of OTC in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of OTC is determined by quantitative fluorogenic RT-PCR (i.e., the TAQMAN™ System) or the DUAL-GLO® Luciferase assay.

The expression levels of OTC mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of OTC expression level may also comprise using nucleic acid probes in solution.

In some embodiments, the level of mRNA expression is assessed using branched DNA (bDNA) assays, quantitative PCR (qPCR), RT-qPCR, multiplex qPCR or RT-qPCR, RNA-seq, or microarray analysis. Such methods can also be used for the detection of OTC nucleic acids.

The level of OTC protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, FACS, immunodiffusion (single or double), immunoelectrophoresis, western blotting, radioimmunoassay (RIA), enzyme-linked immunosorbent assays (ELISAs), immunofluorescent assays, electrochemiluminescence assays, Luminex, MSD, FISH, and the like. Such assays can also be used for the detection of proteins indicative of the presence or replication of OTC proteins.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Modulation of OTC Expression Using regRNA-Targeting ASOs

Two human OTC regRNA targets (RR1 and RR2) were identified for human OTC. 69 ASOs targeting RR1 and 133 ASOs targeting RR2 were synthesized. These 202 initial ASOs were screened in primary human hepatocytes at 5 μM for efficacy in increasing OTC mRNA. ASOs that showed efficacy were further tested for dose dependent efficacy at 1.25 μM, 2.5 μM, and 5 μM in primary human hepatocytes, and primary human doner hepatocytes. Positive ASOs that exhibited dose dependent efficacy were selected for ASO basewalking and tiling around the regRNA hit areas. Based on the initial screening, 31 RR1 ASOs and 35 RR2 ASOs were selected for basewalking and tiling around the initial ASO hits. These additional ASOs were further tested for dose dependent efficacy. ASOs were selected for chemistry fine tuning by altering the chemistry, type, and position of chemical modification of the selected ASOs. 71 ASOs targeting RR1 and 67 ASOs targeting RR2 were identified.

This process was repeated for mouse OTC regRNA to identify ASOs that alter mouse OTC expression. Four mouse OTC regRNA targets were identified for mouse OTC. 126 ASOs targeting the regRNA were synthesized. These 126 initial ASOs were screened in primary mouse hepatocytes for efficacy in increasing OTC mRNA. Positive ASOs that exhibited dose dependent efficacy were selected for ASO basewalking and tiling around the regRNA hit areas. Based on the initial screening, 24 ASOs were selected for basewalking and tiling around the initial ASO hits. These additional ASOs were further tested for dose dependent efficacy. Four ASOs were selected for chemistry fine tuning by altering the chemistry, type, and position of chemical modification of the selected ASOs.

A selection of the human and mouse ASOs and chemical modifications are shown in Table 2, 3, 4, and FIGS. 18A, 18B, 18C, 18D, and 18E.

This example was designed to assess modulation of OTC expression in human hepatocytes using ASOs targeting eRNAs transcribed from an enhancer of human OTC.

Hepatocytes from four donors (HUM4178, HUM181511A, HUM190171, HUM181371) were cultured in vitro. Cells were plated in growth media and treated 4-6 hours after plating with final concentrations of 1.25 μM, 2.5 μM, 5 μM, or 10 μM hOTC-ASOe1-1d, hOTC-ASOe1-1h, hOTC-ASOe2-1, or hOTC-ASOe1-1a (see FIGS. 18A, 18B, 18D, and 18E for human OTC sequences and chemical modifications of selected ASOs and FIG. 18C for mouse OTC sequences and chemical modifications of selected ASOs). Cells were collected 48 hr post treatment and processed for RNA isolation, cDNA synthesis and QPCR analysis. Taqman probe Hs00166892_m1 (OTC) 60× was used for OTC expression. OTC levels were normalized to B2M expression.

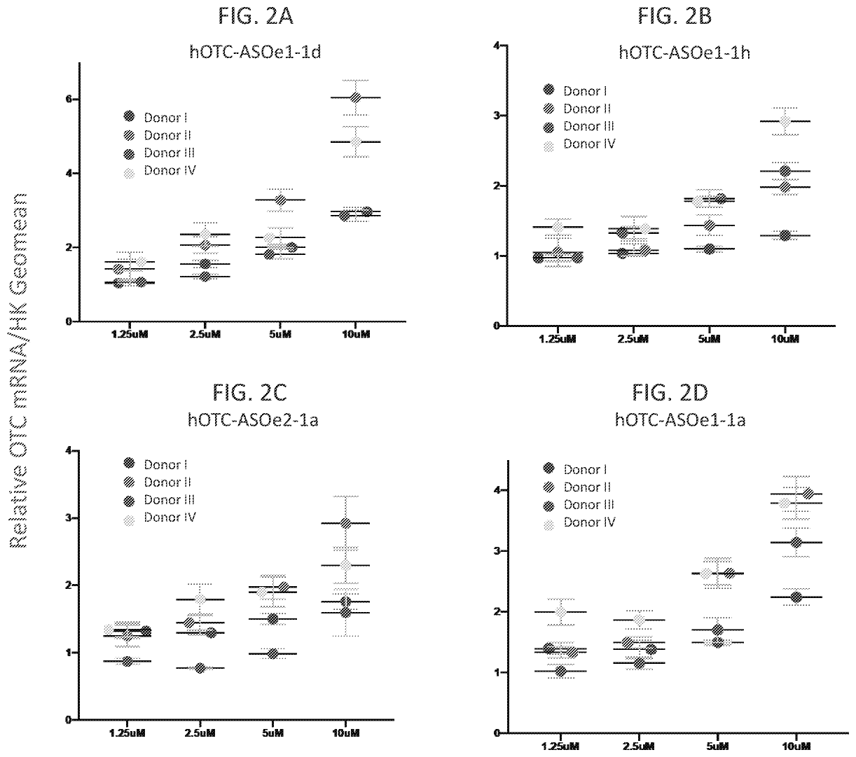

FIG. 2A shows OTC mRNA after treatment with hOTC-ASOe1-11. FIG. 2B shows OTC mRNA after treatment with hOTC-ASOe1-8. FIG. 2C shows OTC mRNA after treatment with hOTC-ASOe2-1. FIG. 2D shows OTC mRNA after treatment with hOTC-ASOe1-1. Treatment with each ASO resulted in a dose-dependent increase in OTC expression in each donor. Thus, four different RNA actuators targeting the same regRNA increased human OTC mRNA dose-dependent matter.

Hepatocytes from an OTC-deficient donor were cultured in vitro. Cells were plated in growth media and treated 4 hours post plating with a final concentration of 5 uM ASO hOTC-ASOe1-10 and hOTC-ASOe1-2c. A non-targeting control (NTC) ASO comprising a random sequence was used as the negative control. The supernatant was collected for ureagenesis analysis and cell lysate was collected for mRNA at Day 2 post treatment. For mRNA analysis, the tagman probe Hs00166892_m1 was used for OTC expression. OTC levels were normalized to B2M expression. For the Ureagenesis, the collected supernatant was measured by Urea Nitrogen (BUN) Colorimetric Detection Kit (Thermofisher, catalog #: EIABUN) and normalized by Albumin ELISA (Bethyl, Catalog #: E88-129). Statistics were performed using one way ANOVA in Prism (GraphPad).

The urea assay was also repeated in wild type hepatocytes with hOTC-ASOe1-2a in a dose study. Cells were plated in growth media and treated 4 hours post plating with a final concentration of 1.25 uM, 2.5 uM, 5 uM, and 10 uM ASO hOTC-ASOe1-2a. A non-targeting control (NTC) ASO comprising a random sequence was used as the negative control. The supernatant was collected for ureagenesis analysis and cell lysate was collected for mRNA at Day 6 post treatment. Samples were processed as described above.

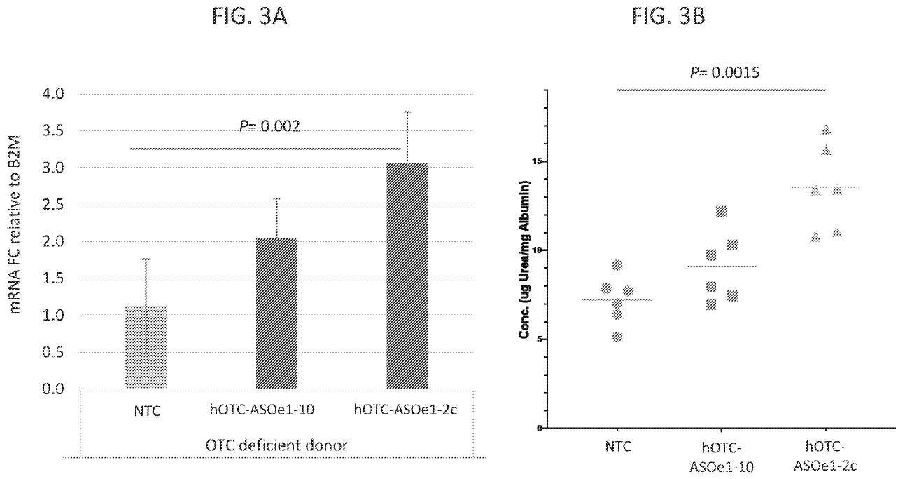

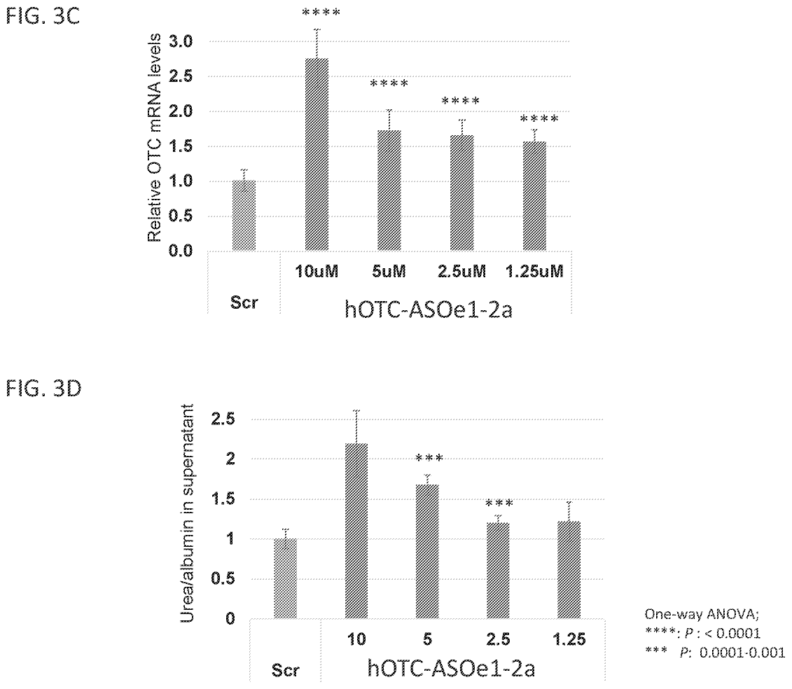

As shown in FIGS. 3A and 3B, treatment with both ASOs resulted in increased ureagenesis in patient cells (FIG. 3B), which correlated with OTC mRNA upregulation (FIG. 3A). A normal range in the ureagenesis assay is 18-30 ug urea/mg Albumin. One ASO increased the average concentration to approximately 13 ug urea/mg Albumin, almost twice as much as the 7 ug urea/mg Albumin as the negative control sample, and almost within the normal ureagenesis range. In addition, hOTC-ASOe1-2a induced a dose dependent increase in OTC mRNA (FIG. 3C) and urea (FIG. 3D) in WT hepatocytes.

The majority of regRNAs do not have large sequence areas that are conserved between human and mouse genomes. For in vivo proof of concept, regRNAs around the mouse Otc region were identified and ASOs targeting those mouse regRNA (promoter and enhancer) were designed and screened in both wildtype (B6EiC3SnF1/J, [WT]) primary mouse hepatocytes and Otc deficient donor (B6EiC3Sn a/A-Otc^{spf-ash}/J, [OTCD]) primary mouse hepatocytes.

Primary hepatocytes were isolated from male mice of mouse strains B6EiC3SnF1/J (control WT) and Otc deficient donor (B6EiC3Sn a/A-Otc^{spf-ash}/J, catalog: 001811) from JAX lab. The spf^{ash} mouse has a variant c.386G>A, p.Arg129His in the Otc gene that impacts splicing, resulting in decreased Otc mRNA levels (5-12% of wt control) in spf/ash livers. Thus, male spf^{ash} mice have a mild biochemical phenotype with low OTC activity (5%-10% of wild-type).

Primary hepatocytes were seeded at 20,000 cells per well on day 0. Cells were treated with a final concentration of 5 μM mouse ASO on day 2. Cells were incubated for 2 days and lysate was collected on Day 2 post treatment for mRNA analysis. The tagman probe Mm01288053_m1 was used for mouse OTC expression. Ppia and Hprt were used as housekeeper genes for gene expression normalization. Statistics were performed using one-way ANOVA in Prism (GraphPad).

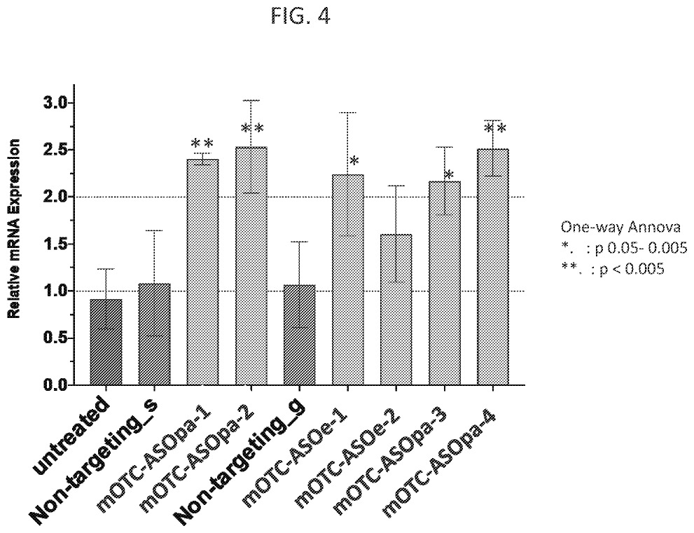

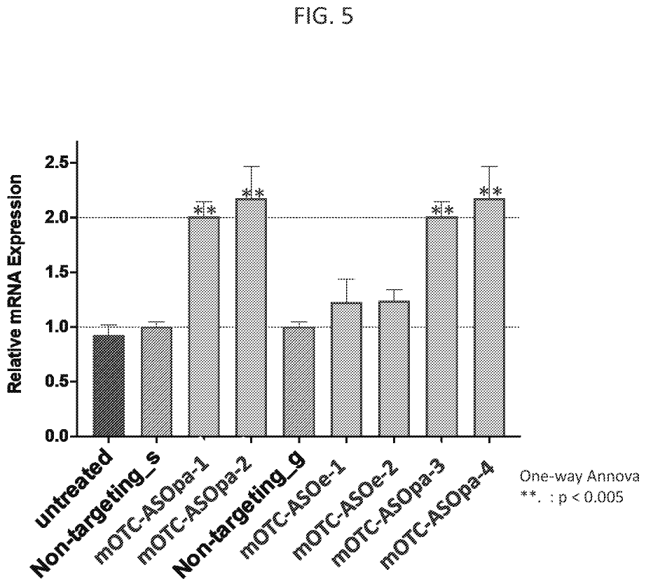

Five of the six ASOs increased Otc mRNA in WT hepatocytes in vitro, one-way ANOVA * : p 0.05-0.005; **: p<0.005. (FIG. 4). Four of the six ASOs increased Otc mRNA in OTCD hepatocytes in vitro, one-way ANOVA * : p 0.05-0.005; **: p<0.005. (FIG. 5). Thus, ASO targeting regRNA can be used to increase OTC expression in diseased mice liver cells. ASO mediated OTC upregulation in OTC deficient mouse cells allow these to be tested in a disease model, and a have an in vivo phenotypic readout.

Additional chemical modifications were made to hOTC-ASOe1-1. The modification are provided in Table 3 and FIG. 18D. The new ASOs were assessed in hepatocytes as previously described at 5 uM, 9 uM, or 10 uM concentrations. Table 5 provides the OTC mRNA fold change and standard deviation for the indicated ASO.

TABLE 5

| Name | Name | mRNA FC | mRNA SD | Dose |
|---|---|---|---|---|
| hOTC-ASOe1-1a | CO-3172 | 5.257 | 0.448 | 9 uM |
| hOTC-ASOe1-1i | CO-3721 | 3.304 | 0.520 | 5 uM |
| hOTC-ASOe1-1j | CO-3722 | 1.941 | 0.307 | 5 uM |
| hOTC-ASOe1-1k | CO-3723 | 2.448 | 0.412 | 5 uM |
| hOTC-ASOe1-1l | CO-3737 | 1.524 | 0.134 | 10 uM |
| hOTC-ASOe1-1m | CO-3738 | 1.401 | 0.091 | 10 uM |
| hOTC-ASOe1-1n | CO-3739 | 1.466 | 0.058 | 10 uM |
| hOTC-ASOe1-1g | CO-3740 | 2.286 | 0.274 | 5 uM |
| hOTC-ASOe1-1h | CO-3741 | 3.109 | 0.360 | 10 uM |
| hOTC-ASOe1-1b | CO-3777 | 1.841 | 0.238 | 5 uM |
| hOTC-ASOe1-1o | CO-3778 | 1.688 | 0.062 | 5 uM |
| hOTC-ASOe1-1p | CO-3779 | 1.508 | 0.192 | 5 uM |
| hOTC-ASOe1-1q | CO-3780 | 1.762 | 0.214 | 5 uM |
| hOTC-ASOe1-1c | CO-3781 | 2.723 | 0.280 | 10 uM |
| hOTC-ASOe1-1r | CO-3782 | 1.544 | 0.206 | 5 uM |
| hOTC-ASOe1-1d | CO-3783 | 7.262 | 0.852 | 10 uM |
| hOTC-ASOe1-1e | CO-3784 | 3.863 | 0.648 | 5 uM |
| hOTC-ASOe1-1s | CO-3796 | 1.670 | 0.194 | 5 uM |
| hOTC-ASOe1-1t | CO-3797 | 0.859 | 0.150 | 5 uM |
| hOTC-ASOe1-1u | CO-3798 | 0.828 | 0.105 | 5 uM |
| hOTC-ASOe1-1v | CO-3799 | 0.931 | 0.087 | 5 uM |
| hOTC-ASOe1-1f | CO-3800 | 2.154 | 0.223 | 10 uM |
| hOTC-ASOe1-1x | CO-4334 | 1.996 | 0.244 | 5 uM |
| hOTC-ASOe1-1y | CO-4335 | 1.683 | 0.274 | 5 uM |
| hOTC-ASOe1-1z | CO-4336 | 1.889 | 0.361 | 5 uM |
| hOTC-ASOe1-1aa | CO-4337 | 1.956 | 0.107 | 5 uM |
| hOTC-ASOe1-1ab | CO-4338 | 2.158 | 0.293 | 5 uM |
| hOTC-ASOe1-1ac | CO-4339 | 2.261 | 0.184 | 5 uM |
| hOTC-ASOe1-1ad | CO-4340 | 1.402 | 0.196 | 5 uM |
| hOTC-ASOe1-1ae | CO-4341 | 1.917 | 0.287 | 5 uM |
| hOTC-ASOe1-1af | CO-4342 | 1.603 | 0.274 | 5 uM |
| hOTC-ASOe1-1ag | CO-5049 | 1.701 | 0.793 | 5 uM |
| hOTC-ASOe1-1ah | CO-5050 | 1.879 | 0.249 | 5 uM |
| hOTC-ASOe1-1ai | CO-5055 | 4.137 | 0.754 | 10 uM |
| hOTC-ASOe1-1aj | CO-5056 | 1.503 | 0.705 | 5 uM |
| hOTC-ASOe1-1ak | CO-5057 | 1.031 | 0.174 | 1 uM |
| hOTC-ASOe1-1al | CO-5058 | 4.576 | 1.360 | 10 uM |
| hOTC-ASOe1-1am | CO-5059 | 5.781 | 1.158 | 10 uM |
| hOTC-ASOe1-1an | CO-5060 | 3.459 | 0.475 | 10 uM |
| hOTC-ASOe1-1ao | CO-5061 | 1.745 | 0.357 | 5 uM |
| hOTC-ASOe1-1aq | CO-5063 | 1.693 | 0.313 | 5 uM |

Additional chemical modifications were made to hOTC-ASOe2-2. The modification are provided in Table 4 and FIG. 18E. The new ASOs were assessed in hepatocytes as previously described at 5 uM, 9 uM, or 10 uM concentrations. Table 6 provides the OTC mRNA fold change and standard deviation for the indicated ASO.

TABLE 6

| Name | Name | mRNA FC | mRNA SD | Dose |
|---|---|---|---|---|
| hOTC-ASoe2-2a | CO-3392 | 2.976 | 0.455 | 9 uM |
| hOTC-ASOe2-2b | CO-4256 | 3.143 | 0.370 | 9 uM |
| hOTC-ASOe2-2f | CO-4257 | 2.156 | 0.167 | 5 uM |
| hOTC-ASOe2-2g | CO-4258 | 2.452 | 0.306 | 5 uM |
| hOTC-ASOe2-2c | CO-4259 | 3.506 | 0.457 | 9 uM |
| hOTC-ASOe2-2h | CO-4260 | 2.403 | 0.315 | 5 uM |
| hOTC-ASOe2-2i | CO-4261 | 1.947 | 0.253 | 5 uM |
| hOTC-ASOe2-2j | CO-4262 | 2.018 | 0.586 | 5 uM |
| hOTC-ASOe2-2k | CO-4263 | 2.301 | 0.273 | 5 uM |
| hOTC-ASOe2-2l | CO-4264 | 2.641 | 0.397 | 5 uM |
| hOTC-ASOe2-2m | CO-4265 | 1.939 | 0.285 | 5 uM |
| hOTC-ASOe2-2d | CO-4266 | 3.508 | 0.541 | 9 uM |
| hOTC-ASOe2-2e | CO-4267 | 4.770 | 0.471 | 9 uM |
| hOTC-ASOe2-2n | CO-4268 | 2.501 | 0.386 | 5 uM |
| hOTC-ASOe2-2o | CO-5048 | 1.601 | 0.111 | 5 uM |
| hOTC-ASOe2-2p | CO-5065 | 2.047 | 0.126 | 5 uM |
| hOTC-ASOe2-2q | CO-5066 | NA | NA | NA |
| hOTC-ASOe2-2r | CO-5067 | NA | NA | NA |
| hOTC-ASOe2-2s | CO-5068 | 3.649 | 0.699 | 10 uM |
| hOTC-ASOe2-2t | CO-5069 | 2.611 | 0.524 | 10 uM |
| hOTC-ASOe2-2u | CO-5070 | 2.430 | 0.304 | 5 uM |
| hOTC-ASOe2-2v | CO-5071 | 1.292 | 0.079 | 1 uM |
| hOTC-ASOe2-2w | CO-5319 | NA | NA | 9 uM |

Dose responses of two ASOs, hOTC-ASOe1-1d and hOTC-ASOe2-2e were also assessed. Cells were incubated with increasing concentrations of each ASO as described above. OCT mRNA was determined via qRT-PCR.

As shown in Table 7, treatment of hepatocytes with increasing amounts of hOTC-ASOe1-1d resulted in a dose dependent increase in OTC mRNA.

TABLE 7
hOTC-ASOe1-1d

| uM | mRNA FC | SD |
|---|---|---|
| 0.3125 | 1.4045176 | 0.37007858 |
| 0.625 | 1.60468269 | 0.2979248 |
| 1.25 | 3.22348797 | 1.14389163 |
| 2.5 | 3.40537098 | 0.92008063 |
| 5 | 3.50587612 | 1.226357401 |
| 10 | 7.26151884 | 0.852175589 |

As shown in Table 8, treatment of hepatocytes with increasing amounts of hOTC-ASOe2-2e resulted in a dose dependent increase in OTC mRNA.

TABLE 8
hOTC-ASOe2-2e

| uM | mRNA FC | SD |
|---|---|---|
| 0.3125 | 1.0517238 | 0.199258644 |
| 0.625 | 0.918363407 | 0.111768572 |
| 1.25 | 1.240839568 | 0.247210529 |
| 2.5 | 1.597027106 | 0.371885129 |
| 5 | 2.808190832 | 0.757972233 |
| 10 | 4.41848237 | 0.314668045 |

Additional ASOs were generated and tested in hepatocytes as described above. The ASO sequences, X chromosome start and end location, and the OTC mRNA fold change (FC) and standard deviation (SD) are provided in Table 9.

The ASOs of SEQ ID NOs: 143-892 target human OTC eRNA-1 (SEQ ID NO: 1). All bases are 2′-O-methoxyethyl and all cytidines have a 5-methyl (5-methyl on cytidine).

The ASOs of SEQ ID NOs: 893-1029 target human OTC eRNA-2 (SEQ ID NO: 2). The ASOs are 2′-O-methoxyethyl with LNA at bases 6, 11 and 16. Such ASOs can also be described as 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl and all cytidines have a 5-methyl (5-methyl on cytidine).

The ASOs of SEQ ID NOs: 1030-1072 target human OTC paRNA-1 (SEQ ID NO: 1077). All bases are 2′-O-methoxyethyl and all cytidines have a 5-methyl (5-methyl on cytidine).

TABLE 9

| SEQ | | | | | | X Chr | X Chr |
|---|---|---|---|---|---|---|---|
| ID | | | | Dose | | Start | End |
| NO | Name | FC | SD | (uM) | Sequence | Location | Location |
| 143 | CO-1398 | 4.142 | 0.149 | 5 uM | TTAATACAGCTCTGGAGTGG | 38342325 | 38342344 |
| 144 | CO-1399 | 1.539 | 0.146 | 5 uM | GCCTCTCACCACATGTGCCA | 38342434 | 38342453 |
| 145 | CO-1400 | 1.099 | 0.192 | 200 nM | TCACAACAAAGCTATGAGTA | 38342543 | 38342562 |
| 146 | CO-1401 | 1.105 | 0.138 | 5 nM | ACTCCTTGGTTCCCTGGAAG | 38342652 | 38342671 |
| 147 | CO-1402 | 1.100 | 0.210 | 200 nM | CAAAGCCCCATGAAAAGGGC | 38342761 | 38342780 |
| 148 | CO-1403 | 1.321 | 0.161 | 200 nM | ACCAGTGGAGCAGCCTTGAG | 38342870 | 38342889 |
| 149 | CO-1404 | 1.504 | 0.292 | 200 nM | CAAGAGCACCTCCCAAGTAG | 38342979 | 38342998 |
| 150 | CO-1405 | 1.276 | 0.104 | 5 nM | TTTTGAAGCCATCCAAGACA | 38343088 | 38343107 |
| 151 | CO-1406 | 0.953 | 0.092 | 5 nM | GAAAATGATCTCATGGGATC | 38343197 | 38343216 |
| 152 | CO-1407 | 1.155 | 0.089 | 5 nM | GATTTCCCTGTTTGCTCTGT | 38343304 | 38343323 |
| 153 | CO-1408 | 1.063 | 0.068 | 5 nM | GGCAGTTGAGATGCAAAGCA | 38343485 | 38343504 |
| 154 | CO-1409 | 1.066 | 0.140 | 5 nM | TTTTTATTTTGTGATCTGGT | 38343605 | 38343624 |
| 155 | CO-1410 | 0.844 | 0.091 | 5 nM | TCTGTTAATCATTTGCCCTG | 38343705 | 38343724 |
| 156 | CO-1411 | 1.021 | 0.211 | 5 nM | ACATTTCCATTGAAATCATT | 38343727 | 38343746 |
| 157 | CO-1412 | 1.055 | 0.136 | 200 nM | GCTAAAATTCTGAACAGAGT | 38343805 | 38343824 |
| 158 | CO-1413 | 1.041 | 0.073 | 200 nM | AAATCAGTGAGGATTATCTC | 38343905 | 38343924 |
| 159 | CO-1415 | 0.876 | 0.215 | 5 nM | ACCCAGGCTGGAATGCAGTG | 38344105 | 38344124 |
| 160 | CO-1416 | 1.059 | 0.262 | 200 nM | AAAAGAGCTCCTGATTTACA | 38344205 | 38344224 |
| 161 | CO-1417 | 1.171 | 0.181 | 200 nM | TAGTAAAGGAAATCACCCAT | 38344305 | 38344324 |
| 162 | CO-1418 | 0.984 | 0.145 | 200 nM | GTTTTATAAGCTGTAAAGCT | 38344405 | 38344424 |
| 163 | CO-1419 | 0.983 | 0.204 | 5 nM | TCCTCTATGTCCCAGGTCAT | 38344485 | 38344504 |
| 164 | CO-1420 | 0.956 | 0.076 | 5 nM | GTTGTAGCCTTACATTTTCC | 38344605 | 38344624 |
| 165 | CO-1421 | 1.045 | 0.172 | 5 nM | AAGTTCAATTTACCATTTGT | 38344705 | 38344724 |
| 166 | CO-1422 | 1.098 | 0.366 | 5 nM | ATTGGATATGTAACTTGCAA | 38344805 | 38344824 |
| 167 | CO-1423 | 1.042 | 0.397 | 200 nM | ACCCCTATTCTTCTTCTTTT | 38344905 | 38344924 |
| 168 | CO-1424 | 1.142 | 0.250 | 5 nM | CTGGTCTTGAACAGTCCTCC | 38344980 | 38344999 |
| 169 | CO-1425 | 1.164 | 0.294 | 5 nM | GCTCAAGCGATCCTCCAACC | 38345105 | 38345124 |
| 170 | CO-1426 | 0.962 | 0.170 | 200 nM | GGATTCGGTTAAGTTAGATT | 38345205 | 38345224 |
| 171 | CO-1427 | 0.994 | 0.177 | 200 nM | TCTTTGTTTAAACCATAAAC | 38345305 | 38345324 |
| 172 | CO-1428 | 1.026 | 0.070 | 200 nM | CAGGCAGGAAGGAGGTCACT | 38345345 | 38345364 |
| 173 | CO-1429 | 1.288 | 0.293 | 200 nM | TTAATACAGCTCTGGAGTGG | 38342325 | 38342344 |
| 174 | CO-1430 | 1.154 | 0.234 | 200 nM | GCCTCTCACCACATGTGCCA | 38342434 | 38342453 |
| 175 | CO-1431 | 1.134 | 0.151 | 200 nM | TCACAACAAAGCTATGAGTA | 38342543 | 38342562 |
| 176 | CO-1432 | 1.455 | 0.322 | 200 nM | ACTCCTTGGTTCCCTGGAAG | 38342652 | 38342671 |
| 177 | CO-1433 | 1.065 | 0.143 | 5 nM | CAAAGCCCCATGAAAAGGGC | 38342761 | 38342780 |
| 178 | CO-1434 | 1.176 | 0.155 | 5 nM | ACCAGTGGAGCAGCCTTGAG | 38342870 | 38342889 |
| 179 | CO-1435 | 1.222 | 0.178 | 5 nM | CAAGAGCACCTCCCAAGTAG | 38342979 | 38342998 |
| 180 | CO-1436 | 1.144 | 0.181 | 5 nM | TTTTGAAGCCATCCAAGACA | 38343088 | 38343107 |
| 181 | CO-1437 | 2.673 | 0.381 | 10 uM | GAAAATGATCTCATGGGATC | 38343197 | 38343216 |
| 182 | CO-1438 | 1.046 | 0.251 | 5 nM | GATTTCCCTGTTTGCTCTGT | 38343304 | 38343323 |
| 183 | CO-1439 | 1.251 | 0.162 | 200 nM | GGCAGTTGAGATGCAAAGCA | 38343485 | 38343504 |
| 184 | CO-1440 | 0.963 | 0.172 | 5 nM | TTTTTATTTTGTGATCTGGT | 38343605 | 38343624 |
| 185 | CO-1441 | 0.970 | 0.104 | 5 nM | TCTGTTAATCATTTGCCCTG | 38343705 | 38343724 |
| 186 | CO-1442 | 1.277 | 0.226 | 5 nM | ACATTTCCATTGAAATCATT | 38343727 | 38343746 |
| 187 | CO-1443 | 1.154 | 0.253 | 200 nM | GCTAAAATTCTGAACAGAGT | 38343805 | 38343824 |
| 188 | CO-1444 | 1.088 | 0.202 | 200 nM | AAATCAGTGAGGATTATCTC | 38343905 | 38343924 |
| 189 | CO-1445 | 0.996 | 0.161 | 200 nM | TTAGACAAACTACCATTACC | 38344005 | 38344024 |
| 190 | CO-1446 | 0.987 | 0.215 | 200 nM | ACCCAGGCTGGAATGCAGTG | 38344105 | 38344124 |
| 191 | CO-1447 | 1.103 | 0.164 | 200 nM | AAAAGAGCTCCTGATTTACA | 38344205 | 38344224 |
| 192 | CO-1448 | 1.145 | 0.184 | 5 nM | TAGTAAAGGAAATCACCCAT | 38344305 | 38344324 |
| 193 | CO-1449 | 1.186 | 0.141 | 5 nM | GTTTTATAAGCTGTAAAGCT | 38344405 | 38344424 |
| 194 | CO-1450 | 1.334 | 0.175 | 5 nM | TCCTCTATGTCCCAGGTCAT | 38344485 | 38344504 |
| 195 | CO-1451 | 1.196 | 0.198 | 5 nM | GTTGTAGCCTTACATTTTCC | 38344605 | 38344624 |
| 196 | CO-1452 | 1.247 | 0.174 | 200 nM | AAGTTCAATTTACCATTTGT | 38344705 | 38344724 |
| 197 | CO-1453 | 1.137 | 0.183 | 200 nM | ATTGGATATGTAACTTGCAA | 38344805 | 38344824 |
| 198 | CO-1454 | 1.100 | 0.194 | 200 nM | ACCCCTATTCTTCTTCTTTT | 38344905 | 38344924 |
| 199 | CO-1455 | 1.021 | 0.103 | 200 nM | CTGGTCTTGAACAGTCCTCC | 38344980 | 38344999 |
| 200 | CO-1456 | 1.030 | 0.207 | 200 nM | GCTCAAGCGATCCTCCAACC | 38345105 | 38345124 |
| 201 | CO-1457 | 1.178 | 0.222 | 200 nM | GGATTCGGTTAAGTTAGATT | 38345205 | 38345224 |
| 202 | CO-1458 | 1.021 | 0.132 | 200 nM | TCTTTGTTTAAACCATAAAC | 38345305 | 38345324 |
| 203 | CO-1459 | 0.954 | 0.141 | 200 nM | CAGGCAGGAAGGAGGTCACT | 38345345 | 38345364 |
| 204 | CO-1823 | 1.098 | 0.107 | 200 nM | TTGAACCTCTGTGATTTCCCT | 38343292 | 38343311 |
| 205 | CO-1824 | 0.893 | 0.057 | 200 nM | ATTGTTGAGACACTGGTGAAC | 38343270 | 38343289 |
| 206 | CO-1825 | 0.874 | 0.131 | 200 nM | GTAGAAATGGGCAGAGAAGGC | 38343050 | 38343069 |
| 207 | CO-1826 | 1.452 | 0.305 | 200 nM | AATCAATAGCAGGTCAAGAGC | 38342965 | 38342984 |
| 208 | CO-1827 | 0.967 | 0.152 | 200 nM | GTAGATATAGCCCAATAAAGC | 38342944 | 38342963 |
| 209 | CO-1828 | 1.904 | 0.289 | 200 nM | TCATTAGCTTAAGGTGAGGGC | 38342909 | 38342928 |
| 210 | CO-1829 | 2.912 | 0.191 | 200 nM | GAATATGTAGCACCTATGACC | 38342722 | 38342741 |
| 211 | CO-1830 | 2.917 | 0.501 | 200 nM | GTACTGCTTGCCAGATGATTC | 38342573 | 38342592 |
| 212 | CO-1831 | 3.081 | 0.532 | 200 nM | ACACTGAGCTAGGTACTGTGC | 38342488 | 38342507 |
| 213 | CO-1832 | 3.906 | 0.716 | 200 nM | AGAATAAGCCAAGAACTGAGC | 38342403 | 38342422 |
| 214 | CO-1833 | 4.323 | 1.048 | 10 | AATACAGCTCTGGAGTGGGGT | 38342327 | 38342346 |
| 215 | CO-1868 | 2.088 | 0.449 | 200 nM | TTGAACCTCTGTGATTTCCCT | 38343292 | 38343311 |
| 216 | CO-1869 | 1.567 | 0.251 | 200 nM | ATTGTTGAGACACTGGTGAAC | 38343270 | 38343289 |
| 217 | CO-1870 | 1.214 | 0.193 | 200 nM | GTAGAAATGGGCAGAGAAGGC | 38343050 | 38343069 |
| 218 | CO-1871 | 1.103 | 0.258 | 200 nM | AATCAATAGCAGGTCAAGAGC | 38342965 | 38342984 |
| 219 | CO-1872 | 1.153 | 0.285 | 200 nM | GTAGATATAGCCCAATAAAGC | 38342944 | 38342963 |
| 220 | CO-1873 | 1.815 | 0.358 | 200 nM | TCATTAGCTTAAGGTGAGGGC | 38342909 | 38342928 |
| 221 | CO-1874 | 2.604 | 0.442 | 200 nM | GAATATGTAGCACCTATGACC | 38342722 | 38342741 |
| 222 | CO-1875 | 2.177 | 1.366 | 200 nM | GTACTGCTTGCCAGATGATTC | 38342573 | 38342592 |
| 223 | CO-1876 | 3.243 | 0.269 | 200 nM | ACACTGAGCTAGGTACTGTGC | 38342488 | 38342507 |
| 224 | CO-1877 | 3.229 | 0.245 | 200 nM | AGAATAAGCCAAGAACTGAGC | 38342403 | 38342422 |
| 225 | CO-1878 | 2.859 | 0.099 | 200 nM | AATACAGCTCTGGAGTGGGGT | 38342327 | 38342346 |
| 226 | CO-1879 | 3.284 | 0.403 | 200 nM | TCTAACCTTCAAGCTGTCCTT | 38345233 | 38345253 |
| 227 | CO-1880 | 4.799 | 0.801 | 200 nM | ACTTAACCGAATCCATCTTGC | 38345211 | 38345230 |
| 228 | CO-1881 | 2.966 | 0.504 | 200 nM | TCTAACTTAACCGAATCCATC | 38345207 | 38345226 |
| 229 | CO-1882 | 1.195 | 0.203 | 200 nM | AATTTAGTTTGGTGTGGTGGC | 38345050 | 38345069 |
| 230 | CO-1883 | 1.968 | 0.350 | 200 nM | TCTAAGGCTGGAGGACTGTTC | 38344971 | 38344990 |
| 231 | CO-1884 | 2.817 | 0.450 | 200 nM | AGCTAATGGGTGATTTCCTTT | 38344300 | 38344319 |
| 232 | CO-1885 | 1.638 | 0.284 | 200 nM | ACTCTCAGATCGCTTGAACCC | 38344050 | 38344069 |
| 233 | CO-1886 | 2.333 | 0.265 | 200 nM | AACTACCATTACCATTACCAT | 38344012 | 38344031 |
| 234 | CO-1887 | 4.070 | 0.936 | 200 nM | ATAAATACCTAGTGGTGATGT | 38343955 | 38343974 |
| 235 | CO-1888 | 1.515 | 0.108 | 200 nM | TTATGAAGGACCCTGTCTACC | 38343827 | 38343846 |
| 236 | CO-1889 | 4.995 | 1.122 | 10 | TACTTGAATTTGATGATTGGC | 38343777 | 38343796 |
| 237 | CO-1890 | 1.083 | 0.231 | 200 nM | TCTATATGACAAGAGAGAAGC | 38343749 | 38343768 |
| 238 | CO-1891 | 1.135 | 0.180 | 200 nM | AGTTTATTGATCTGGTGGTGC | 38345417 | 38345437 |
| 239 | CO-1892 | 1.121 | 0.340 | 200 nM | AAGAGTTCAACAGCATGATCC | 38344673 | 38344692 |
| 240 | CO-1893 | 2.973 | 0.713 | 200 nM | ACTCAATCTCAACCTCAAGCC | 38344549 | 38344568 |
| 241 | CO-2207 | 1.626 | 0.167 | 5 uM | TAATGACCCAACCTTGTGTC | 38343508 | 38343527 |
| 242 | CO-2208 | 1.526 | 0.222 | 5 uM | GATTAGGAAATGCACAACAC | 38343564 | 38343583 |
| 243 | CO-2209 | 1.535 | 0.353 | 5 uM | CAAGTTTCCATACCTGGTTC | 38343625 | 38343644 |
| 244 | CO-2210 | 1.445 | 0.254 | 5 uM | TTTTGAGCTTAGATATGGAC | 38343653 | 38343672 |
| 245 | CO-2211 | 1.387 | 0.150 | 5 uM | GATTTTAAGCAGAATCCAGA | 38343676 | 38343695 |
| 246 | CO-2212 | 1.358 | 0.124 | 5 uM | CAAGTATAATCTCGCTTCTC | 38343763 | 38343782 |
| 247 | CO-2213 | 1.812 | 0.384 | 5 uM | GGTAGACAGGGTCCTTCATA | 38343828 | 38343847 |
| 248 | CO-2214 | 1.486 | 0.412 | 5 uM | CTGCCATACCCTTTCAATTG | 38343862 | 38343881 |
| 249 | CO-2215 | 1.213 | 0.030 | 5 uM | CTTGCTACATCACCACTAGG | 38343962 | 38343981 |
| 250 | CO-2216 | 1.164 | 0.185 | 5 uM | GGTAATGGTAGTTTGTCTAA | 38344005 | 38344024 |
| 251 | CO-2217 | 1.307 | 0.097 | 5 uM | GCGATCTGAGAGTTACTTTC | 38344043 | 38344062 |
| 252 | CO-2218 | 2.003 | 0.466 | 10 | TTTTTTCTCTCCACGTGTGT | 38344273 | 38344292 |
| 253 | CO-2219 | 1.155 | 0.162 | 5 uM | GTGTGGAAACTGGCAATAAG | 38344344 | 38344363 |
| 254 | CO-2220 | 1.741 | 0.383 | 10 | TATTGTTTTGCGGCTTGGAC | 38344366 | 38344385 |
| 255 | CO-2221 | 1.002 | 0.260 | 5 uM | TCTAACGTGCTGAAGGACCC | 38344464 | 38344483 |
| 256 | CO-2222 | 1.196 | 0.126 | 5 uM | GTCTAAGGCTTGAGGTTGAG | 38344556 | 38344575 |
| 257 | CO-2223 | 1.172 | 0.198 | 5 uM | TTCAGATCTGTGATCCACTG | 38344584 | 38344603 |
| 258 | CO-2224 | 1.286 | 0.245 | 5 uM | AAGATTCTCTCCCTATGTCT | 38344634 | 38344653 |
| 259 | CO-2225 | 1.205 | 0.110 | 5 uM | GTTGAACTCTTTGCATAACC | 38344664 | 38344683 |
| 260 | CO-2226 | 1.468 | 0.178 | 5 uM | CTCCTGACTATGTTTTTCAC | 38344750 | 38344769 |
| 261 | CO-2227 | 1.383 | 0.090 | 5 uM | GTGTTTTCCCAGTCTGTTGC | 38344783 | 38344802 |
| 262 | CO-2228 | 1.437 | 0.349 | 5 uM | AAAGGCGGTTTCACAATAGT | 38345159 | 38345178 |
| 263 | CO-2229 | 1.106 | 0.223 | 5 uM | CATCTGATCTTGTGGCTAAT | 38345393 | 38345412 |
| 264 | CO-2230 | 1.114 | 0.075 | 5 uM | GGCACCACCAGATCAATAAA | 38345419 | 38345438 |
| 265 | CO-2231 | 1.945 | 0.256 | 10 | GGGTATCAGTACTGCACTTG | 38345450 | 38345469 |
| 266 | CO-2232 | 1.074 | 0.161 | 5 uM | TAATGACCCAACCTTGTGTC | 38343508 | 38343527 |
| 267 | CO-2233 | 0.946 | 0.077 | 5 uM | GATTAGGAAATGCACAACAC | 38343564 | 38343583 |
| 268 | CO-2234 | 1.838 | 0.208 | 10 | CAAGTTTCCATACCTGGTTC | 38343625 | 38343644 |
| 269 | CO-2235 | 1.446 | 0.320 | 5 uM | TTTTGAGCTTAGATATGGAC | 38343653 | 38343672 |
| 270 | CO-2236 | 1.044 | 0.074 | 5 uM | GATTTTAAGCAGAATCCAGA | 38343676 | 38343695 |
| 271 | CO-2237 | 1.068 | 0.152 | 5 uM | CAAGTATAATCTCGCTTCTC | 38343763 | 38343782 |
| 272 | CO-2238 | 1.051 | 0.052 | 5 uM | GGTAGACAGGGTCCTTCATA | 38343828 | 38343847 |
| 273 | CO-2239 | 1.008 | 0.159 | 5 uM | CTGCCATACCCTTTCAATTG | 38343862 | 38343881 |
| 274 | CO-2240 | 0.927 | 0.229 | 5 uM | CTTGCTACATCACCACTAGG | 38343962 | 38343981 |
| 275 | CO-2241 | 1.397 | 0.149 | 5 uM | GGTAATGGTAGTTTGTCTAA | 38344005 | 38344024 |
| 276 | CO-2242 | 1.066 | 0.199 | 5 uM | GCGATCTGAGAGTTACTTTC | 38344043 | 38344062 |
| 277 | CO-2243 | 1.290 | 0.208 | 5 uM | TTTTTTCTCTCCACGTGTGT | 38344273 | 38344292 |
| 278 | CO-2244 | 1.206 | 0.317 | 5 uM | GTGTGGAAACTGGCAATAAG | 38344344 | 38344363 |
| 279 | CO-2245 | 1.168 | 0.277 | 5 uM | TATTGTTTTGCGGCTTGGAC | 38344366 | 38344385 |
| 280 | CO-2246 | 1.266 | 0.242 | 5 uM | TCTAACGTGCTGAAGGACCC | 38344464 | 38344483 |
| 281 | CO-2247 | 1.256 | 0.398 | 5 uM | GTCTAAGGCTTGAGGTTGAG | 38344556 | 38344575 |
| 282 | CO-2248 | 1.004 | 0.258 | 5 uM | TTCAGATCTGTGATCCACTG | 38344584 | 38344603 |
| 283 | CO-2249 | 0.978 | 0.096 | 5 uM | AAGATTCTCTCCCTATGTCT | 38344634 | 38344653 |
| 284 | CO-2250 | 1.058 | 0.117 | 5 uM | GTTGAACTCTTTGCATAACC | 38344664 | 38344683 |
| 285 | CO-2251 | 3.341 | 0.273 | 5 uM | CTCCTGACTATGTTTTTCAC | 38344750 | 38344769 |
| 286 | CO-2252 | 0.924 | 0.040 | 5 uM | GTGTTTTCCCAGTCTGTTGC | 38344783 | 38344802 |
| 287 | CO-2253 | 2.384 | 0.262 | 10 uM | AAAGGCGGTTTCACAATAGT | 38345159 | 38345178 |
| 288 | CO-2254 | 1.031 | 0.167 | 5 uM | CATCTGATCTTGTGGCTAAT | 38345393 | 38345412 |
| 289 | CO-2255 | 0.950 | 0.064 | 5 uM | GGCACCACCAGATCAATAAA | 38345419 | 38345438 |
| 290 | CO-2256 | 3.041 | 0.374 | 10 uM | GGGTATCAGTACTGCACTTG | 38345450 | 38345469 |
| 291 | CO-2257 | 0.957 | 0.079 | 5 uM | TAGTCACAGGGAGCATTAGG | 38343246 | 38343265 |
| 292 | CO-2258 | 1.211 | 0.145 | 5 uM | GACAGTGCTCCTAGACCAAA | 38343156 | 38343175 |
| 293 | CO-2259 | 1.151 | 0.119 | 5 uM | GAGGCACCAACTACAAAGAT | 38343009 | 38343028 |
| 294 | CO-2260 | 1.191 | 0.188 | 5 uM | ATAGGTCCCATCTTTACAGG | 38342837 | 38342856 |
| 295 | CO-2261 | 1.066 | 0.110 | 5 uM | AGGATTCCCATGGTCTATCT | 38342805 | 38342824 |
| 296 | CO-2262 | 1.072 | 0.105 | 5 uM | GCATAACAATGAAGGTGACC | 38342619 | 38342638 |
| 297 | CO-2263 | 1.353 | 0.450 | 5 uM | CGCTTACTTCTTAATGGTAA | 38342593 | 38342612 |
| 298 | CO-2264 | 1.136 | 0.220 | 5 uM | CTACAGTACTCTCTATTCAT | 38342508 | 38342527 |
| 299 | CO-2265 | 1.152 | 0.049 | 5 uM | GGTAGTAGTTAACAAAAGCT | 38342456 | 38342475 |
| 300 | CO-2266 | 1.040 | 0.204 | 5 uM | CGGACACCTCAACACTTTTA | 38342366 | 38342385 |
| 301 | CO-2267 | 0.997 | 0.219 | 5 uM | TAGTCACAGGGAGCATTAGG | 38343246 | 38343265 |
| 302 | CO-2268 | 0.864 | 0.135 | 5 uM | GACAGTGCTCCTAGACCAAA | 38343156 | 38343175 |
| 303 | CO-2269 | 1.137 | 0.108 | 5 uM | GAGGCACCAACTACAAAGAT | 38343009 | 38343028 |
| 304 | CO-2270 | 0.937 | 0.066 | 5 uM | ATAGGTCCCATCTTTACAGG | 38342837 | 38342856 |
| 305 | CO-2271 | 0.957 | 0.040 | 5 uM | AGGATTCCCATGGTCTATCT | 38342805 | 38342824 |
| 306 | CO-2272 | 1.024 | 0.039 | 5 uM | GCATAACAATGAAGGTGACC | 38342619 | 38342638 |
| 307 | CO-2273 | 2.975 | 0.586 | 10 uM | CGCTTACTTCTTAATGGTAA | 38342593 | 38342612 |
| 308 | CO-2274 | 4.308 | 1.626 | 5 uM | CTACAGTACTCTCTATTCAT | 38342508 | 38342527 |
| 309 | CO-2275 | 3.661 | 0.446 | 5 uM | GGTAGTAGTTAACAAAAGCT | 38342456 | 38342475 |
| 310 | CO-2276 | 1.300 | 0.101 | 5 uM | CGGACACCTCAACACTTTTA | 38342366 | 38342385 |
| 311 | CO-2277 | 3.020 | | 5 | TAATACAGCTCTGGAGTG | 38342326 | 38342345 |
| 312 | CO-2278 | 1.980 | | 5 | AATACAGCTCTGGAGT | 38342327 | 38342346 |
| 313 | CO-2280 | 1.590 | | 1 | CTCTCACCACATGTGC | 38342436 | 38342456 |
| 314 | CO-2480 | 1.366 | 0.291 | 10 uM | GAAAATGATCTCATGGGATC | 38343197 | 38343216 |
| 315 | CO-2499 | 5.114 | 0.383 | 2.5 uM | AATACAGCTCTGGAGTGGGGT | 38342327 | 38342346 |
| 316 | CO-2500 | 3.126 | 0.247 | 5 uM | AATACAGCTCTGGAGTGGGGT | 38342327 | 38342346 |
| 317 | CO-2501 | 1.193 | 0.089 | 10 uM | AGAAAGACGGACACCTCAAC | 38342359 | 38342378 |
| 318 | CO-2502 | 1.397 | 0.182 | 2. uM | GGTATGAAAGAAAGACGGAC | 38342351 | 38342370 |
| 319 | CO-2503 | 1.380 | 0.196 | 2. uM | CTTTTAATACAGCTCTGGAG | 38342322 | 38342341 |
| 320 | CO-2504 | 1.270 | 0.141 | 2. uM | TTCACTTTTAATACAGCTCT | 38342318 | 38342337 |
| 321 | CO-2505 | 1.365 | 0.291 | 5 uM | TGCCCAGCCTGAATTTCACT | 38342304 | 38342323 |
| 322 | CO-2506 | 1.104 | 0.103 | 1.25 uM | AGAAAGACGGACACCTCAAC | 38342359 | 38342378 |
| 323 | CO-2507 | 1.162 | 0.127 | 10 uM | GGTATGAAAGAAAGACGGAC | 38342351 | 38342370 |
| 324 | CO-2508 | 1.107 | 0.112 | 10 uM | CTTTTAATACAGCTCTGGAG | 38342322 | 38342341 |
| 325 | CO-2509 | 1.248 | 0.317 | 1.25 uM | TTCACTTTTAATACAGCTCT | 38342318 | 38342337 |
| 326 | CO-2510 | 1.096 | 0.143 | 10 uM | TGCCCAGCCTGAATTTCACT | 38342304 | 38342323 |
| 327 | CO-2511 | 1.317 | 0.192 | 5 uM | TGTAATCAGATTTCACCGTG | 38352941 | 38352961 |
| 328 | CO-2512 | 1.317 | 0.416 | 5 uM | CTCAGTCCTTCTGTAATCAG | 38352930 | 38352949 |
| 329 | CO-2513 | 1.192 | 0.238 | 5 uM | AAGCTTTCATTTCTCAGTCC | 38352918 | 38352937 |
| 330 | CO-2514 | 1.252 | 0.300 | 5 uM | CTCTAATTCCAGCAAAGCTT | 38352904 | 38352923 |
| 331 | CO-2515 | 1.075 | 0.133 | 5 uM | TCTTATTTGAGACAGCTGCC | 38352885 | 38352904 |
| 332 | CO-2516 | 1.114 | 0.131 | 5 uM | GTGCCACGCTCTGCTTTACT | 38352866 | 38352885 |
| 333 | CO-2517 | 1.040 | 0.189 | 5 uM | TGCAGAATGTAGTGCCACGC | 38352855 | 38352874 |
| 334 | CO-2518 | 2.258 | 0.191 | 5 | GGCCTCTTTCTGCAGAATGT | 38352845 | 38352864 |
| 335 | CO-2519 | 1.351 | 0.070 | 5 uM | ACTATATTCTGCAGTAAGGC | 38352828 | 38352847 |
| 336 | CO-2520 | 1.250 | 0.292 | 5 uM | GGAATCATGGTGATGCATAA | 38352807 | 38352826 |
| 337 | CO-2521 | 1.266 | 0.426 | 5 uM | GAGACTTGGGTTTGATTTAG | 38352788 | 38352807 |
| 338 | CO-2522 | 1.222 | 0.197 | 5 uM | GTGATGGTCAGAGACTTGGG | 38352778 | 38352797 |
| 339 | CO-2523 | 1.297 | 0.255 | 5 uM | ATTTTCGGTAAGTGATGGTC | 38352767 | 38352786 |
| 340 | CO-2524 | 1.243 | 0.226 | 5 uM | GTCACAACTTCATGGTTCGA | 38352746 | 38352765 |
| 341 | CO-2525 | 1.206 | 0.515 | 5 uM | TGCAGCTTTTAGAAATGGTC | 38352729 | 38352748 |
| 342 | CO-2526 | 1.278 | 0.334 | 5 uM | TGAGGATCCTGTTAAACAAT | 38352710 | 38352729 |
| 343 | CO-2527 | 1.489 | 0.470 | 5 uM | GCTGTTTAATCTGAGGATCC | 38352699 | 38352719 |
| 344 | CO-2528 | 1.114 | 0.321 | 5 uM | ACACAATTAAAAGAAGATGC | 38352681 | 38352700 |
| 345 | CO-2529 | 1.365 | 0.480 | 5 uM | CATAGAATCGTCCTTTACAC | 38352665 | 38352684 |
| 346 | CO-2530 | 1.265 | 0.212 | 5 uM | GGCTAACTTGCTGTGGAGTT | 38352637 | 38352656 |
| 347 | CO-2531 | 1.426 | 0.576 | 5 uM | TAGTTTTTAGGTGGCCCCCG | 38352615 | 38352634 |
| 348 | CO-2532 | 1.206 | 0.503 | 5 uM | GCAACTGAACACATTTCTTA | 38352597 | 38352616 |
| 349 | CO-2533 | 1.155 | 0.310 | 5 uM | CTCCCTCACTGCAACTGAAC | 38352587 | 38352606 |
| 350 | CO-2534 | 1.478 | 0.539 | 5 uM | GATATTACCTTTGCTCCCTC | 38352574 | 38352593 |
| 351 | CO-2535 | 1.312 | 0.483 | 5 uM | AGCTGAAGGGTGATATTACC | 38352563 | 38352582 |
| 352 | CO-2536 | 1.345 | 0.396 | 5 uM | GGGACTTTGATAAGGAAGCT | 38352547 | 38352566 |
| 353 | CO-2537 | 1.312 | 0.340 | 5 uM | GCCCTGCAGTATCTCTAACC | 38352525 | 38352544 |
| 354 | CO-2538 | 1.344 | 0.660 | 5 uM | AAATGCTCCTACACCCTGCC | 38352508 | 38352527 |
| 355 | CO-2539 | 1.187 | 0.279 | 5 uM | GCATAAAGTTCAAATGCTCC | 38352497 | 38352516 |
| 356 | CO-2540 | 1.385 | 0.297 | 5 uM | CGGTGGAGCTTGGCATAAAG | 38352485 | 38352504 |
| 357 | CO-2541 | 1.453 | 0.371 | 5 uM | CCAAAAAAAATACACAGCGG | 38352468 | 38352487 |
| 358 | CO-2542 | 1.300 | 0.384 | 5 uM | AAAAGAGTCAGGATTTCTTC | 38352449 | 38352468 |
| 359 | CO-2543 | 1.378 | 0.510 | 5 uM | GGCCAGGCAATAAAAGAGTC | 38352438 | 38352458 |
| 360 | CO-2544 | 1.423 | 0.479 | 5 uM | AAAATGAGGAGGCCAGGCAA | 38352428 | 38352447 |
| 361 | CO-2545 | 1.389 | 0.729 | 5 uM | GCGTGTGACAGTATAAATAT | 38352401 | 38352420 |
| 362 | CO-2546 | 1.400 | 0.471 | 5 uM | GATTTTGTACATGCGTGTGA | 38352389 | 38352408 |
| 363 | CO-2547 | 1.250 | 0.185 | 5 uM | TGAGTACTTGTCAATTGATT | 38352373 | 38352392 |
| 364 | CO-2548 | 1.320 | 0.190 | 5 uM | CACCTTCACAGCAGCCGGTA | 38352322 | 38352341 |
| 365 | CO-2549 | 1.408 | 0.483 | 5 uM | GTTACTTCTACTCACCTTCA | 38352310 | 38352329 |
| 366 | CO-2550 | 1.358 | 0.462 | 5 uM | TTCTGAGTGAGTTACTTCTA | 38352300 | 38352319 |
| 367 | CO-2551 | 1.397 | 0.521 | 5 uM | GCCAATTTGCATTTTCTGAG | 38352287 | 38352306 |
| 368 | CO-2552 | 1.254 | 0.332 | 5 uM | TTTTTGCAGTGCCAATTTGC | 38352277 | 38352296 |
| 369 | CO-2553 | 1.398 | 0.441 | 5 uM | CTACATCTGTGTTTTTGCAG | 38352266 | 38352285 |
| 370 | CO-2554 | 1.352 | 0.119 | 5 uM | ACTGTCACATCTACATCTGT | 38352256 | 38352275 |
| 371 | CO-2555 | 1.291 | 0.343 | 5 uM | GTACTACTCAGGACTGTCAC | 38352244 | 38352263 |
| 372 | CO-2556 | 1.438 | 0.382 | 5 uM | AAATGGAAATAGTACTACTC | 38352233 | 38352252 |
| 373 | CO-2557 | 1.226 | 0.267 | 5 uM | GACTGGCAATTAGAGGTAGA | 38352203 | 38352222 |
| 374 | CO-2558 | 1.209 | 0.116 | 5 uM | TTTTGTGGAAAGACTGGCAA | 38352192 | 38352211 |
| 375 | CO-2559 | 1.150 | 0.419 | 5 uM | TGCACCCCCAAAATAGCTTC | 38352150 | 38352170 |
| 376 | CO-2560 | 1.237 | 0.450 | 5 uM | CTCTATCATCTTGCACCCCC | 38352139 | 38352158 |
| 377 | CO-2561 | 1.426 | 0.275 | 5 uM | TACTATACCTTCTCTATCAT | 38352128 | 38352147 |
| 378 | CO-2562 | 1.554 | 0.332 | 5 uM | GGAGCTCCAGGACTGAGATA | 38352104 | 38352123 |
| 379 | CO-2563 | 1.342 | 0.532 | 5 uM | CAAGGCACAAAGGGAGCTCC | 38352092 | 38352111 |
| 380 | CO-2564 | 1.343 | 0.537 | 5 uM | TTGAAGACTTATGTGCAAGG | 38352077 | 38352096 |
| 381 | CO-2565 | 1.457 | 0.430 | 5 uM | CGGCCAGCAATTATTTCTTT | 38352056 | 38352075 |
| 382 | CO-2566 | 1.250 | 0.403 | 5 uM | GGTGCTGTTAGAATCAATAG | 38351717 | 38351736 |
| 383 | CO-2567 | 1.232 | 0.334 | 5 uM | CTGACTGTCAGGTGCTGTTA | 38351707 | 38351726 |
| 384 | CO-2568 | 1.458 | 0.368 | 5 uM | CCCACTTGTACTGACTGTCA | 38351697 | 38351716 |
| 385 | CO-2569 | 1.432 | 0.196 | 5 uM | GTATCCCCACTTCCCACTTG | 38351685 | 38351704 |
| 386 | CO-2570 | 1.288 | 0.305 | 5 uM | GAAGAGCATATGGTATCCCC | 38351673 | 38351692 |
| 387 | CO-2571 | 1.338 | 0.499 | 5 uM | GATCTGAATAGGCTGCTAGG | 38351652 | 38351671 |
| 388 | CO-2572 | 1.324 | 0.297 | 5 uM | ATGGCCCCTTAGTGATCTGA | 38351639 | 38351658 |
| 389 | CO-2573 | 1.312 | 0.307 | 5 uM | GTGAAGTTGCAGATGGCCCC | 38351627 | 38351646 |
| 390 | CO-2574 | 1.270 | 0.402 | 5 uM | ACCTGATTTCTAACTGAGGA | 38351594 | 38351613 |
| 391 | CO-2575 | 1.307 | 0.355 | 5 uM | AGAATTGACTTTGAATCACC | 38351577 | 38351597 |
| 392 | CO-2576 | 1.303 | 0.197 | 5 uM | GCAGCTCGGTATCTGATACA | 38351558 | 38351577 |
| 393 | CO-2577 | 1.261 | 0.540 | 5 uM | TGAAACTTCAGTGCAGCTCG | 38351546 | 38351565 |
| 394 | CO-2579 | 1.205 | 0.224 | 5 uM | GAACTCTGCAAAGATGATGT | 38351513 | 38351532 |
| 395 | CO-2580 | 1.146 | 0.188 | 5 uM | TACTGAACTGTGAACTCTGC | 38351502 | 38351521 |
| 396 | CO-2581 | 1.346 | 0.345 | 5 uM | CATACCACCTTTACTGAACT | 38351491 | 38351510 |
| 397 | CO-2582 | 1.377 | 0.234 | 5 uM | GTTTATAAATCATACCACCT | 38351481 | 38351500 |
| 398 | CO-2583 | 1.269 | 0.178 | 5 uM | GGCTTGTATTATGGATATTT | 38351461 | 38351480 |
| 399 | CO-2584 | 1.197 | 0.372 | 5 uM | CAACTTTGTCTGGACCTCTC | 38351436 | 38351455 |
| 400 | CO-2585 | 1.229 | 0.356 | 5 uM | TCTTCGAAGCCAGACAACTT | 38351422 | 38351441 |
| 401 | CO-2586 | 1.091 | 0.090 | 5 uM | CTACCCTTCCTACCTTAGAG | 38351392 | 38351411 |
| 402 | CO-2587 | 1.320 | 0.542 | 5 uM | ACCAGAAGAGGCAGCCCTAC | 38351349 | 38351368 |
| 403 | CO-2588 | 0.604 | 0.027 | 10 uM | CTGGGAGTGTCTGGTAGTCA | 38343232 | 38343251 |
| 404 | CO-2589 | 0.864 | 0.079 | 10 uM | GGATCCCCATCACTCAGGTC | 38343212 | 38343231 |
| 405 | CO-2590 | 1.193 | 0.114 | 10 uM | TGAAAATGATCTCATGGGAT | 38343196 | 38343215 |
| 406 | CO-2591 | 1.009 | 0.179 | 10 uM | CAATGAAAATGATCTCATGG | 38343193 | 38343213 |
| 407 | CO-2592 | 1.057 | 0.064 | 10 uM | TGGTCAGTAGAAAGCAATGA | 38343179 | 38343198 |
| 408 | CO-2593 | 1.043 | 0.083 | 10 uM | CCAAACCCTGGTCAGTAGAA | 38343171 | 38343190 |
| 409 | CO-2598 | 1.361 | 0.200 | 5 uM | TTAATACAGCTCTGGAGTGG | 38342325 | 38342344 |
| 410 | CO-2599 | 1.707 | 0.187 | 10 | AATACAGCTCTGGAGTGGGGT | 38342327 | 38342346 |
| 411 | CO-2773 | 4.217 | 0.327 | 5 | AATACAGCTCTGGAGTGGGG | 38342327 | 38342346 |
| 412 | CO-2774 | 10.060 | 1.451 | 5 | AATACAGCTCTGGAGTGGGG | 38342327 | 38342346 |
| 413 | CO-2775 | 7.892 | 1.077 | 5 uM | TAATACAGCTCTGGAGTGGG | 38342326 | 38342345 |
| 414 | CO-2776 | 1.300 | 0.072 | 2.5 uM | TAATACAGCTCTGGAGTGGG | 38342326 | 38342345 |
| 415 | CO-2777 | 2.015 | 0.378 | 10 uM | TTAATACAGCTCTGGAGTGG | 38342325 | 38342344 |
| 416 | CO-2778 | 2.067 | 0.596 | 10 uM | AATACAGCTCTGGAGTGGGGT | 38342327 | 38342346 |
| 417 | CO-3133 | 1.338 | 0.152 | 10 uM | TCGATGCATAGCTGGGATTA | 38342143 | 38342162 |
| 418 | CO-3134 | 1.219 | 0.109 | 5 uM | AGCCTCTCGATGCATAGCTG | 38342137 | 38342156 |
| 419 | CO-3135 | 1.268 | 0.152 | 10 uM | CCTCAGCCTCTCGATGCATA | 38342133 | 38342152 |
| 420 | CO-3137 | 1.361 | 0.241 | 10 uM | TTAATACAGCTCTGGAGTGG | 38342325 | 38342345 |
| 421 | CO-3138 | 1.229 | 0.058 | 2.5 uM | TTAATACAGCTCTGGAGTGG | 38342325 | 38342345 |
| 422 | CO-3139 | 1.299 | 0.225 | 5 uM | TTAATACAGCTCTGGAGTGG | 38342325 | 38342345 |
| 423 | CO-3140 | 3.309 | 0.520 | 5 | GAAAATGATCTCATGGGATC | 38343197 | 38343217 |
| 424 | CO-3141 | 1.456 | 0.142 | 1.25 uM | GAAAATGATCTCATGGGATC | 38343197 | 38343217 |
| 425 | CO-3142 | 1.508 | 0.332 | 10 uM | GAAAATGATCTCATGGGATC | 38343197 | 38343217 |
| 426 | CO-3143 | 1.165 | 0.394 | 5 uM | AATACAGCTCTGGAGTGGGGT | 38342327 | 38342347 |
| 427 | CO-3144 | 2.532 | 0.828 | 10 uM | AATACAGCTCTGGAGTGGGGT | 38342327 | 38342347 |
| 428 | CO-3166 | 4.566 | 0.257 | 5 | TACAGCTCTGGAGTGGGGTG | 38342329 | 38342348 |
| 429 | CO-3167 | 5.954 | 1.072 | 5 | ATACAGCTCTGGAGTGGGGT | 38342328 | 38342347 |
| 430 | CO-3168 | 1.421 | 0.203 | 10 uM | TTTAATACAGCTCTGGAGTG | 38342324 | 38342343 |
| 431 | CO-3170 | 3.770 | 0.440 | 9 | TTAATACAGCTCTGGAGTGGG | 38342325 | 38342345 |
| 432 | CO-3171 | 3.517 | 0.352 | 9 | TTAATACAGCTCTGGAGTGGGG | 38342325 | 38342346 |
| 433 | CO-3172 | 5.257 | 0.448 | 9 | TTAATACAGCTCTGGAGTGGGGT | 38342325 | 38342347 |
| 434 | CO-3281 | 1.587 | 0.269 | 5 uM | TGTAATCAGATTTCACCGTG | 38352941 | 38352960 |
| 435 | CO-3282 | 1.299 | 0.314 | 5 uM | CTCAGTCCTTCTGTAATCAG | 38352930 | 38352949 |
| 436 | CO-3283 | 1.421 | 0.424 | 5 uM | AAGCTTTCATTTCTCAGTCC | 38352918 | 38352937 |
| 437 | CO-3284 | 1.480 | 0.302 | 5 uM | CTCTAATTCCAGCAAAGCTT | 38352904 | 38352923 |
| 438 | CO-3285 | 1.451 | 0.008 | 5 uM | TCTTATTTGAGACAGCTGCC | 38352885 | 38352904 |
| 439 | CO-3286 | 1.382 | 0.062 | 5 uM | GTGCCACGCTCTGCTTTACT | 38352866 | 38352885 |
| 440 | CO-3287 | 1.291 | 0.501 | 5 uM | TGCAGAATGTAGTGCCACGC | 38352855 | 38352874 |
| 441 | CO-3288 | 1.630 | 0.424 | 5 uM | GGCCTCTTTCTGCAGAATGT | 38352845 | 38352864 |
| 442 | CO-3289 | 1.046 | 0.240 | 5 uM | ACTATATTCTGCAGTAAGGC | 38352828 | 38352847 |
| 443 | CO-3290 | 0.871 | 0.242 | 5 uM | GGAATCATGGTGATGCATAA | 38352807 | 38352826 |
| 444 | CO-3291 | 1.365 | 0.057 | 5 uM | GAGACTTGGGTTTGATTTAG | 38352788 | 38352807 |
| 445 | CO-3292 | 0.980 | 0.183 | 5 uM | GTGATGGTCAGAGACTTGGG | 38352778 | 38352797 |
| 446 | CO-3293 | 0.971 | 0.265 | 5 uM | ATTTTCGGTAAGTGATGGTC | 38352767 | 38352786 |
| 447 | CO-3294 | 1.385 | 0.139 | 5 uM | GTCACAACTTCATGGTTCGA | 38352746 | 38352765 |
| 448 | CO-3295 | 2.015 | 0.522 | 5 uM | TGCAGCTTTTAGAAATGGTC | 38352729 | 38352748 |
| 449 | CO-3296 | 1.143 | 0.222 | 5 uM | TGAGGATCCTGTTAAACAAT | 38352710 | 38352729 |
| 450 | CO-3297 | 1.392 | 0.232 | 5 uM | GCTGTTTAATCTGAGGATCC | 38352699 | 38352718 |
| 451 | CO-3298 | 1.183 | 0.185 | 0.625 uM | CATAGAATCGTCCTTTACAC | 38352665 | 38352684 |
| 452 | CO-3299 | 1.397 | 0.120 | 5 uM | GGCTAACTTGCTGTGGAGTT | 38352637 | 38352656 |
| 453 | CO-3300 | 1.208 | 0.193 | 5 uM | TAGTTTTTAGGTGGCCCCCG | 38352615 | 38352634 |
| 454 | CO-3301 | 1.584 | 0.215 | 5 uM | GCAACTGAACACATTTCTTA | 38352597 | 38352616 |
| 455 | CO-3302 | 1.190 | 0.561 | 5 uM | CTCCCTCACTGCAACTGAAC | 38352587 | 38352606 |
| 456 | CO-3303 | 0.712 | 0.054 | 5 uM | GATATTACCTTTGCTCCCTC | 38352574 | 38352593 |
| 457 | CO-3304 | 1.192 | 0.271 | 5 uM | AGCTGAAGGGTGATATTACC | 38352563 | 38352582 |
| 458 | CO-3305 | 1.797 | 0.088 | 5 uM | GGGACTTTGATAAGGAAGCT | 38352547 | 38352566 |
| 459 | CO-3306 | 1.324 | 0.042 | 5 uM | GCCCTGCAGTATCTCTAACC | 38352525 | 38352544 |
| 460 | CO-3307 | 1.347 | 0.097 | 5 uM | AAATGCTCCTACACCCTGCC | 38352508 | 38352527 |
| 461 | CO-3308 | 1.225 | 0.201 | 5 uM | GCATAAAGTTCAAATGCTCC | 38352497 | 38352516 |
| 462 | CO-3309 | 1.469 | 0.633 | 5 uM | CGGTGGAGCTTGGCATAAAG | 38352485 | 38352504 |
| 463 | CO-3310 | 1.381 | 0.240 | 5 uM | CCAAAAAAAATACACAGCGG | 38352468 | 38352487 |
| 464 | CO-3311 | 1.287 | 0.091 | 5 uM | GGCAATAAAAGAGTCAGGAT | 38352443 | 38352462 |
| 465 | CO-3312 | 1.264 | 0.336 | 5 uM | AATGAGGAGGCCAGGCAATA | 38352430 | 38352449 |
| 466 | CO-3313 | 1.220 | 0.109 | 5 uM | GCGTGTGACAGTATAAATAT | 38352401 | 38352420 |
| 467 | CO-3314 | 0.860 | 0.128 | 5 uM | GATTTTGTACATGCGTGTGA | 38352389 | 38352408 |
| 468 | CO-3315 | 1.506 | 0.070 | 5 uM | TGAGTACTTGTCAATTGATT | 38352373 | 38352392 |
| 469 | CO-3316 | 1.470 | 0.715 | 5 uM | ATATCCTCAATGAGTACTTG | 38352363 | 38352382 |
| 470 | CO-3317 | 1.228 | 0.329 | 5 uM | GCCTTGCATATATTATATCC | 38352349 | 38352368 |
| 471 | CO-3318 | 0.932 | 0.253 | 5 uM | AGCCAGTACCGCAGTGCCTT | 38352334 | 38352353 |
| 472 | CO-3319 | 1.307 | 0.017 | 5 uM | CACCTTCACAGCAGCCAGTA | 38352322 | 38352341 |
| 473 | CO-3320 | 1.265 | 0.091 | 5 uM | GTTACTTCTACTCACCTTCA | 38352310 | 38352329 |
| 474 | CO-3321 | 1.102 | 0.010 | 5 uM | GCATTTTCTGAGTGAGTTAC | 38352295 | 38352314 |
| 475 | CO-3322 | 1.330 | 0.151 | 5 uM | TTTTTGCAGTGCCAATTTGC | 38352277 | 38352296 |
| 476 | CO-3323 | 1.127 | 0.396 | 5 uM | CTACATCTGTGTTTTTGCAG | 38352266 | 38352285 |
| 477 | CO-3324 | 0.970 | 0.225 | 5 uM | ACTGTCACATCTACATCTGT | 38352256 | 38352275 |
| 478 | CO-3325 | 1.063 | 0.036 | 5 uM | GTACTACTCAGGACTGTCAC | 38352244 | 38352263 |
| 479 | CO-3326 | 1.366 | 0.291 | 5 uM | GACTGGCAATTAGAGGTAGA | 38352203 | 38352222 |
| 480 | CO-3327 | 1.260 | 0.261 | 5 uM | TTTTGTGGAAAGACTGGCAA | 38352192 | 38352211 |
| 481 | CO-3328 | 1.286 | 0.104 | 5 uM | TGCACCCCCAAAATAGCTTC | 38352150 | 38352169 |
| 482 | CO-3329 | 1.346 | 0.276 | 5 uM | CTCTATCATCTTGCACCCCC | 38352139 | 38352158 |
| 483 | CO-3330 | 1.124 | 0.291 | 5 uM | TACTATACCTTCTCTATCAT | 38352128 | 38352147 |
| 484 | CO-3331 | 1.253 | 0.047 | 5 uM | GGAGCTCCAGGACTGAGATA | 38352104 | 38352123 |
| 485 | CO-3332 | 1.444 | 0.242 | 5 uM | GCAAGGCACAAAGGGAGCTC | 38352091 | 38352110 |
| 486 | CO-3333 | 1.293 | 0.092 | 5 uM | TTGAAGACTTATGTGCAAGG | 38352077 | 38352096 |
| 487 | CO-3334 | 1.848 | 0.273 | 5 uM | CGGCCAGCAATTATTTCTTT | 38352056 | 38352075 |
| 488 | CO-3335 | 0.979 | 0.266 | 5 uM | GTCGGGTGCTGTTAGAATCA | 38351713 | 38351732 |
| 489 | CO-3336 | 2.017 | 0.054 | 5 uM | CCCACTTGTACTGACTGTCG | 38351697 | 38351716 |
| 490 | CO-3337 | 1.142 | 0.403 | 5 uM | GTATCCCCACTTCCCACTTG | 38351685 | 38351704 |
| 491 | CO-3338 | 1.108 | 0.151 | 5 uM | GAAGAGCATATGGTATCCCC | 38351673 | 38351692 |
| 492 | CO-3339 | 1.110 | 0.069 | 5 uM | GATCTGAATAGGCTGCTAGG | 38351652 | 38351671 |
| 493 | CO-3340 | 1.515 | 0.323 | 5 uM | ATGGCCCCTTAGTGATCTGA | 38351639 | 38351658 |
| 494 | CO-3341 | 1.023 | 0.161 | 5 uM | GTGAAGTTGCAGATGGCCCC | 38351627 | 38351646 |
| 495 | CO-3342 | 1.003 | 0.270 | 5 uM | ACCTGATTTCTAACTGAGGA | 38351594 | 38351613 |
| 496 | CO-3343 | 1.416 | 0.175 | 5 uM | AGAATTGACTTTGAATCACC | 38351577 | 38351596 |
| 497 | CO-3344 | 1.238 | 0.324 | 5 uM | TATCTGATACAGAATTGACT | 38351567 | 38351586 |
| 498 | CO-3345 | 1.395 | 0.127 | 5 uM | TGCAACTCGGTATCTGATAC | 38351557 | 38351576 |
| 499 | CO-3346 | 1.109 | 0.209 | 5 uM | GTCATCTTCCCTCTCTGAAA | 38351531 | 38351550 |
| 500 | CO-3347 | 1.071 | 0.256 | 5 uM | GAACTCTGCAAAGATGATGT | 38351513 | 38351532 |
| 501 | CO-3348 | 1.181 | 0.182 | 5 uM | TACTGAACTGTGAACTCTGC | 38351502 | 38351521 |
| 502 | CO-3349 | 0.931 | 0.248 | 5 uM | CATACCACCTTTACTGAACT | 38351491 | 38351510 |
| 503 | CO-3350 | 1.511 | 0.252 | 5 uM | GTTTATAAATCATACCACCT | 38351481 | 38351500 |
| 504 | CO-3351 | 1.151 | 0.323 | 5 uM | GGCTTGTATTATGGATATTT | 38351461 | 38351480 |
| 505 | CO-3352 | 1.620 | 0.244 | 5 uM | CAACTTTGTCTGGACCTCTC | 38351436 | 38351455 |
| 506 | CO-3353 | 1.113 | 0.172 | 5 uM | TCTTCGAAGCCAGACAACTT | 38351422 | 38351441 |
| 507 | CO-3354 | 1.536 | 0.182 | 5 uM | CTACCCTTCCTACCTTAGAG | 38351392 | 38351411 |
| 508 | CO-3355 | 1.103 | 0.286 | 5 uM | TTGACCAGAAGAGGCAGCCC | 38351346 | 38351365 |
| 509 | CO-3361 | 0.939 | 0.158 | 5 | GCTCTTGACCTGCTATTGAT | 38342966 | 38342985 |
| 510 | CO-3362 | 1.215 | 0.389 | 5 | TAGTTGGTGCCTCTCTTCAG | 38343002 | 38343021 |
| 511 | CO-3363 | 1.080 | 0.251 | 5 | TTTTGGTGAAACTTGAAACC | 38343029 | 38343048 |
| 512 | CO-3364 | 1.183 | 0.261 | 5 | CTGCCCATTTCTACTTTTTG | 38343044 | 38343063 |
| 513 | CO-3365 | 0.923 | 0.166 | 5 | CACTGCTTGGTGAATGCCTT | 38343066 | 38343085 |
| 514 | CO-3366 | 1.079 | 0.253 | 5 | CTTGGATGGCTTCAAAAGTC | 38343085 | 38343104 |
| 515 | CO-3367 | 1.062 | 0.223 | 5 | GCAATATTCTCCCCTTGAGC | 38343118 | 38343137 |
| 516 | CO-3368 | 0.916 | 0.163 | 5 | CCCAGTAATAATTTTCATGG | 38343137 | 38343156 |
| 517 | CO-3369 | 1.717 | 0.147 | 5 | TTGGTCTAGGAGCACTGTCC | 38343155 | 38343174 |
| 518 | CO-3370 | 0.967 | 0.162 | 5 | GCTTTCTACTGACCAGGGTT | 38343174 | 38343193 |
| 519 | CO-3371 | 0.814 | 0.199 | 5 | ATCCCATGAGATCATTTTCA | 38343196 | 38343215 |
| 520 | CO-3372 | 1.160 | 0.198 | 5 | ACTCCCAGGACCTGAGTGAT | 38343220 | 38343239 |
| 521 | CO-3373 | 1.027 | 0.260 | 5 | CTAATGCTCCCTGTGACTAC | 38343245 | 38343264 |
| 522 | CO-3374 | 1.241 | 0.329 | 5 | TCACCAGTGTCTCAACAATC | 38343269 | 38343288 |
| 523 | CO-3375 | 1.227 | 0.196 | 5 | ACAGAGGTTCAAAGTTCACC | 38343284 | 38343303 |
| 524 | CO-3376 | 1.039 | 0.114 | 5 | ACAGAGCAAACAGGGAAATC | 38343304 | 38343323 |
| 525 | CO-3377 | 1.149 | 0.322 | 5 | CTGAAAGCTATCAGGCACAG | 38343320 | 38343339 |
| 526 | CO-3378 | 1.064 | 0.226 | 5 | GGAGATTTGTTAGCAGACTG | 38343337 | 38343356 |
| 527 | CO-3379 | 1.454 | 1.073 | 5 | GGTTAAACTGCATAAAGGAG | 38343353 | 38343372 |
| 528 | CO-3380 | 0.930 | 0.309 | 5 | CCTCCCCATTGGAAGTACAG | 38343374 | 38343393 |
| 529 | CO-3381 | 1.082 | 0.195 | 5 | CCATAGGCTGATTCCAATTC | 38343393 | 38343412 |
| 530 | CO-3382 | 0.920 | 0.193 | 5 | GAGCTATCTCTTCTCCCATA | 38343408 | 38343427 |
| 531 | CO-3383 | 1.494 | 0.359 | 5 | GTTCCCACACAGAATCCTAG | 38343427 | 38343446 |
| 532 | CO-3384 | 1.417 | 0.363 | 5 | GCCCTTTTATCTCTTCAAGT | 38343445 | 38343464 |
| 533 | CO-3385 | 1.120 | 0.113 | 5 | CGAGGTTCTCTTTCAAGGAT | 38343465 | 38343484 |
| 534 | CO-3386 | 1.531 | 0.436 | 5 | TGAGATGCAAAGCACGAGGT | 38343479 | 38343498 |
| 535 | CO-3387 | 1.752 | 0.400 | 5 | GTCAGAGGCAGTTGAGATGC | 38343491 | 38343510 |
| 536 | CO-3388 | 1.079 | 0.339 | 5 | ACCCAACCTTGTGTCAGAGG | 38343503 | 38343522 |
| 537 | CO-3389 | 1.014 | 0.220 | 5 | ATGACCCAACCTTGTGTCAG | 38343506 | 38343525 |
| 538 | CO-3390 | 0.955 | 0.148 | 5 | GGTTAATGACCCAACCTTGT | 38343511 | 38343530 |
| 539 | CO-3391 | 1.087 | 0.172 | 5 | ATATGGTTAATGACCCAACC | 38343515 | 38343534 |
| 540 | CO-3392 | 2.976 | 0.455 | 9 uM | TATGAAGTAAGAAAGGGGTA | 38343540 | 38343559 |
| 541 | CO-3393 | 1.028 | 0.166 | 5 | GACAAGATTAGGAAATGCAC | 38343569 | 38343588 |
| 542 | CO-3394 | 1.000 | 0.175 | 5 | CACAGAAGACAAGATTAGGA | 38343576 | 38343595 |
| 543 | CO-3395 | 0.991 | 0.141 | 5 | TCTGCACAGAAGACAAGATT | 38343580 | 38343599 |
| 544 | CO-3396 | 1.060 | 0.176 | 5 | TCTCAAGTTTCCATACCTGG | 38343628 | 38343647 |
| 545 | CO-3397 | 0.989 | 0.224 | 5 | TATGGACAAAAGTCTCAAGT | 38343640 | 38343659 |
| 546 | CO-3398 | 1.717 | 0.172 | 5 | GACCCCCTCTCCATCCCTTT | 38343884 | 38343903 |
| 547 | CO-3399 | 1.051 | 0.196 | 5 | GATGCCCAATTTCTCTTTTC | 38344512 | 38344531 |
| 548 | CO-3400 | 1.326 | 0.139 | 5 | CCTTTCTCTGGATCATGCTG | 38344683 | 38344702 |
| 549 | CO-3401 | 1.325 | 0.301 | 5 | TGGTAGGTATAGAGTCTCAC | 38345027 | 38345046 |
| 550 | CO-3402 | 0.984 | 0.250 | 5 | CCTTTCTCTGGATCATGCTG | 38344683 | 38344702 |
| 551 | CO-3403 | 1.055 | 0.148 | 5 | GCCTGACCCCTATTCTTCTT | 38344910 | 38344929 |
| 552 | CO-3404 | 1.910 | 0.210 | 5 | CCACTGAGCTTGCCTGACCC | 38344921 | 38344940 |
| 553 | CO-3405 | 2.051 | 0.202 | 5 | TGGTAGGTATAGAGTCTCAC | 38345027 | 38345046 |
| 554 | CO-3406 | 1.051 | 0.331 | 5 | GCAAAGGCGGTTTCACAATA | 38345161 | 38345180 |
| 555 | CO-3407 | 1.221 | 0.456 | 5 | GATGGATTCGGTCAAGTTAG | 38345208 | 38345227 |
| 556 | CO-3408 | 1.133 | 0.289 | 5 | GGAAGGAGGTCACTTTGGGA | 38345339 | 38345358 |
| 557 | CO-3409 | 0.998 | 0.146 | 5 | CCAGGCAGGAAGGAGGTCAC | 38345346 | 38345365 |
| 558 | CO-3410 | 1.450 | 0.209 | 5 | TCAGTACTGCACTTGATGGA | 38345445 | 38345464 |
| 559 | CO-3411 | 3.834 | 0.898 | 5 | GCTTGGGGTATCAGTACTGC | 38345455 | 38345474 |
| 560 | CO-3532 | 1.768 | 0.249 | 2.5 | TACAGCTCTGGAGTGGG | | |
| 561 | CO-3533 | 1.948 | 0.637 | 10 | GCTCTGGAGTGGGGTG | | |
| 562 | CO-3534 | 2.268 | 0.283 | 5 | ACAGCTCTGGAGTGGG | | |
| 563 | CO-3535 | 1.300 | 0.221 | 5 | TAATACAGCTCTGGAG | 38342326 | 38342341 |
| 564 | CO-3536 | 1.321 | 0.426 | 1.25 | TTTAATACAGCTCTGG | 38342324 | 38342339 |
| 565 | CO-3537 | 1.349 | 0.671 | 1.25 | CACTTTTAATACAGCT | | |
| 566 | CO-3719 | 1.229 | 0.096 | 5 | AATACAGCTCTGGAGTGGGGT | 38342327 | 38342346 |
| 567 | CO-3721 | 3.304 | 0.520 | 5 | TTAATACAGCTCTGGAGTGGGGT | 38342325 | 38342347 |
| 568 | CO-3722 | 1.941 | 0.307 | 5 | TTAATACAGCTCTGGAGTGGGGT | 38342325 | 38342347 |
| 569 | CO-3723 | 2.448 | 0.412 | 5 | TTAATACAGCTCTGGAGTGGGGT | 38342325 | 38342347 |
| 570 | CO-3737 | 1.524 | 0.134 | 10 | TTAATACAGCTCTGGAGTGGGGT | 38342325 | 38342347 |
| 571 | CO-3738 | 1.401 | 0.091 | 10 | TTAATACAGCTCTGGAGTGGGGT | 38342325 | 38342347 |
| 572 | CO-3739 | 1.466 | 0.058 | 10 | TTAATACAGCTCTGGAGTGGGGT | 38342325 | 38342347 |
| 573 | CO-3740 | 2.286 | 0.274 | 5 | TTAATACAGCTCTGGAGTGGGGT | 38342325 | 38342347 |
| 574 | CO-3741 | 3.109 | 0.360 | 10 | TTAATACAGCTCTGGAGTGGGGT | 38342325 | 38342347 |
| 575 | CO-3742 | 1.537 | 0.129 | 2.5 | TTTTAATACAGCTCTGGAGTGGG | 38342323 | 38342345 |
| 576 | CO-3743 | 1.235 | 0.202 | 5 | TTTTAATACAGCTCTGGAGTGGG | 38342323 | 38342345 |
| 577 | CO-3744 | 1.321 | 0.055 | 10 | TTTTAATACAGCTCTGGAGTGGGGT | 38342323 | 38342347 |
| 578 | CO-3745 | 2.036 | 0.205 | 5 | TTTTAATACAGCTCTGGAGTGGGGT | 38342323 | 38342347 |
| 579 | CO-3746 | 3.303 | 0.402 | 10 | TTTTAATACAGCTCTGGAGTGGGGT | 38342323 | 38342349 |
| | | | | | GG | | |
| 580 | CO-3747 | 2.408 | 0.189 | 5 | TTTTAATACAGCTCTGGAGTGGGGT | 38342323 | 38342349 |
| | | | | | GG | | |
| 581 | CO-3777 | 1.841 | 0.238 | 5 uM | TTAATACAGCTCTGGAGTGGGGT | 38342325 | 38342347 |
| 582 | CO-3778 | 1.688 | 0.062 | 5 uM | TTAATACAGCTCTGGAGTGGGGT | 38342325 | 38342347 |
| 583 | CO-3779 | 1.508 | 0.192 | 5 uM | TTAATACAGCTCTGGAGTGGGGT | 38342325 | 38342347 |
| 584 | CO-3780 | 1.762 | 0.214 | 5 uM | TTAATACAGCTCTGGAGTGGGGT | 38342325 | 38342347 |
| 585 | CO-3781 | 2.723 | 0.280 | 10 | TTAATACAGCTCTGGAGTGGGGT | 38342325 | 38342347 |
| 586 | CO-3782 | 1.544 | 0.206 | 5 uM | TTAATACAGCTCTGGAGTGGGGT | 38342325 | 38342347 |
| 587 | CO-3783 | 7.262 | 0.852 | 10 | TTAATACAGCTCTGGAGTGGGGT | 38342325 | 38342347 |
| 588 | CO-3784 | 3.863 | 0.648 | 5 | TTAATACAGCTCTGGAGTGGGGT | 38342325 | 38342347 |
| 589 | CO-3785 | 1.228 | 0.173 | 5 uM | AATACAGCTCTGGAGTGGGGT | 38342327 | 38342347 |
| 590 | CO-3786 | 1.231 | 0.166 | 5 uM | AATACAGCTCTGGAGTGGGGT | 38342327 | 38342347 |
| 591 | CO-3787 | 1.234 | 0.135 | 5 uM | AATACAGCTCTGGAGTGGGGT | 38342327 | 38342347 |
| 592 | CO-3788 | 1.679 | 0.179 | 5 uM | AATACAGCTCTGGAGTGGGGT | 38342327 | 38342347 |
| 593 | CO-3789 | 3.725 | 0.260 | 5 | AATACAGCTCTGGAGTGGGGT | 38342327 | 38342347 |
| 594 | CO-3790 | 1.130 | 0.172 | 5 uM | AATACAGCTCTGGAGTGGG | 38342327 | 38342345 |
| 595 | CO-3791 | 1.468 | 0.086 | 5 uM | AATACAGCTCTGGAGTGGG | 38342327 | 38342345 |
| 596 | CO-3792 | 2.222 | 0.301 | 5 uM | AATACAGCTCTGGAGTGGG | 38342327 | 38342345 |
| 597 | CO-3793 | 1.632 | 0.253 | 5 uM | TACAGCTCTGGAGTGGGGT | 38342329 | 38342347 |
| 598 | CO-3794 | 1.454 | 0.175 | 5 uM | TACAGCTCTGGAGTGGGGT | 38342329 | 38342347 |
| 599 | CO-3795 | 3.062 | 0.350 | 5 | TACAGCTCTGGAGTGGGGT | 38342329 | 38342347 |
| 600 | CO-3796 | 1.670 | 0.194 | 5 uM | TTAATACAGCTCTGGAGTGGGGT | 38342325 | 38342347 |
| 601 | CO-3797 | 0.859 | 0.150 | 5 uM | TTAATACAGCTCTGGAGTGGGGT | 38342325 | 38342347 |
| 602 | CO-3798 | 0.828 | 0.105 | 5 uM | TTAATACAGCTCTGGAGTGGGGT | 38342325 | 38342347 |
| 603 | CO-3799 | 0.931 | 0.087 | 5 uM | TTAATACAGCTCTGGAGTGGGGT | 38342325 | 38342347 |
| 604 | CO-3800 | 2.154 | 0.223 | 10 | TTAATACAGCTCTGGAGTGGGGT | 38342325 | 38342347 |
| 605 | CO-4256 | 3.143 | 0.370 | 9 uM | TATGAAGTAAGAAAGGGGTA | 38343540 | 38343559 |
| 606 | CO-4257 | 2.156 | 0.167 | 5 | TATGAAGTAAGAAAGGGGTA | 38343540 | 38343559 |
| 607 | CO-4258 | 2.452 | 0.306 | 5 | TATGAAGTAAGAAAGGGGTA | 38343540 | 38343559 |
| 608 | CO-4259 | 3.506 | 0.457 | 9 uM | TATGAAGTAAGAAAGGGGTA | 38343540 | 38343559 |
| 609 | CO-4260 | 2.403 | 0.315 | 5 | TATGAAGTAAGAAAGGGGTA | 38343540 | 38343559 |
| 610 | CO-4261 | 1.947 | 0.253 | 5 | TATGAAGTAAGAAAGGGGTA | 38343540 | 38343559 |
| 611 | CO-4262 | 2.018 | 0.586 | 5 | TATGAAGTAAGAAAGGGGTA | 38343540 | 38343559 |
| 612 | CO-4263 | 2.301 | 0.273 | 5 | TATGAAGTAAGAAAGGGGTA | 38343540 | 38343559 |
| 613 | CO-4264 | 2.641 | 0.367 | 5 | TATGAAGTAAGAAAGGGGTA | 38343540 | 38343559 |
| 614 | CO-4265 | 1.939 | 0.285 | 5 | TATGAAGTAAGAAAGGGGTA | 38343540 | 38343559 |
| 615 | CO-4266 | 3.508 | 0.541 | 9 uM | TATGAAGTAAGAAAGGGGTA | 38343540 | 38343559 |
| 616 | CO-4267 | 4.770 | 0.471 | 9 uM | TATGAAGTAAGAAAGGGGTA | 38343540 | 38343559 |
| 617 | CO-4268 | 2.501 | 0.386 | 5 | TATGAAGTAAGAAAGGGGTA | 38343540 | 38343559 |
| 618 | CO-4269 | 1.890 | 0.797 | 5 | GCTTGGGGTATCAGTACTGC | 38345455 | 38345474 |
| 619 | CO-4270 | 1.956 | 0.480 | 5 | GCTTGGGGTATCAGTACTGC | 38345455 | 38345474 |
| 620 | CO-4271 | 3.143 | 0.431 | 9 uM | GCTTGGGGTATCAGTACTGC | 38345455 | 38345474 |
| 621 | CO-4272 | 2.147 | 0.196 | 5 | GCTTGGGGTATCAGTACTGC | 38345455 | 38345474 |
| 622 | CO-4273 | 2.735 | 0.460 | 5 | GCTTGGGGTATCAGTACTGC | 38345455 | 38345474 |
| 623 | CO-4274 | 2.844 | 0.276 | 9 uM | GCTTGGGGTATCAGTACTGC | 38345455 | 38345474 |
| 624 | CO-4275 | 2.157 | 0.355 | 5 | GCTTGGGGTATCAGTACTGC | 38345455 | 38345474 |
| 625 | CO-4276 | 2.093 | 0.338 | 5 | GCTTGGGGTATCAGTACTGC | 38345455 | 38345474 |
| 626 | CO-4277 | 1.892 | 0.305 | 5 | GCTTGGGGTATCAGTACTGC | 38345455 | 38345474 |
| 627 | CO-4278 | 1.899 | 0.396 | 5 | GCTTGGGGTATCAGTACTGC | 38345455 | 38345474 |
| 628 | CO-4279 | 2.410 | 0.179 | 9 uM | GCTTGGGGTATCAGTACTGC | 38345455 | 38345474 |
| 629 | CO-4280 | 2.682 | 0.255 | 5 | GCTTGGGGTATCAGTACTGC | 38345455 | 38345474 |
| 630 | CO-4281 | 2.183 | 0.415 | 5 | GCTTGGGGTATCAGTACTGC | 38345455 | 38345474 |
| 631 | CO-4282 | 2.843 | 0.210 | 5 | TTATGAAGTAAGAAAGGGGTAA | 38343539 | 38343560 |
| 632 | CO-4283 | 2.650 | 0.558 | 5 | TTATGAAGTAAGAAAGGGGTAA | 38343539 | 38343560 |
| 633 | CO-4284 | 2.450 | 0.358 | 5 | TTATGAAGTAAGAAAGGGGTAA | 38343539 | 38343560 |
| 634 | CO-4285 | 3.247 | 0.413 | 9 uM | TTATGAAGTAAGAAAGGGGTAA | 38343539 | 38343560 |
| 635 | CO-4286 | 2.546 | 0.279 | 5 | TTTATGAAGTAAGAAAGGGGTAAA | 38343538 | 38343561 |
| 636 | CO-4287 | 2.374 | 0.410 | 5 | TTTATGAAGTAAGAAAGGGGTAAA | 38343538 | 38343561 |
| 637 | CO-4288 | 2.520 | 0.458 | 5 | TTTATGAAGTAAGAAAGGGGTAAA | 38343538 | 38343561 |
| 638 | CO-4289 | 3.047 | 0.626 | 5 | TTTATGAAGTAAGAAAGGGGTAAA | 38343538 | 38343561 |
| 639 | CO-4290 | 2.261 | 0.370 | 5 | TGCTTGGGGTATCAGTACTGCA | 38345454 | 38345475 |
| 640 | CO-4291 | 2.165 | 0.693 | 5 | TGCTTGGGGTATCAGTACTGCA | 38345454 | 38345475 |
| 641 | CO-4292 | 1.526 | 0.116 | 5 | TGCTTGGGGTATCAGTACTGCA | 38345454 | 38345475 |
| 642 | CO-4293 | 2.152 | 0.276 | 5 | TGCTTGGGGTATCAGTACTGCA | 38345454 | 38345475 |
| 643 | CO-4294 | 1.319 | 0.245 | 5 | GTGCTTGGGGTATCAGTACTGCAC | 38345453 | 38345476 |
| 644 | CO-4295 | 1.616 | 0.360 | 5 | GTGCTTGGGGTATCAGTACTGCAC | 38345453 | 38345476 |
| 645 | CO-4296 | 1.265 | 0.419 | 5 | GTGCTTGGGGTATCAGTACTGCAC | 38345453 | 38345476 |
| 646 | CO-4297 | 1.249 | 0.456 | 5 | GTGCTTGGGGTATCAGTACTGCAC | 38345453 | 38345476 |
| 647 | CO-4298 | 1.418 | 0.196 | 5 | AATATGGTTAATGACCCAAC | 38343516 | 38343535 |
| 648 | CO-4299 | 1.264 | 0.296 | 5 | AATATGGTTAATGACCCAAC | 38343516 | 38343535 |
| 649 | CO-4300 | 1.330 | 0.198 | 5 | AATATGGTTAATGACCCAAC | 38343516 | 38343535 |
| 650 | CO-4301 | 1.340 | 0.169 | 5 | AATATGGTTAATGACCCAAC | 38343516 | 38343535 |
| 651 | CO-4302 | 1.971 | 0.339 | 5 | GAAGTAAGAAAGGGGTAAAT | 38343537 | 38343556 |
| 652 | CO-4303 | 2.344 | 0.401 | 5 | GAAGTAAGAAAGGGGTAAAT | 38343537 | 38343556 |
| 653 | CO-4304 | 2.059 | 0.348 | 5 | GAAGTAAGAAAGGGGTAAAT | 38343537 | 38343556 |
| 654 | CO-4305 | 1.925 | 0.308 | 5 | GAAGTAAGAAAGGGGTAAAT | 38343537 | 38343556 |
| 655 | CO-4306 | 1.990 | 0.351 | 5 | TGAAGTAAGAAAGGGGTAAA | 38343538 | 38343557 |
| 656 | CO-4307 | 2.424 | 0.372 | 5 | TGAAGTAAGAAAGGGGTAAA | 38343538 | 38343557 |
| 657 | CO-4308 | 2.097 | 0.196 | 5 | TGAAGTAAGAAAGGGGTAAA | 38343538 | 38343557 |
| 658 | CO-4309 | 3.671 | 0.207 | 9 uM | TGAAGTAAGAAAGGGGTAAA | 38343538 | 38343557 |
| 659 | CO-4310 | 2.085 | 0.454 | 5 | ATGAAGTAAGAAAGGGGTAA | 38343539 | 38343558 |
| 660 | CO-4311 | 1.798 | 0.388 | 5 | ATGAAGTAAGAAAGGGGTAA | 38343539 | 38343558 |
| 661 | CO-4312 | 2.004 | 0.198 | 5 | ATGAAGTAAGAAAGGGGTAA | 38343539 | 38343558 |
| 662 | CO-4313 | 2.305 | 0.209 | 5 | ATGAAGTAAGAAAGGGGTAA | 38343539 | 38343558 |
| 663 | CO-4314 | 1.848 | 0.254 | 5 | TTATGAAGTAAGAAAGGGGT | 38343541 | 38343560 |
| 664 | CO-4315 | 2.065 | 0.533 | 5 | TTATGAAGTAAGAAAGGGGT | 38343541 | 38343560 |
| 665 | CO-4316 | 1.956 | 0.263 | 5 | TTATGAAGTAAGAAAGGGGT | 38343541 | 38343560 |
| 666 | CO-4317 | 1.834 | 0.171 | 5 | TTATGAAGTAAGAAAGGGGT | 38343541 | 38343560 |
| 667 | CO-4318 | 1.480 | 0.290 | 5 | GGGGTATCAGTACTGCACTT | 38345451 | 38345470 |
| 668 | CO-4319 | 1.441 | 0.539 | 5 | GGGGTATCAGTACTGCACTT | 38345451 | 38345470 |
| 669 | CO-4320 | 1.468 | 0.314 | 5 | GGGGTATCAGTACTGCACTT | 38345451 | 38345470 |
| 670 | CO-4321 | 1.751 | 0.495 | 5 | GGGGTATCAGTACTGCACTT | 38345451 | 38345470 |
| 671 | CO-4322 | 1.845 | 0.327 | 5 | TGCTTGGGGTATCAGTACTG | 38345456 | 38345475 |
| 672 | CO-4323 | 1.548 | 0.247 | 5 | TGCTTGGGGTATCAGTACTG | 38345456 | 38345475 |
| 673 | CO-4324 | 1.681 | 0.370 | 5 | TGCTTGGGGTATCAGTACTG | 38345456 | 38345475 |
| 674 | CO-4325 | 1.662 | 0.220 | 5 | TGCTTGGGGTATCAGTACTG | 38345456 | 38345475 |
| 675 | CO-4326 | 1.533 | 0.187 | 5 | TCAGTGCTTGGGGTATCAGT | 38345460 | 38345479 |
| 676 | CO-4327 | 1.162 | 0.078 | 5 | TCAGTGCTTGGGGTATCAGT | 38345460 | 38345479 |
| 677 | CO-4328 | 2.116 | 0.396 | 5 | TCAGTGCTTGGGGTATCAGT | 38345460 | 38345479 |
| 678 | CO-4329 | 1.740 | 0.197 | 5 | TCAGTGCTTGGGGTATCAGT | 38345460 | 38345479 |
| 679 | CO-4330 | 1.399 | 0.308 | 5 | AATCAGTGCTTGGGGTATCA | 38345462 | 38345481 |
| 680 | CO-4331 | 1.350 | 0.116 | 5 | AATCAGTGCTTGGGGTATCA | 38345462 | 38345481 |
| 681 | CO-4332 | 1.772 | 0.252 | 5 | AATCAGTGCTTGGGGTATCA | 38345462 | 38345481 |
| 682 | CO-4333 | 1.423 | 0.211 | 5 | AATCAGTGCTTGGGGTATCA | 38345462 | 38345481 |
| 683 | CO-4334 | 1.996 | 0.244 | 5 | TTAATACAGCTCTGGAGTGGGGT | 38342325 | 38342347 |
| 684 | CO-4335 | 1.683 | 0.274 | 5 | TTAATACAGCTCTGGAGTGGGGT | 38342325 | 38342347 |
| 685 | CO-4336 | 1.889 | 0.361 | 5 | TTAATACAGCTCTGGAGTGGGGT | 38342325 | 38342347 |
| 686 | CO-4337 | 1.956 | 0.107 | 5 | TTAATACAGCTCTGGAGTGGGGT | 38342325 | 38342347 |
| 687 | CO-4338 | 2.158 | 0.293 | 5 | TTAATACAGCTCTGGAGTGGGGT | 38342325 | 38342347 |
| 688 | CO-4339 | 2.261 | 0.184 | 5 | TTAATACAGCTCTGGAGTGGGGT | 38342325 | 38342347 |
| 689 | CO-4340 | 1.402 | 0.196 | 5 | TTAATACAGCTCTGGAGTGGGGT | 38342325 | 38342347 |
| 690 | CO-4341 | 1.917 | 0.287 | 5 | TTAATACAGCTCTGGAGTGGGGT | 38342325 | 38342347 |
| 691 | CO-4342 | 1.603 | 0.274 | 5 | TTAATACAGCTCTGGAGTGGGGT | 38342325 | 38342347 |
| 692 | CO-4343 | 1.322 | 0.218 | 5 | TTAATACAGCTCTGGAGTGGG | 38342325 | 38342347 |
| 693 | CO-4344 | 1.955 | 0.271 | 5 | AATACAGCTCTGGAGTGGGGT | 38342327 | 38342347 |
| 694 | CO-4345 | 1.137 | 0.374 | 5 | AATACAGCTCTGGAGTGGG | 38342327 | 38342347 |
| 695 | CO-5046 | 2.188 | 0.328 | 5 | TGAAGTAAGAAAGGGGTAAA | 38343538 | 38343557 |
| 696 | CO-5048 | 1.601 | 0.111 | 5 | TATGAAGTAAGAAAGGGGTA | 38343540 | 38343559 |
| 697 | CO-5049 | 1.701 | 0.793 | 5 | TTAATACAGCTCTGGAGTGGGGT | 38342325 | 38342347 |
| 698 | CO-5050 | 1.879 | 0.249 | 5 | TTAATACAGCTCTGGAGTGGGGT | 38342325 | 38342347 |
| 699 | CO-5055 | 4.137 | 0.754 | 10 | TTAATACAGCTCTGGAGTGGGGT | 38342325 | 38342347 |
| 700 | CO-5056 | 1.503 | 0.705 | 5 | TTAATACAGCTCTGGAGTGGGGT | 38342325 | 38342347 |
| 701 | CO-5057 | 1.031 | 0.174 | 1 | TTAATACAGCTCTGGAGTGGGGT | 38342325 | 38342347 |
| 702 | CO-5058 | 4.576 | 1.360 | 10 | TTAATACAGCTCTGGAGTGGGGT | 38342325 | 38342347 |
| 703 | CO-5059 | 5.781 | 1.158 | 10 | TTAATACAGCTCTGGAGTGGGGT | 38342325 | 38342347 |
| 704 | CO-5060 | 3.459 | 0.475 | 10 | TTAATACAGCTCTGGAGTGGGGT | 38342325 | 38342347 |
| 705 | CO-5061 | 1.745 | 0.357 | 5 | TTAATACAGCTCTGGAGTGGGGT | 38342325 | 38342347 |
| 706 | CO-5063 | 1.693 | 0.313 | 5 | TTAATACAGCTCTGGAGTGGGGT | 38342325 | 38342347 |
| 707 | CO-5065 | 2.047 | 0.126 | 5 | TATGAAGTAAGAAAGGGGTA | 38343540 | 38343559 |
| 708 | CO-5068 | 3.649 | 0.699 | 10 | TATGAAGTAAGAAAGGGGTA | 38343540 | 38343559 |
| 709 | CO-5069 | 2.611 | 0.524 | 10 | TATGAAGTAAGAAAGGGGTA | 38343540 | 38343559 |
| 710 | CO-5070 | 2.430 | 0.304 | 5 | TATGAAGTAAGAAAGGGGTA | 38343540 | 38343559 |
| 711 | CO-5071 | 1.292 | 0.079 | 1 | TATGAAGTAAGAAAGGGGTA | 38343540 | 38343559 |
| 712 | CO-5072 | 2.012 | 0.323 | 5 | TGAAGTAAGAAAGGGGTAAA | 38343538 | 38343557 |
| 713 | CO-8944 | 0.894 | 0.135 | 5 uM | TCACACTCTGTTCAGAATTT | 38343801 | 38343820 |
| 714 | CO-8945 | 0.843 | 0.360 | 5 uM | GAATTTGATGATTGGCATTT | 38343782 | 38343801 |
| 715 | CO-8946 | 1.081 | 0.563 | 5 uM | ACTTGAATTTGATGATTGGC | 38343778 | 38343797 |
| 716 | CO-8947 | 0.865 | 0.214 | 5 uM | GAAGCGAGATTATACTTGAA | 38343765 | 38343784 |
| 717 | CO-8948 | 1.088 | 0.382 | 5 uM | AGAGAGAAGCGAGATTATAC | 38343760 | 38343779 |
| 718 | CO-8949 | 1.145 | 0.362 | 5 uM | GGTCTATATGACAAGAGAGA | 38343747 | 38343766 |
| 719 | CO-8950 | 1.160 | 0.583 | 5 uM | TCAATGGAAATGTGGTCTAT | 38343734 | 38343753 |
| 720 | CO-8951 | 1.418 | 0.658 | 5 uM | GAACCAGGTATGGAAACTTG | 38343625 | 38343644 |
| 721 | CO-8952 | 0.883 | 0.415 | 5 uM | GTGCATTTCCTAATCTTGTC | 38343569 | 38343588 |
| 722 | CO-8953 | 0.918 | 0.407 | 5 uM | TTGTGTTGTGCATTTCCTAA | 38343562 | 38343581 |
| 723 | CO-8954 | 0.951 | 0.351 | 5 uM | CATCTCAACTGCCTCTGACA | 38343492 | 38343511 |
| 724 | CO-8955 | 0.907 | 0.486 | 5 uM | TGCATCTCAACTGCCTCTGA | 38343490 | 38343509 |
| 725 | CO-8956 | 1.186 | 0.625 | 5 uM | CTTTGCATCTCAACTGCCTC | 38343487 | 38343506 |
| 726 | CO-8957 | 0.947 | 0.405 | 5 uM | TGAAAGAGAACCTCGTGCTT | 38343470 | 38343489 |
| 727 | CO-8958 | 1.322 | 0.754 | 5 uM | TTGAAAGAGAACCTCGTGCT | 38343469 | 38343488 |
| 728 | CO-8959 | 1.282 | 0.590 | 5 uM | GGGAACTTGAAGAGATAAAA | 38343441 | 38343460 |
| 729 | CO-8960 | 0.976 | 0.254 | 5 uM | TCTGTGTGGGAACTTGAAGA | 38343434 | 38343453 |
| 730 | CO-8961 | 1.060 | 0.517 | 5 uM | GCTCTAGGATTCTGTGTGGG | 38343424 | 38343443 |
| 731 | CO-8962 | 0.993 | 0.432 | 5 uM | ATAGCTCTAGGATTCTGTGT | 38343421 | 38343440 |
| 732 | CO-8963 | 0.860 | 0.315 | 5 uM | GATAGCTCTAGGATTCTGTG | 38343420 | 38343439 |
| 733 | CO-8964 | 1.165 | 0.364 | 5 uM | AGATAGCTCTAGGATTCTGT | 38343419 | 38343438 |
| 734 | CO-8965 | 1.275 | 0.541 | 5 uM | GAAGAGATAGCTCTAGGATT | 38343415 | 38343434 |
| 735 | CO-8966 | 0.967 | 0.292 | 5 uM | GGAGAAGAGATAGCTCTAGG | 38343412 | 38343431 |
| 736 | CO-8967 | 1.364 | 0.900 | 5 uM | ATGGGAGAAGAGATAGCTCT | 38343409 | 38343428 |
| 737 | CO-8968 | 0.964 | 0.391 | 5 uM | GGAGGAATTGGAATCAGCCT | 38343389 | 38343408 |
| 738 | CO-8969 | 1.075 | 0.542 | 5 uM | CTCCTTTATGCAGTTTAACC | 38343353 | 38343372 |
| 739 | CO-8970 | 0.923 | 0.390 | 5 uM | GTTTGCTCTGTGCCTGATAG | 38343313 | 38343332 |
| 740 | CO-8971 | 0.897 | 0.289 | 5 uM | ATTTCCCTGTTTGCTCTGTG | 38343305 | 38343324 |
| 741 | CO-8972 | 1.432 | 0.844 | 5 uM | GAACCTCTGTGATTTCCCTG | 38343294 | 38343313 |
| 742 | CO-8973 | 1.204 | 0.488 | 5 uM | TTGAACCTCTGTGATTTCCC | 38343292 | 38343311 |
| 743 | CO-8974 | 1.396 | 0.702 | 5 uM | ACTTTGAACCTCTGTGATTT | 38343289 | 38343308 |
| 744 | CO-8975 | 1.345 | 0.458 | 5 uM | GAACTTTGAACCTCTGTGAT | 38343287 | 38343306 |
| 745 | CO-8976 | 0.718 | 0.243 | 5 uM | GGTGAACTTTGAACCTCTGT | 38343284 | 38343303 |
| 746 | CO-8977 | 0.785 | 0.317 | 5 uM | GACACTGGTGAACTTTGAAC | 38343278 | 38343297 |
| 747 | CO-8978 | 1.165 | 0.560 | 5 uM | ATTGTTGAGACACTGGTGAA | 38343270 | 38343289 |
| 748 | CO-8979 | 1.145 | 0.473 | 5 uM | GATTGTTGAGACACTGGTGA | 38343269 | 38343288 |
| 749 | CO-8980 | 1.278 | 0.511 | 5 uM | AGTCACAGGGAGCATTAGGG | 38343247 | 38343266 |
| 750 | CO-8981 | 0.971 | 0.387 | 5 uM | GTAGTCACAGGGAGCATTAG | 38343245 | 38343264 |
| 751 | CO-8982 | 0.973 | 0.484 | 5 uM | GGTAGTCACAGGGAGCATTA | 38343244 | 38343263 |
| 752 | CO-8983 | 1.122 | 0.408 | 5 uM | TGGTAGTCACAGGGAGCATT | 38343243 | 38343262 |
| 753 | CO-8984 | 0.784 | 0.319 | 5 uM | GTCTGGTAGTCACAGGGAGC | 38343240 | 38343259 |
| 754 | CO-8985 | 0.870 | 0.419 | 5 uM | GGAGTGTCTGGTAGTCACAG | 38343235 | 38343254 |
| 755 | CO-8986 | 0.927 | 0.507 | 5 uM | GGTCCTGGGAGTGTCTGGTA | 38343228 | 38343247 |
| 756 | CO-8987 | 1.039 | 0.389 | 5 uM | CTCAGGTCCTGGGAGTGTCT | 38343224 | 38343243 |
| 757 | CO-8988 | 1.201 | 0.341 | 5 uM | ACTCAGGTCCTGGGAGTGTC | 38343223 | 38343242 |
| 758 | CO-8989 | 1.043 | 0.421 | 5 uM | CCCTGGTCAGTAGAAAGCAA | 38343176 | 38343195 |
| 759 | CO-8990 | 1.002 | 0.405 | 5 uM | ACCCTGGTCAGTAGAAAGCA | 38343175 | 38343194 |
| 760 | CO-8991 | 1.095 | 0.185 | 5 uM | AACCCTGGTCAGTAGAAAGC | 38343174 | 38343193 |
| 761 | CO-8992 | 0.785 | 0.259 | 5 uM | GGAGAATATTGCCATGAAAA | 38343126 | 38343145 |
| 762 | CO-8993 | 0.876 | 0.373 | 5 uM | AGCAGTGACTTTTGAAGCCA | 38343079 | 38343098 |
| 763 | CO-8994 | 1.044 | 0.431 | 5 uM | AGAGAAGGCATTCACCAAGC | 38343062 | 38343081 |
| 764 | CO-8995 | 1.118 | 0.485 | 5 uM | GAGAGGCACCAACTACAAAG | 38343007 | 38343026 |
| 765 | CO-8996 | 1.087 | 0.476 | 5 uM | GAAGAGAGGCACCAACTACA | 38343004 | 38343023 |
| 766 | CO-8997 | 0.969 | 0.408 | 5 uM | GTCTGAAGAGAGGCACCAAC | 38343000 | 38343019 |
| 767 | CO-8998 | 1.246 | 0.507 | 5 uM | AGGGTCTGAAGAGAGGCACC | 38342997 | 38343016 |
| 768 | CO-8999 | 1.367 | 0.861 | 5 uM | AGTAGGGTCTGAAGAGAGGC | 38342994 | 38343013 |
| 769 | CO-9000 | 0.844 | 0.255 | 5 uM | AATCAATAGCAGGTCAAGAG | 38342965 | 38342984 |
| 770 | CO-9001 | 1.078 | 0.472 | 5 uM | TGCTTAGTAGATATAGCCCA | 38342938 | 38342957 |
| 771 | CO-9002 | 1.054 | 0.481 | 5 uM | GAGCCCCTGCTTAGTAGATA | 38342931 | 38342950 |
| 772 | CO-9003 | 1.065 | 0.409 | 5 uM | AGAGCCCCTGCTTAGTAGAT | 38342930 | 38342949 |
| 773 | CO-9004 | 1.047 | 0.429 | 5 uM | TTAATCATTAGCTTAAGGTG | 38342905 | 38342924 |
| 774 | CO-9005 | 0.898 | 0.454 | 5 uM | GTTTAATCATTAGCTTAAGG | 38342903 | 38342922 |
| 775 | CO-9006 | 0.947 | 0.366 | 5 uM | TGAGAGAAGAAGGCTGTGTT | 38342886 | 38342905 |
| 776 | CO-9007 | 0.994 | 0.242 | 5 uM | AGAGTTGTTACCAGTGGAGC | 38342861 | 38342880 |
| 777 | CO-9008 | 1.062 | 0.390 | 5 uM | ACAGAGTTGTTACCAGTGGA | 38342859 | 38342878 |
| 778 | CO-9009 | 1.108 | 0.581 | 5 uM | CAGGCCACAGAGTTGTTACC | 38342853 | 38342872 |
| 779 | CO-9010 | 0.961 | 0.384 | 5 uM | ACAGGCCACAGAGTTGTTAC | 38342852 | 38342871 |
| 780 | CO-9011 | 1.057 | 0.396 | 5 uM | TCTCTTGAATCTTGAAGACA | 38342786 | 38342805 |
| 781 | CO-9012 | 1.149 | 0.423 | 5 uM | ATGACCTAGGCTCCAAACAC | 38342737 | 38342756 |
| 782 | CO-9013 | 1.108 | 0.424 | 5 uM | GTAGCACCTATGACCTAGGC | 38342728 | 38342747 |
| 783 | CO-9014 | 1.097 | 0.446 | 5 uM | TGGTGAATATGTAGCACCTA | 38342718 | 38342737 |
| 784 | CO-9015 | 1.480 | 0.729 | 5 uM | GAAGAAATGGTGAACTCCAG | 38342668 | 38342687 |
| 785 | CO-9016 | 0.819 | 0.553 | 5 uM | CCTGGAAGAAATGGTGAACT | 38342664 | 38342683 |
| 786 | CO-9017 | 0.806 | 0.098 | 5 uM | GGTTCCCTGGAAGAAATGGT | 38342659 | 38342678 |
| 787 | CO-9018 | 1.066 | 0.602 | 5 uM | TGGTTCCCTGGAAGAAATGG | 38342658 | 38342677 |
| 788 | CO-9019 | 0.955 | 0.296 | 5 uM | CTCCTTGGTTCCCTGGAAGA | 38342653 | 38342672 |
| 789 | CO-9020 | 0.919 | 0.394 | 5 uM | TGAACTCCTTGGTTCCCTGG | 38342649 | 38342668 |
| 790 | CO-9021 | 1.029 | 0.418 | 5 uM | AAGGTGACCTGGAAATCCAT | 38342630 | 38342649 |
| 791 | CO-9022 | 0.939 | 0.287 | 5 uM | TTCGCTTACTTCTTAATGGT | 38342591 | 38342610 |
| 792 | CO-9023 | 0.995 | 0.060 | 5 uM | GATTCGCTTACTTCTTAATG | 38342589 | 38342608 |
| 793 | CO-9024 | 1.171 | 0.523 | 5 uM | GATGATTCGCTTACTTCTTA | 38342586 | 38342605 |
| 794 | CO-9025 | 1.113 | 0.314 | 5 uM | CAGATGATTCGCTTACTTCT | 38342584 | 38342603 |
| 795 | CO-9026 | 0.970 | 0.235 | 5 uM | GCCAGATGATTCGCTTACTT | 38342582 | 38342601 |
| 796 | CO-9027 | 1.144 | 0.394 | 5 uM | TTGCCAGATGATTCGCTTAC | 38342580 | 38342599 |
| 797 | CO-9028 | 1.013 | 0.326 | 5 uM | GCTTGCCAGATGATTCGCTT | 38342578 | 38342597 |
| 798 | CO-9029 | 1.086 | 0.348 | 5 uM | TGCTTGCCAGATGATTCGCT | 38342577 | 38342596 |
| 799 | CO-9030 | 1.298 | 0.463 | 5 uM | CTGCTTGCCAGATGATTCGC | 38342576 | 38342595 |
| 800 | CO-9031 | 1.252 | 0.497 | 5 uM | TAGTACTGCTTGCCAGATGA | 38342571 | 38342590 |
| 801 | CO-9032 | 0.959 | 0.469 | 5 uM | GCTGCTAGTACTGCTTGCCA | 38342566 | 38342585 |
| 802 | CO-9033 | 1.006 | 0.458 | 5 uM | GAGCTGCTAGTACTGCTTGC | 38342564 | 38342583 |
| 803 | CO-9034 | 1.119 | 0.471 | 5 uM | GCTATGAGTAGGAGCTGCTA | 38342553 | 38342572 |
| 804 | CO-9035 | 0.953 | 0.346 | 5 uM | TTGAACACTGAGCTAGGTAC | 38342484 | 38342503 |
| 805 | CO-9036 | 1.472 | 0.351 | 5 uM | TATTGAACACTGAGCTAGGT | 38342482 | 38342501 |
| 806 | CO-9037 | 1.102 | 0.577 | 5 uM | ACATGTGCCAATGGTAGTAG | 38342444 | 38342463 |
| 807 | CO-9038 | 1.075 | 0.444 | 5 uM | CCACATGTGCCAATGGTAGT | 38342442 | 38342461 |
| 808 | CO-9039 | 1.187 | 0.385 | 5 uM | TCACCACATGTGCCAATGGT | 38342439 | 38342458 |
| 809 | CO-9040 | 1.130 | 0.443 | 5 uM | GGAAAGTGATTAGAATAAGC | 38342392 | 38342411 |
| 810 | CO-9041 | 1.070 | 0.365 | 5 uM | GTAGGAAAGTGATTAGAATA | 38342389 | 38342408 |
| 811 | CO-9042 | 1.055 | 0.327 | 5 uM | GTATGAAAGAAAGACGGACA | 38342352 | 38342371 |
| 812 | CO-9043 | 1.217 | 0.669 | 5 uM | GGGTATGAAAGAAAGACGGA | 38342350 | 38342369 |
| 813 | CO-9044 | 1.087 | 0.568 | 5 uM | ACTTTTAATACAGCTCTGGA | 38342321 | 38342340 |
| 814 | CO-9045 | 1.220 | 0.531 | 5 uM | TCTCGATGCATAGCTGGGAT | 38342141 | 38342160 |
| 815 | CO-9046 | 0.961 | 0.186 | 5 uM | CCTCTCGATGCATAGCTGGG | 38342139 | 38342158 |
| 816 | CO-9047 | 1.041 | 0.335 | 5 uM | GCCTCTCGATGCATAGCTGG | 38342138 | 38342157 |
| 817 | CO-9048 | 0.928 | 0.252 | 5 uM | CAACACTTTTATTTGTAGGA | 38342375 | 38342394 |
| 818 | CO-9049 | 1.164 | 0.566 | 5 uM | CACCTCAACACTTTTATTTG | 38342370 | 38342389 |
| 819 | CO-9050 | 1.095 | 0.274 | 5 uM | GACGGACACCTCAACACTTT | 38342364 | 38342383 |
| 820 | CO-9051 | 1.022 | 0.406 | 5 uM | AAGAAAGACGGACACCTCAA | 38342358 | 38342377 |
| 821 | CO-9052 | 0.989 | 0.346 | 5 uM | TATGAAAGAAAGACGGACAC | 38342353 | 38342372 |
| 822 | CO-9053 | 1.817 | 1.016 | 5 uM | GGGGGTATGAAAGAAAGACG | 38342348 | 38342367 |
| 823 | CO-9054 | 1.425 | 0.702 | 5 uM | GGGTGGGGGTATGAAAGAAA | 38342344 | 38342363 |
| 824 | CO-9055 | 1.500 | 0.732 | 5 uM | AGTGGGGTGGGGGTATGAAA | 38342340 | 38342359 |
| 825 | CO-9056 | 1.236 | 0.518 | 5 uM | CTCTGGAGTGGGGTGGGGGT | 38342334 | 38342353 |
| 826 | CO-9057 | 0.860 | 0.381 | 5 uM | ACAGCTCTGGAGTGGGGTGG | 38342330 | 38342349 |
| 827 | CO-9058 | 0.852 | 0.326 | 5 uM | TTTCACTTTTAATACAGCTC | 38342317 | 38342336 |
| 828 | CO-9059 | 0.923 | 0.343 | 5 uM | CTGAATTTCACTTTTAATAC | 38342312 | 38342331 |
| 829 | CO-9060 | 0.948 | 0.387 | 5 uM | CAGCCTGAATTTCACTTTTA | 38342308 | 38342327 |
| 830 | CO-9061 | 1.087 | 0.503 | 5 uM | ATGCCCAGCCTGAATTTCAC | 38342303 | 38342322 |
| 831 | CO-9062 | 1.230 | 0.336 | 5 uM | CACCATGCCCAGCCTGAATT | 38342299 | 38342318 |
| 832 | CO-9063 | 1.170 | 0.285 | 5 uM | GAGCCACCATGCCCAGCCTG | 38342295 | 38342314 |
| 833 | CO-9064 | 1.123 | 0.359 | 5 uM | GCGTGAGCCACCATGCCCAG | 38342291 | 38342310 |
| 834 | CO-9065 | 0.980 | 0.292 | 5 uM | TTACAGGCGTGAGCCACCAT | 38342285 | 38342304 |
| 835 | CO-9066 | 1.048 | 0.336 | 5 uM | GGGATTACAGGCGTGAGCCA | 38342281 | 38342300 |
| 836 | CO-9067 | 1.003 | 0.389 | 5 uM | TGCTGGGATTACAGGCGTGA | 38342277 | 38342296 |
| 837 | CO-9068 | 0.894 | 0.322 | 5 uM | TCAAATGCCTCAATTGAAAG | 38343852 | 38343871 |
| 838 | CO-9069 | 0.933 | 0.300 | 5 uM | TATGAAGGACCCTGTCTACC | 38343828 | 38343847 |
| 839 | CO-9070 | 0.991 | 0.368 | 5 uM | GCATTTCACACTCTGTTCAG | 38343796 | 38343815 |
| 840 | CO-9071 | 1.128 | 0.263 | 5 uM | TATATGACAAGAGAGAAGCG | 38343751 | 38343770 |
| 841 | CO-9072 | 0.836 | 0.374 | 5 uM | CAGAATAATGATTTCAATGG | 38343721 | 38343740 |
| 842 | CO-9073 | 1.117 | 0.392 | 5 uM | AATCAATAACTTCCAGGGCA | 38343692 | 38343711 |
| 843 | CO-9074 | 1.065 | 0.331 | 5 uM | TTGTCCATATCTAAGCTCAA | 38343651 | 38343670 |
| 844 | CO-9075 | 1.203 | 0.423 | 5 uM | AGAACCAGGTATGGAAACTT | 38343624 | 38343643 |
| 845 | CO-9076 | 1.060 | 0.328 | 5 uM | GTGCAGAAATTTACCAGATC | 38343593 | 38343612 |
| 846 | CO-9077 | 1.154 | 0.789 | 5 uM | GTGTTGTGCATTTCCTAATC | 38343564 | 38343583 |
| 847 | CO-9078 | 0.931 | 0.505 | 5 uM | TTTACCCCTTTCTTACTTCA | 38343538 | 38343557 |
| 848 | CO-9079 | 1.138 | 0.304 | 5 uM | CACAAGGTTGGGTCATTAAC | 38343510 | 38343529 |
| 849 | CO-9080 | 1.395 | 0.111 | 5 uM | CTTGAAAGAGAACCTCGTGC | 38343468 | 38343487 |
| 850 | CO-9081 | 0.972 | 0.232 | 5 uM | CTAGGATTCTGTGTGGGAAC | 38343427 | 38343446 |
| 851 | CO-9082 | 1.023 | 0.351 | 5 uM | ATTGGAATCAGCCTATGGGA | 38343395 | 38343414 |
| 852 | CO-9083 | 1.020 | 0.273 | 5 uM | AACCTCTGTACTTCCAATGG | 38343369 | 38343388 |
| 853 | CO-9084 | 0.935 | 0.291 | 5 uM | GTGCCTGATAGCTTTCAGTC | 38343322 | 38343341 |
| 854 | CO-9085 | 0.913 | 0.447 | 5 uM | GTGAACTTTGAACCTCTGTG | 38343285 | 38343304 |
| 855 | CO-9086 | 1.342 | 0.354 | 5 uM | GCATTAGGGCTGATTGTTGA | 38343258 | 38343277 |
| 856 | CO-9087 | 1.250 | 0.399 | 5 uM | TGGGAGTGTCTGGTAGTCAC | 38343233 | 38343252 |
| 857 | CO-9088 | 1.149 | 0.393 | 5 uM | CATGGGATCCCCATCACTCA | 38343208 | 38343227 |
| 858 | CO-9089 | 1.071 | 0.226 | 5 uM | GTCAGTAGAAAGCAATGAAA | 38343181 | 38343200 |
| 859 | CO-9090 | 1.035 | 0.397 | 5 uM | ACAGTGCTCCTAGACCAAAC | 38343157 | 38343176 |
| 860 | CO-9091 | 1.125 | 0.353 | 5 uM | GCTCAAGGGGAGAATATTGC | 38343118 | 38343137 |
| 861 | CO-9092 | 0.895 | 0.347 | 5 uM | AGTGACTTTTGAAGCCATCC | 38343082 | 38343101 |
| 862 | CO-9093 | 1.004 | 0.188 | 5 uM | TGGGCAGAGAAGGCATTCAC | 38343057 | 38343076 |
| 863 | CO-9094 | 1.182 | 0.140 | 5 uM | GGTTTCAAGTTTCACCAAAA | 38343029 | 38343048 |
| 864 | CO-9095 | 1.121 | 0.344 | 5 uM | CTGAAGAGAGGCACCAACTA | 38343002 | 38343021 |
| 865 | CO-9096 | 1.170 | 0.339 | 5 uM | GTCAAGAGCACCTCCCAAGT | 38342977 | 38342996 |
| 866 | CO-9097 | 1.060 | 0.360 | 5 uM | CTGCTTAGTAGATATAGCCC | 38342937 | 38342956 |
| 867 | CO-9098 | 1.162 | 0.396 | 5 uM | CATTAGCTTAAGGTGAGGGC | 38342910 | 38342929 |
| 868 | CO-9099 | 0.963 | 0.195 | 5 uM | TACCAGTGGAGCAGCCTTGA | 38342869 | 38342888 |
| 869 | CO-9100 | 1.036 | 0.255 | 5 uM | TAGGTCCCATCTTTACAGGC | 38342838 | 38342857 |
| 870 | CO-9101 | 0.873 | 0.523 | 5 uM | ACAGGATTCCCATGGTCTAT | 38342803 | 38342822 |
| 871 | CO-9102 | 1.233 | 0.241 | 5 uM | CCCCATGAAAAGGGCTTGTT | 38342766 | 38342785 |
| 872 | CO-9103 | 1.080 | 0.273 | 5 uM | GTGAATATGTAGCACCTATG | 38342720 | 38342739 |
| 873 | CO-9104 | 0.881 | 0.270 | 5 uM | ATTCTGGAGGAATAAATCAA | 38342695 | 38342714 |
| 874 | CO-9105 | 0.950 | 0.417 | 5 uM | GAAATGGTGAACTCCAGTTG | 38342671 | 38342690 |
| 875 | CO-9106 | 1.023 | 0.349 | 5 uM | AAATCCATGAACTCCTTGGT | 38342642 | 38342661 |
| 876 | CO-9107 | 0.901 | 0.162 | 5 uM | TGCATAACAATGAAGGTGAC | 38342618 | 38342637 |
| 877 | CO-9108 | 0.850 | 0.099 | 5 uM | CTTGCCAGATGATTCGCTTA | 38342579 | 38342598 |
| 878 | CO-9109 | 1.015 | 0.452 | 5 uM | CTATGAGTAGGAGCTGCTAG | 38342554 | 38342573 |
| 879 | CO-9110 | 1.177 | 0.504 | 5 uM | TTACATTTCATACTCACAAC | 38342530 | 38342549 |
| 880 | CO-9111 | 1.081 | 0.534 | 5 uM | CTGTGCTACAGTACTCTCTA | 38342503 | 38342522 |
| 881 | CO-9112 | 1.023 | 0.093 | 5 uM | ATTTATTGAACACTGAGCTA | 38342479 | 38342498 |
| 882 | CO-9113 | 0.961 | 0.382 | 5 uM | GCCAATGGTAGTAGTTAACA | 38342450 | 38342469 |
| 883 | CO-9114 | 0.998 | 0.346 | 5 uM | AGCCAGGGATGGGGCCTCTC | 38342421 | 38342440 |
| 884 | CO-9115 | 0.961 | 0.325 | 5 uM | GAAAGTGATTAGAATAAGCC | 38342393 | 38342412 |
| 885 | CO-9116 | 0.914 | 0.385 | 5 uM | TCCGCCCGCCTCAAAGTGCT | 38342261 | 38342280 |
| 886 | CO-9117 | 0.965 | 0.459 | 5 uM | GGCTAGTCTCGAACTCCTGA | 38342233 | 38342252 |
| 887 | CO-9118 | 1.187 | 0.403 | 5 uM | TGTATTTTTAGTAGAGCCGG | 38342194 | 38342213 |
| 888 | CO-9119 | 1.081 | 0.643 | 5 uM | GTGTCCGCCACCATGCCTGG | 38342166 | 38342185 |
| 889 | CO-9120 | 1.248 | 0.376 | 5 uM | CAGCCTCTCGATGCATAGCT | 38342136 | 38342155 |
| 890 | CO-9121 | 1.125 | 0.512 | 5 uM | CTCCCGGGTTCAAGCAATTC | 38342108 | 38342127 |
| 891 | CO-9122 | 0.949 | 0.348 | 5 uM | TGGCACTATCTCGGCTCACT | 38342077 | 38342096 |
| 892 | CO-9123 | 0.933 | 0.151 | 5 uM | ATGGTGTCTCGCTCTGTCGC | 38342041 | 38342060 |
| 893 | CO-9158 | 1.558 | 0.318 | 5 uM | GTGCTCTTGACCTGCTATTG | 38342968 | 38342987 |
| 894 | CO-9159 | 1.302 | 0.266 | 5 uM | GGGAGGTGCTCTTGACCTGC | 38342973 | 38342992 |
| 895 | CO-9160 | 1.753 | 0.150 | 5 uM | TTGGTGCCTCTCTTCAGACC | 38342999 | 38343018 |
| 896 | CO-9161 | 1.321 | 0.165 | 5 uM | GTAGTTGGTGCCTCTCTTCA | 38343003 | 38343022 |
| 897 | CO-9162 | 1.509 | 0.449 | 5 uM | GTGAATGCCTTCTCTGCCCA | 38343057 | 38343076 |
| 898 | CO-9163 | 1.931 | 0.717 | 5 uM | ATTCTCCCCTTGAGCCCAGG | 38343113 | 38343132 |
| 899 | CO-9164 | 1.922 | 0.523 | 5 uM | TGCTTTCTACTGACCAGGGT | 38343175 | 38343194 |
| 900 | CO-9165 | 2.145 | 0.895 | 5 uM | GACACTCCCAGGACCTGAGT | 38343223 | 38343242 |
| 901 | CO-9166 | 1.381 | 0.421 | 5 uM | TGCTCCCTGTGACTACCAGA | 38343241 | 38343260 |
| 902 | CO-9167 | 1.336 | 0.503 | 5 uM | TCAGCCCTAATGCTCCCTGT | 38343251 | 38343270 |
| 903 | CO-9168 | 1.273 | 0.478 | 5 uM | GTGTCTCAACAATCAGCCCT | 38343263 | 38343282 |
| 904 | CO-9169 | 1.115 | 0.394 | 5 uM | CCAGTGTCTCAACAATCAGC | 38343266 | 38343285 |
| 905 | CO-9170 | 1.271 | 0.181 | 5 uM | CTCCCATAGGCTGATTCCAA | 38343396 | 38343415 |
| 906 | CO-9171 | 1.449 | 0.698 | 5 uM | GAGATGCAAAGCACGAGGTT | 38343478 | 38343497 |
| 907 | CO-9172 | 1.367 | 0.358 | 5 uM | GAGGCAGTTGAGATGCAAAG | 38343487 | 38343506 |
| 908 | CO-9173 | 1.390 | 0.485 | 5 uM | CAGAGGCAGTTGAGATGCAA | 38343489 | 38343508 |
| 909 | CO-9174 | 1.379 | 0.439 | 5 uM | TGTGTCAGAGGCAGTTGAGA | 38343494 | 38343513 |
| 910 | CO-9175 | 1.232 | 0.195 | 5 uM | GATTAGGAAATGCACAACAC | 38343564 | 38343583 |
| 911 | CO-9176 | 1.456 | 0.212 | 5 uM | GAACAGAGTGTGAAATGCCA | 38343794 | 38343813 |
| 912 | CO-9177 | 1.153 | 0.194 | 5 uM | TCACCCATTAGCTGGCTCCT | 38344293 | 38344312 |
| 913 | CO-9178 | 1.343 | 0.139 | 5 uM | CGGTATTGTTTTGCGGCTTG | 38344369 | 38344388 |
| 914 | CO-9179 | 1.462 | 0.428 | 5 uM | GAGACGGTATTGTTTTGCGG | 38344373 | 38344392 |
| 915 | CO-9180 | 1.326 | 0.415 | 5 uM | GCTCTGTTGTGAGACGGTAT | 38344383 | 38344402 |
| 916 | CO-9181 | 1.346 | 0.432 | 5 uM | CTAACGTGCTGAAGGACCCA | 38344463 | 38344482 |
| 917 | CO-9182 | 1.148 | 0.296 | 5 uM | GGTCATATCTAACGTGCTGA | 38344471 | 38344490 |
| 918 | CO-9183 | 1.311 | 0.374 | 5 uM | GTCCCAGGTCATATCTAACG | 38344477 | 38344496 |
| 919 | CO-9184 | 1.624 | 0.533 | 5 uM | CCACATGTGGATGCCCAATT | 38344521 | 38344540 |
| 920 | CO-9185 | 1.269 | 0.311 | 5 uM | TTGAGGTTGAGATTGAGTTT | 38344547 | 38344566 |
| 921 | CO-9186 | 0.998 | 0.124 | 5 uM | GGCTTGAGGTTGAGATTGAG | 38344550 | 38344569 |
| 922 | CO-9187 | 1.210 | 0.254 | 5 uM | TGTCTAAGGCTTGAGGTTGA | 38344557 | 38344576 |
| 923 | CO-9188 | 1.107 | 0.280 | 5 uM | GTTTTGTCTAAGGCTTGAGG | 38344561 | 38344580 |
| 924 | CO-9189 | 1.353 | 0.176 | 5 uM | CTCTCCCTATGTCTTCTGAA | 38344628 | 38344647 |
| 925 | CO-9190 | 1.315 | 0.329 | 5 uM | CTGACCCCTATTCTTCTTCT | 38344908 | 38344927 |
| 926 | CO-9191 | 1.320 | 0.513 | 5 uM | CACATTCCTGGTCTTGAACA | 38344987 | 38345006 |
| 927 | CO-9192 | 1.621 | 0.393 | 5 uM | TTGGTAGGTATAGAGTCTCA | 38345028 | 38345047 |
| 928 | CO-9193 | 1.173 | 0.094 | 5 uM | AGGAAGGAGGTCACTTTGGG | 38345340 | 38345359 |
| 929 | CO-9194 | 1.409 | 0.400 | 5 uM | AGTACTGCACTTGATGGATC | 38345443 | 38345462 |
| 930 | CO-9195 | 1.242 | 0.442 | 5 uM | GTGTCAGAGGCAGTTGAGAT | 38343493 | 38343512 |
| 931 | CO-9196 | 1.322 | 0.613 | 5 uM | CCTTGTGTCAGAGGCAGTTG | 38343497 | 38343516 |
| 932 | CO-9197 | 1.236 | 0.231 | 5 uM | CCAACCTTGTGTCAGAGGCA | 38343501 | 38343520 |
| 933 | CO-9198 | 2.230 | 1.457 | 5 uM | ATGGTTAATGACCCAACCTT | 38343513 | 38343532 |
| 934 | CO-9199 | 1.413 | 0.443 | 5 uM | ATAAATATGGTTAATGACCC | 38343519 | 38343538 |
| 935 | CO-9200 | 0.998 | 0.674 | 5 uM | GGGGTAAATAAATATGGTTA | 38343526 | 38343545 |
| 936 | CO-9201 | 1.055 | 0.058 | 5 uM | GAAAGGGGTAAATAAATATG | 38343530 | 38343549 |
| 937 | CO-9202 | 1.497 | 0.186 | 5 uM | AGTAAGAAAGGGGTAAATAA | 38343535 | 38343554 |
| 938 | CO-9203 | 1.671 | 0.353 | 5 uM | CAATTTATGAAGTAAGAAAG | 38343545 | 38343564 |
| 939 | CO-9204 | 1.735 | 0.716 | 5 uM | AACACAATTTATGAAGTAAG | 38343549 | 38343568 |
| 940 | CO-9205 | 1.823 | 0.377 | 5 uM | GCACAACACAATTTATGAAG | 38343553 | 38343572 |
| 941 | CO-9206 | 2.200 | 0.427 | 5 uM | GAAATGCACAACACAATTTA | 38343558 | 38343577 |
| 942 | CO-9207 | 1.842 | 0.221 | 5 uM | TTAGGAAATGCACAACACAA | 38343562 | 38343581 |
| 943 | CO-9208 | 1.153 | 0.477 | 5 uM | AAGATTAGGAAATGCACAAC | 38343566 | 38343585 |
| 944 | CO-9209 | 1.121 | 0.154 | 5 uM | GAAGACAAGATTAGGAAATG | 38343572 | 38343591 |
| 945 | CO-9210 | 1.569 | 0.484 | 5 uM | TGCACAGAAGACAAGATTAG | 38343578 | 38343597 |
| 946 | CO-9211 | 1.274 | 0.145 | 5 uM | TTTCTGCACAGAAGACAAGA | 38343582 | 38343601 |
| 947 | CO-9212 | 1.564 | 0.344 | 5 uM | GGTAAATTTCTGCACAGAAG | 38343588 | 38343607 |
| 948 | CO-9213 | 1.396 | 0.280 | 5 uM | GAGGTGCTCTTGACCTGCTA | 38342971 | 38342990 |
| 949 | CO-9214 | 1.545 | 0.247 | 5 uM | GTGCCTCTCTTCAGACCCTA | 38342996 | 38343015 |
| 950 | CO-9215 | 1.178 | 0.228 | 5 uM | CTACTTTTTGGTGAAACTTG | 38343034 | 38343053 |
| 951 | CO-9216 | 1.417 | 0.513 | 5 uM | TGCTTGGTGAATGCCTTCTC | 38343063 | 38343082 |
| 952 | CO-9217 | 1.332 | 0.402 | 5 uM | GTCTTGGATGGCTTCAAAAG | 38343087 | 38343106 |
| 953 | CO-9218 | 1.341 | 0.236 | 5 uM | TATTCTCCCCTTGAGCCCAG | 38343114 | 38343133 |
| 954 | CO-9219 | 1.494 | 0.430 | 5 uM | TAGGAGCACTGTCCCAGTAA | 38343149 | 38343168 |
| 955 | CO-9220 | 1.249 | 0.434 | 5 uM | TTGCTTTCTACTGACCAGGG | 38343176 | 38343195 |
| 956 | CO-9221 | 1.290 | 0.330 | 5 uM | ACCTGAGTGATGGGGATCCC | 38343211 | 38343230 |
| 957 | CO-9222 | 1.111 | 0.251 | 5 uM | ATGCTCCCTGTGACTACCAG | 38343242 | 38343261 |
| 958 | CO-9223 | 1.085 | 0.233 | 5 uM | GTTCACCAGTGTCTCAACAA | 38343271 | 38343290 |
| 959 | CO-9224 | 1.255 | 0.313 | 5 uM | ACAGGGAAATCACAGAGGTT | 38343295 | 38343314 |
| 960 | CO-9225 | 1.307 | 0.340 | 5 uM | GCAGACTGAAAGCTATCAGG | 38343325 | 38343344 |
| 961 | CO-9226 | 1.551 | 0.423 | 5 uM | CCCCATTGGAAGTACAGAGG | 38343371 | 38343390 |
| 962 | CO-9227 | 1.283 | 0.434 | 5 uM | TCTCCCATAGGCTGATTCCA | 38343397 | 38343416 |
| 963 | CO-9228 | 1.333 | 0.357 | 5 uM | CACACAGAATCCTAGAGCTA | 38343422 | 38343441 |
| 964 | CO-9229 | 1.371 | 0.349 | 5 uM | CAAGGATGCCCTTTTATCTC | 38343452 | 38343471 |
| 965 | CO-9230 | 1.565 | 0.475 | 5 uM | GATGCAAAGCACGAGGTTCT | 38343476 | 38343495 |
| 966 | CO-9231 | 1.424 | 0.575 | 5 uM | GATCTGGTAAATTTCTGCAC | 38343593 | 38343612 |
| 967 | CO-9232 | 1.693 | 0.884 | 5 uM | GTTTCCATACCTGGTTCTTT | 38343622 | 38343641 |
| 968 | CO-9233 | 1.338 | 0.573 | 5 uM | TTGAGCTTAGATATGGACAA | 38343651 | 38343670 |
| 969 | CO-9234 | 1.371 | 0.323 | 5 uM | TTGCCCTGGAAGTTATTGAT | 38343693 | 38343712 |
| 970 | CO-9235 | 1.361 | 0.308 | 5 uM | CCATTGAAATCATTATTCTG | 38343721 | 38343740 |
| 971 | CO-9236 | 1.130 | 0.475 | 5 uM | CTCGCTTCTCTCTTGTCATA | 38343753 | 38343772 |
| 972 | CO-9237 | 1.310 | 0.438 | 5 uM | CAGAGTGTGAAATGCCAATC | 38343791 | 38343810 |
| 973 | CO-9238 | 1.292 | 0.376 | 5 uM | GAAGAAGGTAGACAGGGTCC | 38343834 | 38343853 |
| 974 | CO-9239 | 1.057 | 0.224 | 5 uM | CATACCCTTTCAATTGAGGC | 38343858 | 38343877 |
| 975 | CO-9240 | 1.468 | 0.555 | 5 uM | GAGGATTATCTCTGACCCCC | 38343897 | 38343916 |
| 976 | CO-9241 | 1.370 | 0.475 | 5 uM | TTATTCAAACAGGTGGGCCC | 38343939 | 38343958 |
| 977 | CO-9242 | 1.323 | 0.388 | 5 uM | ATTGATTCAGGGGTGTTTTC | 38343981 | 38344000 |
| 978 | CO-9243 | 1.251 | 0.249 | 5 uM | GGTAATGGTAATGGTAGTTT | 38344011 | 38344030 |
| 979 | CO-9244 | 1.317 | 0.625 | 5 uM | CAAGCGATCTGAGAGTTACT | 38344046 | 38344065 |
| 980 | CO-9245 | 1.184 | 0.344 | 5 uM | GAGAATCTGAGAATTACTTT | 38344185 | 38344204 |
| 981 | CO-9246 | 1.449 | 0.481 | 5 uM | TCACCAAAAGAGCTCCTGAT | 38344210 | 38344229 |
| 982 | CO-9247 | 1.164 | 0.181 | 5 uM | TTTCTCTCCACGTGTGTGTG | 38344270 | 38344289 |
| 983 | CO-9248 | 1.058 | 0.228 | 5 uM | ATCACCCATTAGCTGGCTCC | 38344294 | 38344313 |
| 984 | CO-9249 | 1.174 | 0.206 | 5 uM | TAAATTAGAATTATTATAGT | 38344321 | 38344340 |
| 985 | CO-9250 | 1.139 | 0.127 | 5 uM | CTGTGTGGAAACTGGCAATA | 38344346 | 38344365 |
| 986 | CO-9251 | 1.024 | 0.116 | 5 uM | GACGGTATTGTTTTGCGGCT | 38344371 | 38344390 |
| 987 | CO-9252 | 1.216 | 0.305 | 5 uM | CCCAGAATGTTTTATAAGCT | 38344413 | 38344432 |
| 988 | CO-9253 | 0.985 | 0.232 | 5 uM | CTTCATGTGTTAATATTCTA | 38344438 | 38344457 |
| 989 | CO-9254 | 1.155 | 0.340 | 5 uM | TAACGTGCTGAAGGACCCAA | 38344462 | 38344481 |
| 990 | CO-9255 | 1.093 | 0.052 | 5 uM | GAGTCCTCTATGTCCCAGGT | 38344488 | 38344507 |
| 991 | CO-9256 | 1.345 | 0.247 | 5 uM | TCCACATGTGGATGCCCAAT | 38344522 | 38344541 |
| 992 | CO-9257 | 1.338 | 0.325 | 5 uM | GCTTGAGGTTGAGATTGAGT | 38344549 | 38344568 |
| 993 | CO-9258 | 1.486 | 0.250 | 5 uM | GATCCACTGTGAGTTTTGTC | 38344573 | 38344592 |
| 994 | CO-9259 | 1.242 | 0.348 | 5 uM | GCCTTACATTTTCCATTCAG | 38344599 | 38344618 |
| 995 | CO-9260 | 1.199 | 0.401 | 5 uM | GATTCTCTCCCTATGTCTTC | 38344632 | 38344651 |
| 996 | CO-9261 | 1.304 | 0.181 | 5 uM | CATGCTGTTGAACTCTTTGC | 38344670 | 38344689 |
| 997 | CO-9262 | 1.111 | 0.241 | 5 uM | GATCAAGTTCAATTTACCAT | 38344709 | 38344728 |
| 998 | CO-9263 | 1.067 | 0.119 | 5 uM | TATGTTTTTCACAAAGCAAA | 38344742 | 38344761 |
| 999 | CO-9264 | 1.427 | 0.099 | 5 uM | TGCTTGTCTTTTCAGTCTCC | 38344766 | 38344785 |
| 1000 | CO-9265 | 1.189 | 0.211 | 5 uM | TTATTGGATATGTAACTTGC | 38344807 | 38344826 |
| 1001 | CO-9266 | 1.437 | 0.138 | 5 uM | GTTCTACATATATATTCTGG | 38344838 | 38344857 |
| 1002 | CO-9267 | 1.277 | 0.370 | 5 uM | TACTTGAATTGTTTATTTTC | 38344877 | 38344896 |
| 1003 | CO-9268 | 1.094 | 0.137 | 5 uM | GAGCTTGCCTGACCCCTATT | 38344916 | 38344935 |
| 1004 | CO-9269 | 0.993 | 0.127 | 5 uM | GTCTTGAACAGTCCTCCAGC | 38344977 | 38344996 |
| 1005 | CO-9270 | 0.978 | 0.159 | 5 uM | TTTGGTAGGTATAGAGTCTC | 38345029 | 38345048 |
| 1006 | CO-9271 | 0.971 | 0.102 | 5 uM | AAATAGCTAGGAGTATAGGT | 38345075 | 38345094 |
| 1007 | CO-9272 | 1.269 | 0.048 | 5 uM | AGGCGGTTTCACAATAGTGT | 38345157 | 38345176 |
| 1008 | CO-9273 | 1.200 | 0.256 | 5 uM | TTCACTGTCTGTCATAATTT | 38345182 | 38345201 |
| 1009 | CO-9274 | 1.405 | 0.252 | 5 uM | ATGGATTCGGTTAAGTTAGA | 38345207 | 38345226 |
| 1010 | CO-9275 | 1.217 | 0.252 | 5 uM | CCATAAACTATAAACTAAGT | 38345293 | 38345312 |
| 1011 | CO-9276 | 1.122 | 0.059 | 5 uM | GGGGTGTTACCGTCTTTGTT | 38345317 | 38345336 |
| 1012 | CO-9277 | 1.017 | 0.192 | 5 uM | GCAGGAAGGAGGTCACTTTG | 38345342 | 38345361 |
| 1013 | CO-9278 | 0.975 | 0.039 | 5 uM | CACCACCAGATCAATAAACT | 38345417 | 38345436 |
| 1014 | CO-9279 | 1.277 | 0.270 | 5 uM | GTACTGCACTTGATGGATCA | 38345442 | 38345461 |
| 1015 | CO-9280 | 1.051 | 0.085 | 5 uM | CTAAAATCAGTGCTTGGGGT | 38345466 | 38345485 |
| 1016 | CO-9281 | 1.003 | 0.295 | 5 uM | ATATGGTTTAGGAGTTATGC | 38345526 | 38345545 |
| 1017 | CO-9282 | 1.197 | 0.142 | 5 uM | GGTCAGAGACTTGGGTTTGA | 38352783 | 38352802 |
| 1018 | CO-9283 | 1.215 | 0.124 | 5 uM | GATGGTCAGAGACTTGGGTT | 38352780 | 38352799 |
| 1019 | CO-9284 | 1.041 | 0.164 | 5 uM | GGTAAGTGATGGTCAGAGAC | 38352773 | 38352792 |
| 1020 | CO-9285 | 1.087 | 0.082 | 5 uM | GCAGCTTTTAGAAATGGTCA | 38352730 | 38352749 |
| 1021 | CO-9286 | 1.139 | 0.149 | 5 uM | GTTTAATCTGAGGATCCTGT | 38352702 | 38352721 |
| 1022 | CO-9287 | 1.242 | 0.180 | 5 uM | TGGCTAACTTGCTGTGGAGT | 38352636 | 38352655 |
| 1023 | CO-9288 | 1.523 | 0.077 | 5 uM | GGTGGAGCTTGGCATAAAGT | 38352486 | 38352505 |
| 1024 | CO-9289 | 1.386 | 0.190 | 5 uM | GGCCAGGCAATAAAAGAGTC | 38352438 | 38352457 |
| 1025 | CO-9290 | 1.297 | 0.090 | 5 uM | TCACAGCAGCCGGTACCGCA | 38352327 | 38352346 |
| 1026 | CO-9291 | 1.237 | 0.339 | 5 uM | GGCAATTAGAGGTAGAAAAG | 38352207 | 38352226 |
| 1027 | CO-9292 | 1.084 | 0.056 | 5 uM | GTGGAAAGACTGGCAATTAG | 38352196 | 38352215 |
| 1028 | CO-9293 | 1.101 | 0.240 | 5 uM | AAAGGGAGCTCCAGGACTGA | 38352100 | 38352119 |
| 1029 | CO-9294 | 1.045 | 0.021 | 5 uM | GTCAGGTGCTGTTAGAATCA | 38351713 | 38351732 |
| 1030 | CO-9295 | 1.061 | 0.222 | 5 uM | GACTGTCAGGTGCTGTTAGA | 38351709 | 38351728 |
| 1031 | CO-9296 | 1.274 | 0.135 | 5 uM | GTGATCTGAATAGGCTGCTA | 38351650 | 38351669 |
| 1032 | CO-9297 | 1.196 | 0.178 | 5 uM | GATGGCCCCTTAGTGATCTG | 38351638 | 38351657 |
| 1033 | CO-9298 | 1.174 | 0.220 | 5 uM | GTCTGGACCTCTCCTATGGG | 38351443 | 38351462 |
| 1034 | CO-9299 | 0.947 | 0.145 | 5 uM | CTCCTACCCTTCCTACCTTA | 38351389 | 38351408 |
| 1035 | CO-9300 | 1.099 | 0.167 | 5 uM | TCTGTAATCAGATTTCACCG | 38352939 | 38352958 |
| 1036 | CO-9301 | 1.013 | 0.303 | 5 uM | AGACAGCTGCCTCTAATTCC | 38352894 | 38352913 |
| 1037 | CO-9302 | 0.993 | 0.255 | 5 uM | TAGTGCCACGCTCTGCTTTA | 38352864 | 38352883 |
| 1038 | CO-9303 | 1.167 | 0.364 | 5 uM | ATATTCTGCAGTAAGGCCTC | 38352831 | 38352850 |
| 1039 | CO-9304 | .192 | 0.166 | 5 uM | GGGTTTGATTTAGGAATCAT | 38352795 | 38352814 |
| 1040 | CO-9305 | 0.872 | 0.545 | 5 uM | TTTCGGTAAGTGATGGTCAG | 38352769 | 38352788 |
| 1041 | CO-9306 | 1.162 | 0.441 | 5 uM | TGGTCACAACTTCATGGTTC | 38352744 | 38352763 |
| 1042 | CO-9307 | 1.405 | 0.288 | 5 uM | ATGCTGTTTAATCTGAGGAT | 38352697 | 38352716 |
| 1043 | CO-9308 | 1.226 | 0.56 | 5 uM | TTCAAGGGCATAGAATCGTC | 38352657 | 38352676 |
| 1044 | CO-9309 | 1.035 | 0.073 | 5 uM | CTTAGTTTTTAGGTGGCCCC | 38352613 | 38352632 |
| 1045 | CO-9310 | 1.136 | 0.027 | 5 uM | GGGTGATATTACCTTTGCTC | 38352570 | 38352589 |
| 1046 | CO-9311 | 0.998 | 0.226 | 5 uM | ACCAGGGGACTTTGATAAGG | 38352542 | 38352561 |
| 1047 | CO-9312 | 1.184 | 0.251 | 5 uM | ATGCTCCTACACCCTGCCCT | 38352510 | 38352529 |
| 1048 | CO-9313 | 1.168 | 0.283 | 5 uM | AGCGGTGGAGCTTGGCATAA | 38352483 | 38352502 |
| 1049 | CO-9314 | 1.236 | 0.380 | 5 uM | CCAGGCAATAAAAGAGTCAG | 38352440 | 38352459 |
| 1050 | CO-9315 | 1.413 | 0.548 | 5 uM | ACATGCGTGTGACAGTATAA | 38352397 | 38352416 |
| 1051 | CO-9316 | 1.248 | 0.400 | 5 uM | ATCCTCAATGAGTACTTGTC | 38352365 | 38352384 |
| 1052 | CO-9317 | 1.279 | 0.473 | 5 uM | TTCACAGCAGCCGGTACCGC | 38352326 | 38352345 |
| 1053 | CO-9318 | 1.109 | 0.376 | 5 uM | CTGAGTGAGTTACTTCTACT | 38352302 | 38352321 |
| 1054 | CO-9319 | 1.122 | 0.453 | 5 uM | TGTGTTTTTGCAGTGCCAAT | 38352273 | 38352292 |
| 1055 | CO-9320 | 1.216 | 0.229 | 5 uM | AATAGTACTACTCAGGACTG | 38352240 | 38352259 |
| 1056 | CO-9321 | 1.253 | 0.317 | 5 uM | AAGACTGGCAATTAGAGGTA | 38352201 | 38352220 |
| 1057 | CO-9322 | 0.993 | 0.202 | 5 uM | AGGGCACTTCTTTCTATTTG | 38352171 | 38352190 |
| 1058 | CO-9323 | 1.111 | 0.364 | 5 uM | CTATCATCTTGCACCCCCAA | 38352141 | 38352160 |
| 1059 | CO-9324 | 1.268 | 0.222 | 5 uM | GGACTGAGATATTTTTACTA | 38352113 | 38352132 |
| 1060 | CO-9325 | 1.421 | 0.446 | 5 uM | GAAGACTTATGTGCAAGGCA | 38352079 | 38352098 |
| 1061 | CO-9326 | 0.962 | 0.256 | 5 uM | CAGGTGCTGTTAGAATCAAT | 38351715 | 38351734 |
| 1062 | CO-9327 | 1.011 | 0.167 | 5 uM | GCATATGGTATCCCCACTTC | 38351678 | 38351697 |
| 1063 | CO-9328 | 1.683 | 0.418 | 5 uM | TCTGAATAGGCTGCTAGGGG | 38351654 | 38351673 |
| 1064 | CO-9329 | 1.171 | 0.211 | 5 uM | TGAAGTTGCAGATGGCCCCT | 38351628 | 38351647 |
| 1065 | CO-9330 | 1.183 | 0.433 | 5 uM | CTGATTTCTAACTGAGGATA | 38351596 | 38351615 |
| 1066 | CO-9331 | 1.213 | 0.475 | 5 uM | GTGCAGCTCGGTATCTGATA | 38351556 | 38351575 |
| 1067 | CO-9332 | 1.195 | 0.203 | 5 uM | GATGATGTCATCTTCCCTCT | 38351525 | 38351544 |
| 1068 | CO-9333 | 1.102 | 0.294 | 5 uM | ACCACCTTTACTGAACTGTG | 38351494 | 38351513 |
| 1069 | CO-9334 | 1.113 | 0.156 | 5 uM | TCCTATGGGCTTGTATTATG | 38351454 | 38351473 |
| 1070 | CO-9335 | 1.110 | 0.223 | 5 uM | CTTCTTCGAAGCCAGACAAC | 38351420 | 38351439 |
| 1071 | CO-9336 | 1.480 | 0.312 | 5 uM | ACCCTTCCTACCTTAGAGGG | 38351394 | 38351413 |
| 1072 | CO-9337 | 1.346 | 0.380 | 5 uM | AATTGACCAGAAGAGGCAGC | 38351344 | 38351363 |

### Example 2: Modulation of SERPING1 Expression Using paRNA- or eRNa-Targeting ASOs

This example was designed to assess modulation of SERPING1 expression in murine hepatocytes using ASOs targeting a paRNA transcribed from murine SERPING1 promoter.

See FIG. 19 for sequences and chemical modifications of selected mouse Serping1 ASOs.

Female C57BU/6 mice, ˜6-7 weeks old, were treated with a single 5 mg/kg IP dose of IFNy (125 ug per mouse) or PBS as a negative control, and sacrificed at 30 min, 1 hr, 2 hr, 6 hrs, 10 hrs, and 24 hrs post-treatment. Male C57BU/6 mice, ˜7 weeks old, were treated twice with 15 mg/kg IP dose of Tofacitinib (12 hours apart), and sacrificed at two hrs and 6 hrs post-treatment. Livers from mice in both experiments were collected at the listed timepoints and processed for RNA isolation and cDNA synthesis for relative RNA measurements (Taqman qPCR (Mm00437835_m1)).

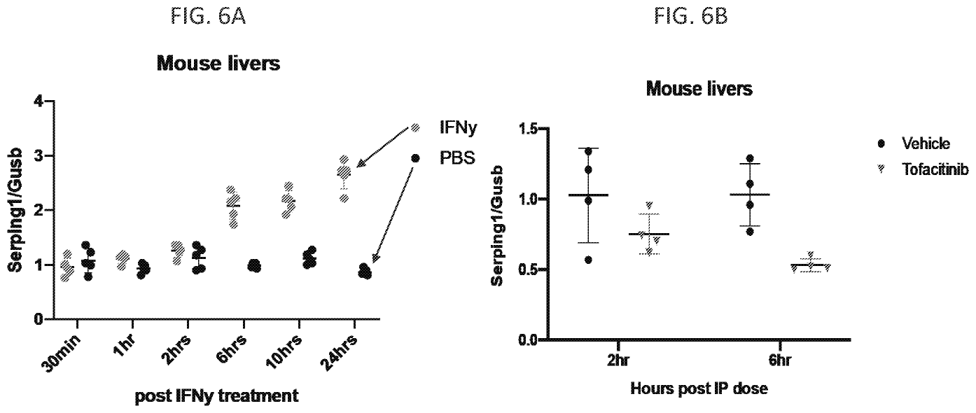

Serping1 mRNA was upregulated in a time dependent manner with IFNy, with highest fold-change (approximately 3-fold induction) occurring at 24 hrs post dose (FIG. 6A). Serping1 mRNA was downregulated with the Jak1 inhibitor Tofacitinib, with 50% decrease occurring at 6 hrs (FIG. 6B). Thus, Serping1 is likely controlled by the IFNy-Jak pathway.

Female C57Bl/6 mice, ˜6-7 weeks old, were treated with a single 5 mg/kg IP dose of IFNg (125 ug per mouse), and sacrificed at 24 hrs, 48 hrs, and 72 hrs post-treatment. Blood serum was collected for mRNA and protein analysis via Western Blot. Serping1 antibody used was rabbit monoclonal [EPR8015] to SERPING1(ab134918). Protein levels were normalized to Transferrin protein (Rabbit Abcam 82411). Serping1 mRNA was normalized to Hmbs as the housekeeping gene.

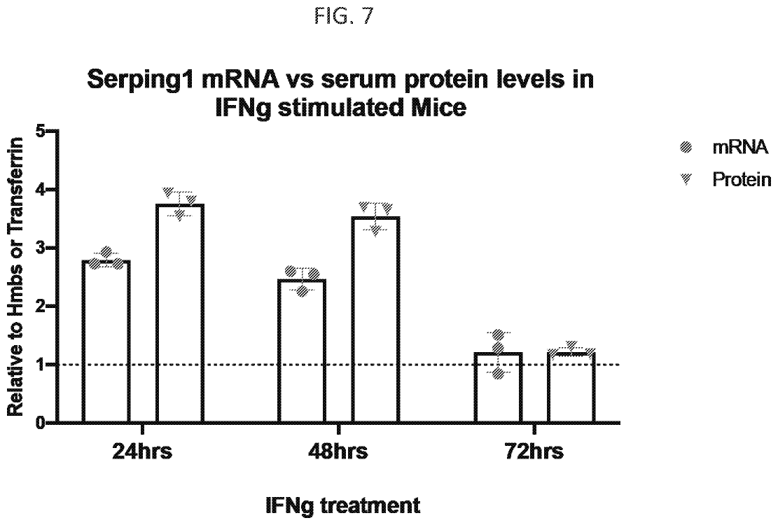

A sustained increase in Serping1 mRNA and protein in serum was observed from 24-48 hrs (FIG. 7). Blood serum mRNA levels after IFNg treatment are provided in Table 10.

TABLE 10

| Timepoint | Serping1 mRNA with IFNy |
|---|---|
| 24 hr | 2.65 |
| 48 hr | 2.16 |
| 72 hr | 1.3 |

Next, female C57Bl/6 mice, ˜6-7 weeks old, were treated with a single 5 mg/kg IP dose of IFNy (125 ug per mouse) or PBS as a negative control, and sacrificed at 6 hrs and 24 hrs post-treatment. Livers from the 6 hrs and 24 hrs timepoints were processed via Qiagen Trizol method and measured via SYBR green PCR. Serping1 mRNA and regRNA expression levels were determined using PCR.

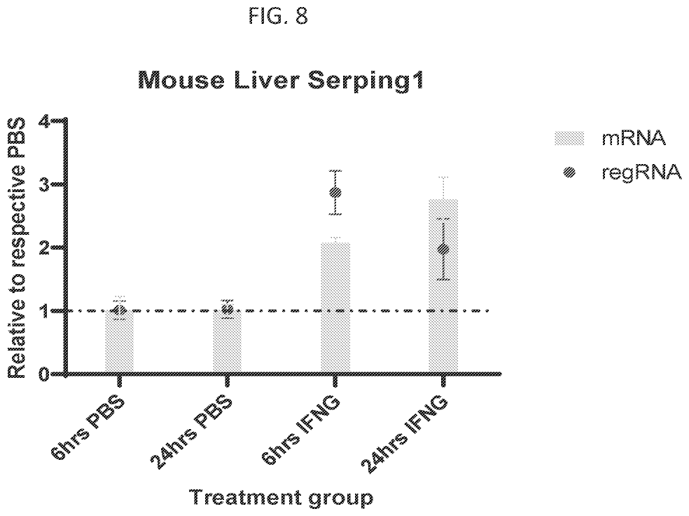

As shown in FIG. 8, regRNA levels increased first, followed by the increase in mRNA after induction by IFNg.

Next, cryopreserved Mouse hepatocytes (Lonza) were plated onto collagen-coated plates, allowed to attach for 24 hrs, and were stimulated with 1000 ng/ml IFNy and collected at 0.5 hr, 2 hrs, 4 hrs, 8 hrs, 24 hrs, and 30 hrs post-treatment. Cells were lysed in Qiagen RLT buffer and processed using Quick-RNA Zymo kits and mRNA measured via SYBR green qPCR using regRNA-specific primers.

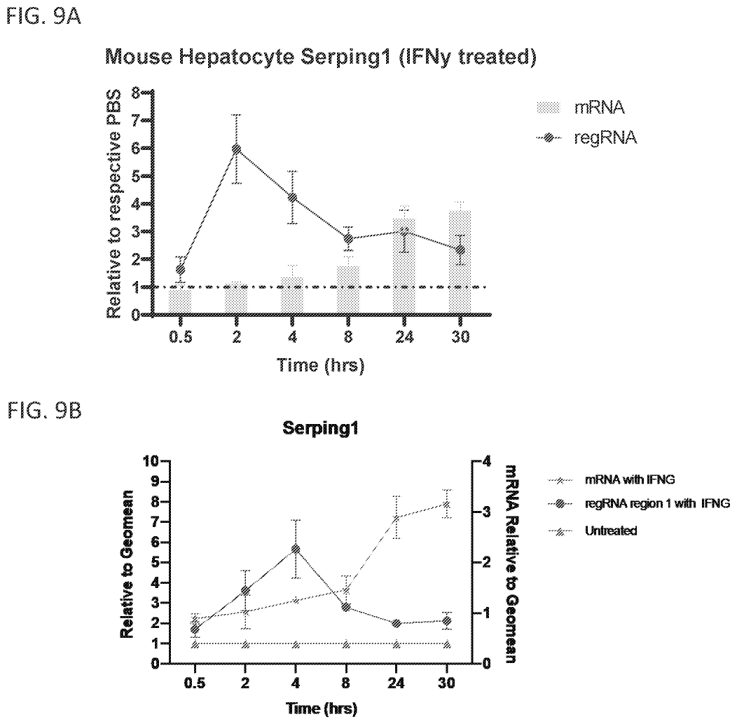

As shown in FIGS. 9A and 9B, IFNy stimulation increased Serping1 regRNA prior to upregulating Serping1 mRNA. Serping1 regRNA levels peaked at 2 hrs, while Serping1 mRNA peaked at 30 hrs. Thus, IFNg treatment lead to time-dependent increase in Serping1 mRNA in mouse hepatocytes.

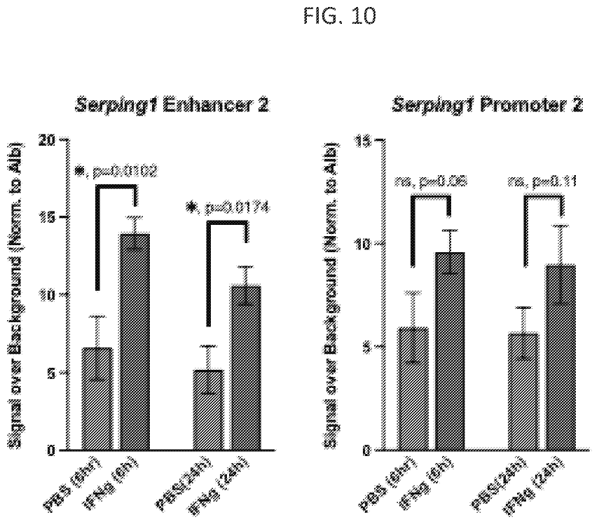

Female C57Bl/6 mice, ˜6-7 weeks old, were treated with a single 5 mg/kg IP dose of IFNg (125 ug per mouse) and collected at 6 hrs, and 24 hrs post-treatment. Livers powders from 6 hrs and 24 hrs timepoints were processed for ATAC-seq. Epigenomic data revealed 2 hotspots, enhancer 2 and promoter 2 as ideal regions for targeting and upregulation (FIG. 10).

Next, Cryopreserved Mouse hepatocytes (Lonza) were treated with selected ASOs (mSerping1pa-ASO-1, mSerping1pa-ASO-2, and mSerping1pa-ASO-3) in power media in a dose-response via free uptake method on Day 1 (24 hrs post plating) and harvested on Day 3. Scramble ASO (NTC-3S) was used as a control. Cells were lysed in RLT Qiagen buffer and processed via RNAeasy Plus 96 Kit and mRNA measured via Taqman qPCR. mSerping1pa-ASO-1 is an optimized sequence of mSerping1pa-ASO-2.

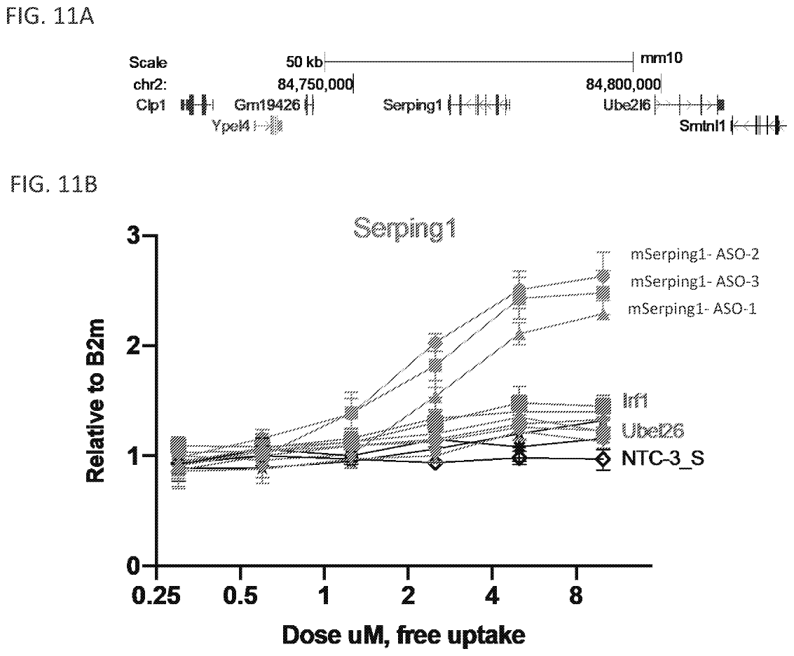

Serping1 mRNA was upregulated in a dose-dependent manner with the selected ASOs targeting the paRNA (FIG. 11B), while neighboring genes Irf1 and Ubel26 were not upregulated. A schematic of the Serping1 chromosomal neighborhood is shown in FIG. 11A.

Next, optimized versions of the lead ASO sequences were designed and tested in freshly isolated Mouse hepatocytes. Cells treated with ASOs in power media in a dose-response via free uptake method on Day 1 (24 hrs post plating) and harvested on Day 3. Scramble ASO (NTC-3S) was used as a control. mSERPING1-ASOpa-6 is a IONIS murine sequence targeting Serping1 (Bhattacharjee et al., 2013).

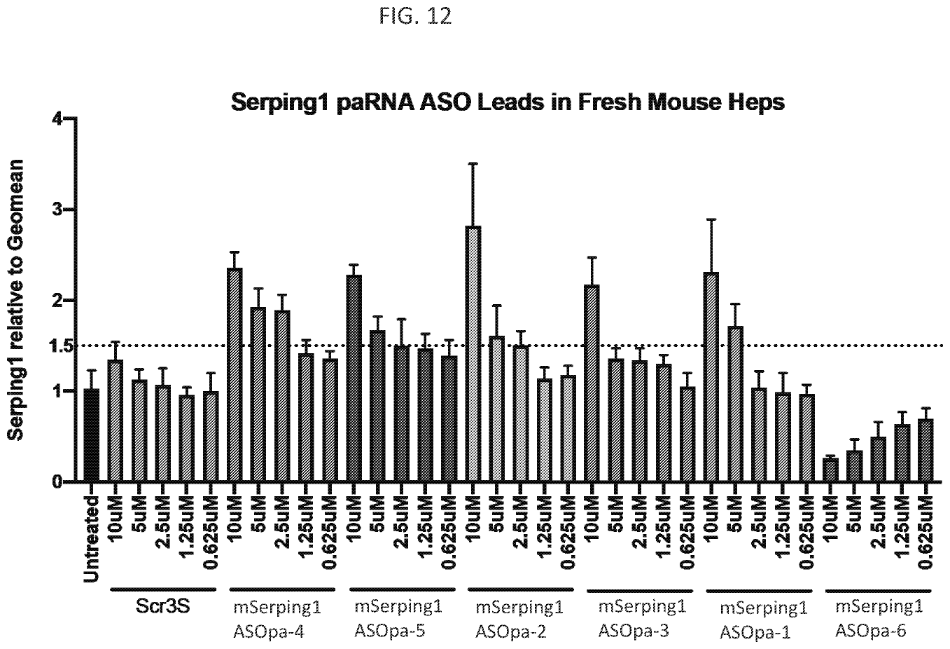

As shown in FIG. 12, Serping1 was upregulated in a dose-dependent manner with ASOs targeting regRNAs. Fold change in Serping1 mRNA after treatment with the ASOs is shown in Table 11.

TABLE 11

| ASO | FC at 5 uM |
|---|---|
| NTC-3S | 1.13 |
| mSERPING1-ASOpa-4 | 1.93 |
| mSERPING1-ASOpa-5 | 1.67 |
| mSERPING1-ASOpa-2 | 1.61 |
| mSERPING1-ASOpa-3 | 1.36 |
| mSERPING1-ASOpa-1 | 1.72 |

A longer time assay was also performed. Freshly isolated Mouse hepatocytes were treated with 10 μM of selected ASOs (mSerping1pa-ASO-2, mSerping1pa-ASO-3, mSerping1pa-ASO-4) in power media via free uptake method on Day 1 (24 hrs post plating) and collected at 8 hrs, 24 hrs, 48 hrs, and 72 hrs for RNA processing. Scramble ASO (NTC-Scr3S) was used as a control. mRNA was normalized to NTC.

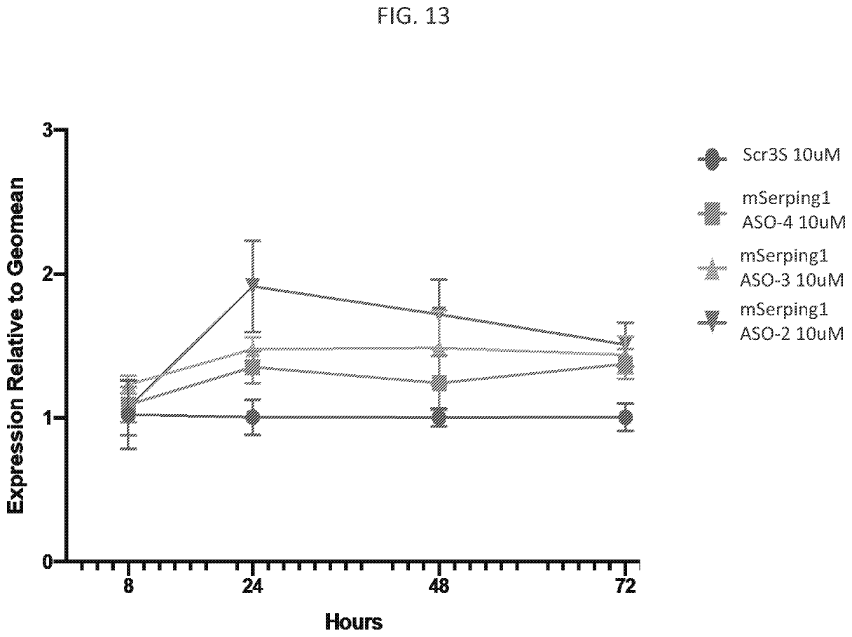

The selected ASOs increased Serping1 to ˜1.5-2× at 24 hrs (FIG. 13)

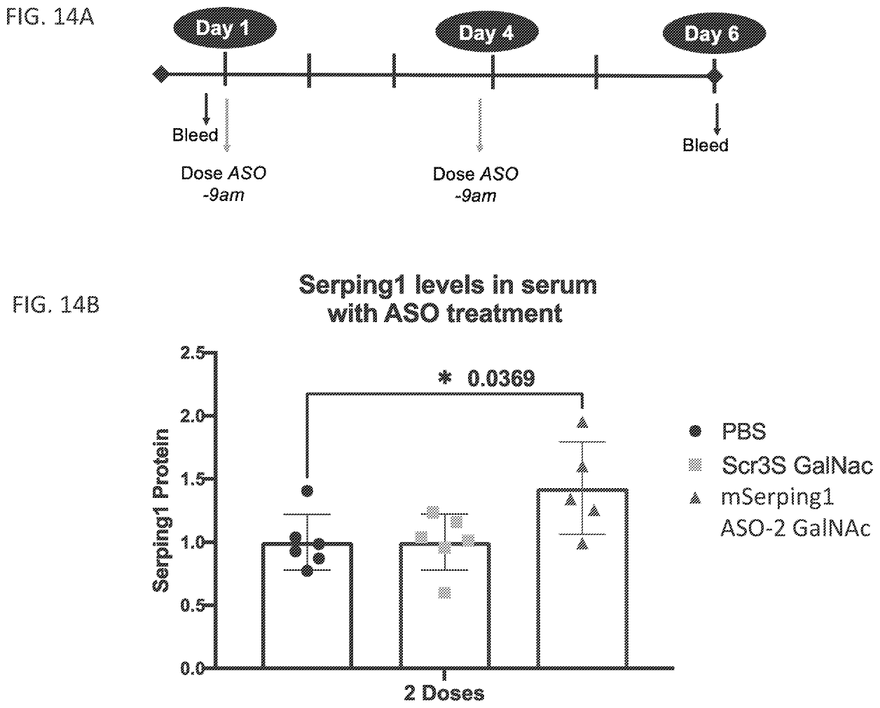

An in vivo assay was next performed. Male C57/B16 mice, ˜8 weeks, were treated with a selected ASO conjugated to GalNAc (mSerping1 ASO-2 GalNAc) via SC injection on days 1 and 4, collected serum collected on day 6. PBS and scramble ASO NTC were used as controls. Serum bleeds were used to measure Serping1 protein via western blot. A schematic of the study design is shown in FIG. 14A.

After 2 doses of ASO conjugated to GalNAc, Serping1 protein levels increase ˜1.5-fold as compared to negative controls (FIG. 14B).

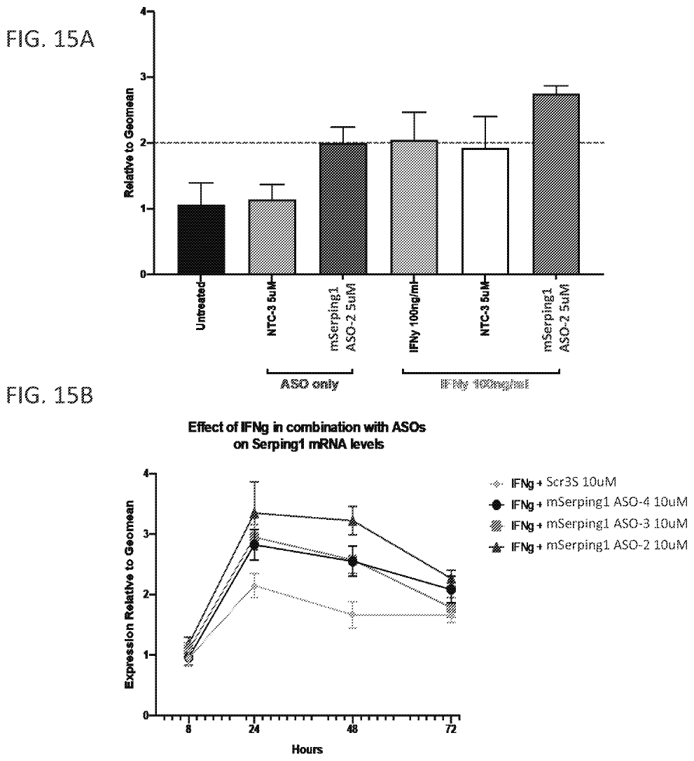

The additive effect of IFNg plus ASO treatment on Serping1 mRNA expression was next assessed. Cryopreserved Mouse hepatocytes were treated with 5 μM mSerping1 ASO-2 in power media plus 100 ng/ml IFNg via free uptake method on Day 2. Cells were collected on Day 4 for mRNA analysis. Untreated mice and scramble ASO NTC were used as controls. 5 μM mSerping1 ASO-2 in combination with IFNg lead to the highest fold change, approximately 2.75 fold relative to the negative controls (FIG. 15A).

A time course assay for the combination therapy was also performed. Freshly isolated mouse hepatocytes were treated with 10 μM mSerping1 ASO-2, mSerping1 ASO-3, or mSerping1 ASO-4 in power media plus 1000 ng/ml IFNg via free uptake method on Day 1 (24 hrs post plating) and collected at 8 hr, 24 hr, 48 hr, and 72 hr for RNA processing. Scramble ASO NTC plus IFNg was used as control.

Higher concentrations of the ASOs also resulted in approximately 3-fold increase in Serping1 mRNA over control mice (FIG. 15B).

Next, the rescued effect of a Jak1 inhibitor plus ASO on Serping1 mRNA was assessed. Cryopreserved mouse hepatocytes were treated with 5 μM mSerping1 ASO-2 in power media plus 3 μM Jak1 inhibitor tofacitinib via free uptake method on Day 2. Cells were collected on Day 4 for mRNA analysis.

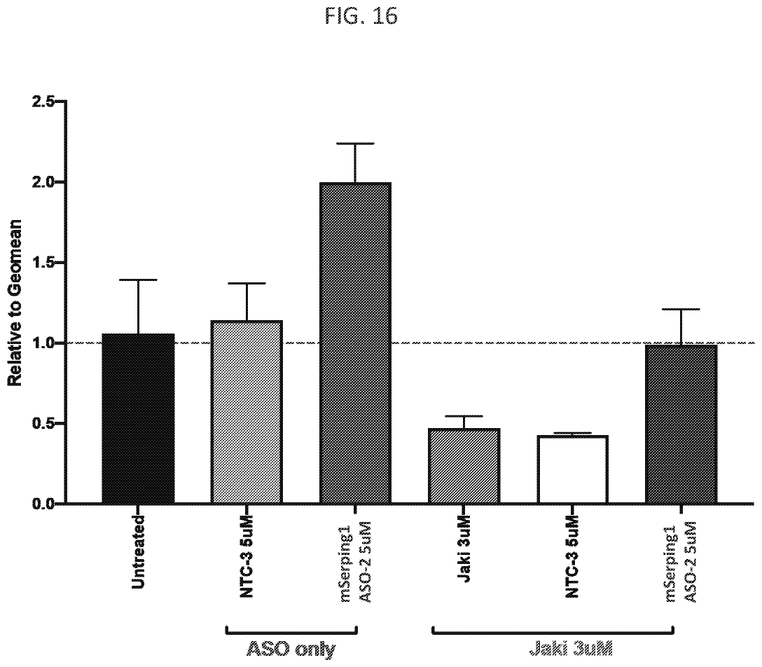

mSerping1 ASO-2 in combination with Jak1 inhibition resulted in recovery of Serping1 mRNA to normal levels (FIG. 16).

A similar rescue experiment was performed in an Serping1 knockdown (KD) system using 1 μM Jak1 inhibitor tofacitinib. This system mimics HAE disease since in HAE, there is only one healthy copy of Serping1, so the absolute levels are 50% of WT individuals.

Freshly isolated Mouse hepatocytes were treated with 10 μM and 5 μM mSerping1 ASO-2 and mSerping1 ASO-3 in power media with Jak1 at 1 μM via free uptake method on Day 1. Cells were collected on Day 4 for mRNA analysis.

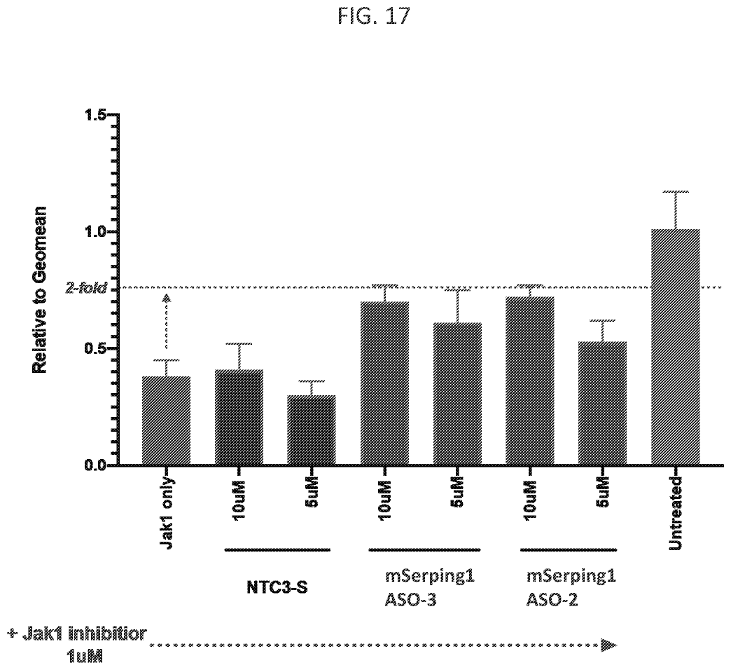

The Jak1 inhibitor decreased Serping1 to 50% of normal expression, similar to the HAE disease. After treatment with the selected ASOs, Serping1 levels were restored >1.5 fold, nearing WT levels (FIG. 17).

Additional ASO were tiled around mSERPING1-ASOpa-1 (CO-3149), mSERPING1-ASOpa-2 (CO-2043), and mSERPING1-ASOpa-3 (CO-2051). The new sequences are provided below:

| SEQ | | | |
|---|---|---|---|
| ID | | | |
| NO | Name | INFO | Sequence |
| 1079 | CO-3263 | Tiled for CO-3149 | GGCGGAGACAGAGAGAAGGT |
| 1080 | CO-3264 | Tiled for CO-3149 | CAGAGGGCGGAGACAGAGAG |
| 1081 | CO-3265 | Tiled for CO-3149 | AAAACAGAGGGCGGAGACAG |
| 1082 | CO-3266 | Tiled for CO-3149 | ACAAAACAGAGGGCGGAGAC |
| 1083 | CO-3267 | Tiled for CO-2051 | GAGGGCTTGGCAGGGAGATC |
| 1084 | CO-3268 | Tiled for CO-2051 | AAGTTAAAGAGGGCTTGGCA |
| 1085 | CO-3269 | Tiled for CO-2051 | GAACACAAAGTTAAAGAGGG |
| 1086 | CO-3270 | Tiled for CO-2051 | GAGGAACACAAAGTTAAAGA |
| 1087 | CO-3271 | Tiled for CO-2051 | GGGAGGAACACAAAGTTAAA |
| 1089 | CO-3272 | Tiled for CO-2051 | CTTTGGGAGGAACACAAAGT |
| 1090 | CO-3273 | Tiled for CO-2051 | GGGCTTTGGGAGGAACACAA |
| 1091 | CO-3274 | Tiled for CO-2051 | GGGGGCTTTGGGAGGAACAC |
| 1092 | CO-3275 | Tiled for CO-2051 | GACCAGGTTTGGTAATAGGG |
| 1093 | CO-3276 | Tiled for CO-2051 | CAAGAACTTGGACCAGGTTT |
| 1094 | CO-3277 | CO-2043 extension to 22 nt | GCGGAGCAGGGAGATTGAACAG |
| 1095 | CO-3278 | CO-2043 extension to 24 nt | AGCGGAGCAGGGAGATTGAACAGA |
| 1096 | CO-3279 | CO-2051 extension to 22 nt | TTTGGGAGGAACACAAAGTTAA |
| 1097 | CO-3280 | CO-2051 extension to 24 nt | CTTTGGGAGGAACACAAAGTTAAA |

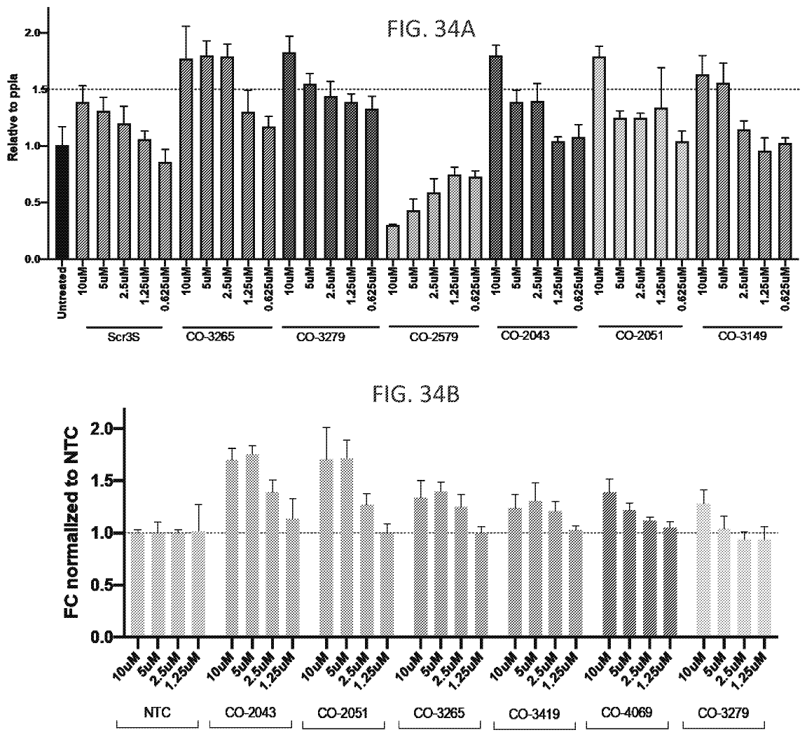

ASOs were tested as previously described. Briefly, mouse hepatocytes were plated and treated with ASOs 24 hours after plating on day 1. Cells were harvested 48 hours post treatment. As shown in FIG. 34A, ASOs CO-3265, CO-3279, CO-2043, and CO-2051 increased Serping1 mRNA expression in a dose dependent manner.

Next, select Serping1 ASOs were tested in C1NH+/− hepatocytes from a C57BL/6J mouse. C1NH+/− hepatocytes are deficient for Serping1 expression. As shown in FIG. 34B, the ASOs CO-2043, CO-2051, CO-3265, CO-3419, CO-4069, and CO-3279 increased Serping1 gene expression in C1NH+/− hepatocytes in a dose dependent manner.

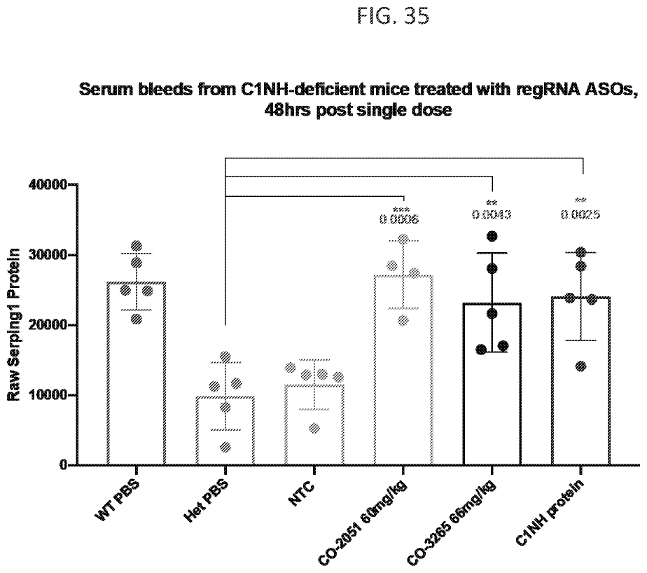

A GAlNAc-ASO was also tested in C1NH-deficient mice. Mice were bled and dosed with ASOs CO-2051 and CO-3265 on days 1 and 3 and sacrificed on day 6. As shown in FIG. 35, both ASOs increased Serping1 mRNA in the mice.

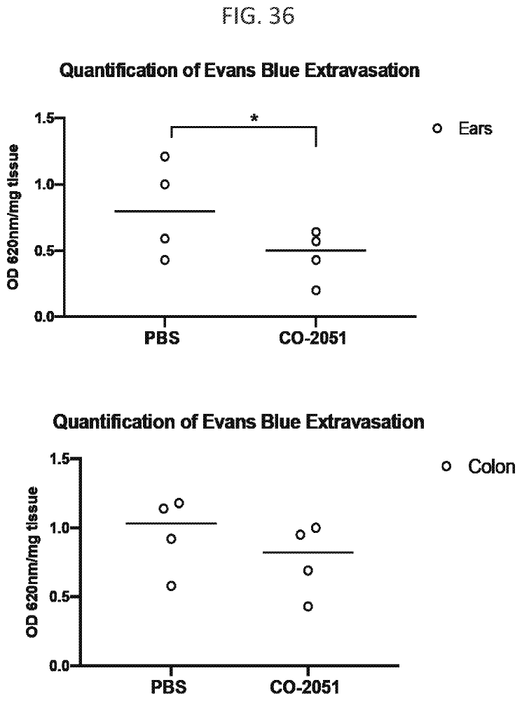

Next, a vascular permeability assay was performed. C57 B16 mice were injected subcutaneously with ASO at a dose of 260 mg/kg/wk. Evan's blue treatment was based of reference J Clin Invest. 2002; 109(8):1057-1063, injected IP at 150 mg/kg and carried out on Day 6 and Day 8. Quantification of dye occurred on mice terminated on Day 8. At necropsy, tissues were dried, weighed, and added to 1 mL formamide. Dye was extracted from tissue and measured at OD 620 nm. As shown in FIG. 36, CO-2051 decreased the amount of dye extravasation in both the ears and colons of C1NH+/− mice.

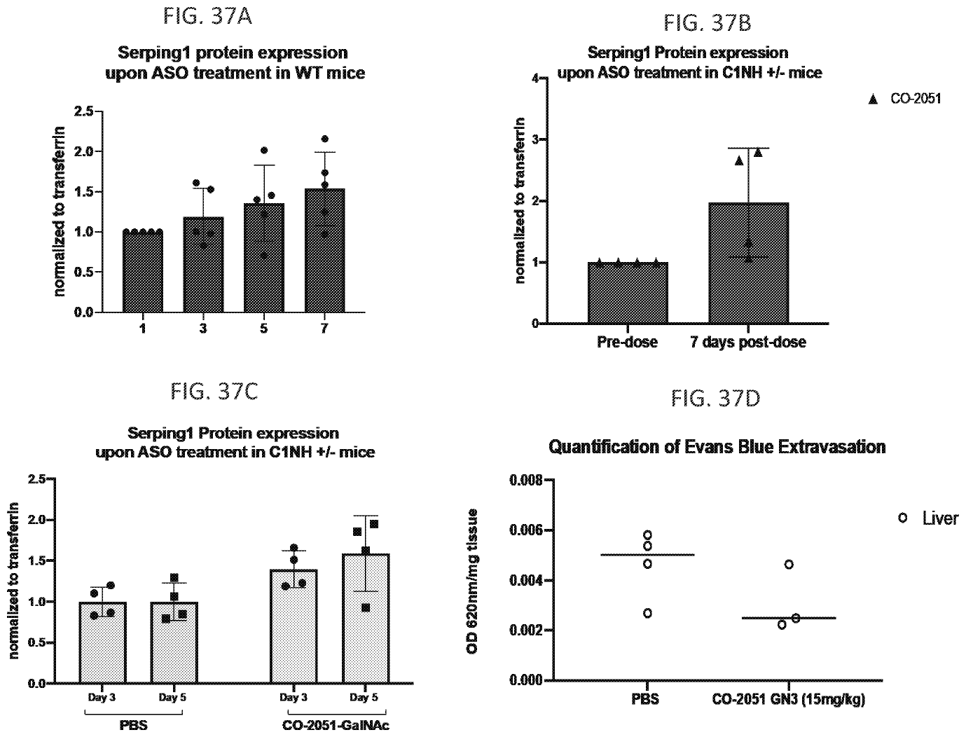

CO-2051 also increased Serping1 mRNA in both WT and C1NH+/− mice. WT or C1NH+/− mice were treated with 260 mg/kg ASO. Blood was collected and processed to serum on days 1, 3, 5 and 7 for protein measurement via Western Blot using a constant loading volume (ex. 1 uL Serum). Serping1 and transferrin abeam antibodies were added using standard method. Respective bands were imaged using LiCOR scanner and quantified using ImageStudio Analysis software. As shown in FIGS. 37A and 3B, Serping1 upregulation observed with the naked ASO in WT and diseased mice.

The assay was repeated and a sustained protein upregulation was observed with a lower dose of GalNAc-ASO CO-2051 (15 mg/kg) (FIGS. 37C and 37D).

### Example 3: Tiling and Optimizing Human OTC regRNA-Targeting ASOs

Additional ASOs made by base-walking and extension around hOTC-ASOe1-2a were synthesized and characterized. In addition, ASOs were fined tuned by altering the chemistry, type, and position of the chemical modification. ASOs synthesized and further characterized were ASO sequences hOTC-ASOe1-1a, hOTC-ASOe1-3a, hOTC-ASOe1-4a, hOTC-ASOe1-1h, and hOTC-ASOe1-1d.

Additional ASOs made by base-walking and extension around hOTC-ASOe2-2a were also synthesized and characterized. In addition, ASO's were fined tuned by altering the chemistry, type, and position of the chemical modification. ASOs synthesized and further characterized were ASO sequences hOTC-ASO-e2-2a, hOTC-ASO-e2-2b, hOTC-ASO-e2-2c, hOTC-ASO-e2-2d, hOTC-ASO-e2-2e, hOTC-ASO-e2-4, hOTC-ASO-e2-5, hOTC-ASO-e2-6, and hOTC-ASO-e2-7. See Tables 2, 3, 4, and FIG. 18A, 18B, 18D, and 18E for human OTC sequences and chemical modifications of selected ASOs.

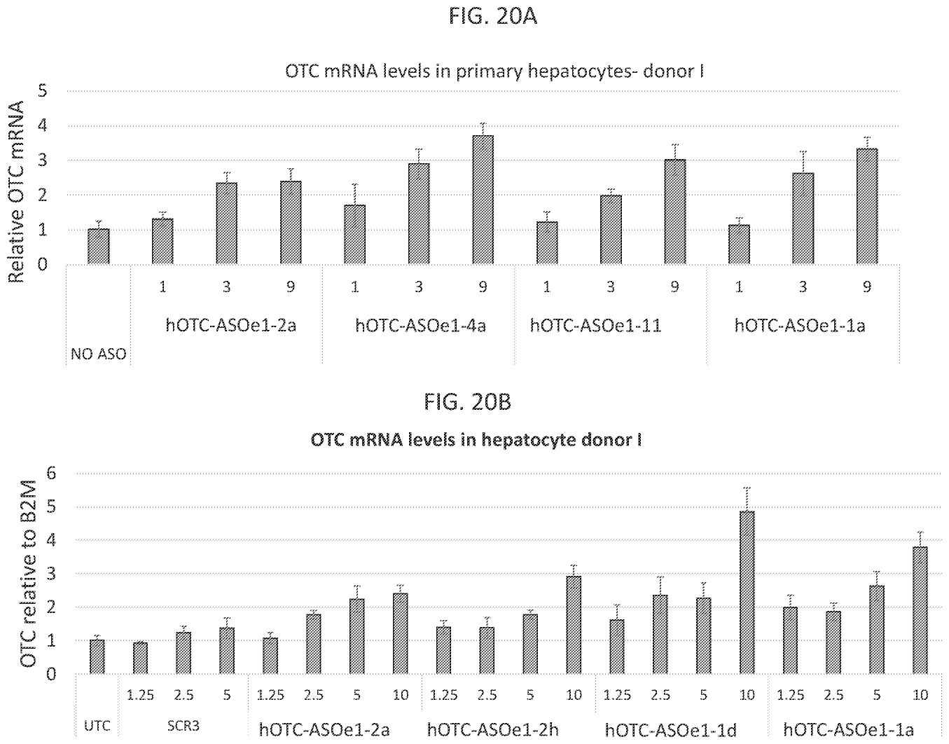

Hepatocytes from one donor were cultured in vitro. Cells were plated in growth media and treated 4-6 hours after plating with final concentrations of 1 μM, 3 μM, or 9 μM of hOTC-ASO-e1-4a (FIG. 20A, base walking ASOs), or 1.25 μM, 2.5 μM, 5 μM, or 10 μM hOTC-ASOe1-1d, hOTC-ASOe1-1h, or hOTC-ASOe1-1a (FIG. 20B, fine tuning ASOs).

Hepatocytes from one donor were cultured in vitro. Cells were plated in growth media and treated 4-6 hours after plating with final concentrations of 1 μM, 3 μM, or 9 μM of hOTC-ASO-e2-2a, hOTC-ASO-e2-2b, hOTC-ASO-e2-2c, hOTC-ASO-e2-2d, hOTC-ASO-e2-2e, hOTC-ASO-e2-4, hOTC-ASO-e2-5, hOTC-ASO-e2-6, and hOTC-ASO-e2-7.

Cells were collected 48 hr post treatment and processed for RNA isolation, cDNA synthesis and QPCR analysis. Taqman probe Hs00166892_m1 (OTC) 60× was used for OTC expression. OTC levels were normalized to B2M expression.

The base-walking and extension around hOTC-ASOe1-2a lead to 3-fold improvement in potency as compared to the parent sequence hOTC-ASOe1-2a (FIG. 20A). Additional fine tuning by altering the type, chemistry, and position of modification resulted in increased efficacy as compared to the parent sequence hOTC-ASOe1-2a as shown by the dose-dependent increase of OTC mRNA in primary hepatocytes (FIG. 20B).

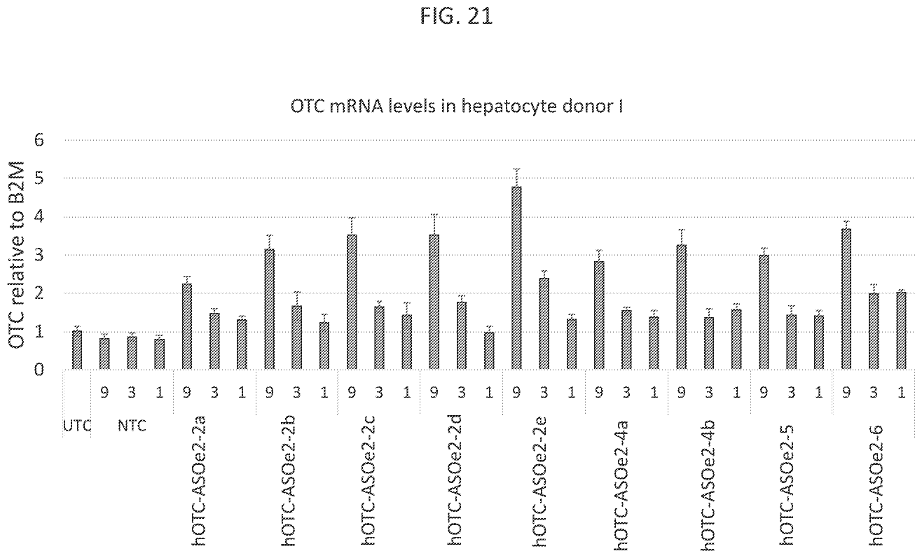

Fine tuning by altering the type, chemistry, and position of modification based on hOTC-ASOe2-2a also resulted in increased efficacy as compared to the parent sequence, as shown by the dose-dependent increase of OTC mRNA in primary hepatocytes (FIG. 21).

Next, selected ASOs were characterized in the OTC-deficient donor cell line. Hepatocytes from an OTC-deficient donor were cultured in vitro. Cells were plated in growth media and treated 4 hours post plating with a final concentration of 1 μM, 3 μM, and 9 μM ASO hOTC-ASOe1-10, hOTC-ASOe1-2a, hOTC-ASOe1-12, hOTC-ASOe1-11, and hOTC-ASOe1-1a. A non-targeting control (NTC) ASO comprising a random sequence was used as the negative control (SRC3). The supernatant was collected for ureagenesis analysis and cell lysate was collected for mRNA at Day 2 and Day 6 post treatment. For mRNA analysis, the taqman probe Hs00166892_m1 was used for OTC expression. OTC levels were normalized to B2M expression. For the Ureagenesis, the collected supernatant was measured by Urea Nitrogen (BUN) Colorimetric Detection Kit (Thermofisher, catalog #: EIABUN) and normalized by Albumin ELISA (Bethyl, Catalog #: E88-129). Statistics were performed using one way ANOVA in Prism (GraphPad).

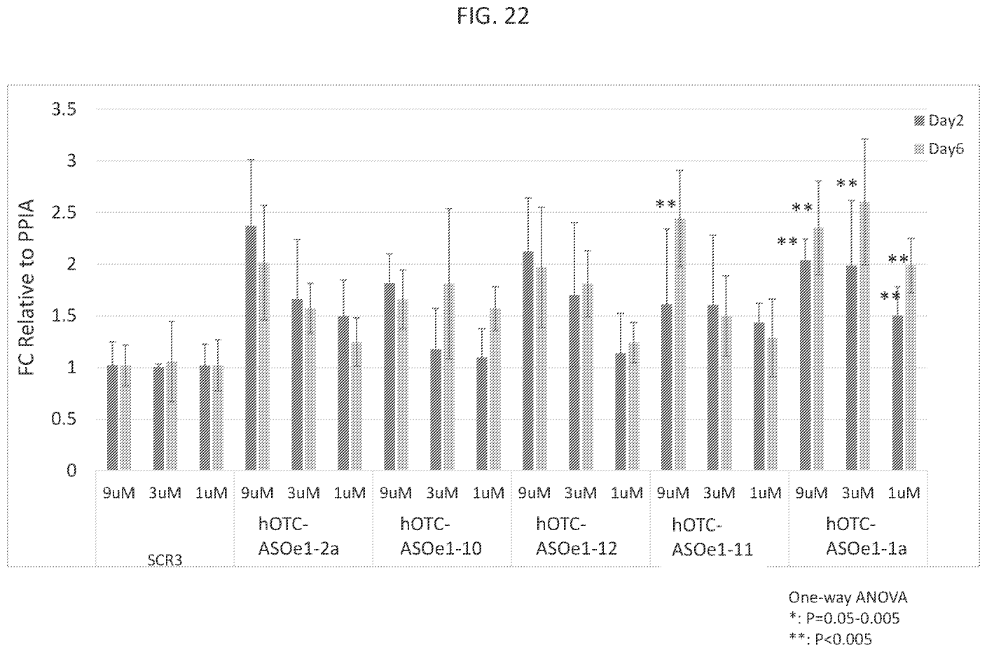

As shown in FIG. 22, a dose-dependent increase in OTC mRNA was observed after treatment with multiple ASOs at Day 2 and Day 6.

Next, an in vitro PBMC assay was run to assess ASO toxicity.

Peripheral blood mononuclear cells (PBMCs) were isolated from fresh human whole blood (provided by Research Blood Components LLC). A volume of 15 ml of whole blood was mixed with 15 ml of PBS+2% FBS, added to a SepMate Isolation Tube (STEMCELL Technologies) containing 15 ml of Ficoll and centrifuged at 800 g for 20 mins. The resulting top layer was removed, and the remaining mononuclear cell layer was washed with 20 ml of PBS+2% FBS, followed by centrifugation at 300 g for 8 minutes. Two additional washes with PBS+2% FBS were performed. After the third wash, the cell pellet was resuspended in red cell lysis buffer (Abcam, ab204733) for 10 minutes, followed by centrifugation at 400 g for 5 minutes. The pellet was then resuspended in 10 ml of PBS+2% FBS, centrifuged at 120 g for 10 minutes and the final PBMC pellet was resuspended in RPMI 1640 (Sigma Aldrich). Isolated PBMCs were seeded at a density of 100,000 cells per well in a V-bottom 96-well plate and treated with 0.7 uM or 1.4 uM of hOTC-ASOe1-1a or NTC. After 24 hours, plates were centrifuged at 1200 rpm for 5 minutes and supernatant was collected for cytokine analysis. Human TNFα, IL6, IL1β, IFNα and IFNβ were quantified using a Luminex platform, in collaboration with Dana Farber Cancer Institute.

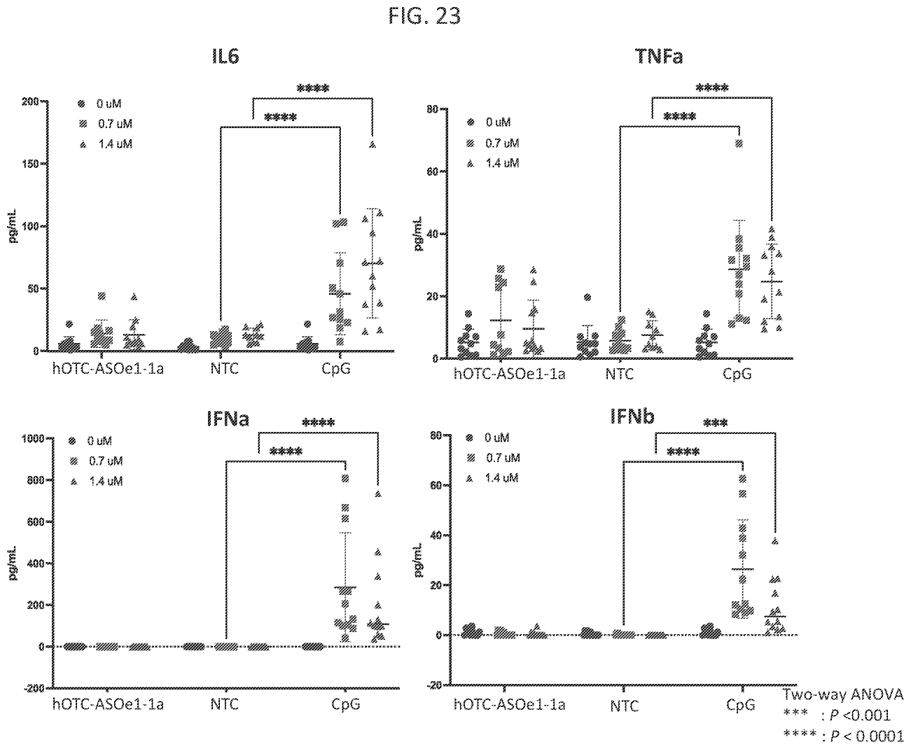

As shown in FIG. 23, treatment of cells with hOTC-ASOe1-1a did not induce cytokine release by the PBMCs.

### Example 4: Tiling and Optimizing Mouse OTC regRNA-Targeting ASOs

Mouse ASOs were made that targeted an additional mouse regRNA and were tested. ASO synthesized and characterized were mOTC-ASOe-3, mOTC-ASOe-4, mOTC-ASOe-5, and mOTC-ASOe-6

The newly synthesized mouse ASO were tested in mouse primary hepatocytes as described above. Briefly, primary hepatocytes were seeded at 20,000 cells per well on day 0. Cells were treated with 10, 5, 2.5, 1.25, or 0.625 μM mouse ASO on day 2. Cells were incubated for 2 days and lysate was collected on Day 2 post treatment for mRNA analysis. The tagman probe Mm01288053_m1 was used for mouse OTC expression. Ppia and Hprt were used as housekeeper genes for gene expression normalization. Statistics were performed using one-way ANOVA in Prism (GraphPad)

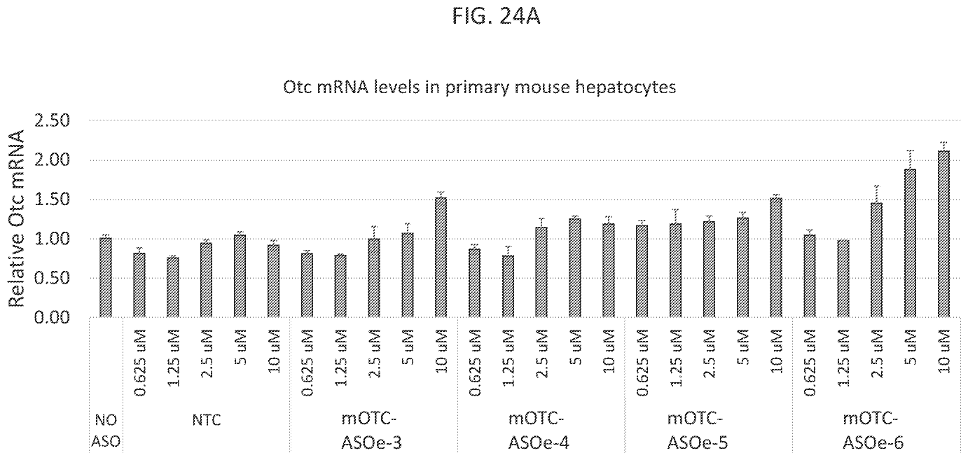

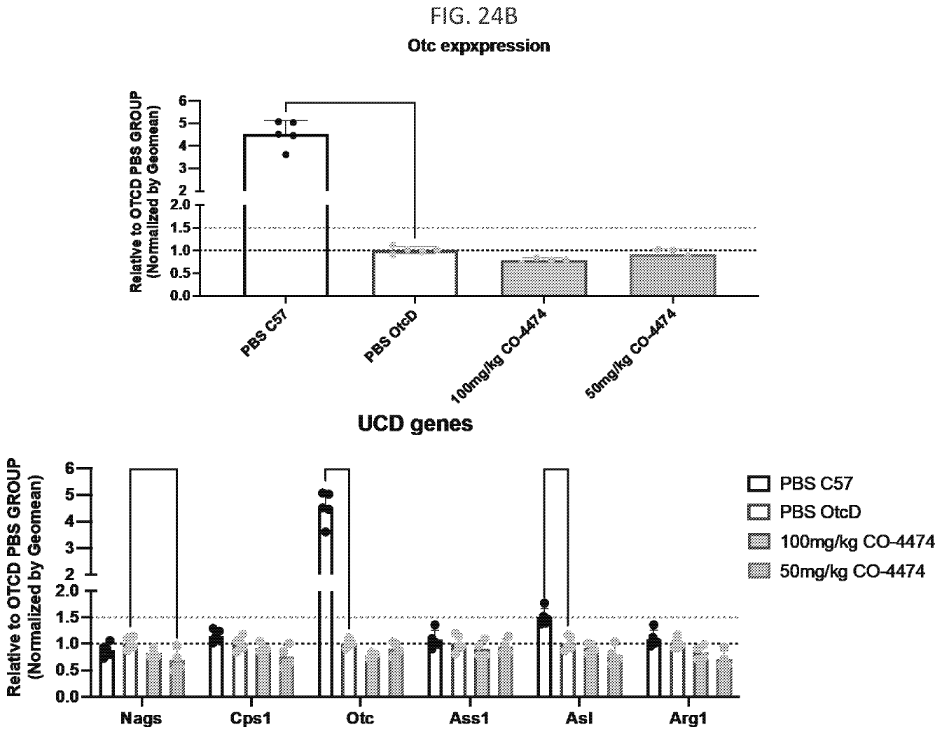

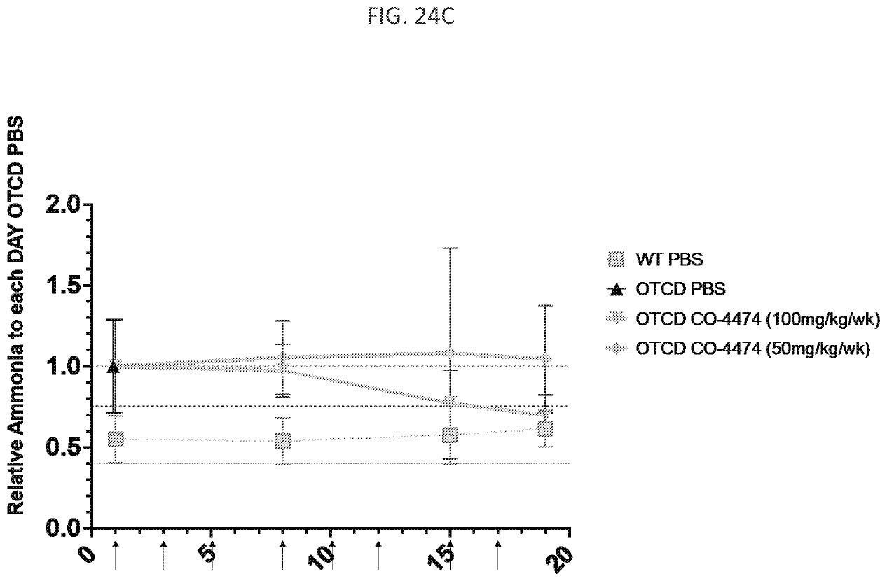

As shown in FIG. 24A, the new mouse ASOs increased mouse Otc expression in a dose dependent manner.

A terminal GalNAc was conjugated to mOTC-ASOe-3, resulting in ASO CO-4474. This ASO was tested in OTC deficient mice (OTC^{def}) in an ammonium challenge assay. Briefly, 10 Male B6EiC3Sn a/A-Otc^{spf-ash}/J Mice (homozygous) and 10 C57 WT mice were treated with ammonium once a week for 4 weeks and dosed with ASOs on days 1, 3, 5, 8, 10, 12, 15, and 17. Mice were dosed with either 100 mg/kg/week ASO or 50 mg/kg/week ASO. Samples were collected at the end of the study for OTC mRNA quantification as previously described.

As shown in FIG. 24B, the Otc regRNA-targeting ASO CO-4474 did not increase mouse Otc mRNA in Otc^{def} mice. In addition, the Otc regRNA-targeting ASO CO-4474 did not change the other mouse UCD gene expression. However, as shown in FIG. 24C, CO-4474 decreased ammonia to WT levels. Thus, the mouse Otc ASO was able to rescue the Otc deficiency phenotype.

### Example 5: regRNA-Targeting ASO Results in Increased Epigenomic H3K27 Acetylation

Next, the relative enhancer activity in human hepatocytes post ASO treatment was assessed.

Primary human hepatocytes from a single donor (HUM181371, Lonza) were cultured in vitro. 7.5×10⁶ cells were plated using 10 cm² collagen coated plate in plating medium and plates agitated every 15 minutes to ensure cell density was even across entire plate. Plating medium was changed to growth medium four hours post plating and growth medium changed every 48 hrs for six days. On day four medium change, 2 μM ASO was diluted in growth medium. 7.5×10⁶ hepatocytes were treated with either a non-targeting control (NTC) ASO or hOTC-ASOe1-10 targeting non-coding RNAs (regRNAs) transcribed (minus strand) from the OTC enhancer. Hepatocytes were treated for 48 hr with ASO and crosslinked for 15 minutes by adding 11% formaldehyde (final 1%) to culture medium on day six. Formaldehyde was quenched by the addition of 200 mM glycine for 5 minutes and cells scraped and washed 3× with ice cold 1×PBS.

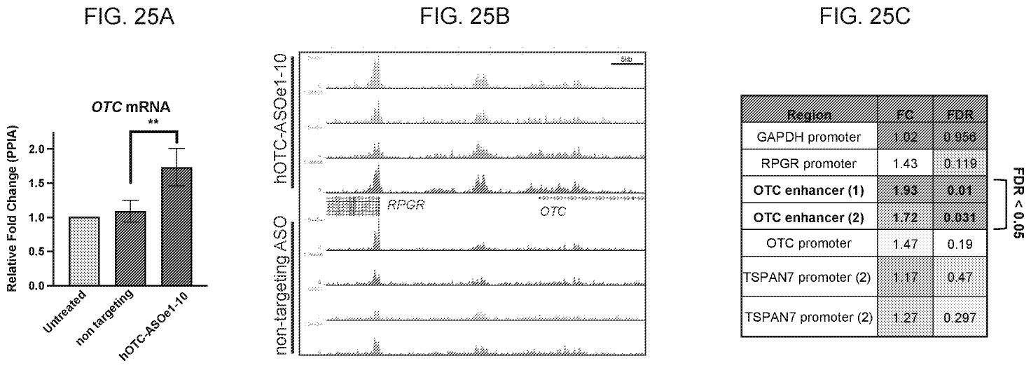

Prior to crosslinking, a small periphery cell scraping was collected for RNA isolation, cDNA synthesis (random hexamer) and qPCR analysis (OTC mRNA and PPIA TaqMan probes #Hs00166892_m1 and #Hs04194521_s1, respectively) to validate OTC mRNA upregulation in hOTC-ASOe1-10 treated hepatocytes compared to NTC ASO treatment. Cycle threshold (CT) values were normalized to endogenous control gene's (i.e. PPIA) CT value (=dCT) and relative fold changes was calculated by subtracting hOTC-ASOe1-10 dCT from NTC ASO dCT values (FIG. 25A).

H3K27ac chromatin immunoprecipitation followed by high-throughput sequencing (ChIP-seq) was performed on crosslinked hepatocyte samples treated with either NTC ASO or hOTC-ASOe1-10. Cell pellets were lysed with ice cold LB1 (50 mM Hepes-KOH, pH 7.5, 140 mM NaCl, 140 mM, 1 mM EDTA, pH 8.0, 10% Glycerol solution, 0.5% NP-40, 0.25% Triton X-100) plus fresh protease inhibitor for 10 minutes at 4° C. and subsequently incubated with LB2 (10 mM Tris-HCL pH 8.0, 200 mM NaCl, 1 mM EDTA, pH 8.0, 1 mM EGTA, pH 8.0) plus fresh protease inhibitors for 10 minutes at 4° C. Nuclei were centrifuged 1350 rcf, 5 minutes, 4° C. and resuspended in 1 mL of sonication buffer (50 mM Hepes-KOH, pH 7.5, 140 mM NaCl, 1 mM EDTA, pH 8.0, 1% Triton X-100, 0.1% Na-deoxycholate, 0.1% SDS) plus fresh protease inhibitors. Chromatin was fragmented using a Covaris focused ultrasonicator and conditions: 10′ time, fill level 5, duty cycle 5, peak incidence power 140, cycles/burst 200. Fragmented chromatin was centrifuged 20,000 rcf, 5 minutes, 4° C. and supernatant transferred to DNA low bind tube. 50 μL was saved for input. 5 g of anti-H3K27ac (abeam #ab4729) was pre-incubated the day before with blocked (0.5% BSA/1×PBS) Protein A conjugated magnetic beads. Chromatin and bead-antibody bound complexes were combined and incubated overnight, rotating at 4° C. The following day the bound chromatin-beads were washed 2× each, 5 minutes, 4° C. with 1 mL of the following buffers: Sonication Buffer, Wash Buffer 2 (50 mM Hepes-KOH pH7.5, 350 mM NaCl, 1 mM EDTA pH8.0, 1% Triton X-100, 0.2% Na-deoxycholate, 0.1% SDS), and Wash Buffer 3 (20 mM Tris-HCl pH8.0, 1 mM EDTA pH8.0, 250 mM LiCl, 0.5% Na-deoxycholate). Samples were washed 1× with TE+0.2% Triton X-100, followed by 2× with TE. Chromatin was eluted and reverse crosslinked overnight at 65° C. in SDS Elution Buffer (50 mM Tri-HCl pH8.0, 10 mM EDTA pH8.0, 1% SDS). ChIP samples were placed on a magnet and eluted (reverse crosslinked chromatin) was transferred to a new tube. Samples (ChIP and input) were treated with RNase A for 30 minutes, 37° C. followed by proteinase K (20 mg/mL) for 90 minutes at 55° C. DNA was purified by adding 600 uL phenol/chloroform/isoamyl alcohol to each sample and centrifuged at 16,000 rcf, 5 minutes, 4° C. using MaXtract High Density gel tubes (Qiagen #129056). Supernatant was precipitated with Na-acetate and ethanol overnight at −20° C., centrifuged 20,000 rcf, 4° C., washed with 1 mL 75% ethanol and eluted in 25 μL nuclease free water. ChIP DNA and input DNA were subjected to library synthesis for high-throughput sequencing using NEBNext DNA library prep kit following the manufacturer's guidelines. Two biological replicates consisting of two technical replicates each (4 samples for each ASO treatment) were subjected to this assay.

ChIP-seq libraries were paired-end sequenced using a Novoseq SP (150 bp) and aligned to the human hg38 genome using Bowtie2, alignment files were processed using SamTools and peaks called using MACS2 (FIG. 25B). Differential peaks at OTC enhancer, OTC promoter and control regions (GAPDH, RPGR, TSPAN7) were identified between hOTC-ASOe1-10 and NTC ASO treatments through normalization methods and using DESeq2 (FIG. 25C).

FIG. 25A illustrates OTC mRNA upregulation by hOTC-ASOe1-10 as compared to NTC ASO and untreated hepatocytes after 48 hr treatment, indicating that ASO treatment was successful for upregulation of OTC mRNA. These samples were used for the subsequent H3K27ac ChIP-seq experiment.

FIG. 25B depicts a genome browser track image for OTC enhancer, OTC promoter and neighboring gene, RPGR. Enhancer and promoter regions are marked by histone H3K27 acetylation in both experimental hOTC-ASOe1-10 and NTC ASO treated hepatocytes signifying that the OTC enhancer is active in cultured human hepatocytes.

FIG. 25C shows fold-change (FC) quantification of the H3K27ac epigenetic mark for hOTC-ASOe1-10 treatment compared to non-targeting ASO treatment and the false discovery rate (FDR). The data show that 48 hr treatment with hOTC-ASOe1-10 significantly increases histone acetylation (FC, 1.72-1.93) at the OTC enhancer compared to that of non-targeting ASO treatment. Negative control region, such as GAPDH, neighboring genes, RPGR and TSPAN7 promoters do not exhibit significant increased H3K27ac deposition. Without wishing to be bound by theory, this result suggests that observed epigenetic effect (increased H3K27ac) from hOTC-ASOe1-10 is specific to the target region (OTC enhancer) which is predicted to regulate the OTC gene. Thus, without wishing to be bound by theory, the ASOs described herein modulate OTC gene expression by modifying the epigenomic signature at the OTC enhancer.

### Example 6: ASO Treatment does not Alter Chromatin Accessibility at OTC Enhancer

Next, hOTC-ASOe1-10 binding of regRNAs directly or indirectly increased chromatin accessibility at the enhancer targeted by ASOs was assessed.

Primary human hepatocytes from a single donor (HUM181371, Lonza) were cultured in vitro. Plating medium was changed to growth medium four hours post plating and growth medium changed every 48 hrs for six days. On day five, medium was changed and 2 μM ASO was diluted in growth medium. Hepatocytes were treated with either non-targeting ASO or hOTC-ASOe1-10 for 24 hr. ATAC-seq was performed using the Omni-ATAC protocol, optimized for primary human hepatocytes in monoculture. Following DNase treatment on plate, hepatocytes were detached and enriched for live cells using a magnetic Dead Cell Removal Kit (Miltenyi #130-090-101). Approximately 50,000 live cells per replicate were used for the Omni-ATAC protocol. Three technical replicates were generated per treatment.

ATAC-seq libraries were paired-end sequenced using a Novoseq SP (150 bp) and aligned to human genome hg38. Aligned reads were processed accordingly and accessible chromatin regions were identified using MACS2 pipeline described in methods used in Corces et al., 2017.

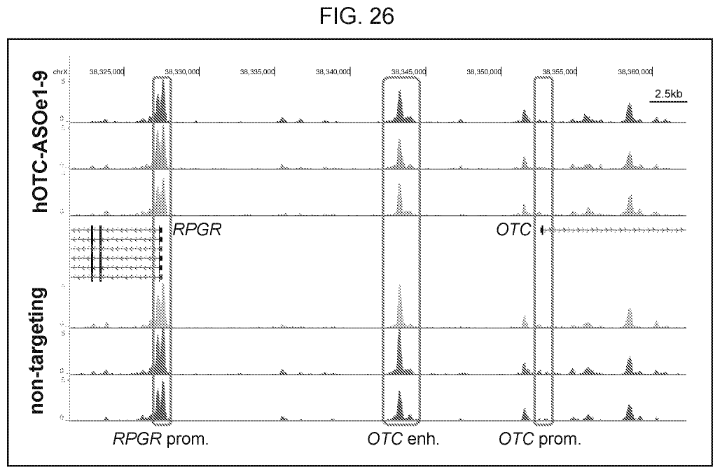

FIG. 26 illustrates accessible chromatin regions at the OTC promoter and enhancer and neighboring RPGR promoter (denoted by boxed regions). These results demonstrate that hOTC-ASOe1-10 causes no changes in chromatin accessibility at the OTC enhancer or promoter suggesting that the ASO acts downstream of transcription factor (activator) binding.

### Example 7: ASO Effects on regRNA Precede OTC mRNA Transcriptional Bursts

Next, the temporal response of regRNA activation upon ASO treatment with induction of OTC mRNA, as well as activation of enhancer histone modification ‘memory’ was assessed.

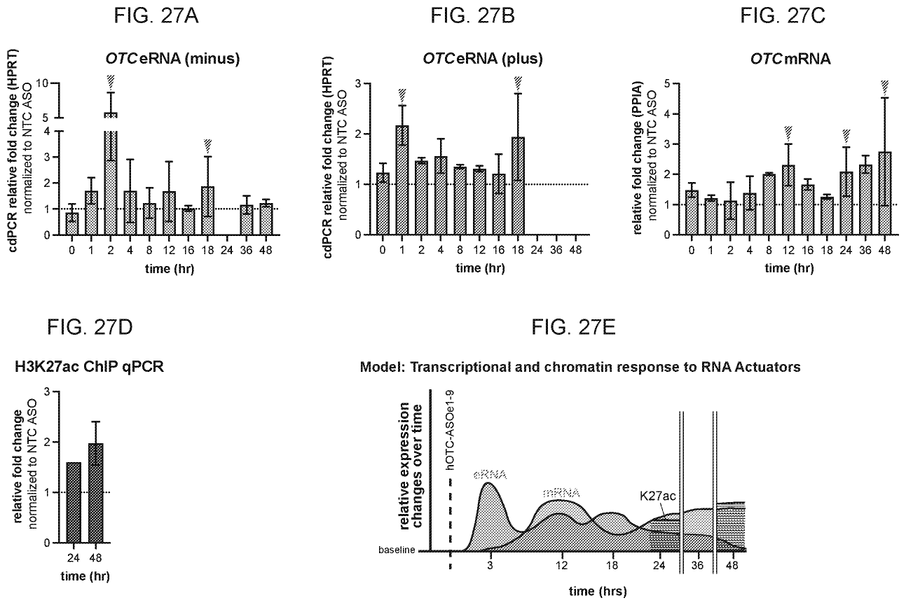

Primary human hepatocytes from a single donor (HUM181371, Lonza) were cultured in vitro. Plating medium was changed to growth medium four hours post plating and growth medium changed every 48 hrs for six days. On day(s) 4-5, cells were treated at varying time points (noted in FIG. 27A-C) with 5 μM ASO diluted in growth medium prior to harvest. All wells were harvested with cell lysis buffer simultaneously for subsequent RNA isolation (MagMax MirVana, ThermoFisher), cDNA synthesis (random hexamer), crystal digital PCR (cdPCR; regRNA detection) and quantitative-PCR (qPCR; mRNA detection). Experiments were done in biological triplicates, each with technical triplicates.

cdPCR was performed using the Naica® Crystal Digital PCR™ system from Stilla Technologies. Concentrations of regRNA were determined using a custom TaqMan assay and normalized to endogenous control HPRT1 (TaqMan assay #4326321E, ThermoFisher). Relative fold change was calculated by normalizing to NTC ASO treated cells at each respective timepoint.

Quantitative-PCR was performed using TaqMan probe #Hs00166892_m1 specific to OTC mRNA and each value was normalized to endogenous control PPIA (TaqMan endogenous control assay #4326316E, ThermoFisher). Relative fold change was calculated by normalizing to NTC ASO treated cells at each respective time point. Technical triplicates were averaged for each biological triplicate and those values plotted in bar graph (n=2 biological). Error bars denote standard deviation.

H3K27ac ChIP followed by qPCR was performed on cultured primary hepatocytes as described in Example 5 above, with the difference of using 5 μM ASO and treated on either day 4 or day 5 and harvested on day 6. ChIP-qPCR experiments were conducted in biological singlets and duplicates (24 and 48 hr respectively). qPCR was performed using SYBR and primers designed to amplify a genomic region of the OTC enhancer. Values plotted are relative fold change of hOTC-ASOe1-10 treated hepatocytes normalized to NTC ASO treated hepatocytes.

eRNAs generated at enhancers (regRNAs) are transcribed bidirectionally and enhancer activity has been shown to be correlated with the amount of eRNA transcribed. Relative expression levels for both regRNAs transcribed from the OTC enhancer were obtained over time post ASO treatment (FIG. 27A and FIG. 27B).

FIG. 27A illustrates relative levels of the targeted (minus strand) regRNA over time following ASO treatment. Effects are measured as early as 2 hours post treatment with the and effect size is reduced by 8-16 hours. Upregulation is observed again at 18 hours (red arrowheads) suggesting that these loci undergo transcriptional bursts as previously described in the literature⁶.

FIG. 27B shows similar effects on the non-targeted (plus) strand with bimodal upregulation early (1 hr) and late (18 hr) (red arrowheads).

FIG. 27C illustrates OTC mRNA effects over time post hOTC-ASOe1-10 ASO treatment. OTC mRNA is upregulated by 12 hr and again later at 24/48 hr (arrowheads). This effect mimics the similar transcriptional ‘burst’ phenomenon observed with the regRNAs (minus and plus). As expected, regRNA upregulation precede OTC mRNA upregulation indicating that the effects of increased regRNA concentration leads to increased OTC mRNA levels.

H3K27ac ChIP-qPCR results (FIG. 27D) demonstrate that H3K27ac is deposited later (24 hr) post hOTC-ASOe1-10 ASO treatment indicating that effects on RNA precede epigenetic changes at this enhancer. hOTC-ASOe1-10 treatment results in increased H3K27ac ChIP signal from 24 to 48 hr. Without wishing to be bound by theory, these results suggest that acetylation of residue K27 on histone H3 may be important for maintaining enhancer activity post initial regRNA/mRNA induction. A temporal model of the transcriptional and chromatin response to OTC ASO is shown in FIG. 27E.

### Example 8: Negative Regulator Protein Binding is Decreased with ASO Treatment

Next, the perturbation of repressor protein complex interactions at the OTC enhancer after treatment with hOTC-ASOe1-10 in human hepatocytes was assessed. No significant change in chromatin accessibility was observed with hOTC-ASOe1-10 treatment (FIG. 26). Without wishing to be bound by theory, this indicated that any effects may be due to displacement of other proteins such as negative regulators.

Candidate negative regulators were selected using publicly available ENCODE ChIP-seq data from HepG2 cells. Briefly, ENCODE transcription factor (TF) data in HepG2 cells was filtered for TF occupancy at the OTC enhancer and further filtering criteria eliminated all TFs not associated with negative regulatory mechanisms. In total five negative regulator proteins were found to be bound to the OTC enhancer in HepG2 cells (ARID1, BCL6, HDAC1, HDAC5 and NCOR1). SP1 is a transcription factor implicated in general transcription activation and found bound at the OTC enhancer and used a control.

Primary human hepatocytes from a single donor (HUM181371, Lonza) were cultured in vitro. 7.5×10⁶ cells were plated using 10 cm² collagen coated plate in plating medium and plates agitated every 15 minutes to ensure cell density was even across entire plate. Plating medium was changed to growth medium four hours post plating and growth media changed every 48 hrs for six days. On day five medium was changed and 5 μM ASO was diluted in growth medium. 1.5×10⁷ hepatocytes were treated with either a NTC ASO or hOTC-ASOe1-10 targeting non-coding RNAs (regRNAs) transcribed (minus strand) from the OTC enhancer (2×10 cm² plates). Hepatocytes were treated for 24 hr with specified ASO and crosslinked for 15 minutes by adding 11% formaldehyde (final 1%) to cultured media on day six. Formaldehyde was quenched by the addition of 200 mM glycine for 5 minutes and cells scraped and washed 3× with ice cold 1×PBS.

Prior to crosslinking, a small periphery cell scraping was collected for RNA isolation to validate OTC mRNA upregulation in hOTC-ASOe1-10 treated hepatocytes compared to NTC ASO treatment as described in Example 1 (FIG. 1A).

Hepatocytes treated with ASO for 24 hr followed by ChIP qPCR were performed in biological triplicates for each repressor TF ChIP'd.

ChIP followed by qPCR for each respective negative regulators were performed on cultured primary hepatocytes as described in example 1 (FIG. 1B) using specific antibodies for ARID1, BCL6, HDAC1, HDAC5, NCOR1 and SP1 (sc-32761X, PA527390, 40967ACTMOTIF, 40970ACTMOTIF, #A301145A, sc-17824X, respectively). ChIP-qPCR experiments were conducted in biological triplicates. qPCR was performed using SYBR and primers designed to amplify a genomic region of the OTC enhancer. Values plotted are relative fold change of hOTC-ASOe1-10 treated hepatocytes normalized to NTC ASO treated hepatocytes.

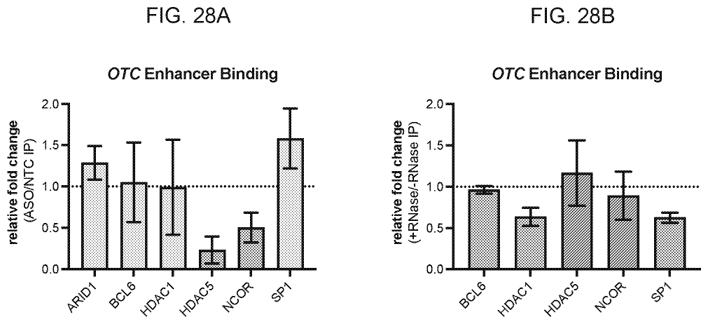

Values plotted in FIG. 28A and FIG. 28B are relative fold changes of hOTC-ASOe1-10 compared to NTC ASO (n=3) and error bars denote standard deviation.

rChIP-qPCR was performed to assess the requirement of RNA for targeted protein-chromatin interaction. Assay was performed using standard ChIP protocol, with the addition of a Rnase A treatment step post immunopurification of chromatin-protein complexes.

FIG. 28A illustrates the relative loss of binding for the denoted negative regulators. Of the five negative regulators, only HDAC5 and NCOR showed diminished binding at the OTC enhancer in hepatocytes treated with hOTC-ASOe1-10 when compared to NTC ASO treatment. This suggests that hOTC-ASOe1-10 binding to regRNAs inhibits (directly or indirectly) those regRNAs and associated chromatin (enhancer) from interacting with repressor complexes that include HDAC5 and NCOR.

FIG. 28B demonstrates that negative regulators do not require RNA molecules to bind to their target. HDAC5 and NCOR1 binding is not reduced at the OTC enhancer in hepatocytes (no treatment) when crosslinked chromatin is treated with RNase to degrade RNA. This result does not suggest that regRNAs do not interact with repressor proteins but rather that interaction is not essential for their recruitment to the OTC enhancer.

### Example 9: Knockdown of Repressor Complexes Reduces Effect of ASO Treatment on OTC mRNA Upregulation

Next, knockdown of bound repressor complexes at the OTC enhancer to reduce the effects observed with ASO treatment was assessed.

Primary human hepatocytes from a single donor (HUM181371, Lonza) were cultured in vitro using a 48 well collagen-coated tissue culture plate. Plating medium was changed to growth medium four hours post plating and growth medium changed every 48 hrs for six days. On day 3, cells were transfected for 18 hr with 10 nM siRNA targeting HDAC5 and NCOR1 (Dharmacon M-003498-02-0005 and M-003518-01-0005 respectively) using Lipofectamine RNAiMax and manufacturer's recommended protocol (ThermoFisher, 13778150). Medium was changed the following day (day 4) with either 5 μM NTC ASO or hOTC-ASOe1-10 diluted in growth medium and cultured for 48 hr and hepatocytes harvested for RNA isolation (MagMax MirVana kit, ThermoFisher #A27828), cDNA synthesis using random hexamers and qPCR analysis to evaluate knockdown efficiency and effects on OTC mRNA (TaqMan probes #Hs01094541_m1, Hs00608351_m1, Hs00166892_m1). Knockdown experiments coupled with ASO treatments were performed in biological triplicates, each with three technical replicates (per treatment). Values plotted in graphs are the average of the technical replicates for each biological experiments (n=3).

Knockdown efficiency for either HDAC5 or NCOR1 siRNA treatments was determined by normalizing each sample's relative CT values to endogenous control (PPIA) and calculating fold change based on samples with no siRNA treatment.

To understand the effects of siHDAC5 or siNCOR1 on hOTC-ASOe1-10 activity, all treatments were normalized to NTC ASO within that respective siRNA experiment to reduce confounding effects of knockdown.

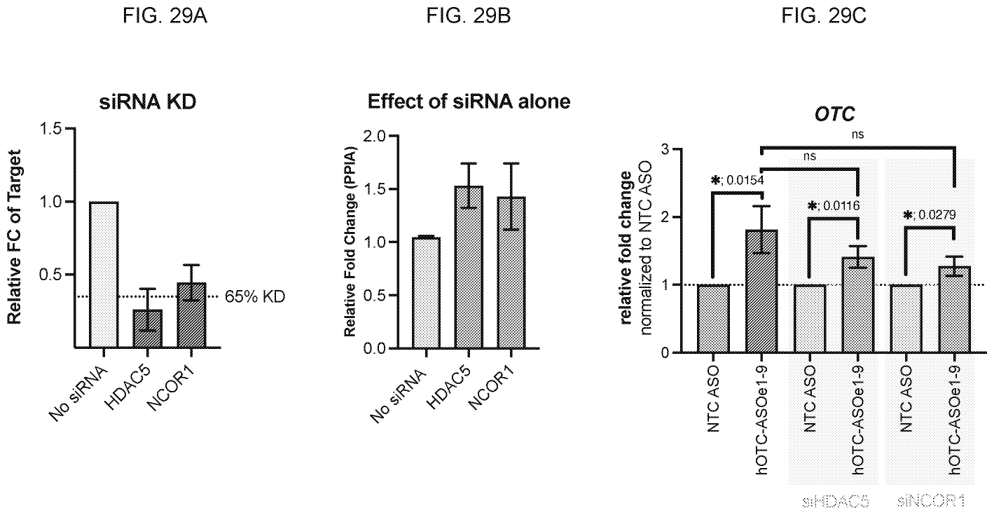

Values plotted in FIG. 29A-29C are averages of technical triplicates from three biological replicate experiments. Error bars denote standard deviation. P-values are calculated with unpaired student's t-test using average of each biological replicate (n=3).

Treatment of siHDAC5 or siNCOR1 resulted in at least a 50% reduction in target mRNA levels as depicted in FIG. 29A compared to untreated hepatocytes.

FIG. 29B shows the effects of HDAC5 or NCOR1 knockdown on OTC mRNA. SiRNA treatment for either of these factors leads to increased OTC mRNA expression in hepatocytes demonstrating that these complexes are involved in OTC mRNA repression. Alleviating this repression mechanism at the OTC enhancer causes a marginal increase in basal OTC levels.

The effects of knockdown on hOTC-ASOe1-10 are displayed in FIG. 29C. hOTC-ASOe1-10 significantly (p-value=0.0154) upregulated OTC mRNA (FC=1.81, no siRNA treatment). Hepatocytes treated with siHDAC5 or siNCOR1 showed significant upregulation of OTC mRNA with hOTC-ASOe1-10 compared to NTC ASO (FC=1.41 and 1.28 respectively). This experiment indicates that hOTC-ASOe1-10 has an effect on OTC mRNA when repressor complex proteins are knocked-down as OTC mRNA levels are already slightly increased.

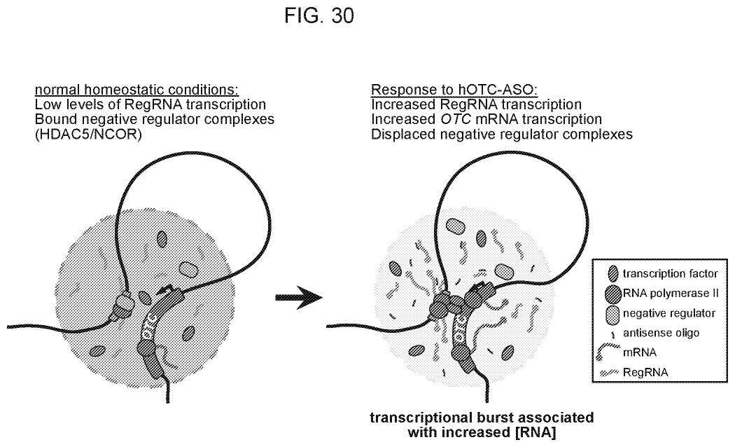

Without wishing to be bound by theory, under normal homeostatic cellular conditions, there are low levels of regRNAs and mRNA transcribed from the OTC enhancer and gene body, respectively. Negative regulators such as HDAC5 and NCOR1 are found bound at the enhancer, likely modulating its low activity, as well as transcriptional activators, priming the locus. hOTC-ASOe1-10 treatment results in increased regRNA levels, possibly through inhibition of repressor complex binding. This activation of the OTC enhancer promotes a positive transcriptional response at the OTC gene thus resulting in transcriptional bursts at the OTC enhancer and promoter (FIG. 30).

### Example 10: Characterization of ASOs in Non-Human Primates

### Materials and Methods

15N-ammonium chloride was obtained from Cambridge isotopes (Tewksbury, MA).

### Ammonia Measurement and Ureagenesis for NHPs

Ammonia challenge and Ureagenesis assay in cynomolgus monkeys [NHPs] were performed in a fasted state, i.e. food withdrawal overnight, prior to ammonia challenge. 15N-ammonia chloride solution was subcutaneously injected to NHPs and multiple blood draws were performed over 0-120 min and immediately plasmas were obtained by centrifugation. Aliquots of plasmas were shipped at 4 degrees to IDEXX to measure ammonia levels. Other aliquots were snap-frozen and shipped to NovaBioAssays (Woburn, MA) to measure 15N-urea/total urea levels.

### ASO Treatment for NHPs

Male cynomolgus monkeys, 2-4 years old, were subcutaneously injected with a single 50 mg/kg ASOs on Day 0 and a second dose were given on Day 21. PBS as a negative control.

### Results

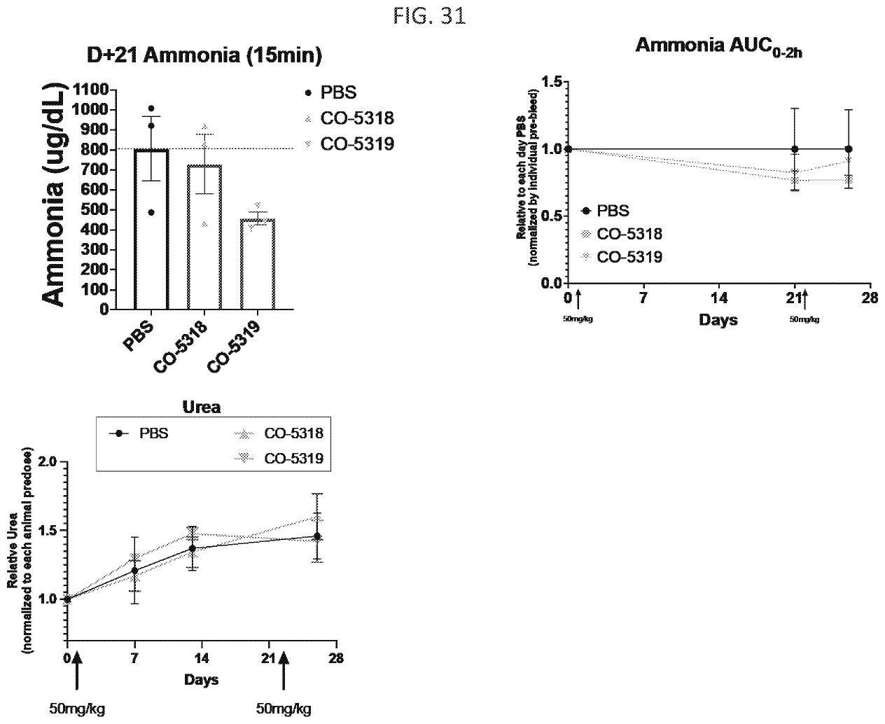

CO-5318 (hOTC-ASOe1-las) and CO-5319 (hOTC-ASOe2-2w) reduced ammonia and increase urea in NHPs (FIG. 31). Thus, the ASOs shows therapeutic efficacy in the NHPs.

### Example 11: Characterization of ASOs in Humanized Mice

### Materials and Methods

### Ammonia Measurement and Ureagenesis for Humanized Yecuris FRG Mouse Study

Ammonia challenge and Ureagenesis assay in female liver-humanized Fah−/− Rag2−/−Il2rg−/− [FRG] mice with C57Bl/6 background, repopulated with healthy human hepatocytes were performed in a fasted state, i.e. food withdrawal for overnight, prior to ammonia challenge. After fasting overnight on days 1, 8, 15, and 22 (terminal harvest) the animals were challenged with 15NH4C1 (15N-ammonia) by intraperitoneal injection. After 30 minutes, urine and blood (processed into plasma) were collected. Aliquots of plasmas were shipped at 4 degrees to IDEXX (North Grafton, MA) to measure ammonia levels. Other aliquots were snap-frozen and shipped to NovaBioAssays (Woburn, MA) to measure 15N-urea/total urea levels.

### ASO Treatment for the Mouse Study

Female humanized Yecuris FRG mice, ˜5 months old, were subcutaneously injected with 50 mg/kg/week ASOs on days 8, 12, 15, and 19. PBS was used for the control.

### Ammonia Measurement and Ureagenesis for the Otc^{spf/ash} Mouse Study

Ammonia challenge and Ureagenesis assay in both wildtype C57BL/6J [WT] and a/A-Otc^{spf-ash}/J, [OTCD] were performed in a fasted state, i.e. food withdrawal overnight, prior to ammonia challenge. 15N-ammonia chloride solution was subcutaneously injected into WT and OTCD mice and blood was drawn 30 min post ammonia chloride injection and immediately plasmas were obtained by centrifugation. Aliquots of plasmas were shipped at 4 degrees to IDEXX to measure ammonia levels. Other aliquots were snap-frozen and shipped to NovaBioAssays (Woburn, MA) to measure 15N-urea/total urea levels.

### ASO Treatment for the Mouse Study

Male C57BL/6J [WT] and a/A-Otc^{spf-ash}/J, [OTCD], ˜6-7 weeks old, were subcutaneously injected with either 50 or 100 mg/kg/week ASOs at Day 1, 3, 5, 8, 10, 12, 15, and 17. PBS as a negative control.

Taqman probes (all from Thermofisher)

| Human | | Mouse | |
|---|---|---|---|
| Target | Probe | Target | Probe |
| OTC | Hs0166892_m1 | Otc | Mm01288053_m1 |
| CPS1 | Hs00157048_m1 | Cps1 | Mm01256489_m1 |
| NAGS | Hs00400246_m1 | Nags | Mm00467530_m1 |
| ASS1 | Hs01597989_g1 | Ass1 | Mm00711256_m1 |
| ARG1 | Hs00968979_m1 | Arg1 | Mm00475988_m1 |
| ASL | Hs00902699_m1 | Asl | Mm01197741_m1 |
| HPRT | 4325801 | Hprt | Mm03024075_m1 |
| PPIA | 4325790 | Ppia | Mm02342430_g1 |
| GUSB | 4325799 | Gusb | Mm01197698_m1 |
| GAPDH | 4325792 | Gapdh | Mm9999915_g1 |
| ACTB | 4325788 | Actb | Mm00607939_s1 |
| B2M | 4326319E | | |

### Results

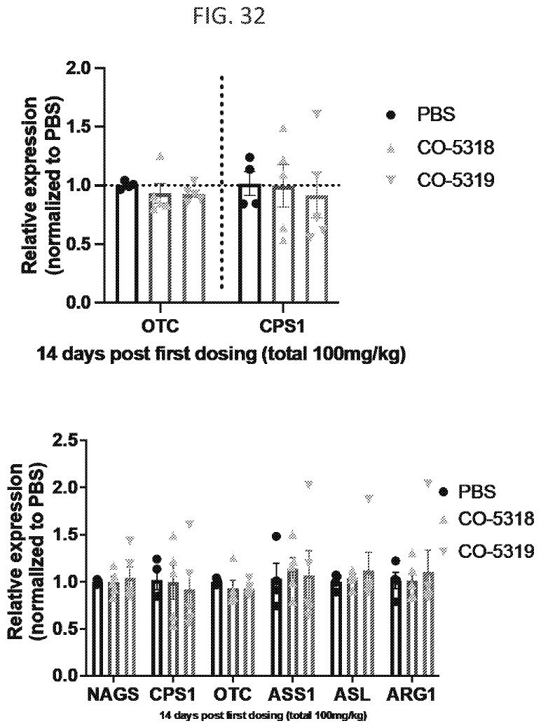

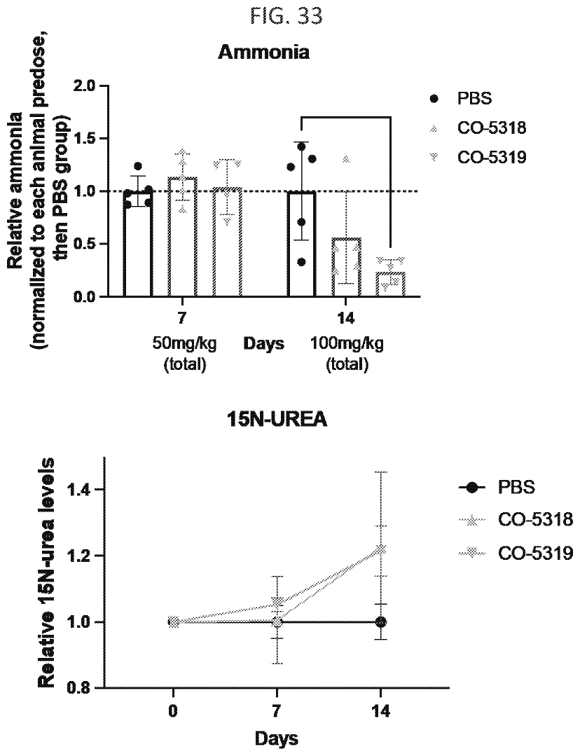

The NH4C1 challenge was given to humanized mice to measure the impact of the ASO on ureagenesis. As shown in FIG. 32, both CO-5318 and CO-5319 didn't change OTC and CPS1 mRNA expression. However, as shown in FIG. 33, CO-5318 and CO-5319 treatment in humanized mice showed a decrease in ammonia and a corresponding increase in urea over time. Two-way ANOVA, *: P<0.05, **: P<0.01, ***: P<0.001, ****: P<0.0001.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antisense oligonucleotide (ASO) complementary to at least 8 contiguous nucleotides of a regulatory RNA of human Ornithine Transcarbamylase (OTC), wherein the regulatory RNA has a nucleotide sequence selected from the group consisting of SEQ ID NOs: 1-4 or 1077.

2. The ASO of claim 1, wherein the ASO is complementary to a sequence in the regRNA that is no more than 200 nucleotides from the 3′ end of the regRNA.

3. The ASO of claim 1, wherein the ASO is complementary to a sequence in the regRNA that is no more than 200 nucleotides from the 5′ end of the regRNA.

4. The ASO of claims 1-3, wherein the regRNA is not a polyadenylated RNA.

5. The ASO of claims 1-3, wherein the ASO does not induce RNAse H-mediated degradation of the regRNA.

6. The ASO of any one of claims 1, 2, 4, or 5, wherein the regulatory RNA has a nucleotide sequence of SEQ ID NO: 1, and the ASO comprises a nucleotide sequence selected from the group consisting of SEQ ID NOs: 6-14, 18-35, 39, 41, 75, 76, 77, 78, 87-124, or 143-892.

7. The ASO of any one of claims 1, 2, 4, or 5, wherein the regulatory RNA has a nucleotide sequence of SEQ ID NO: 2, and the ASO comprises the nucleotide sequence of SEQ ID NO: 15-17, 36-38, 64-74, 125-142, or 893-1029.

8. The ASO of any one of claim 1, or 3-5, wherein the regulatory RNA has a nucleotide sequence of SEQ ID NO: 2, and the ASO comprises the nucleotide sequence of SEQ ID NO: 17.

9. The ASO of any one of claims 1-8, wherein the ASO is no more than 50, 40, 30, or 25 nucleotides in length.

10. The ASO of any one of claims 1-9, wherein the ASO comprises a RNA polynucleotide comprising one or more chemical modifications.

11. The ASO of claim 10, wherein at least 3, 4, or 5 nucleotides at the 5′ end and at least 3, 4, or 5 nucleotides at the 3′ end of the ASO comprise ribonucleotides with one or more chemical modifications.

12. The ASO of claim 10 or 11, wherein the one or more chemical modifications comprise a nucleotide sugar modification comprising one or more of 2′-O—C1-4alkyl such as 2′-O-methyl (2′-OMe), 2′-deoxy (2′-H), 2′-O C1-3alkyl-O C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

13. The ASO of any one of claims 10-12, wherein the one or more chemical modifications comprise an internucleotide linkage modification comprising one or more of phosphorothioate (“PS” or (P(S))), phosphoramidate (P(NR1R2) such as dimethylaminophosphoramidate (P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate P(CH3), boranophosphonate (P(BH3)), or phosphorodithioate (P(S)2).

14. The ASO of any one of claims 10-13, wherein the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

15. The ASO of any one of claims 10-14, wherein the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

16. The ASO of any one of claims 10-15, wherein the ASO comprises the nucleotide sequence of SEQ ID NOs: 18-39 or 67-74.

17. The ASO of any one of claims 10-15, wherein the ASO does not comprise 10 or more contiguous nucleotides of unmodified DNA.

18. The ASO of claim 17, wherein the ASO does not comprise a deoxyribonucleotide.

19. The ASO of any one of claims 10-18, wherein the ASO does not comprise an unmodified ribonucleotide.

20. The ASO of any one of claims 10-19, wherein the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

21. The ASO of claim 20, wherein the ASO further comprises a GalNAc moiety, optionally a GalNAc3 moiety.

22. The ASO of claim 20 or 21, wherein the ASO comprises the nucleotide sequence of SEQ ID NO: 142.

23. The ASO of any one of claims 10-19, wherein the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

24. The ASO of claim 23, wherein the ASO comprises the nucleotide sequence of SEQ ID NO: 21.

25. The ASO of claim 23 or 24, wherein the ASO further comprises a GalNAc moiety, optionally a GalNAc3 moiety.

26. The ASO of claim 25, wherein the ASO comprises the nucleotide sequence of SEQ ID NO: 122.

27. The ASO of any one of claims 10-19, wherein each ribonucleotide of the ASO is modified by 2′-O-methoxyethyl.

28. The ASO of claim 27, wherein the ASO comprises the nucleotide sequence of SEQ ID NO: 25.

29. The ASO of any one of claims 10-19, wherein each nucleotide of the ASO is a ribonucleotide modified by 2′-O-methoxyethyl.

30. The ASO of claim 29, wherein the ASO comprises the nucleotide sequence of SEQ ID NO: 36.

31. The ASO of any one of claims 10-15, wherein the ASO comprises 10 or more contiguous nucleotides of unmodified DNA flanked by at least 3 nucleotides of modified ribonucleotides at each of the 5′ end and the 3′ end.

32. The ASO of claim 31, wherein the ASO comprises the nucleotide sequence of SEQ ID NO: 18.

33. The ASO of any one of claims 10-32, wherein each cytidine in the ASO is modified by 5-methyl.

34. The ASO of any one of claims 1-33, wherein the regRNA is an eRNA.

35. A pharmaceutical composition comprising the ASO of any one of claims 1-34 and a pharmaceutically acceptable carrier or excipient carrier.

36. A method of increasing transcription of OTC in a human cell, the method comprising contacting the cell with the ASO of any one of claims 1-34 or the pharmaceutical composition of claim 35.

37. The method of claim 36, wherein the cell is a hepatocyte.

38. The method of claim 36 or 37, wherein the ASO increases the amount of the regulatory RNA in the cell.

39. The method of any one of claims 36-38, wherein the ASO increases the stability of the regulatory RNA in the cell.

40. A method of treating urea cycle disorder, the method comprising administering to a subject in need thereof an effective amount of the ASO of any one of claims 1-34 or the pharmaceutical composition of claim 35.

41. The method of claim 40, wherein the ASO increases the amount of the regulatory RNA in a cell of the subject.

42. The method of claim 40 or 41, wherein the ASO increases the stability of the regulatory RNA in a cell of the subject.

43. The method of claim 41 or 42, wherein the cell is a hepatocyte.


SEQUENCE LISTING (1099 sequences)

SEQ ID NO: 1 - DNA, 1001 bp, Homo sapiens
  source 1..1001: mol_type = genomic DNA
cacagagcaaacagggaaatcacagaggttcaaagttcaccagtgtctcaacaatcagccctaatgctccctgtgactaccagacactcccaggacctgagtgatggggatcccatgagatcattttcattgctttctactgaccagggtttggtctaggagcactgtcccagtaataattttcatggcaatattctccccttgagcccaggaaacatgtcttggatggcttcaaaagtcactgcttggtgaatgccttctctgcccatttctactttttggtgaaacttgaaaccatctttgtagttggtgcctctcttcagaccctacttgggaggtgctcttgacctgctattgattgctttattgggctatatctactaagcaggggctctgccctcaccttaagctaatgattaaacacagccttcttctctcaaggctgctccactggtaacaactctgtggcctgtaaagatgggacctatttagggtctggaagatagaccatgggaatcctgtcttcaagattcaagagaaacaagcccttttcatggggctttgttgagtgtttggagcctaggtcataggtgctacatattcaccattattgatttattcctccagaatttttcaactggagttcaccatttcttccagggaaccaaggagttcatggatttccaggtcaccttcattgttatgcaatgtattaccattaagaagtaagcgaatcatctggcaagcagtactagcagctcctactcatagctttgttgtgagtatgaaatgtaataatgaatagagagtactgtagcacagtacctagctcagtgttcaataaatgttagcttttgttaactactaccattggcacatgtggtgagaggccccatccctggctcagttcttggcttattctaatcactttcctacaaataaaagtgttgaggtgtccgtctttctttcatacccccaccccactccagagctgtattaaa

SEQ ID NO: 2 - DNA, 2001 bp, Homo sapiens
  source 1..2001: mol_type = genomic DNA
cacagagcaaacagggaaatcacagaggttcaaagttcaccagtgtctcaacaatcagccctaatgctccctgtgactaccagacactcccaggacctgagtgatggggatcccatgagatcattttcattgctttctactgaccagggtttggtctaggagcactgtcccagtaataattttcatggcaatattctccccttgagcccaggaaacatgtcttggatggcttcaaaagtcactgcttggtgaatgccttctctgcccatttctactttttggtgaaacttgaaaccatctttgtagttggtgcctctcttcagaccctacttgggaggtgctcttgacctgctattgattgctttattgggctatatctactaagcaggggctctgccctcaccttaagctaatgattaaacacagccttcttctctcaaggctgctccactggtaacaactctgtggcctgtaaagatgggacctatttagggtctggaagatagaccatgggaatcctgtcttcaagattcaagagaaacaagcccttttcatggggctttgttgagtgtttggagcctaggtcataggtgctacatattcaccattattgatttattcctccagaatttttcaactggagttcaccatttcttccagggaaccaaggagttcatggatttccaggtcaccttcattgttatgcaatgtattaccattaagaagtaagcgaatcatctggcaagcagtactagcagctcctactcatagctttgttgtgagtatgaaatgtaataatgaatagagagtactgtagcacagtacctagctcagtgttcaataaatgttagcttttgttaactactaccattggcacatgtggtgagaggccccatccctggctcagttcttggcttattctaatcactttcctacaaataaaagtgttgaggtgtccgtctttctttcatacccccaccccactccagagctgtattaaaagtgaaattcaggctgggcatggtggctcacgcctgtaatcccagcactttgaggcgggcggatcacgaggtcaggagttcgagactagcctgaccaacgtggtgaaaccccggctctactaaaaatacaaaaattagccaggcatggtggcggacacctgtaatcccagctatgcatcgagaggctgaggcaggagaattgcttgaacccgggaggcggaggttgcagtgagccgagatagtgccactgctctctagcctgggcgacagagcgagacaccatctccattaaaaaaaaaaaaaaaaaaaaaagtgaaatttaagaaaataataaatttaaataaaataaaataaagaataaaaaaagacaaaaaacaacaaacaaagaaatcacactcttgtcgtctaaactagtggaaaataaaaaggaaacagtgcttcttactgaagacctatgatgtgttttcttctttcttcttttctctttttgcctaaataatgtgaaagccaggaactttgttctccctcagtacaactgtctaacgttacaagtttctaatcttttattgtccttctgtgaccctgttatgctaaccaacctatcccatttaagatgaaaataagataaaacaataaatgactgttaagtacttcaaaaatacaaaagactctaaatgccaagagtgattattgccttccggtgtaatgaagatgcataataaaaatggatcataaaatcccttcgctaccatggccactactctttttgccagttaaaatttacaattacagtctgtttattcaaagcaacaggggacatggaaacaactcagtcattttctaaagtaggttcccccaatcacttgaacacagaatgactctagagacatcagtgatacatttttcaaaatccatatgaaaataacacatcatcttcagtgtctaataatgtcactgaaacctccccacccccatattctttttatccaatgtatatgtt

SEQ ID NO: 3 - DNA, 2001 bp, Homo sapiens
  source 1..2001: mol_type = genomic DNA
cgtgctttgcatctcaactgcctctgacacaaggttgggtcattaaccatatttatttacccctttcttacttcataaattgtgttgtgcatttcctaatcttgtcttctgtgcagaaatttaccagatcacaaaataaaaagaaccaggtatggaaacttgagacttttgtccatatctaagctcaaaaaattctggattctgcttaaaatcaataacttccagggcaaatgattaacagaataatgatttcaatggaaatgtggtctatatgacaagagagaagcgagattatacttgaatttgatgattggcatttcacactctgttcagaattttagcctttatgaaggaccctgtctaccttcttcaaatgcctcaattgaaagggtatggcaggaaaagggatggagagggggtcagagataatcctcactgatttcacaatttcccctagggcccacctgtttgaataaatacctagtggtgatgtagcaagaaaacacccctgaatcaatatttttagacaaactaccattaccattaccatctgttaattagaaagtaactctcagatcgcttgaacccaggaggcagaggttgccatgagccgaggttgtgccactgcattccagcctgggtgacagagcgagactctgtctcaaacaaacaaacaaacaaaaaacataaaaaataaaaaataaagtaattctcagattctctgtaaatcaggagctcttttggtgatatatatatatacacacacacacacacacacacacacacacacacacacgtggagagaaaaaaaggagccagctaatgggtgatttcctttactataataattctaatttatttcttattgccagtttccacacaggtccaagccgcaaaacaataccgtctcacaacagagcagagctttacagcttataaaacattctgggaatattagaatattaacacatgaagatttttgggtccttcagcacgttagatatgacctgggacatagaggactcttaagaaaagagaaattgggcatccacatgtggaaaaaaaaactcaatctcaacctcaagccttagacaaaactcacagtggatcacagatctgaatggaaaatgtaaggctacaacactttcagaagacatagggagagaatctttgttaccctgggttatgcaaagagttcaacagcatgatccagagaaaggaaacaaatggtaaattgaacttgatcaaaatttaaaaattttgctttgtgaaaaacatagtcaggagactgaaaagacaagcaacagactgggaaaacacatttgcaagttacatatccaataaaagtgttgtatccagaatatatatgtagaacattcaaaactaaacattaagaaaataaacaattcaagtaaaaaaaaaaaaagaagaagaataggggtcaggcaagctcagtggctcacgcttgtaatcccagtgctttgggagtctaaggctggaggactgttcaagaccaggaatgtgagacaagcctgggcaacatagtgagactctatacctaccaaaaaatttagtttggtgtggtggcacacacctatactcctagctatttgggaggctgaggttggaggatcgcttgagcccaggaggtcaaggttacagtgatctatgatcacactattgtgaaaccgcctttgcaaaattatgacagacagtgaaagaaatctaacttaaccgaatccatcttgcttctaaccttcaagctgtccttgttcattgctgggcataggctgaactaattttgggagaaacttagtttatagtttatggtttaaacaaagacggtaacacccctttcccaaagtgacctccttcctgcctggggactagacttcctttgtaggactaacattagccacaagatcagatgagccagtttattgatctggtggtgccagctgatccatcaagtgcagtactgataccccaagcactgatttt

SEQ ID NO: 4 - DNA, 2597 bp, Homo sapiens
  source 1..2597: mol_type = genomic DNA
aatcaatagcaggtcaagagcacctcccaagtagggtctgaagagaggcaccaactacaaagatggtttcaagtttcaccaaaaagtagaaatgggcagagaaggcattcaccaagcagtgacttttgaagccatccaagacatgtttcctgggctcaaggggagaatattgccatgaaaattattactgggacagtgctcctagaccaaaccctggtcagtagaaagcaatgaaaatgatctcatgggatccccatcactcaggtcctgggagtgtctggtagtcacagggagcattagggctgattgttgagacactggtgaactttgaacctctgtgatttccctgtttgctctgtgcctgatagctttcagtctgctaacaaatctcctttatgcagtttaacctctgtacttccaatggggaggaattggaatcagcctatgggagaagagatagctctaggattctgtgtgggaacttgaagagataaaagggcatccttgaaagagaacctcgtgctttgcatctcaactgcctctgacacaaggttgggtcattaaccatatttatttacccctttcttacttcataaattgtgttgtgcatttcctaatcttgtcttctgtgcagaaatttaccagatcacaaaataaaaagaaccaggtatggaaacttgagacttttgtccatatctaagctcaaaaaattctggattctgcttaaaatcaataacttccagggcaaatgattaacagaataatgatttcaatggaaatgtggtctatatgacaagagagaagcgagattatacttgaatttgatgattggcatttcacactctgttcagaattttagcctttatgaaggaccctgtctaccttcttcaaatgcctcaattgaaagggtatggcaggaaaagggatggagagggggtcagagataatcctcactgatttcacaatttcccctagggcccacctgtttgaataaatacctagtggtgatgtagcaagaaaacacccctgaatcaatatttttagacaaactaccattaccattaccatctgttaattagaaagtaactctcagatcgcttgaacccaggaggcagaggttgccatgagccgaggttgtgccactgcattccagcctgggtgacagagcgagactctgtctcaaacaaacaaacaaacaaaaaacataaaaaataaaaaataaagtaattctcagattctctgtaaatcaggagctcttttggtgatatatatatatacacacacacacacacacacacacacacacacacacacgtggagagaaaaaaaggagccagctaatgggtgatttcctttactataataattctaatttatttcttattgccagtttccacacaggtccaagccgcaaaacaataccgtctcacaacagagcagagctttacagcttataaaacattctgggaatattagaatattaacacatgaagatttttgggtccttcagcacgttagatatgacctgggacatagaggactcttaagaaaagagaaattgggcatccacatgtggaaaaaaaaactcaatctcaacctcaagccttagacaaaactcacagtggatcacagatctgaatggaaaatgtaaggctacaacactttcagaagacatagggagagaatctttgttaccctgggttatgcaaagagttcaacagcatgatccagagaaaggaaacaaatggtaaattgaacttgatcaaaatttaaaaattttgctttgtgaaaaacatagtcaggagactgaaaagacaagcaacagactgggaaaacacatttgcaagttacatatccaataaaagtgttgtatccagaatatatatgtagaacattcaaaactaaacattaagaaaataaacaattcaagtaaaaaaaaaaaaagaagaagaataggggtcaggcaagctcagtggctcacgcttgtaatcccagtgctttgggagtctaaggctggaggactgttcaagaccaggaatgtgagacaagcctgggcaacatagtgagactctatacctaccaaaaaatttagtttggtgtggtggcacacacctatactcctagctatttgggaggctgaggttggaggatcgcttgagcccaggaggtcaaggttacagtgatctatgatcacactattgtgaaaccgcctttgcaaaattatgacagacagtgaaagaaatctaacttaaccgaatccatcttgcttctaaccttcaagctgtccttgttcattgctgggcataggctgaactaattttgggagaaacttagtttatagtttatggtttaaacaaagacggtaacacccctttcccaaagtgacctccttcctgcctggggactagacttcctttgtaggactaacattagccacaagatcagatgagccagtttattgatctggtggtgccagctgatccatcaagtgcagtactgataccccaagcactgattttaggagcagtttagggagggtcagaattttgtagcctccagctgcataactcctaaaccatattttttttttttttgag

SEQ ID NO: 5 - DNA, 1020 bp, Mus musculus
  source 1..1020: mol_type = genomic DNA
cattccaggcctcgacccttccagggtgtgttctctcctcctccctctggggcaaccttctctctgtctccgccctctgttttgttttccccaaaccgcttcgctttcccggcaccggctggcccttctctcccttcacgcccaggtctcggcccatctgttcaatctccctgctccgcttaacagaacaataagccagaggctgcaggaggaggccagggcgagctgggcagcacagctcacagctttgagggaggaggagggggaggggggaggaggaggaggaggaggaggaggaggaggaggaggaggaggaggaggaggaggaggaggaggaggaggaaagctcaagtagaatcaagtgtatggaaactacagaaataacaaacgggataaggaggctggagatctccctgccaagccctctttaactttgtgttcctcccaaagccccctattaccaaacctggtccaagttcttggtcactaggcagcttcaggacagactgagcagacagtcgcagagaatcagtgtgtggggggggggcactctcagctattggctggtttaggacacagcccagttaaaaattaaggatgattgattgctagtgtgagttcctgtaaatccggatgggacggatgggacgagtctcgaactgtgggccagttagcccggagttcctcttggaatggggtaggatgtgaccggttgggataaggaagcttgagtgacgctcttagtgatcttagtgttttgtggatcaagataaaggcagaagaagcacacaacgtgcctaaggtttgacaatggatgagctttttgagattaatgggtaaagcaagcctttagaaataggccaagccttagaagtagttaaatggccttccattgctctcatttggagatatgccagccttgggcccgaatctcctaaaaacctaatggctgctggatcttccccattcctacacgtaatcaccggctccagtttccagccgagccctggcttcaacccc

SEQ ID NO: 6 - DNA, 23 bp, synthetic construct
  source 1..23: mol_type = other DNA
ttaatacagctctggagtggggt

SEQ ID NO: 7 - DNA, 21 bp, synthetic construct
  source 1..21: mol_type = other DNA
aatacagctctggagtggggt

SEQ ID NO: 8 - DNA, 20 bp, synthetic construct
  source 1..20: mol_type = other DNA
tacagctctggagtggggtg

SEQ ID NO: 9 - DNA, 20 bp, synthetic construct
  source 1..20: mol_type = other DNA
atacagctctggagtggggt

SEQ ID NO: 10 - DNA, 19 bp, synthetic construct
  source 1..19: mol_type = other DNA
aatacagctctggagtggg

SEQ ID NO: 11 - DNA, 19 bp, synthetic construct
  source 1..19: mol_type = other DNA
tacagctctggagtggggt

SEQ ID NO: 12 - DNA, 16 bp, synthetic construct
  source 1..16: mol_type = other DNA
acagctctggagtggg

SEQ ID NO: 13 - DNA, 25 bp, synthetic construct
  source 1..25: mol_type = other DNA
ttttaatacagctctggagtggggt

SEQ ID NO: 14 - DNA, 27 bp, synthetic construct
  source 1..27: mol_type = other DNA
ttttaatacagctctggagtggggtgg

SEQ ID NO: 15 - DNA, 20 bp, synthetic construct
  source 1..20: mol_type = other DNA
gcttggggtatcagtactgc

SEQ ID NO: 16 - DNA, 20 bp, synthetic construct
  source 1..20: mol_type = other DNA
tatgaagtaagaaaggggta

SEQ ID NO: 17 - DNA, 20 bp, synthetic construct
  source 1..20: mol_type = other DNA
gggtatcagtactgcacttg

SEQ ID NO: 18 - DNA, 23 bp, synthetic construct
  source 1..23: mol_type = other DNA
  misc_feature 1..23: note = Phosphorothioate linkage
  modified_base 1..5: mod_base = OTHER; note = 2' methoxyethyl nucleic acid
  modified_base 19..23: mod_base = OTHER; note = 2' methoxyethyl nucleic acid
ttaatacagctctggagtggggt

SEQ ID NO: 19 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 10: mod_base = OTHER; note = 2' locked nucleic acid
  modified_base 14: mod_base = OTHER; note = 2' locked nucleic acid
  modified_base 18..23: mod_base = OTHER; note = 2' methoxyethyl nucleic acid
ttaatacagctctggagtggggt

SEQ ID NO: 20 - DNA, 23 bp, synthetic construct
  source 1..23: mol_type = other DNA
  misc_feature 1..23: note = Phosphorothioate linkage
  modified_base 1: mod_base = OTHER; note = 2' methoxyethyl nucleic acid
  modified_base 2: mod_base = OTHER; note = 2' locked nucleic acid
  modified_base 3: mod_base = OTHER; note = 2' methoxyethyl nucleic acid
  modified_base 4: mod_base = OTHER; note = 2' locked nucleic acid
  modified_base 5: mod_base = OTHER; note = 2' methoxyethyl nucleic acid
  modified_base 6: mod_base = OTHER; note = 2' locked nucleic acid
  modified_base 8: mod_base = OTHER; note = 2' locked nucleic acid
  modified_base 10: mod_base = OTHER; note = 2' locked 5-MethylCytosine
  modified_base 12: mod_base = OTHER; note = 2' locked 5-MethylCytosine
  modified_base 14: mod_base = OTHER; note = 2' locked nucleic acid
  modified_base 16: mod_base = OTHER; note = 2' locked nucleic acid
  modified_base 18: mod_base = OTHER; note = 2' locked nucleic acid
  modified_base 19: mod_base = OTHER; note = 2' methoxyethyl nucleic acid
  modified_base 20: mod_base = OTHER; note = 2' locked nucleic acid
  modified_base 21: mod_base = OTHER; note = 2' methoxyethyl nucleic acid
  modified_base 22: mod_base = OTHER; note = 2' locked nucleic acid
  modified_base 23: mod_base = OTHER; note = 2' methoxyethyl nucleic acid
ttaatacagctctggagtggggt

SEQ ID NO: 21 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 22 - DNA, 23 bp, synthetic construct
  source 1..23: mol_type = other DNA
  misc_feature 1..23: note = Phosphorothioate linkage
  modified_base 1: mod_base = OTHER; note = 2' methoxyethyl nucleic acid
  modified_base 2: mod_base = OTHER; note = 2' locked nucleic acid
  modified_base 3: mod_base = OTHER; note = 2' methoxyethyl nucleic acid
  modified_base 4: mod_base = OTHER; note = 2' locked nucleic acid
  modified_base 5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' locked nucleic acid
  modified_base 9: mod_base = OTHER; note = 2' methoxyethyl nucleic acid
  modified_base 10: mod_base = OTHER; note = 2' locked 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13: mod_base = OTHER; note = 2' methoxyethyl nucleic acid
  modified_base 14: mod_base = OTHER; note = 2' locked nucleic acid
  modified_base 15: mod_base = OTHER; note = 2' methoxyethyl nucleic acid
  modified_base 16: mod_base = OTHER; note = 2' locked nucleic acid
  modified_base 17: mod_base = OTHER; note = 2' methoxyethyl nucleic acid
  modified_base 18: mod_base = OTHER; note = 2' locked nucleic acid
  modified_base 19: mod_base = OTHER; note = 2' methoxyethyl nucleic acid
  modified_base 20: mod_base = OTHER; note = 2' locked nucleic acid
  modified_base 21: mod_base = OTHER; note = 2' methoxyethyl nucleic acid
  modified_base 22: mod_base = OTHER; note = 2' locked nucleic acid
  modified_base 23: mod_base = OTHER; note = 2' methoxyethyl nucleic acid
ttaatacagctctggagtggggt

SEQ ID NO: 23 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 10: mod_base = OTHER; note = 2' methoxyethyl 5-MethylCytosine
  modified_base 14: mod_base = OTHER; note = 2' methoxyethyl nucleic acid
  modified_base 18..23: mod_base = OTHER; note = 2' methoxyethyl nucleic acid
ttaatacagctctggagtggggt

SEQ ID NO: 24 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' methoxyethyl 5-MethylCytosine
  modified_base 13..23: mod_base = OTHER; note = 2' methoxyethyl nucleic acid
ttaatacagctctggagtggggt

SEQ ID NO: 25 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 10: mod_base = OTHER; note = 2' methoxyethyl 5-MethylCytosine
  modified_base 14: mod_base = OTHER; note = 2' methoxyethyl nucleic acid
  modified_base 18..23: mod_base = OTHER; note = 2' methoxyethyl nucleic acid
ttaatacagctctggagtggggt

SEQ ID NO: 26 - DNA, 21 bp, synthetic construct
  source 1..21: mol_type = other DNA
  modified_base 1..5: mod_base = OTHER; note = 2' methoxyethyl nucleic acid
  modified_base 5: mod_base = OTHER; note = 2' methoxyethyl 5-MethylCytosine
  modified_base 16..20: mod_base = OTHER; note = 2' methoxyethyl nucleic acid
aatacagctctggagtggggt

SEQ ID NO: 27 - DNA, 21 bp, synthetic construct
  source 1..21: mol_type = other DNA
  modified_base 1..2: mod_base = OTHER; note = 2' methoxyethyl nucleic acid
  modified_base 4: mod_base = OTHER; note = 2' methoxyethyl nucleic acid
  modified_base 18: mod_base = OTHER; note = 2' methoxyethyl nucleic acid
  modified_base 20: mod_base = OTHER; note = 2' methoxyethyl nucleic acid
  modified_base 5: mod_base = OTHER; note = 2' methoxyethyl 5-MethylCytosine
  modified_base 3: mod_base = OTHER; note = 2' locked nucleic acid
  modified_base 7: mod_base = OTHER; note = 2' locked nucleic acid
  modified_base 9: mod_base = OTHER; note = 2' locked nucleic acid
  modified_base 11: mod_base = OTHER; note = 2' locked nucleic acid
  modified_base 13: mod_base = OTHER; note = 2' locked nucleic acid
  modified_base 15: mod_base = OTHER; note = 2' locked nucleic acid
  modified_base 17: mod_base = OTHER; note = 2' locked nucleic acid
  modified_base 19: mod_base = OTHER; note = 2' locked nucleic acid
  modified_base 21: mod_base = OTHER; note = 2' locked nucleic acid
aatacagctctggagtggggt

SEQ ID NO: 28 - DNA, 20 bp, synthetic construct
  source 1..20: mol_type = other DNA
  modified_base 1..5: mod_base = OTHER; note = 2' methoxyethyl nucleic acid
  modified_base 3: mod_base = OTHER; note = 2' methoxyethyl 5-MethylCytosine
  modified_base 16..20: mod_base = OTHER; note = 2' methoxyethyl nucleic acid
tacagctctggagtggggtg

SEQ ID NO: 29 - DNA, 20 bp, synthetic construct
  source 1..20: mol_type = other DNA
  modified_base 1..5: mod_base = OTHER; note = 2' methoxyethyl nucleic acid
  modified_base 4: mod_base = OTHER; note = 2' methoxyethyl 5-MethylCytosine
  modified_base 16..20: mod_base = OTHER; note = 2' methoxyethyl nucleic acid
atacagctctggagtggggt

SEQ ID NO: 30 - DNA, 19 bp, synthetic construct
  source 1..19: mol_type = other DNA
  modified_base 1: mod_base = OTHER; note = 2' methoxyethyl nucleic acid
  modified_base 3: mod_base = OTHER; note = 2' methoxyethyl nucleic acid
  modified_base 15: mod_base = OTHER; note = 2' methoxyethyl nucleic acid
  modified_base 17: mod_base = OTHER; note = 2' methoxyethyl nucleic acid
  modified_base 19: mod_base = OTHER; note = 2' methoxyethyl nucleic acid
  modified_base 5: mod_base = OTHER; note = 2' methoxyethyl 5-MethylCytosine
  modified_base 8: mod_base = OTHER; note = 2' locked 5-MethylCytosine
  modified_base 2: mod_base = OTHER; note = 2' locked nucleic acid
  modified_base 4: mod_base = OTHER; note = 2' locked nucleic acid
  modified_base 6: mod_base = OTHER; note = 2' locked nucleic acid
  modified_base 10: mod_base = OTHER; note = 2' locked nucleic acid
  modified_base 12: mod_base = OTHER; note = 2' locked nucleic acid
  modified_base 14: mod_base = OTHER; note = 2' locked nucleic acid
  modified_base 16: mod_base = OTHER; note = 2' locked nucleic acid
  modified_base 18: mod_base = OTHER; note = 2' locked nucleic acid
aatacagctctggagtggg

SEQ ID NO: 31 - DNA, 19 bp, synthetic construct
  source 1..19: mol_type = other DNA
  modified_base 1: mod_base = OTHER; note = 2' methoxyethyl nucleic acid
  modified_base 5: mod_base = OTHER; note = 2' methoxyethyl nucleic acid
  modified_base 15: mod_base = OTHER; note = 2' methoxyethyl nucleic acid
  modified_base 17: mod_base = OTHER; note = 2' methoxyethyl nucleic acid
  modified_base 19: mod_base = OTHER; note = 2' methoxyethyl nucleic acid
  modified_base 3: mod_base = OTHER; note = 2' methoxyethyl 5-MethylCytosine
  modified_base 6: mod_base = OTHER; note = 2' locked 5-MethylCytosine
  modified_base 8: mod_base = OTHER; note = 2' locked 5-MethylCytosine
  modified_base 2: mod_base = OTHER; note = 2' locked nucleic acid
  modified_base 4: mod_base = OTHER; note = 2' locked nucleic acid
  modified_base 10: mod_base = OTHER; note = 2' locked nucleic acid
  modified_base 12: mod_base = OTHER; note = 2' locked nucleic acid
  modified_base 14: mod_base = OTHER; note = 2' locked nucleic acid
  modified_base 16: mod_base = OTHER; note = 2' locked nucleic acid
  modified_base 18: mod_base = OTHER; note = 2' locked nucleic acid
tacagctctggagtggggt

SEQ ID NO: 32 - DNA, 16 bp, synthetic construct
  source 1..16: mol_type = other DNA
  modified_base 2: mod_base = OTHER; note = 2' locked 5-MethylCytosine
  modified_base 1: mod_base = OTHER; note = 2' locked nucleic acid
  modified_base 3: mod_base = OTHER; note = 2' locked nucleic acid
  modified_base 14..16: mod_base = OTHER; note = 2' locked nucleic acid
acagctctggagtggg

SEQ ID NO: 33 - DNA, 25 bp, synthetic construct
  source 1..25: mol_type = other DNA
  modified_base 1..25: mod_base = OTHER; note = 2' methoxyethyl nucleic acid
  modified_base 9: mod_base = OTHER; note = 2' methoxyethyl 5-MethylCytosine
  modified_base 12: mod_base = OTHER; note = 2' methoxyethyl 5-MethylCytosine
ttttaatacagctctggagtggggt

SEQ ID NO: 34 - DNA, 27 bp, synthetic construct
  source 1..27: mol_type = other DNA
  modified_base 1..8: mod_base = OTHER; note = 2' methoxyethyl nucleic acid
  modified_base 20..27: mod_base = OTHER; note = 2' methoxyethyl nucleic acid
ttttaatacagctctggagtggggtgg

SEQ ID NO: 35 - DNA, 27 bp, synthetic construct
  source 1..27: mol_type = other DNA
  modified_base 1..27: mod_base = OTHER; note = 2' methoxyethyl nucleic acid
  modified_base 9: mod_base = OTHER; note = 2' methoxyethyl 5-MethylCytosine
  modified_base 12: mod_base = OTHER; note = 2' methoxyethyl 5-MethylCytosine
ttttaatacagctctggagtggggtgg

SEQ ID NO: 36 - DNA, 20 bp, synthetic construct
  source 1..20: mol_type = other DNA
  modified_base 1..20: mod_base = OTHER; note = 2' methoxyethyl nucleic acid
  modified_base 2: mod_base = OTHER; note = 2' methoxyethyl 5-MethylCytosine
  modified_base 12: mod_base = OTHER; note = 2' methoxyethyl 5-MethylCytosine
  modified_base 17: mod_base = OTHER; note = 2' methoxyethyl 5-MethylCytosine
  modified_base 20: mod_base = OTHER; note = 2' methoxyethyl 5-MethylCytosine
gcttggggtatcagtactgc

SEQ ID NO: 37 - DNA, 20 bp, synthetic construct
  source 1..20: mol_type = other DNA
  misc_feature 1..20: note = Phosphorothioate linkage
  modified_base 1..5: mod_base = OTHER; note = 2' methoxyethyl nucleic acid
  modified_base 16..20: mod_base = OTHER; note = 2' methoxyethyl nucleic acid
tatgaagtaagaaaggggta

SEQ ID NO: 38 - DNA, 20 bp, synthetic construct
  source 1..20: mol_type = other DNA
  modified_base 1..20: mod_base = OTHER; note = 2' methoxyethyl nucleic acid
  modified_base 7: mod_base = OTHER; note = 2' methoxyethyl 5-MethylCytosine
  modified_base 11: mod_base = OTHER; note = 2' methoxyethyl 5-MethylCytosine
  modified_base 14: mod_base = OTHER; note = 2' methoxyethyl 5-MethylCytosine
  modified_base 16: mod_base = OTHER; note = 2' methoxyethyl 5-MethylCytosine
gggtatcagtactgcacttg

SEQ ID NO: 39 - DNA, 20 bp, synthetic construct
  source 1..20: mol_type = other DNA
  modified_base 1..5: mod_base = OTHER; note = 2' methoxyethyl nucleic acid
  modified_base 16..20: mod_base = OTHER; note = 2' methoxyethyl nucleic acid
ttaatacagctctggagtgg

SEQ ID NO: 40 - DNA, 20 bp, synthetic construct
  source 1..20: mol_type = other DNA
gggttttgtactgtcaattt

SEQ ID NO: 41 - DNA, 20 bp, synthetic construct
  source 1..20: mol_type = other DNA
  modified_base 1..5: mod_base = OTHER; note = 2' fluoro-arabino nucleic acid
  modified_base 16..20: mod_base = OTHER; note = 2' fluoro-arabino nucleic acid
ggatgggagtgctaagtagg

SEQ ID NO: 42 - DNA, 20 bp, synthetic construct
  source 1..20: mol_type = other DNA
ttcaaaaacaaccctggagt

SEQ ID NO: 43 - DNA, 20 bp, synthetic construct
  source 1..20: mol_type = other DNA
ggatgggagtgctaagtagg

SEQ ID NO: 44 - DNA, 20 bp, synthetic construct
  source 1..20: mol_type = other DNA
tgcttaatgcctgatagcct

SEQ ID NO: 45 - DNA, 20 bp, synthetic construct
  source 1..20: mol_type = other DNA
gatgaactttaaacctttgt

SEQ ID NO: 46 - DNA, 20 bp, synthetic construct
  source 1..20: mol_type = other DNA
aacagagggcggagacagag

SEQ ID NO: 47 - DNA, 20 bp, synthetic construct
  source 1..20: mol_type = other DNA
cggagcagggagattgaaca

SEQ ID NO: 48 - DNA, 20 bp, synthetic construct
  source 1..20: mol_type = other DNA
ttgggaggaacacaaagtta

SEQ ID NO: 49 - DNA, 20 bp, synthetic construct
  source 1..20: mol_type = other DNA
aaaacagagggcggagacag

SEQ ID NO: 50 - DNA, 22 bp, synthetic construct
  source 1..22: mol_type = other DNA
tttgggaggaacacaaagttaa

SEQ ID NO: 51 - DNA, 20 bp, synthetic construct
  source 1..20: mol_type = other DNA
aaagtggttgataccctggg

SEQ ID NO: 52 - DNA, 20 bp, synthetic construct
  source 1..20: mol_type = other DNA
  modified_base 1..20: mod_base = OTHER; note = 2' methoxyethyl nucleic acid
  modified_base 11: mod_base = OTHER; note = 2' methoxyethyl 5-MethylCytosine
  modified_base 15: mod_base = OTHER; note = 2' methoxyethyl 5-MethylCytosine
gggttttgtactgtcaattt

SEQ ID NO: 53 - DNA, 20 bp, synthetic construct
  source 1..20: mol_type = other DNA
  modified_base 1..20: mod_base = OTHER; note = 2' methoxyethyl nucleic acid
  modified_base 12: mod_base = OTHER; note = 2' methoxyethyl 5-MethylCytosine
ggatgggagtgctaagtagg

SEQ ID NO: 54 - DNA, 20 bp, synthetic construct
  source 1..20: mol_type = other DNA
  modified_base 1..5: mod_base = OTHER; note = 2' methoxyethyl nucleic acid
  modified_base 3: mod_base = OTHER; note = 2' methoxyethyl 5-MethylCytosine
  modified_base 16..20: mod_base = OTHER; note = 2' methoxyethyl nucleic acid
ttcaaaaacaaccctggagt

SEQ ID NO: 55 - DNA, 20 bp, synthetic construct
  source 1..20: mol_type = other DNA
  modified_base 1..5: mod_base = OTHER; note = 2' methoxyethyl nucleic acid
  modified_base 16..20: mod_base = OTHER; note = 2' methoxyethyl nucleic acid
ggatgggagtgctaagtagg

SEQ ID NO: 56 - DNA, 20 bp, synthetic construct
  source 1..20: mol_type = other DNA
  modified_base 1..5: mod_base = OTHER; note = 2' methoxyethyl nucleic acid
  modified_base 3: mod_base = OTHER; note = 2' methoxyethyl 5-MethylCytosine
  modified_base 18: mod_base = OTHER; note = 2' methoxyethyl 5-MethylCytosine
  modified_base 19: mod_base = OTHER; note = 2' methoxyethyl 5-MethylCytosine
  modified_base 16..20: mod_base = OTHER; note = 2' methoxyethyl nucleic acid
tgcttaatgcctgatagcct

SEQ ID NO: 57 - DNA, 20 bp, synthetic construct
  source 1..20: mol_type = other DNA
  modified_base 1..5: mod_base = OTHER; note = 2' methoxyethyl nucleic acid
  modified_base 16..20: mod_base = OTHER; note = 2' methoxyethyl nucleic acid
gatgaactttaaacctttgt

SEQ ID NO: 58 - DNA, 20 bp, synthetic construct
  source 1..20: mol_type = other DNA
  modified_base 1..20: mod_base = OTHER; note = 2' methoxyethyl nucleic acid
  modified_base 3: mod_base = OTHER; note = 2' methoxyethyl 5-MethylCytosine
  modified_base 10: mod_base = OTHER; note = 2' methoxyethyl 5-MethylCytosine
  modified_base 16: mod_base = OTHER; note = 2' methoxyethyl 5-MethylCytosine
aacagagggcggagacagag

SEQ ID NO: 59 - DNA, 20 bp, synthetic construct
  source 1..20: mol_type = other DNA
  modified_base 1..20: mod_base = OTHER; note = 2' methoxyethyl nucleic acid
  modified_base 1: mod_base = OTHER; note = 2' methoxyethyl 5-MethylCytosine
  modified_base 6: mod_base = OTHER; note = 2' methoxyethyl 5-MethylCytosine
  modified_base 19: mod_base = OTHER; note = 2' methoxyethyl 5-MethylCytosine
cggagcagggagattgaaca

SEQ ID NO: 60 - DNA, 20 bp, synthetic construct
  source 1..20: mol_type = other DNA
  modified_base 1..20: mod_base = OTHER; note = 2' methoxyethyl nucleic acid
  modified_base 11: mod_base = OTHER; note = 2' methoxyethyl 5-MethylCytosine
  modified_base 13: mod_base = OTHER; note = 2' methoxyethyl 5-MethylCytosine
ttgggaggaacacaaagtta

SEQ ID NO: 61 - DNA, 20 bp, synthetic construct
  source 1..20: mol_type = other DNA
  modified_base 1..20: mod_base = OTHER; note = 2' methoxyethyl nucleic acid
  modified_base 5: mod_base = OTHER; note = 2' methoxyethyl 5-MethylCytosine
  modified_base 12: mod_base = OTHER; note = 2' methoxyethyl 5-MethylCytosine
  modified_base 18: mod_base = OTHER; note = 2' methoxyethyl 5-MethylCytosine
aaaacagagggcggagacag

SEQ ID NO: 62 - DNA, 22 bp, synthetic construct
  source 1..22: mol_type = other DNA
  modified_base 1..22: mod_base = OTHER; note = 2' methoxyethyl nucleic acid
  modified_base 12: mod_base = OTHER; note = 2' methoxyethyl 5-MethylCytosine
  modified_base 14: mod_base = OTHER; note = 2' methoxyethyl 5-MethylCytosine
tttgggaggaacacaaagttaa

SEQ ID NO: 63 - DNA, 20 bp, synthetic construct
  source 1..20: mol_type = other DNA
  modified_base 1..5: mod_base = OTHER; note = 2' methoxyethyl nucleic acid
  modified_base 3: mod_base = OTHER; note = 2' methoxyethyl 5-MethylCytosine
  modified_base 19: mod_base = OTHER; note = 2' methoxyethyl 5-MethylCytosine
  modified_base 16..20: mod_base = OTHER; note = 2' methoxyethyl nucleic acid
tacaagtctcttggcaaaca

SEQ ID NO: 64 - DNA, 22 bp, synthetic construct
  source 1..22: mol_type = other DNA
ttatgaagtaagaaaggggtaa

SEQ ID NO: 65 - DNA, 24 bp, synthetic construct
  source 1..24: mol_type = other DNA
tttatgaagtaagaaaggggtaaa

SEQ ID NO: 66 - DNA, 20 bp, synthetic construct
  source 1..20: mol_type = other DNA
tgaagtaagaaaggggtaaa

SEQ ID NO: 67 - DNA, 20 bp, synthetic construct
  source 1..20: mol_type = other DNA
  misc_feature 1..20: note = Phosphorothioate linkage
  modified_base 1..20: mod_base = OTHER; note = 2' methoxyethyl nucleic acid
tatgaagtaagaaaggggta

SEQ ID NO: 68 - DNA, 20 bp, synthetic construct
  source 1..20: mol_type = other DNA
  misc_feature 1..20: note = Phosphorothioate linkage
  modified_base 1..3: mod_base = OTHER; note = 2' methoxyethyl nucleic acid
  modified_base 4: mod_base = OTHER; note = 2' locked nucleic acid
  modified_base 5: mod_base = OTHER; note = 2' methoxyethyl nucleic acid
  modified_base 8: mod_base = OTHER; note = 2' locked nucleic acid
  modified_base 12: mod_base = OTHER; note = 2' locked nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 69 - DNA, 20 bp, synthetic construct
  source 1..20: mol_type = other DNA
  misc_feature 1..20: note = Phosphorothioate linkage
  modified_base 1..3: mod_base = OTHER; note = 2' methoxyethyl nucleic acid
  modified_base 4: mod_base = OTHER; note = 2' locked nucleic acid
  modified_base 5..7: mod_base = OTHER; note = 2' methoxyethyl nucleic acid
  modified_base 8: mod_base = OTHER; note = 2' locked nucleic acid
  modified_base 9..11: mod_base = OTHER; note = 2' methoxyethyl nucleic acid
  modified_base 12: mod_base = OTHER; note = 2' locked nucleic acid
  modified_base 13..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 70 - DNA, 20 bp, synthetic construct
  source 1..20: mol_type = other DNA
  misc_feature 1..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 13..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 71 - DNA, 22 bp, synthetic construct
  source 1..22: mol_type = other DNA
  modified_base 1..6: mod_base = OTHER; note = 2' methoxyethyl nucleic acid
  modified_base 17..22: mod_base = OTHER; note = 2' methoxyethyl nucleic acid
ttatgaagtaagaaaggggtaa

SEQ ID NO: 72 - DNA, 22 bp, synthetic construct
  source 1..22: mol_type = other DNA
  modified_base 1..5: mod_base = OTHER; note = 2' methoxyethyl nucleic acid
  modified_base 6: mod_base = OTHER; note = 2' locked nucleic acid
  modified_base 9: mod_base = OTHER; note = 2' locked nucleic acid
  modified_base 14: mod_base = OTHER; note = 2' locked nucleic acid
  modified_base 17: mod_base = OTHER; note = 2' locked nucleic acid
  modified_base 18..22: mod_base = OTHER; note = 2' methoxyethyl nucleic acid
ttatgaagtaagaaaggggtaa

SEQ ID NO: 73 - DNA, 24 bp, synthetic construct
  source 1..24: mol_type = other DNA
  modified_base 1..5: mod_base = OTHER; note = 2' methoxyethyl nucleic acid
  modified_base 8: mod_base = OTHER; note = 2' locked nucleic acid
  modified_base 11: mod_base = OTHER; note = 2' locked nucleic acid
  modified_base 14: mod_base = OTHER; note = 2' locked nucleic acid
  modified_base 17: mod_base = OTHER; note = 2' locked nucleic acid
  modified_base 20..24: mod_base = OTHER; note = 2' methoxyethyl nucleic acid
tttatgaagtaagaaaggggtaaa

SEQ ID NO: 74 - DNA, 20 bp, synthetic construct
  source 1..20: mol_type = other DNA
  modified_base 1..5: mod_base = OTHER; note = 2' methoxyethyl nucleic acid
  modified_base 6: mod_base = OTHER; note = 2' locked nucleic acid
  modified_base 9: mod_base = OTHER; note = 2' locked nucleic acid
  modified_base 12: mod_base = OTHER; note = 2' locked nucleic acid
  modified_base 15: mod_base = OTHER; note = 2' locked nucleic acid
  modified_base 16..20: mod_base = OTHER; note = 2' methoxyethyl nucleic acid
tgaagtaagaaaggggtaaa

SEQ ID NO: 75 - DNA, 21 bp, synthetic construct
  source 1..21: mol_type = other DNA
ttaatacagctctggagtggg

SEQ ID NO: 76 - DNA, 22 bp, synthetic construct
  source 1..22: mol_type = other DNA
  modified_base 1..5: mod_base = OTHER; note = 2' methoxyethyl nucleic acid
  modified_base 18..22: mod_base = OTHER; note = 2' methoxyethyl nucleic acid
ttaatacagctctggagtgggg

SEQ ID NO: 77 - DNA, 20 bp, synthetic construct
  source 1..20: mol_type = other DNA
ttaatacagctctggagtgg

SEQ ID NO: 78 - DNA, 21 bp, synthetic construct
  source 1..21: mol_type = other DNA
  modified_base 1..5: mod_base = OTHER; note = 2' methoxyethyl nucleic acid
  modified_base 18..21: mod_base = OTHER; note = 2' methoxyethyl nucleic acid
ttaatacagctctggagtggg

SEQ ID NO: 79 - DNA, 20 bp, synthetic construct
  source 1..20: mol_type = other DNA
taacagacacttccatggcc

SEQ ID NO: 80 - DNA, 20 bp, synthetic construct
  source 1..20: mol_type = other DNA
caagccttagggaaaactgt

SEQ ID NO: 81 - DNA, 20 bp, synthetic construct
  source 1..20: mol_type = other DNA
gctttaccagatcacaaaat

SEQ ID NO: 82 - DNA, 20 bp, synthetic construct
  source 1..20: mol_type = other DNA
ggagatggctctagaattct

SEQ ID NO: 83 - DNA, 20 bp, synthetic construct
  source 1..20: mol_type = other DNA
  modified_base 1..5: mod_base = OTHER; note = 2' methoxyethyl nucleic acid
  modified_base 4: mod_base = OTHER; note = 2' methoxyethyl 5-MethylCytosine
  modified_base 19: mod_base = OTHER; note = 2' methoxyethyl 5-MethylCytosine
  modified_base 20: mod_base = OTHER; note = 2' methoxyethyl 5-MethylCytosine
  modified_base 16..20: mod_base = OTHER; note = 2' methoxyethyl nucleic acid
taacagacacttccatggcc

SEQ ID NO: 84 - DNA, 20 bp, synthetic construct
  source 1..20: mol_type = other DNA
  modified_base 1..5: mod_base = OTHER; note = 2' methoxyethyl nucleic acid
  modified_base 1: mod_base = OTHER; note = 2' methoxyethyl 5-MethylCytosine
  modified_base 5: mod_base = OTHER; note = 2' methoxyethyl 5-MethylCytosine
  modified_base 17: mod_base = OTHER; note = 2' methoxyethyl 5-MethylCytosine
  modified_base 16..20: mod_base = OTHER; note = 2' methoxyethyl nucleic acid
caagccttagggaaaactgt

SEQ ID NO: 85 - DNA, 21 bp, synthetic construct
  source 1..21: mol_type = other DNA
  modified_base 1..5: mod_base = OTHER; note = 2' methoxyethyl nucleic acid
  modified_base 2: mod_base = OTHER; note = 2' methoxyethyl 5-MethylCytosine
  modified_base 16..20: mod_base = OTHER; note = 2' methoxyethyl nucleic acid
gctttaccagatccacaaaat

SEQ ID NO: 86 - DNA, 20 bp, synthetic construct
  source 1..20: mol_type = other DNA
  modified_base 1..5: mod_base = OTHER; note = 2' methoxyethyl nucleic acid
  modified_base 2: mod_base = OTHER; note = 2' methoxyethyl 5-MethylCytosine
  modified_base 16..20: mod_base = OTHER; note = 2' methoxyethyl nucleic acid
ggagatggctctagaattct

SEQ ID NO: 87 - DNA, 23 bp, synthetic construct
  source 1..23: mol_type = other DNA
  misc_feature 1..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 88 - DNA, 23 bp, synthetic construct
  source 1..23: mol_type = other DNA
  misc_feature 1..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 89 - DNA, 23 bp, synthetic construct
  source 1..23: mol_type = other DNA
  misc_feature 1..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 90 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 18..23: mod_base = OTHER; note = 2' methoxyethyl nucleic acid
ttaatacagctctggagtggggt

SEQ ID NO: 91 - DNA, 23 bp, synthetic construct
  source 1..23: mol_type = other DNA
  modified_base 1..6: mod_base = OTHER; note = 2' methoxyethyl nucleic acid
  modified_base 7: mod_base = OTHER; note = 2' methoxyethyl 5-MethylCytosine
  modified_base 17..23: mod_base = OTHER; note = 2' methoxyethyl nucleic acid
ttaatacagctctggagtggggt

SEQ ID NO: 92 - DNA, 23 bp, synthetic construct
  source 1..23: mol_type = other DNA
  misc_feature 1..23: note = Phosphorothioate linkage
  modified_base 1..6: mod_base = OTHER; note = 2' methoxyethyl nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 16..23: mod_base = OTHER; note = 2' methoxyethyl nucleic acid
ttaatacagctctggagtggggt

SEQ ID NO: 93 - DNA, 23 bp, synthetic construct
  source 1..23: mol_type = other DNA
  misc_feature 1..23: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 10: mod_base = OTHER; note = 2' methoxyethyl 5-MethylCytosine
  modified_base 14: mod_base = OTHER; note = 2' locked nucleic acid
  modified_base 18: mod_base = OTHER; note = 2' locked nucleic acid
  modified_base 19..23: mod_base = OTHER; note = 2' methoxyethyl nucleic acid
ttaatacagctctggagtggggt

SEQ ID NO: 94 - DNA, 23 bp, synthetic construct
  source 1..23: mol_type = other DNA
  misc_feature 1..23: note = Phosphorothioate linkage
  modified_base 1: mod_base = OTHER; note = 2' methoxyethyl nucleic acid
  modified_base 2: mod_base = OTHER; note = 2' locked nucleic acid
  modified_base 3..5: mod_base = OTHER; note = 2' methoxyethyl nucleic acid
  modified_base 6: mod_base = OTHER; note = 2' locked nucleic acid
  modified_base 10: mod_base = OTHER; note = 2' locked 5-MethylCytosine
  modified_base 14: mod_base = OTHER; note = 2' locked nucleic acid
  modified_base 18: mod_base = OTHER; note = 2' locked nucleic acid
  modified_base 18: mod_base = OTHER; note = 2' locked nucleic acid
  modified_base 19..21: mod_base = OTHER; note = 2' methoxyethyl nucleic acid
  modified_base 22: mod_base = OTHER; note = 2' locked nucleic acid
  modified_base 23: mod_base = OTHER; note = 2' methoxyethyl nucleic acid
ttaatacagctctggagtggggt

SEQ ID NO: 95 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 9: mod_base = OTHER; note = 2' locked nucleic acid
  modified_base 12: mod_base = OTHER; note = 2' locked 5-MethylCytosine
  modified_base 15: mod_base = OTHER; note = 2' locked nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 96 - DNA, 23 bp, synthetic construct
  source 1..23: mol_type = other DNA
  misc_feature 1..23: note = Phosphorothioate linkage
  modified_base 1: mod_base = OTHER; note = 2' methoxyethyl nucleic acid
  modified_base 2: mod_base = OTHER; note = 2' locked nucleic acid
  modified_base 3..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' locked 5-MethylCytosine
  modified_base 11: mod_base = OTHER; note = 2' methoxyethyl nucleic acid
  modified_base 12: mod_base = OTHER; note = 2' methoxyethyl 5-MethylCytosine
  modified_base 13: mod_base = OTHER; note = 2' methoxyethyl nucleic acid
  modified_base 14: mod_base = OTHER; note = 2' locked nucleic acid
  modified_base 15..17: mod_base = OTHER; note = 2' methoxyethyl nucleic acid
  modified_base 18: mod_base = OTHER; note = 2' locked nucleic acid
  modified_base 19..21: mod_base = OTHER; note = 2' methoxyethyl nucleic acid
  modified_base 22: mod_base = OTHER; note = 2' locked nucleic acid
  modified_base 23: mod_base = OTHER; note = 2' methoxyethyl nucleic acid
ttaatacagctctggagtggggt

SEQ ID NO: 97 - DNA, 23 bp, synthetic construct
  source 1..23: mol_type = other DNA
  misc_feature 1..4: note = Phosphorothioate linkage
  misc_feature 6..20: note = Phosphorothioate linkage
  misc_feature 21..23: note = Phosphorothioate linkage
  modified_base 1..6: mod_base = OTHER; note = 2' methoxyethyl nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..23: mod_base = OTHER; note = 2' methoxyethyl nucleic acid
ttaatacagctctggagtggggt

SEQ ID NO: 98 - DNA, 23 bp, synthetic construct
  source 1..23: mol_type = other DNA
  misc_feature 1..4: note = Phosphorothioate linkage
  misc_feature 5..6: note = Phosphorothioate linkage
  misc_feature 7..8: note = Phosphorothioate linkage
  misc_feature 9..10: note = Phosphorothioate linkage
  misc_feature 11..13: note = Phosphorothioate linkage
  misc_feature 14..15: note = Phosphorothioate linkage
  misc_feature 16..17: note = Phosphorothioate linkage
  misc_feature 18..19: note = Phosphorothioate linkage
  misc_feature 20..23: note = Phosphorothioate linkage
  modified_base 1..6: mod_base = OTHER; note = 2' methoxyethyl nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..23: mod_base = OTHER; note = 2' methoxyethyl nucleic acid
ttaatacagctctggagtggggt

SEQ ID NO: 99 - DNA, 23 bp, synthetic construct
  source 1..23: mol_type = other DNA
  misc_feature 1..5: note = Phosphorothioate linkage
  misc_feature 19..23: note = Phosphorothioate linkage
  modified_base 1..6: mod_base = OTHER; note = 2' methoxyethyl nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..23: mod_base = OTHER; note = 2' methoxyethyl nucleic acid
ttaatacagctctggagtggggt

SEQ ID NO: 100 - DNA, 23 bp, synthetic construct
  source 1..23: mol_type = other DNA
  modified_base 1..6: mod_base = OTHER; note = 2' methoxyethyl nucleic acid
  modified_base 7: mod_base = OTHER; note = 2' methoxyethyl 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..23: mod_base = OTHER; note = 2' methoxyethyl nucleic acid
ttaatacagctctggagtggggt

SEQ ID NO: 101 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 9: mod_base = OTHER; note = 2' locked nucleic acid
  modified_base 11: mod_base = OTHER; note = 2' methoxyethyl nucleic acid
  modified_base 12: mod_base = OTHER; note = 2' locked 5-MethylCytosine
  modified_base 13: mod_base = OTHER; note = 2' methoxyethyl nucleic acid
  modified_base 15: mod_base = OTHER; note = 2' locked nucleic acid
  modified_base 17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 102 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 9: mod_base = OTHER; note = 2' methoxyethyl nucleic acid
  modified_base 11: mod_base = OTHER; note = 2' methoxyethyl nucleic acid
  modified_base 12: mod_base = OTHER; note = 2' locked 5-MethylCytosine
  modified_base 13: mod_base = OTHER; note = 2' methoxyethyl nucleic acid
  modified_base 15: mod_base = OTHER; note = 2' methoxyethyl nucleic acid
  modified_base 17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 103 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..6: mod_base = OTHER; note = 2' methoxyethyl nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11..14: mod_base = OTHER; note = 2' methoxyethyl nucleic acid
  modified_base 15: mod_base = OTHER; note = 2' locked nucleic acid
  modified_base 16..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 104 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 9: mod_base = OTHER; note = 2' locked nucleic acid
  modified_base 15: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 105 - DNA, 23 bp, synthetic construct
  source 1..23: mol_type = other DNA
  misc_feature 1..23: note = Phosphorothioate linkage
  modified_base 1..3: mod_base = OTHER; note = 2' methoxyethyl nucleic acid
  modified_base 4: mod_base = OTHER; note = 2' locked nucleic acid
  modified_base 5..6: mod_base = OTHER; note = 2' methoxyethyl nucleic acid
  modified_base 7: mod_base = OTHER; note = 2' methoxyethyl 5-MethylCytosine
  modified_base 8: mod_base = OTHER; note = 2' locked nucleic acid
  modified_base 9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..15: mod_base = OTHER; note = 2' methoxyethyl nucleic acid
  modified_base 16: mod_base = OTHER; note = 2' locked nucleic acid
  modified_base 17..19: mod_base = OTHER; note = 2' methoxyethyl nucleic acid
  modified_base 20: mod_base = OTHER; note = 2' locked nucleic acid
  modified_base 21..23: mod_base = OTHER; note = 2' methoxyethyl nucleic acid
ttaatacagctctggagtggggt

SEQ ID NO: 106 - DNA, 23 bp, synthetic construct
  source 1..23: mol_type = other DNA
  misc_feature 1..23: note = Phosphorothioate linkage
  modified_base 1..3: mod_base = OTHER; note = 2' methoxyethyl nucleic acid
  modified_base 4: mod_base = OTHER; note = 2' locked nucleic acid
  modified_base 5: mod_base = OTHER; note = 2' methoxyethyl nucleic acid
  modified_base 8: mod_base = OTHER; note = 2' locked nucleic acid
  modified_base 12: mod_base = OTHER; note = 2' locked 5-MethylCytosine
  modified_base 16: mod_base = OTHER; note = 2' locked nucleic acid
  modified_base 19: mod_base = OTHER; note = 2' methoxyethyl nucleic acid
  modified_base 20: mod_base = OTHER; note = 2' locked nucleic acid
  modified_base 21..23: mod_base = OTHER; note = 2' methoxyethyl nucleic acid
ttaatacagctctggagtggggt

SEQ ID NO: 107 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..6: mod_base = OTHER; note = 2' methoxyethyl nucleic acid
  modified_base 7: mod_base = OTHER; note = 2' locked 5-MethylCytosine
  modified_base 8..9: mod_base = OTHER; note = 2' methoxyethyl nucleic acid
  modified_base 10: mod_base = OTHER; note = 2' methoxyethyl 5-MethylCytosine
  modified_base 11: mod_base = OTHER; note = 2' locked nucleic acid
  modified_base 12: mod_base = OTHER; note = 2' methoxyethyl 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..18: mod_base = OTHER; note = 2' methoxyethyl nucleic acid
  modified_base 19: mod_base = OTHER; note = 2' locked nucleic acid
  modified_base 20..23: mod_base = OTHER; note = 2' methoxyethyl nucleic acid
ttaatacagctctggagtggggt

SEQ ID NO: 108 - DNA, 23 bp, synthetic construct
  source 1..23: mol_type = other DNA
  misc_feature 1..23: note = Phosphorothioate linkage
  modified_base 1..4: mod_base = OTHER; note = 2' methoxyethyl nucleic acid
  modified_base 5: mod_base = OTHER; note = 2' locked nucleic acid
  modified_base 6: mod_base = OTHER; note = 2' methoxyethyl nucleic acid
  modified_base 7: mod_base = OTHER; note = 2' locked 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' locked nucleic acid
  modified_base 12: mod_base = OTHER; note = 2' methoxyethyl 5-MethylCytosine
  modified_base 13: mod_base = OTHER; note = 2' locked nucleic acid
  modified_base 14..16: mod_base = OTHER; note = 2' methoxyethyl 5-MethylCytosine
  modified_base 17: mod_base = OTHER; note = 2' locked nucleic acid
  modified_base 18..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 109 - DNA, 23 bp, synthetic construct
  source 1..23: mol_type = other DNA
  misc_feature 1..4: note = Phosphorothioate linkage
  misc_feature 6..18: note = Phosphorothioate linkage
  misc_feature 20..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' locked 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 110 - DNA, 23 bp, synthetic construct
  source 1..23: mol_type = other DNA
  misc_feature 1..3: note = Phosphorothioate linkage
  misc_feature 5..19: note = Phosphorothioate linkage
  misc_feature 21..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 111 - DNA, 23 bp, synthetic construct
  source 1..23: mol_type = other DNA
  misc_feature 1..3: note = Phosphorothioate linkage
  misc_feature 5..19: note = Phosphorothioate linkage
  misc_feature 21..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 5-MethylCytosine
  modified_base 12: mod_base = OTHER; note = 2' locked nucleic acid
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 112 - DNA, 23 bp, synthetic construct
  source 1..23: mol_type = other DNA
  misc_feature 1..4: note = Phosphorothioate linkage
  misc_feature 5..19: note = Phosphorothioate linkage
  misc_feature 20..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 113 - DNA, 23 bp, synthetic construct
  source 1..23: mol_type = other DNA
  misc_feature 1..3: note = Phosphorothioate linkage
  misc_feature 5..19: note = Phosphorothioate linkage
  misc_feature 22..23: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 114 - DNA, 23 bp, synthetic construct
  source 1..23: mol_type = other DNA
  misc_feature 5..19: note = Phosphorothioate linkage
  modified_base 1..5: mod_base = OTHER; note = 2' methoxyethyl nucleic acid
  modified_base 7: mod_base = OTHER; note = 5-MethylCytosine
  modified_base 10: mod_base = OTHER; note = 5-MethylCytosine
  modified_base 12: mod_base = OTHER; note = 5-MethylCytosine
  modified_base 19..23: mod_base = OTHER; note = 2' methoxyethyl nucleic acid
ttaatacagctctggagtggggt

SEQ ID NO: 115 - DNA, 23 bp, synthetic construct
  source 1..23: mol_type = other DNA
  misc_feature 1..19: note = Phosphorothioate linkage
  misc_feature 20..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 116 - DNA, 23 bp, synthetic construct
  source 1..23: mol_type = other DNA
  misc_feature 1..8: note = Phosphorothioate linkage
  misc_feature 9..15: note = Phosphorothioate linkage
  misc_feature 16..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 117 - DNA, 23 bp, synthetic construct
  source 1..23: mol_type = other DNA
  misc_feature 1..5: note = Phosphorothioate linkage
  misc_feature 6..9: note = Phosphorothioate linkage
  misc_feature 10..14: note = Phosphorothioate linkage
  misc_feature 15..18: note = Phosphorothioate linkage
  misc_feature 19..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 118 - DNA, 23 bp, synthetic construct
  source 1..23: mol_type = other DNA
  misc_feature 1..2: note = Phosphorothioate linkage
  misc_feature 5..19: note = Phosphorothioate linkage
  misc_feature 22..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 119 - DNA, 23 bp, synthetic construct
  source 1..23: mol_type = other DNA
  misc_feature 1..3: note = Phosphorothioate linkage
  misc_feature 4..7: note = Phosphorothioate linkage
  misc_feature 8..10: note = Phosphorothioate linkage
  misc_feature 11..13: note = Phosphorothioate linkage
  misc_feature 14..16: note = Phosphorothioate linkage
  misc_feature 17..20: note = Phosphorothioate linkage
  misc_feature 21..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 120 - DNA, 23 bp, synthetic construct
  source 1..23: mol_type = other DNA
  misc_feature 5..19: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 121 - DNA, 23 bp, synthetic construct
  source 1..23: mol_type = other DNA
  misc_feature 1..3: note = Phosphorothioate linkage
  misc_feature 4..5: note = Phosphorothioate linkage
  misc_feature 6..8: note = Phosphorothioate linkage
  misc_feature 9..10: note = Phosphorothioate linkage
  misc_feature 11..13: note = Phosphorothioate linkage
  misc_feature 14..15: note = Phosphorothioate linkage
  misc_feature 16..18: note = Phosphorothioate linkage
  misc_feature 19..20: note = Phosphorothioate linkage
  misc_feature 21..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 122 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 123 - DNA, 23 bp, synthetic construct
  source 1..23: mol_type = other DNA
  misc_feature 1..23: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 124 - DNA, 23 bp, synthetic construct
  source 1..23: mol_type = other DNA
  misc_feature 1..22: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl 5-MethylCytosine
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl 5-MethylCytosine
  modified_base 11: mod_base = OTHER; note = 2' methoxyethyl nucleic acid
  modified_base 12: mod_base = OTHER; note = 2' locked 5-MethylCytosine
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
  modified_base 21: mod_base = OTHER; note = 2' locked nucleic acid
  modified_base 22..23: mod_base = OTHER; note = 2' methoxyethyl nucleic acid
ttaatacagctctggagtggggt

SEQ ID NO: 125 - DNA, 20 bp, synthetic construct
  source 1..20: mol_type = other DNA
  misc_feature 1..20: note = Phosphorothioate linkage
  modified_base 1..5: mod_base = OTHER; note = 2' methoxyethyl nucleic acid
  modified_base 9: mod_base = OTHER; note = 2' methoxyethyl nucleic acid
  modified_base 12: mod_base = OTHER; note = 2' methoxyethyl nucleic acid
  modified_base 16..20: mod_base = OTHER; note = 2' methoxyethyl nucleic acid
tatgaagtaagaaaggggta

SEQ ID NO: 126 - DNA, 20 bp, synthetic construct
  source 1..20: mol_type = other DNA
  misc_feature 1..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 9: mod_base = OTHER; note = 2' locked nucleic acid
  modified_base 12: mod_base = OTHER; note = 2' locked nucleic acid
  modified_base 15: mod_base = OTHER; note = 2' locked nucleic acid
  modified_base 16..20: mod_base = OTHER; note = 2' methoxyethyl nucleic acid
tatgaagtaagaaaggggta

SEQ ID NO: 127 - DNA, 20 bp, synthetic construct
  source 1..20: mol_type = other DNA
  misc_feature 1..20: note = Phosphorothioate linkage
  modified_base 1..4: mod_base = OTHER; note = 2' methoxyethyl nucleic acid
  modified_base 5: mod_base = OTHER; note = 2' locked nucleic acid
  modified_base 9: mod_base = OTHER; note = 2' locked nucleic acid
  modified_base 13: mod_base = OTHER; note = 2' locked nucleic acid
  modified_base 16: mod_base = OTHER; note = 2' methoxyethyl nucleic acid
  modified_base 17: mod_base = OTHER; note = 2' locked nucleic acid
  modified_base 18..20: mod_base = OTHER; note = 2' methoxyethyl nucleic acid
tatgaagtaagaaaggggta

SEQ ID NO: 128 - DNA, 20 bp, synthetic construct
  source 1..20: mol_type = other DNA
  misc_feature 1..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..8: mod_base = OTHER; note = 2' methoxyethyl nucleic acid
  modified_base 9: mod_base = OTHER; note = 2' locked nucleic acid
  modified_base 10..11: mod_base = OTHER; note = 2' methoxyethyl nucleic acid
  modified_base 12: mod_base = OTHER; note = 2' locked nucleic acid
  modified_base 13..14: mod_base = OTHER; note = 2' methoxyethyl nucleic acid
  modified_base 15: mod_base = OTHER; note = 2' locked nucleic acid
  modified_base 16..20: mod_base = OTHER; note = 2' methoxyethyl nucleic acid
tatgaagtaagaaaggggta

SEQ ID NO: 129 - DNA, 20 bp, synthetic construct
  source 1..20: mol_type = other DNA
  misc_feature 1..20: note = Phosphorothioate linkage
  modified_base 1..3: mod_base = OTHER; note = 2' methoxyethyl nucleic acid
  modified_base 4: mod_base = OTHER; note = 2' locked nucleic acid
  modified_base 5: mod_base = OTHER; note = 2' methoxyethyl nucleic acid
  modified_base 6: mod_base = OTHER; note = 2' locked nucleic acid
  modified_base 7: mod_base = OTHER; note = 2' methoxyethyl nucleic acid
  modified_base 8: mod_base = OTHER; note = 2' locked nucleic acid
  modified_base 9: mod_base = OTHER; note = 2' methoxyethyl nucleic acid
  modified_base 10: mod_base = OTHER; note = 2' locked nucleic acid
  modified_base 11: mod_base = OTHER; note = 2' methoxyethyl nucleic acid
  modified_base 12: mod_base = OTHER; note = 2' locked nucleic acid
  modified_base 13: mod_base = OTHER; note = 2' methoxyethyl nucleic acid
  modified_base 14: mod_base = OTHER; note = 2' locked nucleic acid
  modified_base 15: mod_base = OTHER; note = 2' methoxyethyl nucleic acid
  modified_base 16: mod_base = OTHER; note = 2' locked nucleic acid
  modified_base 17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
tatgaagtaagaaaggggta

SEQ ID NO: 130 - DNA, 20 bp, synthetic construct
  source 1..20: mol_type = other DNA
  misc_feature 1..20: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4: mod_base = OTHER; note = 2' methoxyethyl nucleic acid
  modified_base 5: mod_base = OTHER; note = 2' locked nucleic acid
  modified_base 6: mod_base = OTHER; note = 2' methoxyethyl nucleic acid
  modified_base 7: mod_base = OTHER; note = 2' locked nucleic acid
  modified_base 8: mod_base = OTHER; note = 2' methoxyethyl nucleic acid
  modified_base 9: mod_base = OTHER; note = 2' locked nucleic acid
  modified_base 10: mod_base = OTHER; note = 2' methoxyethyl nucleic acid
  modified_base 11: mod_base = OTHER; note = 2' locked nucleic acid
  modified_base 12: mod_base = OTHER; note = 2' methoxyethyl nucleic acid
  modified_base 13: mod_base = OTHER; note = 2' locked nucleic acid
  modified_base 14: mod_base = OTHER; note = 2' methoxyethyl nucleic acid
  modified_base 15: mod_base = OTHER; note = 2' locked nucleic acid
  modified_base 16: mod_base = OTHER; note = 2' methoxyethyl nucleic acid
  modified_base 17: mod_base = OTHER; note = 2' locked nucleic acid
  modified_base 18..20: mod_base = OTHER; note = 2' methoxyethyl nucleic acid
tatgaagtaagaaaggggta

SEQ ID NO: 131 - DNA, 20 bp, synthetic construct
  source 1..20: mol_type = other DNA
  misc_feature 1..20: note = Phosphorothioate linkage
  modified_base 1..2: mod_base = OTHER; note = 2' methoxyethyl nucleic acid
  modified_base 3: mod_base = OTHER; note = 2' locked nucleic acid
  modified_base 4..5: mod_base = OTHER; note = 2' methoxyethyl nucleic acid
  modified_base 6: mod_base = OTHER; note = 2' locked nucleic acid
  modified_base 7..8: mod_base = OTHER; note = 2' methoxyethyl nucleic acid
  modified_base 9: mod_base = OTHER; note = 2' locked nucleic acid
  modified_base 10..11: mod_base = OTHER; note = 2' methoxyethyl nucleic acid
  modified_base 12: mod_base = OTHER; note = 2' locked nucleic acid
  modified_base 13..14: mod_base = OTHER; note = 2' methoxyethyl nucleic acid
  modified_base 15: mod_base = OTHER; note = 2' locked nucleic acid
  modified_base 16..17: mod_base = OTHER; note = 2' methoxyethyl nucleic acid
  modified_base 18: mod_base = OTHER; note = 2' locked nucleic acid
  modified_base 19..20: mod_base = OTHER; note = 2' methoxyethyl nucleic acid
tatgaagtaagaaaggggta

SEQ ID NO: 132 - DNA, 20 bp, synthetic construct
  source 1..20: mol_type = other DNA
  misc_feature 1..20: note = Phosphorothioate linkage
  modified_base 1..4: mod_base = OTHER; note = 2' methoxyethyl nucleic acid
  modified_base 5: mod_base = OTHER; note = 2' locked nucleic acid
  modified_base 6..8: mod_base = OTHER; note = 2' methoxyethyl nucleic acid
  modified_base 9: mod_base = OTHER; note = 2' locked nucleic acid
  modified_base 10..12: mod_base = OTHER; note = 2' methoxyethyl nucleic acid
  modified_base 13: mod_base = OTHER; note = 2' locked nucleic acid
  modified_base 14..16: mod_base = OTHER; note = 2' methoxyethyl nucleic acid
  modified_base 17: mod_base = OTHER; note = 2' locked nucleic acid
  modified_base 18..20: mod_base = OTHER; note = 2' methoxyethyl nucleic acid
tatgaagtaagaaaggggta

SEQ ID NO: 133 - DNA, 20 bp, synthetic construct
  source 1..20: mol_type = other DNA
  misc_feature 1..20: note = Phosphorothioate linkage
  modified_base 1..4: mod_base = OTHER; note = 2' methoxyethyl nucleic acid
  modified_base 5: mod_base = OTHER; note = 2' locked nucleic acid
  modified_base 6..9: mod_base = OTHER; note = 2' methoxyethyl nucleic acid
  modified_base 10: mod_base = OTHER; note = 2' locked nucleic acid
  modified_base 11..14: mod_base = OTHER; note = 2' methoxyethyl nucleic acid
  modified_base 15: mod_base = OTHER; note = 2' locked nucleic acid
  modified_base 16..20: mod_base = OTHER; note = 2' methoxyethyl nucleic acid
tatgaagtaagaaaggggta

SEQ ID NO: 134 - DNA, 20 bp, synthetic construct
  source 1..20: mol_type = other DNA
  misc_feature 1..3: note = Phosphorothioate linkage
  misc_feature 5..16: note = Phosphorothioate linkage
  misc_feature 18..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 135 - DNA, 20 bp, synthetic construct
  source 1..20: mol_type = other DNA
  misc_feature 1..4: note = Phosphorothioate linkage
  misc_feature 5..16: note = Phosphorothioate linkage
  misc_feature 17..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 136 - DNA, 20 bp, synthetic construct
  source 1..20: mol_type = other DNA
  misc_feature 1..7: note = Phosphorothioate linkage
  misc_feature 8..13: note = Phosphorothioate linkage
  misc_feature 14..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 137 - DNA, 20 bp, synthetic construct
  source 1..20: mol_type = other DNA
  misc_feature 1..4: note = Phosphorothioate linkage
  misc_feature 5..8: note = Phosphorothioate linkage
  misc_feature 9..12: note = Phosphorothioate linkage
  misc_feature 13..16: note = Phosphorothioate linkage
  misc_feature 17..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 138 - DNA, 20 bp, synthetic construct
  source 1..20: mol_type = other DNA
  misc_feature 1..2: note = Phosphorothioate linkage
  misc_feature 5..16: note = Phosphorothioate linkage
  misc_feature 19..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 139 - DNA, 20 bp, synthetic construct
  source 1..20: mol_type = other DNA
  misc_feature 1..3: note = Phosphorothioate linkage
  misc_feature 4..6: note = Phosphorothioate linkage
  misc_feature 7..9: note = Phosphorothioate linkage
  misc_feature 10..11: note = Phosphorothioate linkage
  misc_feature 12..14: note = Phosphorothioate linkage
  misc_feature 15..17: note = Phosphorothioate linkage
  misc_feature 18..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 140 - DNA, 20 bp, synthetic construct
  source 1..20: mol_type = other DNA
  misc_feature 5..16: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 141 - DNA, 20 bp, synthetic construct
  source 1..20: mol_type = other DNA
  misc_feature 1..3: note = Phosphorothioate linkage
  misc_feature 4..5: note = Phosphorothioate linkage
  misc_feature 6..7: note = Phosphorothioate linkage
  misc_feature 8..9: note = Phosphorothioate linkage
  misc_feature 10..11: note = Phosphorothioate linkage
  misc_feature 12..13: note = Phosphorothioate linkage
  misc_feature 14..15: note = Phosphorothioate linkage
  misc_feature 16..17: note = Phosphorothioate linkage
  misc_feature 18..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggta

SEQ ID NO: 142 - DNA, 22 bp, synthetic construct
  source 1..22: mol_type = other DNA
  misc_feature 1..20: note = Phosphorothioate linkage
  modified_base 1..5: mod_base = OTHER; note = 2' methoxyethyl nucleic acid
  modified_base 6: mod_base = OTHER; note = 2' locked nucleic acid
  modified_base 7..10: mod_base = OTHER; note = 2' methoxyethyl nucleic acid
  modified_base 11: mod_base = OTHER; note = 2' locked nucleic acid
  modified_base 12..15: mod_base = OTHER; note = 2' methoxyethyl nucleic acid
  modified_base 16: mod_base = OTHER; note = 2' locked nucleic acid
  modified_base 17..20: mod_base = OTHER; note = 2' methoxyethyl nucleic acid
tatgaagtaagaaaggggtatg

SEQ ID NO: 143 - DNA, 20 bp, synthetic construct
  source 1..20: mol_type = other DNA
ttaatacagctctggagtgg

SEQ ID NO: 144 - DNA, 20 bp, synthetic construct
  source 1..20: mol_type = other DNA
gcctctcaccacatgtgcca

SEQ ID NO: 145 - DNA, 20 bp, synthetic construct
  source 1..20: mol_type = other DNA
tcacaacaaagctatgagta

SEQ ID NO: 146 - DNA, 20 bp, synthetic construct
  source 1..20: mol_type = other DNA
actccttggttccctggaag

SEQ ID NO: 147 - DNA, 20 bp, synthetic construct
  source 1..20: mol_type = other DNA
caaagccccatgaaaagggc

SEQ ID NO: 148 - DNA, 20 bp, synthetic construct
  source 1..20: mol_type = other DNA
accagtggagcagccttgag

SEQ ID NO: 149 - DNA, 20 bp, synthetic construct
  source 1..20: mol_type = other DNA
caagagcacctcccaagtag

SEQ ID NO: 150 - DNA, 20 bp, synthetic construct
  source 1..20: mol_type = other DNA
ttttgaagccatccaagaca

SEQ ID NO: 151 - DNA, 20 bp, synthetic construct
  source 1..20: mol_type = other DNA
gaaaatgatctcatgggatc

SEQ ID NO: 152 - DNA, 20 bp, synthetic construct
  source 1..20: mol_type = other DNA
gatttccctgtttgctctgt

SEQ ID NO: 153 - DNA, 20 bp, synthetic construct
  source 1..20: mol_type = other DNA
ggcagttgagatgcaaagca

SEQ ID NO: 154 - DNA, 20 bp, synthetic construct
  source 1..20: mol_type = other DNA
tttttattttgtgatctggt

SEQ ID NO: 155 - DNA, 20 bp, synthetic construct
  source 1..20: mol_type = other DNA
tctgttaatcatttgccctg

SEQ ID NO: 156 - DNA, 20 bp, synthetic construct
  source 1..20: mol_type = other DNA
acatttccattgaaatcatt

SEQ ID NO: 157 - DNA, 20 bp, synthetic construct
  source 1..20: mol_type = other DNA
gctaaaattctgaacagagt

SEQ ID NO: 158 - DNA, 20 bp, synthetic construct
  source 1..20: mol_type = other DNA
aaatcagtgaggattatctc

SEQ ID NO: 159 - DNA, 20 bp, synthetic construct
  source 1..20: mol_type = other DNA
acccaggctggaatgcagtg

SEQ ID NO: 160 - DNA, 20 bp, synthetic construct
  source 1..20: mol_type = other DNA
aaaagagctcctgatttaca

SEQ ID NO: 161 - DNA, 20 bp, synthetic construct
  source 1..20: mol_type = other DNA
tagtaaaggaaatcacccat

SEQ ID NO: 162 - DNA, 20 bp, synthetic construct
  source 1..20: mol_type = other DNA
gttttataagctgtaaagct

SEQ ID NO: 163 - DNA, 20 bp, synthetic construct
  source 1..20: mol_type = other DNA
tcctctatgtcccaggtcat

SEQ ID NO: 164 - DNA, 20 bp, synthetic construct
  source 1..20: mol_type = other DNA
gttgtagccttacattttcc

SEQ ID NO: 165 - DNA, 20 bp, synthetic construct
  source 1..20: mol_type = other DNA
aagttcaatttaccatttgt

SEQ ID NO: 166 - DNA, 20 bp, synthetic construct
  source 1..20: mol_type = other DNA
attggatatgtaacttgcaa

SEQ ID NO: 167 - DNA, 20 bp, synthetic construct
  source 1..20: mol_type = other DNA
acccctattcttcttctttt

SEQ ID NO: 168 - DNA, 20 bp, synthetic construct
  source 1..20: mol_type = other DNA
ctggtcttgaacagtcctcc

SEQ ID NO: 169 - DNA, 20 bp, synthetic construct
  source 1..20: mol_type = other DNA
gctcaagcgatcctccaacc

SEQ ID NO: 170 - DNA, 20 bp, synthetic construct
  source 1..20: mol_type = other DNA
ggattcggttaagttagatt

SEQ ID NO: 171 - DNA, 20 bp, synthetic construct
  source 1..20: mol_type = other DNA
tctttgtttaaaccataaac

SEQ ID NO: 172 - DNA, 20 bp, synthetic construct
  source 1..20: mol_type = other DNA
caggcaggaaggaggtcact

SEQ ID NO: 173 - DNA, 20 bp, synthetic construct
  source 1..20: mol_type = other DNA
ttaatacagctctggagtgg

SEQ ID NO: 174 - DNA, 20 bp, synthetic construct
  source 1..20: mol_type = other DNA
gcctctcaccacatgtgcca

SEQ ID NO: 175 - DNA, 20 bp, synthetic construct
  source 1..20: mol_type = other DNA
tcacaacaaagctatgagta

SEQ ID NO: 176 - DNA, 20 bp, synthetic construct
  source 1..20: mol_type = other DNA
actccttggttccctggaag

SEQ ID NO: 177 - DNA, 20 bp, synthetic construct
  source 1..20: mol_type = other DNA
caaagccccatgaaaagggc

SEQ ID NO: 178 - DNA, 20 bp, synthetic construct
  source 1..20: mol_type = other DNA
accagtggagcagccttgag

SEQ ID NO: 179 - DNA, 20 bp, synthetic construct
  source 1..20: mol_type = other DNA
caagagcacctcccaagtag

SEQ ID NO: 180 - DNA, 20 bp, synthetic construct
  source 1..20: mol_type = other DNA
ttttgaagccatccaagaca

SEQ ID NO: 181 - DNA, 20 bp, synthetic construct
  source 1..20: mol_type = other DNA
gaaaatgatctcatgggatc

SEQ ID NO: 182 - DNA, 20 bp, synthetic construct
  source 1..20: mol_type = other DNA
gatttccctgtttgctctgt

SEQ ID NO: 183 - DNA, 20 bp, synthetic construct
  source 1..20: mol_type = other DNA
ggcagttgagatgcaaagca

SEQ ID NO: 184 - DNA, 20 bp, synthetic construct
  source 1..20: mol_type = other DNA
tttttattttgtgatctggt

SEQ ID NO: 185 - DNA, 20 bp, synthetic construct
  source 1..20: mol_type = other DNA
tctgttaatcatttgccctg

SEQ ID NO: 186 - DNA, 20 bp, synthetic construct
  source 1..20: mol_type = other DNA
acatttccattgaaatcatt

SEQ ID NO: 187 - DNA, 20 bp, synthetic construct
  source 1..20: mol_type = other DNA
gctaaaattctgaacagagt

SEQ ID NO: 188 - DNA, 20 bp, synthetic construct
  source 1..20: mol_type = other DNA
aaatcagtgaggattatctc

SEQ ID NO: 189 - DNA, 20 bp, synthetic construct
  source 1..20: mol_type = other DNA
ttagacaaactaccattacc

SEQ ID NO: 190 - DNA, 20 bp, synthetic construct
  source 1..20: mol_type = other DNA
acccaggctggaatgcagtg

SEQ ID NO: 191 - DNA, 20 bp, synthetic construct
  source 1..20: mol_type = other DNA
aaaagagctcctgatttaca

SEQ ID NO: 192 - DNA, 20 bp, synthetic construct
  source 1..20: mol_type = other DNA
tagtaaaggaaatcacccat

SEQ ID NO: 193 - DNA, 20 bp, synthetic construct
  source 1..20: mol_type = other DNA
gttttataagctgtaaagct

SEQ ID NO: 194 - DNA, 20 bp, synthetic construct
  source 1..20: mol_type = other DNA
tcctctatgtcccaggtcat

SEQ ID NO: 195 - DNA, 20 bp, synthetic construct
  source 1..20: mol_type = other DNA
gttgtagccttacattttcc

SEQ ID NO: 196 - DNA, 20 bp, synthetic construct
  source 1..20: mol_type = other DNA
aagttcaatttaccatttgt

SEQ ID NO: 197 - DNA, 20 bp, synthetic construct
  source 1..20: mol_type = other DNA
attggatatgtaacttgcaa

SEQ ID NO: 198 - DNA, 20 bp, synthetic construct
  source 1..20: mol_type = other DNA
acccctattcttcttctttt

SEQ ID NO: 199 - DNA, 20 bp, synthetic construct
  source 1..20: mol_type = other DNA
ctggtcttgaacagtcctcc

SEQ ID NO: 200 - DNA, 20 bp, synthetic construct
  source 1..20: mol_type = other DNA
gctcaagcgatcctccaacc

SEQ ID NO: 201 - DNA, 20 bp, synthetic construct
  source 1..20: mol_type = other DNA
ggattcggttaagttagatt

SEQ ID NO: 202 - DNA, 20 bp, synthetic construct
  source 1..20: mol_type = other DNA
tctttgtttaaaccataaac

SEQ ID NO: 203 - DNA, 20 bp, synthetic construct
  source 1..20: mol_type = other DNA
caggcaggaaggaggtcact

SEQ ID NO: 204 - DNA, 21 bp, synthetic construct
  source 1..21: mol_type = other DNA
ttgaacctctgtgatttccct

SEQ ID NO: 205 - DNA, 21 bp, synthetic construct
  source 1..21: mol_type = other DNA
attgttgagacactggtgaac

SEQ ID NO: 206 - DNA, 21 bp, synthetic construct
  source 1..21: mol_type = other DNA
gtagaaatgggcagagaaggc

SEQ ID NO: 207 - DNA, 21 bp, synthetic construct
  source 1..21: mol_type = other DNA
aatcaatagcaggtcaagagc

SEQ ID NO: 208 - DNA, 21 bp, synthetic construct
  source 1..21: mol_type = other DNA
gtagatatagcccaataaagc

SEQ ID NO: 209 - DNA, 21 bp, synthetic construct
  source 1..21: mol_type = other DNA
tcattagcttaaggtgagggc

SEQ ID NO: 210 - DNA, 21 bp, synthetic construct
  source 1..21: mol_type = other DNA
gaatatgtagcacctatgacc

SEQ ID NO: 211 - DNA, 21 bp, synthetic construct
  source 1..21: mol_type = other DNA
gtactgcttgccagatgattc

SEQ ID NO: 212 - DNA, 21 bp, synthetic construct
  source 1..21: mol_type = other DNA
acactgagctaggtactgtgc

SEQ ID NO: 213 - DNA, 21 bp, synthetic construct
  source 1..21: mol_type = other DNA
agaataagccaagaactgagc

SEQ ID NO: 214 - DNA, 21 bp, synthetic construct
  source 1..21: mol_type = other DNA
aatacagctctggagtggggt

SEQ ID NO: 215 - DNA, 21 bp, synthetic construct
  source 1..21: mol_type = other DNA
ttgaacctctgtgatttccct

SEQ ID NO: 216 - DNA, 21 bp, synthetic construct
  source 1..21: mol_type = other DNA
attgttgagacactggtgaac

SEQ ID NO: 217 - DNA, 21 bp, synthetic construct
  source 1..21: mol_type = other DNA
gtagaaatgggcagagaaggc

SEQ ID NO: 218 - DNA, 21 bp, synthetic construct
  source 1..21: mol_type = other DNA
aatcaatagcaggtcaagagc

SEQ ID NO: 219 - DNA, 21 bp, synthetic construct
  source 1..21: mol_type = other DNA
gtagatatagcccaataaagc

SEQ ID NO: 220 - DNA, 21 bp, synthetic construct
  source 1..21: mol_type = other DNA
tcattagcttaaggtgagggc

SEQ ID NO: 221 - DNA, 21 bp, synthetic construct
  source 1..21: mol_type = other DNA
gaatatgtagcacctatgacc

SEQ ID NO: 222 - DNA, 21 bp, synthetic construct
  source 1..21: mol_type = other DNA
gtactgcttgccagatgattc

SEQ ID NO: 223 - DNA, 21 bp, synthetic construct
  source 1..21: mol_type = other DNA
acactgagctaggtactgtgc

SEQ ID NO: 224 - DNA, 21 bp, synthetic construct
  source 1..21: mol_type = other DNA
agaataagccaagaactgagc

SEQ ID NO: 225 - DNA, 21 bp, synthetic construct
  source 1..21: mol_type = other DNA
aatacagctctggagtggggt

SEQ ID NO: 226 - DNA, 21 bp, synthetic construct
  source 1..21: mol_type = other DNA
tctaaccttcaagctgtcctt

SEQ ID NO: 227 - DNA, 21 bp, synthetic construct
  source 1..21: mol_type = other DNA
acttaaccgaatccatcttgc

SEQ ID NO: 228 - DNA, 21 bp, synthetic construct
  source 1..21: mol_type = other DNA
tctaacttaaccgaatccatc

SEQ ID NO: 229 - DNA, 21 bp, synthetic construct
  source 1..21: mol_type = other DNA
aatttagtttggtgtggtggc

SEQ ID NO: 230 - DNA, 21 bp, synthetic construct
  source 1..21: mol_type = other DNA
tctaaggctggaggactgttc

SEQ ID NO: 231 - DNA, 21 bp, synthetic construct
  source 1..21: mol_type = other DNA
agctaatgggtgatttccttt

SEQ ID NO: 232 - DNA, 21 bp, synthetic construct
  source 1..21: mol_type = other DNA
actctcagatcgcttgaaccc

SEQ ID NO: 233 - DNA, 21 bp, synthetic construct
  source 1..21: mol_type = other DNA
aactaccattaccattaccat

SEQ ID NO: 234 - DNA, 21 bp, synthetic construct
  source 1..21: mol_type = other DNA
ataaatacctagtggtgatgt

SEQ ID NO: 235 - DNA, 21 bp, synthetic construct
  source 1..21: mol_type = other DNA
ttatgaaggaccctgtctacc

SEQ ID NO: 236 - DNA, 21 bp, synthetic construct
  source 1..21: mol_type = other DNA
tacttgaatttgatgattggc

SEQ ID NO: 237 - DNA, 21 bp, synthetic construct
  source 1..21: mol_type = other DNA
tctatatgacaagagagaagc

SEQ ID NO: 238 - DNA, 21 bp, synthetic construct
  source 1..21: mol_type = other DNA
agtttattgatctggtggtgc

SEQ ID NO: 239 - DNA, 21 bp, synthetic construct
  source 1..21: mol_type = other DNA
aagagttcaacagcatgatcc

SEQ ID NO: 240 - DNA, 21 bp, synthetic construct
  source 1..21: mol_type = other DNA
actcaatctcaacctcaagcc

SEQ ID NO: 241 - DNA, 20 bp, synthetic construct
  source 1..20: mol_type = other DNA
taatgacccaaccttgtgtc

SEQ ID NO: 242 - DNA, 20 bp, synthetic construct
  source 1..20: mol_type = other DNA
gattaggaaatgcacaacac

SEQ ID NO: 243 - DNA, 20 bp, synthetic construct
  source 1..20: mol_type = other DNA
caagtttccatacctggttc

SEQ ID NO: 244 - DNA, 20 bp, synthetic construct
  source 1..20: mol_type = other DNA
ttttgagcttagatatggac

SEQ ID NO: 245 - DNA, 20 bp, synthetic construct
  source 1..20: mol_type = other DNA
gattttaagcagaatccaga

SEQ ID NO: 246 - DNA, 20 bp, synthetic construct
  source 1..20: mol_type = other DNA
caagtataatctcgcttctc

SEQ ID NO: 247 - DNA, 20 bp, synthetic construct
  source 1..20: mol_type = other DNA
ggtagacagggtccttcata

SEQ ID NO: 248 - DNA, 20 bp, synthetic construct
  source 1..20: mol_type = other DNA
ctgccataccctttcaattg

SEQ ID NO: 249 - DNA, 20 bp, synthetic construct
  source 1..20: mol_type = other DNA
cttgctacatcaccactagg

SEQ ID NO: 250 - DNA, 20 bp, synthetic construct
  source 1..20: mol_type = other DNA
ggtaatggtagtttgtctaa

SEQ ID NO: 251 - DNA, 20 bp, synthetic construct
  source 1..20: mol_type = other DNA
gcgatctgagagttactttc

SEQ ID NO: 252 - DNA, 20 bp, synthetic construct
  source 1..20: mol_type = other DNA
ttttttctctccacgtgtgt

SEQ ID NO: 253 - DNA, 20 bp, synthetic construct
  source 1..20: mol_type = other DNA
gtgtggaaactggcaataag

SEQ ID NO: 254 - DNA, 20 bp, synthetic construct
  source 1..20: mol_type = other DNA
tattgttttgcggcttggac

SEQ ID NO: 255 - DNA, 20 bp, synthetic construct
  source 1..20: mol_type = other DNA
tctaacgtgctgaaggaccc

SEQ ID NO: 256 - DNA, 20 bp, synthetic construct
  source 1..20: mol_type = other DNA
gtctaaggcttgaggttgag

SEQ ID NO: 257 - DNA, 20 bp, synthetic construct
  source 1..20: mol_type = other DNA
ttcagatctgtgatccactg

SEQ ID NO: 258 - DNA, 20 bp, synthetic construct
  source 1..20: mol_type = other DNA
aagattctctccctatgtct

SEQ ID NO: 259 - DNA, 20 bp, synthetic construct
  source 1..20: mol_type = other DNA
gttgaactctttgcataacc

SEQ ID NO: 260 - DNA, 20 bp, synthetic construct
  source 1..20: mol_type = other DNA
ctcctgactatgtttttcac

SEQ ID NO: 261 - DNA, 20 bp, synthetic construct
  source 1..20: mol_type = other DNA
gtgttttcccagtctgttgc

SEQ ID NO: 262 - DNA, 20 bp, synthetic construct
  source 1..20: mol_type = other DNA
aaaggcggtttcacaatagt

SEQ ID NO: 263 - DNA, 20 bp, synthetic construct
  source 1..20: mol_type = other DNA
catctgatcttgtggctaat

SEQ ID NO: 264 - DNA, 20 bp, synthetic construct
  source 1..20: mol_type = other DNA
ggcaccaccagatcaataaa

SEQ ID NO: 265 - DNA, 20 bp, synthetic construct
  source 1..20: mol_type = other DNA
gggtatcagtactgcacttg

SEQ ID NO: 266 - DNA, 20 bp, synthetic construct
  source 1..20: mol_type = other DNA
taatgacccaaccttgtgtc

SEQ ID NO: 267 - DNA, 20 bp, synthetic construct
  source 1..20: mol_type = other DNA
gattaggaaatgcacaacac

SEQ ID NO: 268 - DNA, 20 bp, synthetic construct
  source 1..20: mol_type = other DNA
caagtttccatacctggttc

SEQ ID NO: 269 - DNA, 20 bp, synthetic construct
  source 1..20: mol_type = other DNA
ttttgagcttagatatggac

SEQ ID NO: 270 - DNA, 20 bp, synthetic construct
  source 1..20: mol_type = other DNA
gattttaagcagaatccaga

SEQ ID NO: 271 - DNA, 20 bp, synthetic construct
  source 1..20: mol_type = other DNA
caagtataatctcgcttctc

SEQ ID NO: 272 - DNA, 20 bp, synthetic construct
  source 1..20: mol_type = other DNA
ggtagacagggtccttcata

SEQ ID NO: 273 - DNA, 20 bp, synthetic construct
  source 1..20: mol_type = other DNA
ctgccataccctttcaattg

SEQ ID NO: 274 - DNA, 20 bp, synthetic construct
  source 1..20: mol_type = other DNA
cttgctacatcaccactagg

SEQ ID NO: 275 - DNA, 20 bp, synthetic construct
  source 1..20: mol_type = other DNA
ggtaatggtagtttgtctaa

SEQ ID NO: 276 - DNA, 20 bp, synthetic construct
  source 1..20: mol_type = other DNA
gcgatctgagagttactttc

SEQ ID NO: 277 - DNA, 20 bp, synthetic construct
  source 1..20: mol_type = other DNA
ttttttctctccacgtgtgt

SEQ ID NO: 278 - DNA, 20 bp, synthetic construct
  source 1..20: mol_type = other DNA
gtgtggaaactggcaataag

SEQ ID NO: 279 - DNA, 20 bp, synthetic construct
  source 1..20: mol_type = other DNA
tattgttttgcggcttggac

SEQ ID NO: 280 - DNA, 20 bp, synthetic construct
  source 1..20: mol_type = other DNA
tctaacgtgctgaaggaccc

SEQ ID NO: 281 - DNA, 20 bp, synthetic construct
  source 1..20: mol_type = other DNA
gtctaaggcttgaggttgag

SEQ ID NO: 282 - DNA, 20 bp, synthetic construct
  source 1..20: mol_type = other DNA
ttcagatctgtgatccactg

SEQ ID NO: 283 - DNA, 20 bp, synthetic construct
  source 1..20: mol_type = other DNA
aagattctctccctatgtct

SEQ ID NO: 284 - DNA, 20 bp, synthetic construct
  source 1..20: mol_type = other DNA
gttgaactctttgcataacc

SEQ ID NO: 285 - DNA, 20 bp, synthetic construct
  source 1..20: mol_type = other DNA
ctcctgactatgtttttcac

SEQ ID NO: 286 - DNA, 20 bp, synthetic construct
  source 1..20: mol_type = other DNA
gtgttttcccagtctgttgc

SEQ ID NO: 287 - DNA, 20 bp, synthetic construct
  source 1..20: mol_type = other DNA
aaaggcggtttcacaatagt

SEQ ID NO: 288 - DNA, 20 bp, synthetic construct
  source 1..20: mol_type = other DNA
catctgatcttgtggctaat

SEQ ID NO: 289 - DNA, 20 bp, synthetic construct
  source 1..20: mol_type = other DNA
ggcaccaccagatcaataaa

SEQ ID NO: 290 - DNA, 20 bp, synthetic construct
  source 1..20: mol_type = other DNA
gggtatcagtactgcacttg

SEQ ID NO: 291 - DNA, 20 bp, synthetic construct
  source 1..20: mol_type = other DNA
tagtcacagggagcattagg

SEQ ID NO: 292 - DNA, 20 bp, synthetic construct
  source 1..20: mol_type = other DNA
gacagtgctcctagaccaaa

SEQ ID NO: 293 - DNA, 20 bp, synthetic construct
  source 1..20: mol_type = other DNA
gaggcaccaactacaaagat

SEQ ID NO: 294 - DNA, 20 bp, synthetic construct
  source 1..20: mol_type = other DNA
ataggtcccatctttacagg

SEQ ID NO: 295 - DNA, 20 bp, synthetic construct
  source 1..20: mol_type = other DNA
aggattcccatggtctatct

SEQ ID NO: 296 - DNA, 20 bp, synthetic construct
  source 1..20: mol_type = other DNA
gcataacaatgaaggtgacc

SEQ ID NO: 297 - DNA, 20 bp, synthetic construct
  source 1..20: mol_type = other DNA
cgcttacttcttaatggtaa

SEQ ID NO: 298 - DNA, 20 bp, synthetic construct
  source 1..20: mol_type = other DNA
ctacagtactctctattcat

SEQ ID NO: 299 - DNA, 20 bp, synthetic construct
  source 1..20: mol_type = other DNA
ggtagtagttaacaaaagct

SEQ ID NO: 300 - DNA, 20 bp, synthetic construct
  source 1..20: mol_type = other DNA
cggacacctcaacactttta

SEQ ID NO: 301 - DNA, 20 bp, synthetic construct
  source 1..20: mol_type = other DNA
tagtcacagggagcattagg

SEQ ID NO: 302 - DNA, 20 bp, synthetic construct
  source 1..20: mol_type = other DNA
gacagtgctcctagaccaaa

SEQ ID NO: 303 - DNA, 20 bp, synthetic construct
  source 1..20: mol_type = other DNA
gaggcaccaactacaaagat

SEQ ID NO: 304 - DNA, 20 bp, synthetic construct
  source 1..20: mol_type = other DNA
ataggtcccatctttacagg

SEQ ID NO: 305 - DNA, 20 bp, synthetic construct
  source 1..20: mol_type = other DNA
aggattcccatggtctatct

SEQ ID NO: 306 - DNA, 20 bp, synthetic construct
  source 1..20: mol_type = other DNA
gcataacaatgaaggtgacc

SEQ ID NO: 307 - DNA, 20 bp, synthetic construct
  source 1..20: mol_type = other DNA
cgcttacttcttaatggtaa

SEQ ID NO: 308 - DNA, 20 bp, synthetic construct
  source 1..20: mol_type = other DNA
ctacagtactctctattcat

SEQ ID NO: 309 - DNA, 20 bp, synthetic construct
  source 1..20: mol_type = other DNA
ggtagtagttaacaaaagct

SEQ ID NO: 310 - DNA, 20 bp, synthetic construct
  source 1..20: mol_type = other DNA
cggacacctcaacactttta

SEQ ID NO: 311 - DNA, 18 bp, synthetic construct
  source 1..18: mol_type = other DNA
taatacagctctggagtg

SEQ ID NO: 312 - DNA, 16 bp, synthetic construct
  source 1..16: mol_type = other DNA
aatacagctctggagt

SEQ ID NO: 313 - DNA, 16 bp, synthetic construct
  source 1..16: mol_type = other DNA
ctctcaccacatgtgc

SEQ ID NO: 314 - DNA, 20 bp, synthetic construct
  source 1..20: mol_type = other DNA
gaaaatgatctcatgggatc

SEQ ID NO: 315 - DNA, 21 bp, synthetic construct
  source 1..21: mol_type = other DNA
aatacagctctggagtggggt

SEQ ID NO: 316 - DNA, 21 bp, synthetic construct
  source 1..21: mol_type = other DNA
aatacagctctggagtggggt

SEQ ID NO: 317 - DNA, 20 bp, synthetic construct
  source 1..20: mol_type = other DNA
agaaagacggacacctcaac

SEQ ID NO: 318 - DNA, 20 bp, synthetic construct
  source 1..20: mol_type = other DNA
ggtatgaaagaaagacggac

SEQ ID NO: 319 - DNA, 20 bp, synthetic construct
  source 1..20: mol_type = other DNA
cttttaatacagctctggag

SEQ ID NO: 320 - DNA, 20 bp, synthetic construct
  source 1..20: mol_type = other DNA
ttcacttttaatacagctct

SEQ ID NO: 321 - DNA, 20 bp, synthetic construct
  source 1..20: mol_type = other DNA
tgcccagcctgaatttcact

SEQ ID NO: 322 - DNA, 20 bp, synthetic construct
  source 1..20: mol_type = other DNA
agaaagacggacacctcaac

SEQ ID NO: 323 - DNA, 20 bp, synthetic construct
  source 1..20: mol_type = other DNA
ggtatgaaagaaagacggac

SEQ ID NO: 324 - DNA, 20 bp, synthetic construct
  source 1..20: mol_type = other DNA
cttttaatacagctctggag

SEQ ID NO: 325 - DNA, 20 bp, synthetic construct
  source 1..20: mol_type = other DNA
ttcacttttaatacagctct

SEQ ID NO: 326 - DNA, 20 bp, synthetic construct
  source 1..20: mol_type = other DNA
tgcccagcctgaatttcact

SEQ ID NO: 327 - DNA, 20 bp, synthetic construct
  source 1..20: mol_type = other DNA
tgtaatcagatttcaccgtg

SEQ ID NO: 328 - DNA, 20 bp, synthetic construct
  source 1..20: mol_type = other DNA
ctcagtccttctgtaatcag

SEQ ID NO: 329 - DNA, 20 bp, synthetic construct
  source 1..20: mol_type = other DNA
aagctttcatttctcagtcc

SEQ ID NO: 330 - DNA, 20 bp, synthetic construct
  source 1..20: mol_type = other DNA
ctctaattccagcaaagctt

SEQ ID NO: 331 - DNA, 20 bp, synthetic construct
  source 1..20: mol_type = other DNA
tcttatttgagacagctgcc

SEQ ID NO: 332 - DNA, 20 bp, synthetic construct
  source 1..20: mol_type = other DNA
gtgccacgctctgctttact

SEQ ID NO: 333 - DNA, 20 bp, synthetic construct
  source 1..20: mol_type = other DNA
tgcagaatgtagtgccacgc

SEQ ID NO: 334 - DNA, 20 bp, synthetic construct
  source 1..20: mol_type = other DNA
ggcctctttctgcagaatgt

SEQ ID NO: 335 - DNA, 20 bp, synthetic construct
  source 1..20: mol_type = other DNA
actatattctgcagtaaggc

SEQ ID NO: 336 - DNA, 20 bp, synthetic construct
  source 1..20: mol_type = other DNA
ggaatcatggtgatgcataa

SEQ ID NO: 337 - DNA, 20 bp, synthetic construct
  source 1..20: mol_type = other DNA
gagacttgggtttgatttag

SEQ ID NO: 338 - DNA, 20 bp, synthetic construct
  source 1..20: mol_type = other DNA
gtgatggtcagagacttggg

SEQ ID NO: 339 - DNA, 20 bp, synthetic construct
  source 1..20: mol_type = other DNA
attttcggtaagtgatggtc

SEQ ID NO: 340 - DNA, 20 bp, synthetic construct
  source 1..20: mol_type = other DNA
gtcacaacttcatggttcga

SEQ ID NO: 341 - DNA, 20 bp, synthetic construct
  source 1..20: mol_type = other DNA
tgcagcttttagaaatggtc

SEQ ID NO: 342 - DNA, 20 bp, synthetic construct
  source 1..20: mol_type = other DNA
tgaggatcctgttaaacaat

SEQ ID NO: 343 - DNA, 20 bp, synthetic construct
  source 1..20: mol_type = other DNA
gctgtttaatctgaggatcc

SEQ ID NO: 344 - DNA, 20 bp, synthetic construct
  source 1..20: mol_type = other DNA
acacaattaaaagaagatgc

SEQ ID NO: 345 - DNA, 20 bp, synthetic construct
  source 1..20: mol_type = other DNA
catagaatcgtcctttacac

SEQ ID NO: 346 - DNA, 20 bp, synthetic construct
  source 1..20: mol_type = other DNA
ggctaacttgctgtggagtt

SEQ ID NO: 347 - DNA, 20 bp, synthetic construct
  source 1..20: mol_type = other DNA
tagtttttaggtggcccccg

SEQ ID NO: 348 - DNA, 20 bp, synthetic construct
  source 1..20: mol_type = other DNA
gcaactgaacacatttctta

SEQ ID NO: 349 - DNA, 20 bp, synthetic construct
  source 1..20: mol_type = other DNA
ctccctcactgcaactgaac

SEQ ID NO: 350 - DNA, 20 bp, synthetic construct
  source 1..20: mol_type = other DNA
gatattacctttgctccctc

SEQ ID NO: 351 - DNA, 20 bp, synthetic construct
  source 1..20: mol_type = other DNA
agctgaagggtgatattacc

SEQ ID NO: 352 - DNA, 20 bp, synthetic construct
  source 1..20: mol_type = other DNA
gggactttgataaggaagct

SEQ ID NO: 353 - DNA, 20 bp, synthetic construct
  source 1..20: mol_type = other DNA
gccctgcagtatctctaacc

SEQ ID NO: 354 - DNA, 20 bp, synthetic construct
  source 1..20: mol_type = other DNA
aaatgctcctacaccctgcc

SEQ ID NO: 355 - DNA, 20 bp, synthetic construct
  source 1..20: mol_type = other DNA
gcataaagttcaaatgctcc

SEQ ID NO: 356 - DNA, 20 bp, synthetic construct
  source 1..20: mol_type = other DNA
cggtggagcttggcataaag

SEQ ID NO: 357 - DNA, 20 bp, synthetic construct
  source 1..20: mol_type = other DNA
ccaaaaaaaatacacagcgg

SEQ ID NO: 358 - DNA, 20 bp, synthetic construct
  source 1..20: mol_type = other DNA
aaaagagtcaggatttcttc

SEQ ID NO: 359 - DNA, 20 bp, synthetic construct
  source 1..20: mol_type = other DNA
ggccaggcaataaaagagtc

SEQ ID NO: 360 - DNA, 20 bp, synthetic construct
  source 1..20: mol_type = other DNA
aaaatgaggaggccaggcaa

SEQ ID NO: 361 - DNA, 20 bp, synthetic construct
  source 1..20: mol_type = other DNA
gcgtgtgacagtataaatat

SEQ ID NO: 362 - DNA, 20 bp, synthetic construct
  source 1..20: mol_type = other DNA
gattttgtacatgcgtgtga

SEQ ID NO: 363 - DNA, 20 bp, synthetic construct
  source 1..20: mol_type = other DNA
tgagtacttgtcaattgatt

SEQ ID NO: 364 - DNA, 20 bp, synthetic construct
  source 1..20: mol_type = other DNA
caccttcacagcagccggta

SEQ ID NO: 365 - DNA, 20 bp, synthetic construct
  source 1..20: mol_type = other DNA
gttacttctactcaccttca

SEQ ID NO: 366 - DNA, 20 bp, synthetic construct
  source 1..20: mol_type = other DNA
ttctgagtgagttacttcta

SEQ ID NO: 367 - DNA, 20 bp, synthetic construct
  source 1..20: mol_type = other DNA
gccaatttgcattttctgag

SEQ ID NO: 368 - DNA, 20 bp, synthetic construct
  source 1..20: mol_type = other DNA
tttttgcagtgccaatttgc

SEQ ID NO: 369 - DNA, 20 bp, synthetic construct
  source 1..20: mol_type = other DNA
ctacatctgtgtttttgcag

SEQ ID NO: 370 - DNA, 20 bp, synthetic construct
  source 1..20: mol_type = other DNA
actgtcacatctacatctgt

SEQ ID NO: 371 - DNA, 20 bp, synthetic construct
  source 1..20: mol_type = other DNA
gtactactcaggactgtcac

SEQ ID NO: 372 - DNA, 20 bp, synthetic construct
  source 1..20: mol_type = other DNA
aaatggaaatagtactactc

SEQ ID NO: 373 - DNA, 20 bp, synthetic construct
  source 1..20: mol_type = other DNA
gactggcaattagaggtaga

SEQ ID NO: 374 - DNA, 20 bp, synthetic construct
  source 1..20: mol_type = other DNA
ttttgtggaaagactggcaa

SEQ ID NO: 375 - DNA, 20 bp, synthetic construct
  source 1..20: mol_type = other DNA
tgcacccccaaaatagcttc

SEQ ID NO: 376 - DNA, 20 bp, synthetic construct
  source 1..20: mol_type = other DNA
ctctatcatcttgcaccccc

SEQ ID NO: 377 - DNA, 20 bp, synthetic construct
  source 1..20: mol_type = other DNA
tactataccttctctatcat

SEQ ID NO: 378 - DNA, 20 bp, synthetic construct
  source 1..20: mol_type = other DNA
ggagctccaggactgagata

SEQ ID NO: 379 - DNA, 20 bp, synthetic construct
  source 1..20: mol_type = other DNA
caaggcacaaagggagctcc

SEQ ID NO: 380 - DNA, 20 bp, synthetic construct
  source 1..20: mol_type = other DNA
ttgaagacttatgtgcaagg

SEQ ID NO: 381 - DNA, 20 bp, synthetic construct
  source 1..20: mol_type = other DNA
cggccagcaattatttcttt

SEQ ID NO: 382 - DNA, 20 bp, synthetic construct
  source 1..20: mol_type = other DNA
ggtgctgttagaatcaatag

SEQ ID NO: 383 - DNA, 20 bp, synthetic construct
  source 1..20: mol_type = other DNA
ctgactgtcaggtgctgtta

SEQ ID NO: 384 - DNA, 20 bp, synthetic construct
  source 1..20: mol_type = other DNA
cccacttgtactgactgtca

SEQ ID NO: 385 - DNA, 20 bp, synthetic construct
  source 1..20: mol_type = other DNA
gtatccccacttcccacttg

SEQ ID NO: 386 - DNA, 20 bp, synthetic construct
  source 1..20: mol_type = other DNA
gaagagcatatggtatcccc

SEQ ID NO: 387 - DNA, 20 bp, synthetic construct
  source 1..20: mol_type = other DNA
gatctgaataggctgctagg

SEQ ID NO: 388 - DNA, 20 bp, synthetic construct
  source 1..20: mol_type = other DNA
atggccccttagtgatctga

SEQ ID NO: 389 - DNA, 20 bp, synthetic construct
  source 1..20: mol_type = other DNA
gtgaagttgcagatggcccc

SEQ ID NO: 390 - DNA, 20 bp, synthetic construct
  source 1..20: mol_type = other DNA
acctgatttctaactgagga

SEQ ID NO: 391 - DNA, 20 bp, synthetic construct
  source 1..20: mol_type = other DNA
agaattgactttgaatcacc

SEQ ID NO: 392 - DNA, 20 bp, synthetic construct
  source 1..20: mol_type = other DNA
gcagctcggtatctgataca

SEQ ID NO: 393 - DNA, 20 bp, synthetic construct
  source 1..20: mol_type = other DNA
tgaaacttcagtgcagctcg

SEQ ID NO: 394 - DNA, 20 bp, synthetic construct
  source 1..20: mol_type = other DNA
gaactctgcaaagatgatgt

SEQ ID NO: 395 - DNA, 20 bp, synthetic construct
  source 1..20: mol_type = other DNA
tactgaactgtgaactctgc

SEQ ID NO: 396 - DNA, 20 bp, synthetic construct
  source 1..20: mol_type = other DNA
cataccacctttactgaact

SEQ ID NO: 397 - DNA, 20 bp, synthetic construct
  source 1..20: mol_type = other DNA
gtttataaatcataccacct

SEQ ID NO: 398 - DNA, 20 bp, synthetic construct
  source 1..20: mol_type = other DNA
ggcttgtattatggatattt

SEQ ID NO: 399 - DNA, 20 bp, synthetic construct
  source 1..20: mol_type = other DNA
caactttgtctggacctctc

SEQ ID NO: 400 - DNA, 20 bp, synthetic construct
  source 1..20: mol_type = other DNA
tcttcgaagccagacaactt

SEQ ID NO: 401 - DNA, 20 bp, synthetic construct
  source 1..20: mol_type = other DNA
ctacccttcctaccttagag

SEQ ID NO: 402 - DNA, 20 bp, synthetic construct
  source 1..20: mol_type = other DNA
accagaagaggcagccctac

SEQ ID NO: 403 - DNA, 20 bp, synthetic construct
  source 1..20: mol_type = other DNA
ctgggagtgtctggtagtca

SEQ ID NO: 404 - DNA, 20 bp, synthetic construct
  source 1..20: mol_type = other DNA
ggatccccatcactcaggtc

SEQ ID NO: 405 - DNA, 20 bp, synthetic construct
  source 1..20: mol_type = other DNA
tgaaaatgatctcatgggat

SEQ ID NO: 406 - DNA, 20 bp, synthetic construct
  source 1..20: mol_type = other DNA
caatgaaaatgatctcatgg

SEQ ID NO: 407 - DNA, 20 bp, synthetic construct
  source 1..20: mol_type = other DNA
tggtcagtagaaagcaatga

SEQ ID NO: 408 - DNA, 20 bp, synthetic construct
  source 1..20: mol_type = other DNA
ccaaaccctggtcagtagaa

SEQ ID NO: 409 - DNA, 20 bp, synthetic construct
  source 1..20: mol_type = other DNA
ttaatacagctctggagtgg

SEQ ID NO: 410 - DNA, 21 bp, synthetic construct
  source 1..21: mol_type = other DNA
aatacagctctggagtggggt

SEQ ID NO: 411 - DNA, 20 bp, synthetic construct
  source 1..20: mol_type = other DNA
aatacagctctggagtgggg

SEQ ID NO: 412 - DNA, 20 bp, synthetic construct
  source 1..20: mol_type = other DNA
aatacagctctggagtgggg

SEQ ID NO: 413 - DNA, 20 bp, synthetic construct
  source 1..20: mol_type = other DNA
taatacagctctggagtggg

SEQ ID NO: 414 - DNA, 20 bp, synthetic construct
  source 1..20: mol_type = other DNA
taatacagctctggagtggg

SEQ ID NO: 415 - DNA, 20 bp, synthetic construct
  source 1..20: mol_type = other DNA
ttaatacagctctggagtgg

SEQ ID NO: 416 - DNA, 21 bp, synthetic construct
  source 1..21: mol_type = other DNA
aatacagctctggagtggggt

SEQ ID NO: 417 - DNA, 20 bp, synthetic construct
  source 1..20: mol_type = other DNA
tcgatgcatagctgggatta

SEQ ID NO: 418 - DNA, 20 bp, synthetic construct
  source 1..20: mol_type = other DNA
agcctctcgatgcatagctg

SEQ ID NO: 419 - DNA, 20 bp, synthetic construct
  source 1..20: mol_type = other DNA
cctcagcctctcgatgcata

SEQ ID NO: 420 - DNA, 20 bp, synthetic construct
  source 1..20: mol_type = other DNA
ttaatacagctctggagtgg

SEQ ID NO: 421 - DNA, 20 bp, synthetic construct
  source 1..20: mol_type = other DNA
ttaatacagctctggagtgg

SEQ ID NO: 422 - DNA, 20 bp, synthetic construct
  source 1..20: mol_type = other DNA
ttaatacagctctggagtgg

SEQ ID NO: 423 - DNA, 20 bp, synthetic construct
  source 1..20: mol_type = other DNA
gaaaatgatctcatgggatc

SEQ ID NO: 424 - DNA, 20 bp, synthetic construct
  source 1..20: mol_type = other DNA
gaaaatgatctcatgggatc

SEQ ID NO: 425 - DNA, 20 bp, synthetic construct
  source 1..20: mol_type = other DNA
gaaaatgatctcatgggatc

SEQ ID NO: 426 - DNA, 21 bp, synthetic construct
  source 1..21: mol_type = other DNA
aatacagctctggagtggggt

SEQ ID NO: 427 - DNA, 21 bp, synthetic construct
  source 1..21: mol_type = other DNA
aatacagctctggagtggggt

SEQ ID NO: 428 - DNA, 20 bp, synthetic construct
  source 1..20: mol_type = other DNA
tacagctctggagtggggtg

SEQ ID NO: 429 - DNA, 20 bp, synthetic construct
  source 1..20: mol_type = other DNA
atacagctctggagtggggt

SEQ ID NO: 430 - DNA, 20 bp, synthetic construct
  source 1..20: mol_type = other DNA
tttaatacagctctggagtg

SEQ ID NO: 431 - DNA, 21 bp, synthetic construct
  source 1..21: mol_type = other DNA
ttaatacagctctggagtggg

SEQ ID NO: 432 - DNA, 22 bp, synthetic construct
  source 1..22: mol_type = other DNA
ttaatacagctctggagtgggg

SEQ ID NO: 433 - DNA, 23 bp, synthetic construct
  source 1..23: mol_type = other DNA
ttaatacagctctggagtggggt

SEQ ID NO: 434 - DNA, 20 bp, synthetic construct
  source 1..20: mol_type = other DNA
tgtaatcagatttcaccgtg

SEQ ID NO: 435 - DNA, 20 bp, synthetic construct
  source 1..20: mol_type = other DNA
ctcagtccttctgtaatcag

SEQ ID NO: 436 - DNA, 20 bp, synthetic construct
  source 1..20: mol_type = other DNA
aagctttcatttctcagtcc

SEQ ID NO: 437 - DNA, 20 bp, synthetic construct
  source 1..20: mol_type = other DNA
ctctaattccagcaaagctt

SEQ ID NO: 438 - DNA, 20 bp, synthetic construct
  source 1..20: mol_type = other DNA
tcttatttgagacagctgcc

SEQ ID NO: 439 - DNA, 20 bp, synthetic construct
  source 1..20: mol_type = other DNA
gtgccacgctctgctttact

SEQ ID NO: 440 - DNA, 20 bp, synthetic construct
  source 1..20: mol_type = other DNA
tgcagaatgtagtgccacgc

SEQ ID NO: 441 - DNA, 20 bp, synthetic construct
  source 1..20: mol_type = other DNA
ggcctctttctgcagaatgt

SEQ ID NO: 442 - DNA, 20 bp, synthetic construct
  source 1..20: mol_type = other DNA
actatattctgcagtaaggc

SEQ ID NO: 443 - DNA, 20 bp, synthetic construct
  source 1..20: mol_type = other DNA
ggaatcatggtgatgcataa

SEQ ID NO: 444 - DNA, 20 bp, synthetic construct
  source 1..20: mol_type = other DNA
gagacttgggtttgatttag

SEQ ID NO: 445 - DNA, 20 bp, synthetic construct
  source 1..20: mol_type = other DNA
gtgatggtcagagacttggg

SEQ ID NO: 446 - DNA, 20 bp, synthetic construct
  source 1..20: mol_type = other DNA
attttcggtaagtgatggtc

SEQ ID NO: 447 - DNA, 20 bp, synthetic construct
  source 1..20: mol_type = other DNA
gtcacaacttcatggttcga

SEQ ID NO: 448 - DNA, 20 bp, synthetic construct
  source 1..20: mol_type = other DNA
tgcagcttttagaaatggtc

SEQ ID NO: 449 - DNA, 20 bp, synthetic construct
  source 1..20: mol_type = other DNA
tgaggatcctgttaaacaat

SEQ ID NO: 450 - DNA, 20 bp, synthetic construct
  source 1..20: mol_type = other DNA
gctgtttaatctgaggatcc

SEQ ID NO: 451 - DNA, 20 bp, synthetic construct
  source 1..20: mol_type = other DNA
catagaatcgtcctttacac

SEQ ID NO: 452 - DNA, 20 bp, synthetic construct
  source 1..20: mol_type = other DNA
ggctaacttgctgtggagtt

SEQ ID NO: 453 - DNA, 20 bp, synthetic construct
  source 1..20: mol_type = other DNA
tagtttttaggtggcccccg

SEQ ID NO: 454 - DNA, 20 bp, synthetic construct
  source 1..20: mol_type = other DNA
gcaactgaacacatttctta

SEQ ID NO: 455 - DNA, 20 bp, synthetic construct
  source 1..20: mol_type = other DNA
ctccctcactgcaactgaac

SEQ ID NO: 456 - DNA, 20 bp, synthetic construct
  source 1..20: mol_type = other DNA
gatattacctttgctccctc

SEQ ID NO: 457 - DNA, 20 bp, synthetic construct
  source 1..20: mol_type = other DNA
agctgaagggtgatattacc

SEQ ID NO: 458 - DNA, 20 bp, synthetic construct
  source 1..20: mol_type = other DNA
gggactttgataaggaagct

SEQ ID NO: 459 - DNA, 20 bp, synthetic construct
  source 1..20: mol_type = other DNA
gccctgcagtatctctaacc

SEQ ID NO: 460 - DNA, 20 bp, synthetic construct
  source 1..20: mol_type = other DNA
aaatgctcctacaccctgcc

SEQ ID NO: 461 - DNA, 20 bp, synthetic construct
  source 1..20: mol_type = other DNA
gcataaagttcaaatgctcc

SEQ ID NO: 462 - DNA, 20 bp, synthetic construct
  source 1..20: mol_type = other DNA
cggtggagcttggcataaag

SEQ ID NO: 463 - DNA, 20 bp, synthetic construct
  source 1..20: mol_type = other DNA
ccaaaaaaaatacacagcgg

SEQ ID NO: 464 - DNA, 20 bp, synthetic construct
  source 1..20: mol_type = other DNA
ggcaataaaagagtcaggat

SEQ ID NO: 465 - DNA, 20 bp, synthetic construct
  source 1..20: mol_type = other DNA
aatgaggaggccaggcaata

SEQ ID NO: 466 - DNA, 20 bp, synthetic construct
  source 1..20: mol_type = other DNA
gcgtgtgacagtataaatat

SEQ ID NO: 467 - DNA, 20 bp, synthetic construct
  source 1..20: mol_type = other DNA
gattttgtacatgcgtgtga

SEQ ID NO: 468 - DNA, 20 bp, synthetic construct
  source 1..20: mol_type = other DNA
tgagtacttgtcaattgatt

SEQ ID NO: 469 - DNA, 20 bp, synthetic construct
  source 1..20: mol_type = other DNA
atatcctcaatgagtacttg

SEQ ID NO: 470 - DNA, 20 bp, synthetic construct
  source 1..20: mol_type = other DNA
gccttgcatatattatatcc

SEQ ID NO: 471 - DNA, 20 bp, synthetic construct
  source 1..20: mol_type = other DNA
agccagtaccgcagtgcctt

SEQ ID NO: 472 - DNA, 20 bp, synthetic construct
  source 1..20: mol_type = other DNA
caccttcacagcagccagta

SEQ ID NO: 473 - DNA, 20 bp, synthetic construct
  source 1..20: mol_type = other DNA
gttacttctactcaccttca

SEQ ID NO: 474 - DNA, 20 bp, synthetic construct
  source 1..20: mol_type = other DNA
gcattttctgagtgagttac

SEQ ID NO: 475 - DNA, 20 bp, synthetic construct
  source 1..20: mol_type = other DNA
tttttgcagtgccaatttgc

SEQ ID NO: 476 - DNA, 20 bp, synthetic construct
  source 1..20: mol_type = other DNA
ctacatctgtgtttttgcag

SEQ ID NO: 477 - DNA, 20 bp, synthetic construct
  source 1..20: mol_type = other DNA
actgtcacatctacatctgt

SEQ ID NO: 478 - DNA, 20 bp, synthetic construct
  source 1..20: mol_type = other DNA
gtactactcaggactgtcac

SEQ ID NO: 479 - DNA, 20 bp, synthetic construct
  source 1..20: mol_type = other DNA
gactggcaattagaggtaga

SEQ ID NO: 480 - DNA, 20 bp, synthetic construct
  source 1..20: mol_type = other DNA
ttttgtggaaagactggcaa

SEQ ID NO: 481 - DNA, 20 bp, synthetic construct
  source 1..20: mol_type = other DNA
tgcacccccaaaatagcttc

SEQ ID NO: 482 - DNA, 20 bp, synthetic construct
  source 1..20: mol_type = other DNA
ctctatcatcttgcaccccc

SEQ ID NO: 483 - DNA, 20 bp, synthetic construct
  source 1..20: mol_type = other DNA
tactataccttctctatcat

SEQ ID NO: 484 - DNA, 20 bp, synthetic construct
  source 1..20: mol_type = other DNA
ggagctccaggactgagata

SEQ ID NO: 485 - DNA, 20 bp, synthetic construct
  source 1..20: mol_type = other DNA
gcaaggcacaaagggagctc

SEQ ID NO: 486 - DNA, 20 bp, synthetic construct
  source 1..20: mol_type = other DNA
ttgaagacttatgtgcaagg

SEQ ID NO: 487 - DNA, 20 bp, synthetic construct
  source 1..20: mol_type = other DNA
cggccagcaattatttcttt

SEQ ID NO: 488 - DNA, 20 bp, synthetic construct
  source 1..20: mol_type = other DNA
gtcgggtgctgttagaatca

SEQ ID NO: 489 - DNA, 20 bp, synthetic construct
  source 1..20: mol_type = other DNA
cccacttgtactgactgtcg

SEQ ID NO: 490 - DNA, 20 bp, synthetic construct
  source 1..20: mol_type = other DNA
gtatccccacttcccacttg

SEQ ID NO: 491 - DNA, 20 bp, synthetic construct
  source 1..20: mol_type = other DNA
gaagagcatatggtatcccc

SEQ ID NO: 492 - DNA, 20 bp, synthetic construct
  source 1..20: mol_type = other DNA
gatctgaataggctgctagg

SEQ ID NO: 493 - DNA, 20 bp, synthetic construct
  source 1..20: mol_type = other DNA
atggccccttagtgatctga

SEQ ID NO: 494 - DNA, 20 bp, synthetic construct
  source 1..20: mol_type = other DNA
gtgaagttgcagatggcccc

SEQ ID NO: 495 - DNA, 20 bp, synthetic construct
  source 1..20: mol_type = other DNA
acctgatttctaactgagga

SEQ ID NO: 496 - DNA, 20 bp, synthetic construct
  source 1..20: mol_type = other DNA
agaattgactttgaatcacc

SEQ ID NO: 497 - DNA, 20 bp, synthetic construct
  source 1..20: mol_type = other DNA
tatctgatacagaattgact

SEQ ID NO: 498 - DNA, 20 bp, synthetic construct
  source 1..20: mol_type = other DNA
tgcaactcggtatctgatac

SEQ ID NO: 499 - DNA, 20 bp, synthetic construct
  source 1..20: mol_type = other DNA
gtcatcttccctctctgaaa

SEQ ID NO: 500 - DNA, 20 bp, synthetic construct
  source 1..20: mol_type = other DNA
gaactctgcaaagatgatgt

SEQ ID NO: 501 - DNA, 20 bp, synthetic construct
  source 1..20: mol_type = other DNA
tactgaactgtgaactctgc

SEQ ID NO: 502 - DNA, 20 bp, synthetic construct
  source 1..20: mol_type = other DNA
cataccacctttactgaact

SEQ ID NO: 503 - DNA, 20 bp, synthetic construct
  source 1..20: mol_type = other DNA
gtttataaatcataccacct

SEQ ID NO: 504 - DNA, 20 bp, synthetic construct
  source 1..20: mol_type = other DNA
ggcttgtattatggatattt

SEQ ID NO: 505 - DNA, 20 bp, synthetic construct
  source 1..20: mol_type = other DNA
caactttgtctggacctctc

SEQ ID NO: 506 - DNA, 20 bp, synthetic construct
  source 1..20: mol_type = other DNA
tcttcgaagccagacaactt

SEQ ID NO: 507 - DNA, 20 bp, synthetic construct
  source 1..20: mol_type = other DNA
ctacccttcctaccttagag

SEQ ID NO: 508 - DNA, 20 bp, synthetic construct
  source 1..20: mol_type = other DNA
ttgaccagaagaggcagccc

SEQ ID NO: 509 - DNA, 20 bp, synthetic construct
  source 1..20: mol_type = other DNA
gctcttgacctgctattgat

SEQ ID NO: 510 - DNA, 20 bp, synthetic construct
  source 1..20: mol_type = other DNA
tagttggtgcctctcttcag

SEQ ID NO: 511 - DNA, 20 bp, synthetic construct
  source 1..20: mol_type = other DNA
ttttggtgaaacttgaaacc

SEQ ID NO: 512 - DNA, 20 bp, synthetic construct
  source 1..20: mol_type = other DNA
ctgcccatttctactttttg

SEQ ID NO: 513 - DNA, 20 bp, synthetic construct
  source 1..20: mol_type = other DNA
cactgcttggtgaatgcctt

SEQ ID NO: 514 - DNA, 20 bp, synthetic construct
  source 1..20: mol_type = other DNA
cttggatggcttcaaaagtc

SEQ ID NO: 515 - DNA, 20 bp, synthetic construct
  source 1..20: mol_type = other DNA
gcaatattctccccttgagc

SEQ ID NO: 516 - DNA, 20 bp, synthetic construct
  source 1..20: mol_type = other DNA
cccagtaataattttcatgg

SEQ ID NO: 517 - DNA, 20 bp, synthetic construct
  source 1..20: mol_type = other DNA
ttggtctaggagcactgtcc

SEQ ID NO: 518 - DNA, 20 bp, synthetic construct
  source 1..20: mol_type = other DNA
gctttctactgaccagggtt

SEQ ID NO: 519 - DNA, 20 bp, synthetic construct
  source 1..20: mol_type = other DNA
atcccatgagatcattttca

SEQ ID NO: 520 - DNA, 20 bp, synthetic construct
  source 1..20: mol_type = other DNA
actcccaggacctgagtgat

SEQ ID NO: 521 - DNA, 20 bp, synthetic construct
  source 1..20: mol_type = other DNA
ctaatgctccctgtgactac

SEQ ID NO: 522 - DNA, 20 bp, synthetic construct
  source 1..20: mol_type = other DNA
tcaccagtgtctcaacaatc

SEQ ID NO: 523 - DNA, 20 bp, synthetic construct
  source 1..20: mol_type = other DNA
acagaggttcaaagttcacc

SEQ ID NO: 524 - DNA, 20 bp, synthetic construct
  source 1..20: mol_type = other DNA
acagagcaaacagggaaatc

SEQ ID NO: 525 - DNA, 20 bp, synthetic construct
  source 1..20: mol_type = other DNA
ctgaaagctatcaggcacag

SEQ ID NO: 526 - DNA, 20 bp, synthetic construct
  source 1..20: mol_type = other DNA
ggagatttgttagcagactg

SEQ ID NO: 527 - DNA, 20 bp, synthetic construct
  source 1..20: mol_type = other DNA
ggttaaactgcataaaggag

SEQ ID NO: 528 - DNA, 20 bp, synthetic construct
  source 1..20: mol_type = other DNA
cctccccattggaagtacag

SEQ ID NO: 529 - DNA, 20 bp, synthetic construct
  source 1..20: mol_type = other DNA
ccataggctgattccaattc

SEQ ID NO: 530 - DNA, 20 bp, synthetic construct
  source 1..20: mol_type = other DNA
gagctatctcttctcccata

SEQ ID NO: 531 - DNA, 20 bp, synthetic construct
  source 1..20: mol_type = other DNA
gttcccacacagaatcctag

SEQ ID NO: 532 - DNA, 20 bp, synthetic construct
  source 1..20: mol_type = other DNA
gcccttttatctcttcaagt

SEQ ID NO: 533 - DNA, 20 bp, synthetic construct
  source 1..20: mol_type = other DNA
cgaggttctctttcaaggat

SEQ ID NO: 534 - DNA, 20 bp, synthetic construct
  source 1..20: mol_type = other DNA
tgagatgcaaagcacgaggt

SEQ ID NO: 535 - DNA, 20 bp, synthetic construct
  source 1..20: mol_type = other DNA
gtcagaggcagttgagatgc

SEQ ID NO: 536 - DNA, 20 bp, synthetic construct
  source 1..20: mol_type = other DNA
acccaaccttgtgtcagagg

SEQ ID NO: 537 - DNA, 20 bp, synthetic construct
  source 1..20: mol_type = other DNA
atgacccaaccttgtgtcag

SEQ ID NO: 538 - DNA, 20 bp, synthetic construct
  source 1..20: mol_type = other DNA
ggttaatgacccaaccttgt

SEQ ID NO: 539 - DNA, 20 bp, synthetic construct
  source 1..20: mol_type = other DNA
atatggttaatgacccaacc

SEQ ID NO: 540 - DNA, 20 bp, synthetic construct
  source 1..20: mol_type = other DNA
tatgaagtaagaaaggggta

SEQ ID NO: 541 - DNA, 20 bp, synthetic construct
  source 1..20: mol_type = other DNA
gacaagattaggaaatgcac

SEQ ID NO: 542 - DNA, 20 bp, synthetic construct
  source 1..20: mol_type = other DNA
cacagaagacaagattagga

SEQ ID NO: 543 - DNA, 20 bp, synthetic construct
  source 1..20: mol_type = other DNA
tctgcacagaagacaagatt

SEQ ID NO: 544 - DNA, 20 bp, synthetic construct
  source 1..20: mol_type = other DNA
tctcaagtttccatacctgg

SEQ ID NO: 545 - DNA, 20 bp, synthetic construct
  source 1..20: mol_type = other DNA
tatggacaaaagtctcaagt

SEQ ID NO: 546 - DNA, 20 bp, synthetic construct
  source 1..20: mol_type = other DNA
gaccccctctccatcccttt

SEQ ID NO: 547 - DNA, 20 bp, synthetic construct
  source 1..20: mol_type = other DNA
gatgcccaatttctcttttc

SEQ ID NO: 548 - DNA, 20 bp, synthetic construct
  source 1..20: mol_type = other DNA
cctttctctggatcatgctg

SEQ ID NO: 549 - DNA, 20 bp, synthetic construct
  source 1..20: mol_type = other DNA
tggtaggtatagagtctcac

SEQ ID NO: 550 - DNA, 20 bp, synthetic construct
  source 1..20: mol_type = other DNA
cctttctctggatcatgctg

SEQ ID NO: 551 - DNA, 20 bp, synthetic construct
  source 1..20: mol_type = other DNA
gcctgacccctattcttctt

SEQ ID NO: 552 - DNA, 20 bp, synthetic construct
  source 1..20: mol_type = other DNA
ccactgagcttgcctgaccc

SEQ ID NO: 553 - DNA, 20 bp, synthetic construct
  source 1..20: mol_type = other DNA
tggtaggtatagagtctcac

SEQ ID NO: 554 - DNA, 20 bp, synthetic construct
  source 1..20: mol_type = other DNA
gcaaaggcggtttcacaata

SEQ ID NO: 555 - DNA, 20 bp, synthetic construct
  source 1..20: mol_type = other DNA
gatggattcggtcaagttag

SEQ ID NO: 556 - DNA, 20 bp, synthetic construct
  source 1..20: mol_type = other DNA
ggaaggaggtcactttggga

SEQ ID NO: 557 - DNA, 20 bp, synthetic construct
  source 1..20: mol_type = other DNA
ccaggcaggaaggaggtcac

SEQ ID NO: 558 - DNA, 20 bp, synthetic construct
  source 1..20: mol_type = other DNA
tcagtactgcacttgatgga

SEQ ID NO: 559 - DNA, 20 bp, synthetic construct
  source 1..20: mol_type = other DNA
gcttggggtatcagtactgc

SEQ ID NO: 560 - DNA, 17 bp, synthetic construct
  source 1..17: mol_type = other DNA
tacagctctggagtggg

SEQ ID NO: 561 - DNA, 16 bp, synthetic construct
  source 1..16: mol_type = other DNA
gctctggagtggggtg

SEQ ID NO: 562 - DNA, 16 bp, synthetic construct
  source 1..16: mol_type = other DNA
acagctctggagtggg

SEQ ID NO: 563 - DNA, 16 bp, synthetic construct
  source 1..16: mol_type = other DNA
taatacagctctggag

SEQ ID NO: 564 - DNA, 16 bp, synthetic construct
  source 1..16: mol_type = other DNA
tttaatacagctctgg

SEQ ID NO: 565 - DNA, 16 bp, synthetic construct
  source 1..16: mol_type = other DNA
cacttttaatacagct

SEQ ID NO: 566 - DNA, 21 bp, synthetic construct
  source 1..21: mol_type = other DNA
aatacagctctggagtggggt

SEQ ID NO: 567 - DNA, 23 bp, synthetic construct
  source 1..23: mol_type = other DNA
ttaatacagctctggagtggggt

SEQ ID NO: 568 - DNA, 23 bp, synthetic construct
  source 1..23: mol_type = other DNA
ttaatacagctctggagtggggt

SEQ ID NO: 569 - DNA, 23 bp, synthetic construct
  source 1..23: mol_type = other DNA
ttaatacagctctggagtggggt

SEQ ID NO: 570 - DNA, 23 bp, synthetic construct
  source 1..23: mol_type = other DNA
ttaatacagctctggagtggggt

SEQ ID NO: 571 - DNA, 23 bp, synthetic construct
  source 1..23: mol_type = other DNA
ttaatacagctctggagtggggt

SEQ ID NO: 572 - DNA, 23 bp, synthetic construct
  source 1..23: mol_type = other DNA
ttaatacagctctggagtggggt

SEQ ID NO: 573 - DNA, 23 bp, synthetic construct
  source 1..23: mol_type = other DNA
ttaatacagctctggagtggggt

SEQ ID NO: 574 - DNA, 23 bp, synthetic construct
  source 1..23: mol_type = other DNA
ttaatacagctctggagtggggt

SEQ ID NO: 575 - DNA, 23 bp, synthetic construct
  source 1..23: mol_type = other DNA
ttttaatacagctctggagtggg

SEQ ID NO: 576 - DNA, 23 bp, synthetic construct
  source 1..23: mol_type = other DNA
ttttaatacagctctggagtggg

SEQ ID NO: 577 - DNA, 25 bp, synthetic construct
  source 1..25: mol_type = other DNA
ttttaatacagctctggagtggggt

SEQ ID NO: 578 - DNA, 25 bp, synthetic construct
  source 1..25: mol_type = other DNA
ttttaatacagctctggagtggggt

SEQ ID NO: 579 - DNA, 27 bp, synthetic construct
  source 1..27: mol_type = other DNA
ttttaatacagctctggagtggggtgg

SEQ ID NO: 580 - DNA, 27 bp, synthetic construct
  source 1..27: mol_type = other DNA
ttttaatacagctctggagtggggtgg

SEQ ID NO: 581 - DNA, 23 bp, synthetic construct
  source 1..23: mol_type = other DNA
ttaatacagctctggagtggggt

SEQ ID NO: 582 - DNA, 23 bp, synthetic construct
  source 1..23: mol_type = other DNA
ttaatacagctctggagtggggt

SEQ ID NO: 583 - DNA, 23 bp, synthetic construct
  source 1..23: mol_type = other DNA
ttaatacagctctggagtggggt

SEQ ID NO: 584 - DNA, 23 bp, synthetic construct
  source 1..23: mol_type = other DNA
ttaatacagctctggagtggggt

SEQ ID NO: 585 - DNA, 23 bp, synthetic construct
  source 1..23: mol_type = other DNA
ttaatacagctctggagtggggt

SEQ ID NO: 586 - DNA, 23 bp, synthetic construct
  source 1..23: mol_type = other DNA
ttaatacagctctggagtggggt

SEQ ID NO: 587 - DNA, 23 bp, synthetic construct
  source 1..23: mol_type = other DNA
ttaatacagctctggagtggggt

SEQ ID NO: 588 - DNA, 23 bp, synthetic construct
  source 1..23: mol_type = other DNA
ttaatacagctctggagtggggt

SEQ ID NO: 589 - DNA, 21 bp, synthetic construct
  source 1..21: mol_type = other DNA
aatacagctctggagtggggt

SEQ ID NO: 590 - DNA, 21 bp, synthetic construct
  source 1..21: mol_type = other DNA
aatacagctctggagtggggt

SEQ ID NO: 591 - DNA, 21 bp, synthetic construct
  source 1..21: mol_type = other DNA
aatacagctctggagtggggt

SEQ ID NO: 592 - DNA, 21 bp, synthetic construct
  source 1..21: mol_type = other DNA
aatacagctctggagtggggt

SEQ ID NO: 593 - DNA, 21 bp, synthetic construct
  source 1..21: mol_type = other DNA
aatacagctctggagtggggt

SEQ ID NO: 594 - DNA, 19 bp, synthetic construct
  source 1..19: mol_type = other DNA
aatacagctctggagtggg

SEQ ID NO: 595 - DNA, 19 bp, synthetic construct
  source 1..19: mol_type = other DNA
aatacagctctggagtggg

SEQ ID NO: 596 - DNA, 19 bp, synthetic construct
  source 1..19: mol_type = other DNA
aatacagctctggagtggg

SEQ ID NO: 597 - DNA, 19 bp, synthetic construct
  source 1..19: mol_type = other DNA
tacagctctggagtggggt

SEQ ID NO: 598 - DNA, 19 bp, synthetic construct
  source 1..19: mol_type = other DNA
tacagctctggagtggggt

SEQ ID NO: 599 - DNA, 19 bp, synthetic construct
  source 1..19: mol_type = other DNA
tacagctctggagtggggt

SEQ ID NO: 600 - DNA, 23 bp, synthetic construct
  source 1..23: mol_type = other DNA
ttaatacagctctggagtggggt

SEQ ID NO: 601 - DNA, 23 bp, synthetic construct
  source 1..23: mol_type = other DNA
ttaatacagctctggagtggggt

SEQ ID NO: 602 - DNA, 23 bp, synthetic construct
  source 1..23: mol_type = other DNA
ttaatacagctctggagtggggt

SEQ ID NO: 603 - DNA, 23 bp, synthetic construct
  source 1..23: mol_type = other DNA
ttaatacagctctggagtggggt

SEQ ID NO: 604 - DNA, 23 bp, synthetic construct
  source 1..23: mol_type = other DNA
ttaatacagctctggagtggggt

SEQ ID NO: 605 - DNA, 20 bp, synthetic construct
  source 1..20: mol_type = other DNA
tatgaagtaagaaaggggta

SEQ ID NO: 606 - DNA, 20 bp, synthetic construct
  source 1..20: mol_type = other DNA
tatgaagtaagaaaggggta

SEQ ID NO: 607 - DNA, 20 bp, synthetic construct
  source 1..20: mol_type = other DNA
tatgaagtaagaaaggggta

SEQ ID NO: 608 - DNA, 20 bp, synthetic construct
  source 1..20: mol_type = other DNA
tatgaagtaagaaaggggta

SEQ ID NO: 609 - DNA, 20 bp, synthetic construct
  source 1..20: mol_type = other DNA
tatgaagtaagaaaggggta

SEQ ID NO: 610 - DNA, 20 bp, synthetic construct
  source 1..20: mol_type = other DNA
tatgaagtaagaaaggggta

SEQ ID NO: 611 - DNA, 20 bp, synthetic construct
  source 1..20: mol_type = other DNA
tatgaagtaagaaaggggta

SEQ ID NO: 612 - DNA, 20 bp, synthetic construct
  source 1..20: mol_type = other DNA
tatgaagtaagaaaggggta

SEQ ID NO: 613 - DNA, 20 bp, synthetic construct
  source 1..20: mol_type = other DNA
tatgaagtaagaaaggggta

SEQ ID NO: 614 - DNA, 20 bp, synthetic construct
  source 1..20: mol_type = other DNA
tatgaagtaagaaaggggta

SEQ ID NO: 615 - DNA, 20 bp, synthetic construct
  source 1..20: mol_type = other DNA
tatgaagtaagaaaggggta

SEQ ID NO: 616 - DNA, 20 bp, synthetic construct
  source 1..20: mol_type = other DNA
tatgaagtaagaaaggggta

SEQ ID NO: 617 - DNA, 20 bp, synthetic construct
  source 1..20: mol_type = other DNA
tatgaagtaagaaaggggta

SEQ ID NO: 618 - DNA, 20 bp, synthetic construct
  source 1..20: mol_type = other DNA
gcttggggtatcagtactgc

SEQ ID NO: 619 - DNA, 20 bp, synthetic construct
  source 1..20: mol_type = other DNA
gcttggggtatcagtactgc

SEQ ID NO: 620 - DNA, 20 bp, synthetic construct
  source 1..20: mol_type = other DNA
gcttggggtatcagtactgc

SEQ ID NO: 621 - DNA, 20 bp, synthetic construct
  source 1..20: mol_type = other DNA
gcttggggtatcagtactgc

SEQ ID NO: 622 - DNA, 20 bp, synthetic construct
  source 1..20: mol_type = other DNA
gcttggggtatcagtactgc

SEQ ID NO: 623 - DNA, 20 bp, synthetic construct
  source 1..20: mol_type = other DNA
gcttggggtatcagtactgc

SEQ ID NO: 624 - DNA, 20 bp, synthetic construct
  source 1..20: mol_type = other DNA
gcttggggtatcagtactgc

SEQ ID NO: 625 - DNA, 20 bp, synthetic construct
  source 1..20: mol_type = other DNA
gcttggggtatcagtactgc

SEQ ID NO: 626 - DNA, 20 bp, synthetic construct
  source 1..20: mol_type = other DNA
gcttggggtatcagtactgc

SEQ ID NO: 627 - DNA, 20 bp, synthetic construct
  source 1..20: mol_type = other DNA
gcttggggtatcagtactgc

SEQ ID NO: 628 - DNA, 20 bp, synthetic construct
  source 1..20: mol_type = other DNA
gcttggggtatcagtactgc

SEQ ID NO: 629 - DNA, 20 bp, synthetic construct
  source 1..20: mol_type = other DNA
gcttggggtatcagtactgc

SEQ ID NO: 630 - DNA, 20 bp, synthetic construct
  source 1..20: mol_type = other DNA
gcttggggtatcagtactgc

SEQ ID NO: 631 - DNA, 22 bp, synthetic construct
  source 1..22: mol_type = other DNA
ttatgaagtaagaaaggggtaa

SEQ ID NO: 632 - DNA, 22 bp, synthetic construct
  source 1..22: mol_type = other DNA
ttatgaagtaagaaaggggtaa

SEQ ID NO: 633 - DNA, 22 bp, synthetic construct
  source 1..22: mol_type = other DNA
ttatgaagtaagaaaggggtaa

SEQ ID NO: 634 - DNA, 22 bp, synthetic construct
  source 1..22: mol_type = other DNA
ttatgaagtaagaaaggggtaa

SEQ ID NO: 635 - DNA, 24 bp, synthetic construct
  source 1..24: mol_type = other DNA
tttatgaagtaagaaaggggtaaa

SEQ ID NO: 636 - DNA, 24 bp, synthetic construct
  source 1..24: mol_type = other DNA
tttatgaagtaagaaaggggtaaa

SEQ ID NO: 637 - DNA, 24 bp, synthetic construct
  source 1..24: mol_type = other DNA
tttatgaagtaagaaaggggtaaa

SEQ ID NO: 638 - DNA, 24 bp, synthetic construct
  source 1..24: mol_type = other DNA
tttatgaagtaagaaaggggtaaa

SEQ ID NO: 639 - DNA, 22 bp, synthetic construct
  source 1..22: mol_type = other DNA
tgcttggggtatcagtactgca

SEQ ID NO: 640 - DNA, 22 bp, synthetic construct
  source 1..22: mol_type = other DNA
tgcttggggtatcagtactgca

SEQ ID NO: 641 - DNA, 22 bp, synthetic construct
  source 1..22: mol_type = other DNA
tgcttggggtatcagtactgca

SEQ ID NO: 642 - DNA, 22 bp, synthetic construct
  source 1..22: mol_type = other DNA
tgcttggggtatcagtactgca

SEQ ID NO: 643 - DNA, 24 bp, synthetic construct
  source 1..24: mol_type = other DNA
gtgcttggggtatcagtactgcac

SEQ ID NO: 644 - DNA, 24 bp, synthetic construct
  source 1..24: mol_type = other DNA
gtgcttggggtatcagtactgcac

SEQ ID NO: 645 - DNA, 24 bp, synthetic construct
  source 1..24: mol_type = other DNA
gtgcttggggtatcagtactgcac

SEQ ID NO: 646 - DNA, 24 bp, synthetic construct
  source 1..24: mol_type = other DNA
gtgcttggggtatcagtactgcac

SEQ ID NO: 647 - DNA, 20 bp, synthetic construct
  source 1..20: mol_type = other DNA
aatatggttaatgacccaac

SEQ ID NO: 648 - DNA, 20 bp, synthetic construct
  source 1..20: mol_type = other DNA
aatatggttaatgacccaac

SEQ ID NO: 649 - DNA, 20 bp, synthetic construct
  source 1..20: mol_type = other DNA
aatatggttaatgacccaac

SEQ ID NO: 650 - DNA, 20 bp, synthetic construct
  source 1..20: mol_type = other DNA
aatatggttaatgacccaac

SEQ ID NO: 651 - DNA, 20 bp, synthetic construct
  source 1..20: mol_type = other DNA
gaagtaagaaaggggtaaat

SEQ ID NO: 652 - DNA, 20 bp, synthetic construct
  source 1..20: mol_type = other DNA
gaagtaagaaaggggtaaat

SEQ ID NO: 653 - DNA, 20 bp, synthetic construct
  source 1..20: mol_type = other DNA
gaagtaagaaaggggtaaat

SEQ ID NO: 654 - DNA, 20 bp, synthetic construct
  source 1..20: mol_type = other DNA
gaagtaagaaaggggtaaat

SEQ ID NO: 655 - DNA, 20 bp, synthetic construct
  source 1..20: mol_type = other DNA
tgaagtaagaaaggggtaaa

SEQ ID NO: 656 - DNA, 20 bp, synthetic construct
  source 1..20: mol_type = other DNA
tgaagtaagaaaggggtaaa

SEQ ID NO: 657 - DNA, 20 bp, synthetic construct
  source 1..20: mol_type = other DNA
tgaagtaagaaaggggtaaa

SEQ ID NO: 658 - DNA, 20 bp, synthetic construct
  source 1..20: mol_type = other DNA
tgaagtaagaaaggggtaaa

SEQ ID NO: 659 - DNA, 20 bp, synthetic construct
  source 1..20: mol_type = other DNA
atgaagtaagaaaggggtaa

SEQ ID NO: 660 - DNA, 20 bp, synthetic construct
  source 1..20: mol_type = other DNA
atgaagtaagaaaggggtaa

SEQ ID NO: 661 - DNA, 20 bp, synthetic construct
  source 1..20: mol_type = other DNA
atgaagtaagaaaggggtaa

SEQ ID NO: 662 - DNA, 20 bp, synthetic construct
  source 1..20: mol_type = other DNA
atgaagtaagaaaggggtaa

SEQ ID NO: 663 - DNA, 20 bp, synthetic construct
  source 1..20: mol_type = other DNA
ttatgaagtaagaaaggggt

SEQ ID NO: 664 - DNA, 20 bp, synthetic construct
  source 1..20: mol_type = other DNA
ttatgaagtaagaaaggggt

SEQ ID NO: 665 - DNA, 20 bp, synthetic construct
  source 1..20: mol_type = other DNA
ttatgaagtaagaaaggggt

SEQ ID NO: 666 - DNA, 20 bp, synthetic construct
  source 1..20: mol_type = other DNA
ttatgaagtaagaaaggggt

SEQ ID NO: 667 - DNA, 20 bp, synthetic construct
  source 1..20: mol_type = other DNA
ggggtatcagtactgcactt

SEQ ID NO: 668 - DNA, 20 bp, synthetic construct
  source 1..20: mol_type = other DNA
ggggtatcagtactgcactt

SEQ ID NO: 669 - DNA, 20 bp, synthetic construct
  source 1..20: mol_type = other DNA
ggggtatcagtactgcactt

SEQ ID NO: 670 - DNA, 20 bp, synthetic construct
  source 1..20: mol_type = other DNA
ggggtatcagtactgcactt

SEQ ID NO: 671 - DNA, 20 bp, synthetic construct
  source 1..20: mol_type = other DNA
tgcttggggtatcagtactg

SEQ ID NO: 672 - DNA, 20 bp, synthetic construct
  source 1..20: mol_type = other DNA
tgcttggggtatcagtactg

SEQ ID NO: 673 - DNA, 20 bp, synthetic construct
  source 1..20: mol_type = other DNA
tgcttggggtatcagtactg

SEQ ID NO: 674 - DNA, 20 bp, synthetic construct
  source 1..20: mol_type = other DNA
tgcttggggtatcagtactg

SEQ ID NO: 675 - DNA, 20 bp, synthetic construct
  source 1..20: mol_type = other DNA
tcagtgcttggggtatcagt

SEQ ID NO: 676 - DNA, 20 bp, synthetic construct
  source 1..20: mol_type = other DNA
tcagtgcttggggtatcagt

SEQ ID NO: 677 - DNA, 20 bp, synthetic construct
  source 1..20: mol_type = other DNA
tcagtgcttggggtatcagt

SEQ ID NO: 678 - DNA, 20 bp, synthetic construct
  source 1..20: mol_type = other DNA
tcagtgcttggggtatcagt

SEQ ID NO: 679 - DNA, 20 bp, synthetic construct
  source 1..20: mol_type = other DNA
aatcagtgcttggggtatca

SEQ ID NO: 680 - DNA, 20 bp, synthetic construct
  source 1..20: mol_type = other DNA
aatcagtgcttggggtatca

SEQ ID NO: 681 - DNA, 20 bp, synthetic construct
  source 1..20: mol_type = other DNA
aatcagtgcttggggtatca

SEQ ID NO: 682 - DNA, 20 bp, synthetic construct
  source 1..20: mol_type = other DNA
aatcagtgcttggggtatca

SEQ ID NO: 683 - DNA, 23 bp, synthetic construct
  source 1..23: mol_type = other DNA
ttaatacagctctggagtggggt

SEQ ID NO: 684 - DNA, 23 bp, synthetic construct
  source 1..23: mol_type = other DNA
ttaatacagctctggagtggggt

SEQ ID NO: 685 - DNA, 23 bp, synthetic construct
  source 1..23: mol_type = other DNA
ttaatacagctctggagtggggt

SEQ ID NO: 686 - DNA, 23 bp, synthetic construct
  source 1..23: mol_type = other DNA
ttaatacagctctggagtggggt

SEQ ID NO: 687 - DNA, 23 bp, synthetic construct
  source 1..23: mol_type = other DNA
ttaatacagctctggagtggggt

SEQ ID NO: 688 - DNA, 23 bp, synthetic construct
  source 1..23: mol_type = other DNA
ttaatacagctctggagtggggt

SEQ ID NO: 689 - DNA, 23 bp, synthetic construct
  source 1..23: mol_type = other DNA
ttaatacagctctggagtggggt

SEQ ID NO: 690 - DNA, 23 bp, synthetic construct
  source 1..23: mol_type = other DNA
ttaatacagctctggagtggggt

SEQ ID NO: 691 - DNA, 23 bp, synthetic construct
  source 1..23: mol_type = other DNA
ttaatacagctctggagtggggt

SEQ ID NO: 692 - DNA, 21 bp, synthetic construct
  source 1..21: mol_type = other DNA
ttaatacagctctggagtggg

SEQ ID NO: 693 - DNA, 21 bp, synthetic construct
  source 1..21: mol_type = other DNA
aatacagctctggagtggggt

SEQ ID NO: 694 - DNA, 19 bp, synthetic construct
  source 1..19: mol_type = other DNA
aatacagctctggagtggg

SEQ ID NO: 695 - DNA, 20 bp, synthetic construct
  source 1..20: mol_type = other DNA
tgaagtaagaaaggggtaaa

SEQ ID NO: 696 - DNA, 20 bp, synthetic construct
  source 1..20: mol_type = other DNA
tatgaagtaagaaaggggta

SEQ ID NO: 697 - DNA, 23 bp, synthetic construct
  source 1..23: mol_type = other DNA
ttaatacagctctggagtggggt

SEQ ID NO: 698 - DNA, 23 bp, synthetic construct
  source 1..23: mol_type = other DNA
ttaatacagctctggagtggggt

SEQ ID NO: 699 - DNA, 23 bp, synthetic construct
  source 1..23: mol_type = other DNA
ttaatacagctctggagtggggt

SEQ ID NO: 700 - DNA, 23 bp, synthetic construct
  source 1..23: mol_type = other DNA
ttaatacagctctggagtggggt

SEQ ID NO: 701 - DNA, 23 bp, synthetic construct
  source 1..23: mol_type = other DNA
ttaatacagctctggagtggggt

SEQ ID NO: 702 - DNA, 23 bp, synthetic construct
  source 1..23: mol_type = other DNA
ttaatacagctctggagtggggt

SEQ ID NO: 703 - DNA, 23 bp, synthetic construct
  source 1..23: mol_type = other DNA
ttaatacagctctggagtggggt

SEQ ID NO: 704 - DNA, 23 bp, synthetic construct
  source 1..23: mol_type = other DNA
ttaatacagctctggagtggggt

SEQ ID NO: 705 - DNA, 23 bp, synthetic construct
  source 1..23: mol_type = other DNA
ttaatacagctctggagtggggt

SEQ ID NO: 706 - DNA, 23 bp, synthetic construct
  source 1..23: mol_type = other DNA
ttaatacagctctggagtggggt

SEQ ID NO: 707 - DNA, 20 bp, synthetic construct
  source 1..20: mol_type = other DNA
tatgaagtaagaaaggggta

SEQ ID NO: 708 - DNA, 20 bp, synthetic construct
  source 1..20: mol_type = other DNA
tatgaagtaagaaaggggta

SEQ ID NO: 709 - DNA, 20 bp, synthetic construct
  source 1..20: mol_type = other DNA
tatgaagtaagaaaggggta

SEQ ID NO: 710 - DNA, 20 bp, synthetic construct
  source 1..20: mol_type = other DNA
tatgaagtaagaaaggggta

SEQ ID NO: 711 - DNA, 20 bp, synthetic construct
  source 1..20: mol_type = other DNA
tatgaagtaagaaaggggta

SEQ ID NO: 712 - DNA, 20 bp, synthetic construct
  source 1..20: mol_type = other DNA
tgaagtaagaaaggggtaaa

SEQ ID NO: 713 - DNA, 20 bp, synthetic construct
  source 1..20: mol_type = other DNA
tcacactctgttcagaattt

SEQ ID NO: 714 - DNA, 20 bp, synthetic construct
  source 1..20: mol_type = other DNA
gaatttgatgattggcattt

SEQ ID NO: 715 - DNA, 20 bp, synthetic construct
  source 1..20: mol_type = other DNA
acttgaatttgatgattggc

SEQ ID NO: 716 - DNA, 20 bp, synthetic construct
  source 1..20: mol_type = other DNA
gaagcgagattatacttgaa

SEQ ID NO: 717 - DNA, 20 bp, synthetic construct
  source 1..20: mol_type = other DNA
agagagaagcgagattatac

SEQ ID NO: 718 - DNA, 20 bp, synthetic construct
  source 1..20: mol_type = other DNA
ggtctatatgacaagagaga

SEQ ID NO: 719 - DNA, 20 bp, synthetic construct
  source 1..20: mol_type = other DNA
tcaatggaaatgtggtctat

SEQ ID NO: 720 - DNA, 20 bp, synthetic construct
  source 1..20: mol_type = other DNA
gaaccaggtatggaaacttg

SEQ ID NO: 721 - DNA, 20 bp, synthetic construct
  source 1..20: mol_type = other DNA
gtgcatttcctaatcttgtc

SEQ ID NO: 722 - DNA, 20 bp, synthetic construct
  source 1..20: mol_type = other DNA
ttgtgttgtgcatttcctaa

SEQ ID NO: 723 - DNA, 20 bp, synthetic construct
  source 1..20: mol_type = other DNA
catctcaactgcctctgaca

SEQ ID NO: 724 - DNA, 20 bp, synthetic construct
  source 1..20: mol_type = other DNA
tgcatctcaactgcctctga

SEQ ID NO: 725 - DNA, 20 bp, synthetic construct
  source 1..20: mol_type = other DNA
ctttgcatctcaactgcctc

SEQ ID NO: 726 - DNA, 20 bp, synthetic construct
  source 1..20: mol_type = other DNA
tgaaagagaacctcgtgctt

SEQ ID NO: 727 - DNA, 20 bp, synthetic construct
  source 1..20: mol_type = other DNA
ttgaaagagaacctcgtgct

SEQ ID NO: 728 - DNA, 20 bp, synthetic construct
  source 1..20: mol_type = other DNA
gggaacttgaagagataaaa

SEQ ID NO: 729 - DNA, 20 bp, synthetic construct
  source 1..20: mol_type = other DNA
tctgtgtgggaacttgaaga

SEQ ID NO: 730 - DNA, 20 bp, synthetic construct
  source 1..20: mol_type = other DNA
gctctaggattctgtgtggg

SEQ ID NO: 731 - DNA, 20 bp, synthetic construct
  source 1..20: mol_type = other DNA
atagctctaggattctgtgt

SEQ ID NO: 732 - DNA, 20 bp, synthetic construct
  source 1..20: mol_type = other DNA
gatagctctaggattctgtg

SEQ ID NO: 733 - DNA, 20 bp, synthetic construct
  source 1..20: mol_type = other DNA
agatagctctaggattctgt

SEQ ID NO: 734 - DNA, 20 bp, synthetic construct
  source 1..20: mol_type = other DNA
gaagagatagctctaggatt

SEQ ID NO: 735 - DNA, 20 bp, synthetic construct
  source 1..20: mol_type = other DNA
ggagaagagatagctctagg

SEQ ID NO: 736 - DNA, 20 bp, synthetic construct
  source 1..20: mol_type = other DNA
atgggagaagagatagctct

SEQ ID NO: 737 - DNA, 20 bp, synthetic construct
  source 1..20: mol_type = other DNA
ggaggaattggaatcagcct

SEQ ID NO: 738 - DNA, 20 bp, synthetic construct
  source 1..20: mol_type = other DNA
ctcctttatgcagtttaacc

SEQ ID NO: 739 - DNA, 20 bp, synthetic construct
  source 1..20: mol_type = other DNA
gtttgctctgtgcctgatag

SEQ ID NO: 740 - DNA, 20 bp, synthetic construct
  source 1..20: mol_type = other DNA
atttccctgtttgctctgtg

SEQ ID NO: 741 - DNA, 20 bp, synthetic construct
  source 1..20: mol_type = other DNA
gaacctctgtgatttccctg

SEQ ID NO: 742 - DNA, 20 bp, synthetic construct
  source 1..20: mol_type = other DNA
ttgaacctctgtgatttccc

SEQ ID NO: 743 - DNA, 20 bp, synthetic construct
  source 1..20: mol_type = other DNA
actttgaacctctgtgattt

SEQ ID NO: 744 - DNA, 20 bp, synthetic construct
  source 1..20: mol_type = other DNA
gaactttgaacctctgtgat

SEQ ID NO: 745 - DNA, 20 bp, synthetic construct
  source 1..20: mol_type = other DNA
ggtgaactttgaacctctgt

SEQ ID NO: 746 - DNA, 20 bp, synthetic construct
  source 1..20: mol_type = other DNA
gacactggtgaactttgaac

SEQ ID NO: 747 - DNA, 20 bp, synthetic construct
  source 1..20: mol_type = other DNA
attgttgagacactggtgaa

SEQ ID NO: 748 - DNA, 20 bp, synthetic construct
  source 1..20: mol_type = other DNA
gattgttgagacactggtga

SEQ ID NO: 749 - DNA, 20 bp, synthetic construct
  source 1..20: mol_type = other DNA
agtcacagggagcattaggg

SEQ ID NO: 750 - DNA, 20 bp, synthetic construct
  source 1..20: mol_type = other DNA
gtagtcacagggagcattag

SEQ ID NO: 751 - DNA, 20 bp, synthetic construct
  source 1..20: mol_type = other DNA
ggtagtcacagggagcatta

SEQ ID NO: 752 - DNA, 20 bp, synthetic construct
  source 1..20: mol_type = other DNA
tggtagtcacagggagcatt

SEQ ID NO: 753 - DNA, 20 bp, synthetic construct
  source 1..20: mol_type = other DNA
gtctggtagtcacagggagc

SEQ ID NO: 754 - DNA, 20 bp, synthetic construct
  source 1..20: mol_type = other DNA
ggagtgtctggtagtcacag

SEQ ID NO: 755 - DNA, 20 bp, synthetic construct
  source 1..20: mol_type = other DNA
ggtcctgggagtgtctggta

SEQ ID NO: 756 - DNA, 20 bp, synthetic construct
  source 1..20: mol_type = other DNA
ctcaggtcctgggagtgtct

SEQ ID NO: 757 - DNA, 20 bp, synthetic construct
  source 1..20: mol_type = other DNA
actcaggtcctgggagtgtc

SEQ ID NO: 758 - DNA, 20 bp, synthetic construct
  source 1..20: mol_type = other DNA
ccctggtcagtagaaagcaa

SEQ ID NO: 759 - DNA, 20 bp, synthetic construct
  source 1..20: mol_type = other DNA
accctggtcagtagaaagca

SEQ ID NO: 760 - DNA, 20 bp, synthetic construct
  source 1..20: mol_type = other DNA
aaccctggtcagtagaaagc

SEQ ID NO: 761 - DNA, 20 bp, synthetic construct
  source 1..20: mol_type = other DNA
ggagaatattgccatgaaaa

SEQ ID NO: 762 - DNA, 20 bp, synthetic construct
  source 1..20: mol_type = other DNA
agcagtgacttttgaagcca

SEQ ID NO: 763 - DNA, 20 bp, synthetic construct
  source 1..20: mol_type = other DNA
agagaaggcattcaccaagc

SEQ ID NO: 764 - DNA, 20 bp, synthetic construct
  source 1..20: mol_type = other DNA
gagaggcaccaactacaaag

SEQ ID NO: 765 - DNA, 20 bp, synthetic construct
  source 1..20: mol_type = other DNA
gaagagaggcaccaactaca

SEQ ID NO: 766 - DNA, 20 bp, synthetic construct
  source 1..20: mol_type = other DNA
gtctgaagagaggcaccaac

SEQ ID NO: 767 - DNA, 20 bp, synthetic construct
  source 1..20: mol_type = other DNA
agggtctgaagagaggcacc

SEQ ID NO: 768 - DNA, 20 bp, synthetic construct
  source 1..20: mol_type = other DNA
agtagggtctgaagagaggc

SEQ ID NO: 769 - DNA, 20 bp, synthetic construct
  source 1..20: mol_type = other DNA
aatcaatagcaggtcaagag

SEQ ID NO: 770 - DNA, 20 bp, synthetic construct
  source 1..20: mol_type = other DNA
tgcttagtagatatagccca

SEQ ID NO: 771 - DNA, 20 bp, synthetic construct
  source 1..20: mol_type = other DNA
gagcccctgcttagtagata

SEQ ID NO: 772 - DNA, 20 bp, synthetic construct
  source 1..20: mol_type = other DNA
agagcccctgcttagtagat

SEQ ID NO: 773 - DNA, 20 bp, synthetic construct
  source 1..20: mol_type = other DNA
ttaatcattagcttaaggtg

SEQ ID NO: 774 - DNA, 20 bp, synthetic construct
  source 1..20: mol_type = other DNA
gtttaatcattagcttaagg

SEQ ID NO: 775 - DNA, 20 bp, synthetic construct
  source 1..20: mol_type = other DNA
tgagagaagaaggctgtgtt

SEQ ID NO: 776 - DNA, 20 bp, synthetic construct
  source 1..20: mol_type = other DNA
agagttgttaccagtggagc

SEQ ID NO: 777 - DNA, 20 bp, synthetic construct
  source 1..20: mol_type = other DNA
acagagttgttaccagtgga

SEQ ID NO: 778 - DNA, 20 bp, synthetic construct
  source 1..20: mol_type = other DNA
caggccacagagttgttacc

SEQ ID NO: 779 - DNA, 20 bp, synthetic construct
  source 1..20: mol_type = other DNA
acaggccacagagttgttac

SEQ ID NO: 780 - DNA, 20 bp, synthetic construct
  source 1..20: mol_type = other DNA
tctcttgaatcttgaagaca

SEQ ID NO: 781 - DNA, 20 bp, synthetic construct
  source 1..20: mol_type = other DNA
atgacctaggctccaaacac

SEQ ID NO: 782 - DNA, 20 bp, synthetic construct
  source 1..20: mol_type = other DNA
gtagcacctatgacctaggc

SEQ ID NO: 783 - DNA, 20 bp, synthetic construct
  source 1..20: mol_type = other DNA
tggtgaatatgtagcaccta

SEQ ID NO: 784 - DNA, 20 bp, synthetic construct
  source 1..20: mol_type = other DNA
gaagaaatggtgaactccag

SEQ ID NO: 785 - DNA, 20 bp, synthetic construct
  source 1..20: mol_type = other DNA
cctggaagaaatggtgaact

SEQ ID NO: 786 - DNA, 20 bp, synthetic construct
  source 1..20: mol_type = other DNA
ggttccctggaagaaatggt

SEQ ID NO: 787 - DNA, 20 bp, synthetic construct
  source 1..20: mol_type = other DNA
tggttccctggaagaaatgg

SEQ ID NO: 788 - DNA, 20 bp, synthetic construct
  source 1..20: mol_type = other DNA
ctccttggttccctggaaga

SEQ ID NO: 789 - DNA, 20 bp, synthetic construct
  source 1..20: mol_type = other DNA
tgaactccttggttccctgg

SEQ ID NO: 790 - DNA, 20 bp, synthetic construct
  source 1..20: mol_type = other DNA
aaggtgacctggaaatccat

SEQ ID NO: 791 - DNA, 20 bp, synthetic construct
  source 1..20: mol_type = other DNA
ttcgcttacttcttaatggt

SEQ ID NO: 792 - DNA, 20 bp, synthetic construct
  source 1..20: mol_type = other DNA
gattcgcttacttcttaatg

SEQ ID NO: 793 - DNA, 20 bp, synthetic construct
  source 1..20: mol_type = other DNA
gatgattcgcttacttctta

SEQ ID NO: 794 - DNA, 20 bp, synthetic construct
  source 1..20: mol_type = other DNA
cagatgattcgcttacttct

SEQ ID NO: 795 - DNA, 20 bp, synthetic construct
  source 1..20: mol_type = other DNA
gccagatgattcgcttactt

SEQ ID NO: 796 - DNA, 20 bp, synthetic construct
  source 1..20: mol_type = other DNA
ttgccagatgattcgcttac

SEQ ID NO: 797 - DNA, 20 bp, synthetic construct
  source 1..20: mol_type = other DNA
gcttgccagatgattcgctt

SEQ ID NO: 798 - DNA, 20 bp, synthetic construct
  source 1..20: mol_type = other DNA
tgcttgccagatgattcgct

SEQ ID NO: 799 - DNA, 20 bp, synthetic construct
  source 1..20: mol_type = other DNA
ctgcttgccagatgattcgc

SEQ ID NO: 800 - DNA, 20 bp, synthetic construct
  source 1..20: mol_type = other DNA
tagtactgcttgccagatga

SEQ ID NO: 801 - DNA, 20 bp, synthetic construct
  source 1..20: mol_type = other DNA
gctgctagtactgcttgcca

SEQ ID NO: 802 - DNA, 20 bp, synthetic construct
  source 1..20: mol_type = other DNA
gagctgctagtactgcttgc

SEQ ID NO: 803 - DNA, 20 bp, synthetic construct
  source 1..20: mol_type = other DNA
gctatgagtaggagctgcta

SEQ ID NO: 804 - DNA, 20 bp, synthetic construct
  source 1..20: mol_type = other DNA
ttgaacactgagctaggtac

SEQ ID NO: 805 - DNA, 20 bp, synthetic construct
  source 1..20: mol_type = other DNA
tattgaacactgagctaggt

SEQ ID NO: 806 - DNA, 20 bp, synthetic construct
  source 1..20: mol_type = other DNA
acatgtgccaatggtagtag

SEQ ID NO: 807 - DNA, 20 bp, synthetic construct
  source 1..20: mol_type = other DNA
ccacatgtgccaatggtagt

SEQ ID NO: 808 - DNA, 20 bp, synthetic construct
  source 1..20: mol_type = other DNA
tcaccacatgtgccaatggt

SEQ ID NO: 809 - DNA, 20 bp, synthetic construct
  source 1..20: mol_type = other DNA
ggaaagtgattagaataagc

SEQ ID NO: 810 - DNA, 20 bp, synthetic construct
  source 1..20: mol_type = other DNA
gtaggaaagtgattagaata

SEQ ID NO: 811 - DNA, 20 bp, synthetic construct
  source 1..20: mol_type = other DNA
gtatgaaagaaagacggaca

SEQ ID NO: 812 - DNA, 20 bp, synthetic construct
  source 1..20: mol_type = other DNA
gggtatgaaagaaagacgga

SEQ ID NO: 813 - DNA, 20 bp, synthetic construct
  source 1..20: mol_type = other DNA
acttttaatacagctctgga

SEQ ID NO: 814 - DNA, 20 bp, synthetic construct
  source 1..20: mol_type = other DNA
tctcgatgcatagctgggat

SEQ ID NO: 815 - DNA, 20 bp, synthetic construct
  source 1..20: mol_type = other DNA
cctctcgatgcatagctggg

SEQ ID NO: 816 - DNA, 20 bp, synthetic construct
  source 1..20: mol_type = other DNA
gcctctcgatgcatagctgg

SEQ ID NO: 817 - DNA, 20 bp, synthetic construct
  source 1..20: mol_type = other DNA
caacacttttatttgtagga

SEQ ID NO: 818 - DNA, 20 bp, synthetic construct
  source 1..20: mol_type = other DNA
cacctcaacacttttatttg

SEQ ID NO: 819 - DNA, 20 bp, synthetic construct
  source 1..20: mol_type = other DNA
gacggacacctcaacacttt

SEQ ID NO: 820 - DNA, 20 bp, synthetic construct
  source 1..20: mol_type = other DNA
aagaaagacggacacctcaa

SEQ ID NO: 821 - DNA, 20 bp, synthetic construct
  source 1..20: mol_type = other DNA
tatgaaagaaagacggacac

SEQ ID NO: 822 - DNA, 20 bp, synthetic construct
  source 1..20: mol_type = other DNA
gggggtatgaaagaaagacg

SEQ ID NO: 823 - DNA, 20 bp, synthetic construct
  source 1..20: mol_type = other DNA
gggtgggggtatgaaagaaa

SEQ ID NO: 824 - DNA, 20 bp, synthetic construct
  source 1..20: mol_type = other DNA
agtggggtgggggtatgaaa

SEQ ID NO: 825 - DNA, 20 bp, synthetic construct
  source 1..20: mol_type = other DNA
ctctggagtggggtgggggt

SEQ ID NO: 826 - DNA, 20 bp, synthetic construct
  source 1..20: mol_type = other DNA
acagctctggagtggggtgg

SEQ ID NO: 827 - DNA, 20 bp, synthetic construct
  source 1..20: mol_type = other DNA
tttcacttttaatacagctc

SEQ ID NO: 828 - DNA, 20 bp, synthetic construct
  source 1..20: mol_type = other DNA
ctgaatttcacttttaatac

SEQ ID NO: 829 - DNA, 20 bp, synthetic construct
  source 1..20: mol_type = other DNA
cagcctgaatttcactttta

SEQ ID NO: 830 - DNA, 20 bp, synthetic construct
  source 1..20: mol_type = other DNA
atgcccagcctgaatttcac

SEQ ID NO: 831 - DNA, 20 bp, synthetic construct
  source 1..20: mol_type = other DNA
caccatgcccagcctgaatt

SEQ ID NO: 832 - DNA, 20 bp, synthetic construct
  source 1..20: mol_type = other DNA
gagccaccatgcccagcctg

SEQ ID NO: 833 - DNA, 20 bp, synthetic construct
  source 1..20: mol_type = other DNA
gcgtgagccaccatgcccag

SEQ ID NO: 834 - DNA, 20 bp, synthetic construct
  source 1..20: mol_type = other DNA
ttacaggcgtgagccaccat

SEQ ID NO: 835 - DNA, 20 bp, synthetic construct
  source 1..20: mol_type = other DNA
gggattacaggcgtgagcca

SEQ ID NO: 836 - DNA, 20 bp, synthetic construct
  source 1..20: mol_type = other DNA
tgctgggattacaggcgtga

SEQ ID NO: 837 - DNA, 20 bp, synthetic construct
  source 1..20: mol_type = other DNA
tcaaatgcctcaattgaaag

SEQ ID NO: 838 - DNA, 20 bp, synthetic construct
  source 1..20: mol_type = other DNA
tatgaaggaccctgtctacc

SEQ ID NO: 839 - DNA, 20 bp, synthetic construct
  source 1..20: mol_type = other DNA
gcatttcacactctgttcag

SEQ ID NO: 840 - DNA, 20 bp, synthetic construct
  source 1..20: mol_type = other DNA
tatatgacaagagagaagcg

SEQ ID NO: 841 - DNA, 20 bp, synthetic construct
  source 1..20: mol_type = other DNA
cagaataatgatttcaatgg

SEQ ID NO: 842 - DNA, 20 bp, synthetic construct
  source 1..20: mol_type = other DNA
aatcaataacttccagggca

SEQ ID NO: 843 - DNA, 20 bp, synthetic construct
  source 1..20: mol_type = other DNA
ttgtccatatctaagctcaa

SEQ ID NO: 844 - DNA, 20 bp, synthetic construct
  source 1..20: mol_type = other DNA
agaaccaggtatggaaactt

SEQ ID NO: 845 - DNA, 20 bp, synthetic construct
  source 1..20: mol_type = other DNA
gtgcagaaatttaccagatc

SEQ ID NO: 846 - DNA, 20 bp, synthetic construct
  source 1..20: mol_type = other DNA
gtgttgtgcatttcctaatc

SEQ ID NO: 847 - DNA, 20 bp, synthetic construct
  source 1..20: mol_type = other DNA
tttacccctttcttacttca

SEQ ID NO: 848 - DNA, 20 bp, synthetic construct
  source 1..20: mol_type = other DNA
cacaaggttgggtcattaac

SEQ ID NO: 849 - DNA, 20 bp, synthetic construct
  source 1..20: mol_type = other DNA
cttgaaagagaacctcgtgc

SEQ ID NO: 850 - DNA, 20 bp, synthetic construct
  source 1..20: mol_type = other DNA
ctaggattctgtgtgggaac

SEQ ID NO: 851 - DNA, 20 bp, synthetic construct
  source 1..20: mol_type = other DNA
attggaatcagcctatggga

SEQ ID NO: 852 - DNA, 20 bp, synthetic construct
  source 1..20: mol_type = other DNA
aacctctgtacttccaatgg

SEQ ID NO: 853 - DNA, 20 bp, synthetic construct
  source 1..20: mol_type = other DNA
gtgcctgatagctttcagtc

SEQ ID NO: 854 - DNA, 20 bp, synthetic construct
  source 1..20: mol_type = other DNA
gtgaactttgaacctctgtg

SEQ ID NO: 855 - DNA, 20 bp, synthetic construct
  source 1..20: mol_type = other DNA
gcattagggctgattgttga

SEQ ID NO: 856 - DNA, 20 bp, synthetic construct
  source 1..20: mol_type = other DNA
tgggagtgtctggtagtcac

SEQ ID NO: 857 - DNA, 20 bp, synthetic construct
  source 1..20: mol_type = other DNA
catgggatccccatcactca

SEQ ID NO: 858 - DNA, 20 bp, synthetic construct
  source 1..20: mol_type = other DNA
gtcagtagaaagcaatgaaa

SEQ ID NO: 859 - DNA, 20 bp, synthetic construct
  source 1..20: mol_type = other DNA
acagtgctcctagaccaaac

SEQ ID NO: 860 - DNA, 20 bp, synthetic construct
  source 1..20: mol_type = other DNA
gctcaaggggagaatattgc

SEQ ID NO: 861 - DNA, 20 bp, synthetic construct
  source 1..20: mol_type = other DNA
agtgacttttgaagccatcc

SEQ ID NO: 862 - DNA, 20 bp, synthetic construct
  source 1..20: mol_type = other DNA
tgggcagagaaggcattcac

SEQ ID NO: 863 - DNA, 20 bp, synthetic construct
  source 1..20: mol_type = other DNA
ggtttcaagtttcaccaaaa

SEQ ID NO: 864 - DNA, 20 bp, synthetic construct
  source 1..20: mol_type = other DNA
ctgaagagaggcaccaacta

SEQ ID NO: 865 - DNA, 20 bp, synthetic construct
  source 1..20: mol_type = other DNA
gtcaagagcacctcccaagt

SEQ ID NO: 866 - DNA, 20 bp, synthetic construct
  source 1..20: mol_type = other DNA
ctgcttagtagatatagccc

SEQ ID NO: 867 - DNA, 20 bp, synthetic construct
  source 1..20: mol_type = other DNA
cattagcttaaggtgagggc

SEQ ID NO: 868 - DNA, 20 bp, synthetic construct
  source 1..20: mol_type = other DNA
taccagtggagcagccttga

SEQ ID NO: 869 - DNA, 20 bp, synthetic construct
  source 1..20: mol_type = other DNA
taggtcccatctttacaggc

SEQ ID NO: 870 - DNA, 20 bp, synthetic construct
  source 1..20: mol_type = other DNA
acaggattcccatggtctat

SEQ ID NO: 871 - DNA, 20 bp, synthetic construct
  source 1..20: mol_type = other DNA
ccccatgaaaagggcttgtt

SEQ ID NO: 872 - DNA, 20 bp, synthetic construct
  source 1..20: mol_type = other DNA
gtgaatatgtagcacctatg

SEQ ID NO: 873 - DNA, 20 bp, synthetic construct
  source 1..20: mol_type = other DNA
attctggaggaataaatcaa

SEQ ID NO: 874 - DNA, 20 bp, synthetic construct
  source 1..20: mol_type = other DNA
gaaatggtgaactccagttg

SEQ ID NO: 875 - DNA, 20 bp, synthetic construct
  source 1..20: mol_type = other DNA
aaatccatgaactccttggt

SEQ ID NO: 876 - DNA, 20 bp, synthetic construct
  source 1..20: mol_type = other DNA
tgcataacaatgaaggtgac

SEQ ID NO: 877 - DNA, 20 bp, synthetic construct
  source 1..20: mol_type = other DNA
cttgccagatgattcgctta

SEQ ID NO: 878 - DNA, 20 bp, synthetic construct
  source 1..20: mol_type = other DNA
ctatgagtaggagctgctag

SEQ ID NO: 879 - DNA, 20 bp, synthetic construct
  source 1..20: mol_type = other DNA
ttacatttcatactcacaac

SEQ ID NO: 880 - DNA, 20 bp, synthetic construct
  source 1..20: mol_type = other DNA
ctgtgctacagtactctcta

SEQ ID NO: 881 - DNA, 20 bp, synthetic construct
  source 1..20: mol_type = other DNA
atttattgaacactgagcta

SEQ ID NO: 882 - DNA, 20 bp, synthetic construct
  source 1..20: mol_type = other DNA
gccaatggtagtagttaaca

SEQ ID NO: 883 - DNA, 20 bp, synthetic construct
  source 1..20: mol_type = other DNA
agccagggatggggcctctc

SEQ ID NO: 884 - DNA, 20 bp, synthetic construct
  source 1..20: mol_type = other DNA
gaaagtgattagaataagcc

SEQ ID NO: 885 - DNA, 20 bp, synthetic construct
  source 1..20: mol_type = other DNA
tccgcccgcctcaaagtgct

SEQ ID NO: 886 - DNA, 20 bp, synthetic construct
  source 1..20: mol_type = other DNA
ggctagtctcgaactcctga

SEQ ID NO: 887 - DNA, 20 bp, synthetic construct
  source 1..20: mol_type = other DNA
tgtatttttagtagagccgg

SEQ ID NO: 888 - DNA, 20 bp, synthetic construct
  source 1..20: mol_type = other DNA
gtgtccgccaccatgcctgg

SEQ ID NO: 889 - DNA, 20 bp, synthetic construct
  source 1..20: mol_type = other DNA
cagcctctcgatgcatagct

SEQ ID NO: 890 - DNA, 20 bp, synthetic construct
  source 1..20: mol_type = other DNA
ctcccgggttcaagcaattc

SEQ ID NO: 891 - DNA, 20 bp, synthetic construct
  source 1..20: mol_type = other DNA
tggcactatctcggctcact

SEQ ID NO: 892 - DNA, 20 bp, synthetic construct
  source 1..20: mol_type = other DNA
atggtgtctcgctctgtcgc

SEQ ID NO: 893 - DNA, 20 bp, synthetic construct
  source 1..20: mol_type = other DNA
gtgctcttgacctgctattg

SEQ ID NO: 894 - DNA, 20 bp, synthetic construct
  source 1..20: mol_type = other DNA
gggaggtgctcttgacctgc

SEQ ID NO: 895 - DNA, 20 bp, synthetic construct
  source 1..20: mol_type = other DNA
ttggtgcctctcttcagacc

SEQ ID NO: 896 - DNA, 20 bp, synthetic construct
  source 1..20: mol_type = other DNA
gtagttggtgcctctcttca

SEQ ID NO: 897 - DNA, 20 bp, synthetic construct
  source 1..20: mol_type = other DNA
gtgaatgccttctctgccca

SEQ ID NO: 898 - DNA, 20 bp, synthetic construct
  source 1..20: mol_type = other DNA
attctccccttgagcccagg

SEQ ID NO: 899 - DNA, 20 bp, synthetic construct
  source 1..20: mol_type = other DNA
tgctttctactgaccagggt

SEQ ID NO: 900 - DNA, 20 bp, synthetic construct
  source 1..20: mol_type = other DNA
gacactcccaggacctgagt

SEQ ID NO: 901 - DNA, 20 bp, synthetic construct
  source 1..20: mol_type = other DNA
tgctccctgtgactaccaga

SEQ ID NO: 902 - DNA, 20 bp, synthetic construct
  source 1..20: mol_type = other DNA
tcagccctaatgctccctgt

SEQ ID NO: 903 - DNA, 20 bp, synthetic construct
  source 1..20: mol_type = other DNA
gtgtctcaacaatcagccct

SEQ ID NO: 904 - DNA, 20 bp, synthetic construct
  source 1..20: mol_type = other DNA
ccagtgtctcaacaatcagc

SEQ ID NO: 905 - DNA, 20 bp, synthetic construct
  source 1..20: mol_type = other DNA
ctcccataggctgattccaa

SEQ ID NO: 906 - DNA, 20 bp, synthetic construct
  source 1..20: mol_type = other DNA
gagatgcaaagcacgaggtt

SEQ ID NO: 907 - DNA, 20 bp, synthetic construct
  source 1..20: mol_type = other DNA
gaggcagttgagatgcaaag

SEQ ID NO: 908 - DNA, 20 bp, synthetic construct
  source 1..20: mol_type = other DNA
cagaggcagttgagatgcaa

SEQ ID NO: 909 - DNA, 20 bp, synthetic construct
  source 1..20: mol_type = other DNA
tgtgtcagaggcagttgaga

SEQ ID NO: 910 - DNA, 20 bp, synthetic construct
  source 1..20: mol_type = other DNA
gattaggaaatgcacaacac

SEQ ID NO: 911 - DNA, 20 bp, synthetic construct
  source 1..20: mol_type = other DNA
gaacagagtgtgaaatgcca

SEQ ID NO: 912 - DNA, 20 bp, synthetic construct
  source 1..20: mol_type = other DNA
tcacccattagctggctcct

SEQ ID NO: 913 - DNA, 20 bp, synthetic construct
  source 1..20: mol_type = other DNA
cggtattgttttgcggcttg

SEQ ID NO: 914 - DNA, 20 bp, synthetic construct
  source 1..20: mol_type = other DNA
gagacggtattgttttgcgg

SEQ ID NO: 915 - DNA, 20 bp, synthetic construct
  source 1..20: mol_type = other DNA
gctctgttgtgagacggtat

SEQ ID NO: 916 - DNA, 20 bp, synthetic construct
  source 1..20: mol_type = other DNA
ctaacgtgctgaaggaccca

SEQ ID NO: 917 - DNA, 20 bp, synthetic construct
  source 1..20: mol_type = other DNA
ggtcatatctaacgtgctga

SEQ ID NO: 918 - DNA, 20 bp, synthetic construct
  source 1..20: mol_type = other DNA
gtcccaggtcatatctaacg

SEQ ID NO: 919 - DNA, 20 bp, synthetic construct
  source 1..20: mol_type = other DNA
ccacatgtggatgcccaatt

SEQ ID NO: 920 - DNA, 20 bp, synthetic construct
  source 1..20: mol_type = other DNA
ttgaggttgagattgagttt

SEQ ID NO: 921 - DNA, 20 bp, synthetic construct
  source 1..20: mol_type = other DNA
ggcttgaggttgagattgag

SEQ ID NO: 922 - DNA, 20 bp, synthetic construct
  source 1..20: mol_type = other DNA
tgtctaaggcttgaggttga

SEQ ID NO: 923 - DNA, 20 bp, synthetic construct
  source 1..20: mol_type = other DNA
gttttgtctaaggcttgagg

SEQ ID NO: 924 - DNA, 20 bp, synthetic construct
  source 1..20: mol_type = other DNA
ctctccctatgtcttctgaa

SEQ ID NO: 925 - DNA, 20 bp, synthetic construct
  source 1..20: mol_type = other DNA
ctgacccctattcttcttct

SEQ ID NO: 926 - DNA, 20 bp, synthetic construct
  source 1..20: mol_type = other DNA
cacattcctggtcttgaaca

SEQ ID NO: 927 - DNA, 20 bp, synthetic construct
  source 1..20: mol_type = other DNA
ttggtaggtatagagtctca

SEQ ID NO: 928 - DNA, 20 bp, synthetic construct
  source 1..20: mol_type = other DNA
aggaaggaggtcactttggg

SEQ ID NO: 929 - DNA, 20 bp, synthetic construct
  source 1..20: mol_type = other DNA
agtactgcacttgatggatc

SEQ ID NO: 930 - DNA, 20 bp, synthetic construct
  source 1..20: mol_type = other DNA
gtgtcagaggcagttgagat

SEQ ID NO: 931 - DNA, 20 bp, synthetic construct
  source 1..20: mol_type = other DNA
ccttgtgtcagaggcagttg

SEQ ID NO: 932 - DNA, 20 bp, synthetic construct
  source 1..20: mol_type = other DNA
ccaaccttgtgtcagaggca

SEQ ID NO: 933 - DNA, 20 bp, synthetic construct
  source 1..20: mol_type = other DNA
atggttaatgacccaacctt

SEQ ID NO: 934 - DNA, 20 bp, synthetic construct
  source 1..20: mol_type = other DNA
ataaatatggttaatgaccc

SEQ ID NO: 935 - DNA, 20 bp, synthetic construct
  source 1..20: mol_type = other DNA
ggggtaaataaatatggtta

SEQ ID NO: 936 - DNA, 20 bp, synthetic construct
  source 1..20: mol_type = other DNA
gaaaggggtaaataaatatg

SEQ ID NO: 937 - DNA, 20 bp, synthetic construct
  source 1..20: mol_type = other DNA
agtaagaaaggggtaaataa

SEQ ID NO: 938 - DNA, 20 bp, synthetic construct
  source 1..20: mol_type = other DNA
caatttatgaagtaagaaag

SEQ ID NO: 939 - DNA, 20 bp, synthetic construct
  source 1..20: mol_type = other DNA
aacacaatttatgaagtaag

SEQ ID NO: 940 - DNA, 20 bp, synthetic construct
  source 1..20: mol_type = other DNA
gcacaacacaatttatgaag

SEQ ID NO: 941 - DNA, 20 bp, synthetic construct
  source 1..20: mol_type = other DNA
gaaatgcacaacacaattta

SEQ ID NO: 942 - DNA, 20 bp, synthetic construct
  source 1..20: mol_type = other DNA
ttaggaaatgcacaacacaa

SEQ ID NO: 943 - DNA, 20 bp, synthetic construct
  source 1..20: mol_type = other DNA
aagattaggaaatgcacaac

SEQ ID NO: 944 - DNA, 20 bp, synthetic construct
  source 1..20: mol_type = other DNA
gaagacaagattaggaaatg

SEQ ID NO: 945 - DNA, 20 bp, synthetic construct
  source 1..20: mol_type = other DNA
tgcacagaagacaagattag

SEQ ID NO: 946 - DNA, 20 bp, synthetic construct
  source 1..20: mol_type = other DNA
tttctgcacagaagacaaga

SEQ ID NO: 947 - DNA, 20 bp, synthetic construct
  source 1..20: mol_type = other DNA
ggtaaatttctgcacagaag

SEQ ID NO: 948 - DNA, 20 bp, synthetic construct
  source 1..20: mol_type = other DNA
gaggtgctcttgacctgcta

SEQ ID NO: 949 - DNA, 20 bp, synthetic construct
  source 1..20: mol_type = other DNA
gtgcctctcttcagacccta

SEQ ID NO: 950 - DNA, 20 bp, synthetic construct
  source 1..20: mol_type = other DNA
ctactttttggtgaaacttg

SEQ ID NO: 951 - DNA, 20 bp, synthetic construct
  source 1..20: mol_type = other DNA
tgcttggtgaatgccttctc

SEQ ID NO: 952 - DNA, 20 bp, synthetic construct
  source 1..20: mol_type = other DNA
gtcttggatggcttcaaaag

SEQ ID NO: 953 - DNA, 20 bp, synthetic construct
  source 1..20: mol_type = other DNA
tattctccccttgagcccag

SEQ ID NO: 954 - DNA, 20 bp, synthetic construct
  source 1..20: mol_type = other DNA
taggagcactgtcccagtaa

SEQ ID NO: 955 - DNA, 20 bp, synthetic construct
  source 1..20: mol_type = other DNA
ttgctttctactgaccaggg

SEQ ID NO: 956 - DNA, 20 bp, synthetic construct
  source 1..20: mol_type = other DNA
acctgagtgatggggatccc

SEQ ID NO: 957 - DNA, 20 bp, synthetic construct
  source 1..20: mol_type = other DNA
atgctccctgtgactaccag

SEQ ID NO: 958 - DNA, 20 bp, synthetic construct
  source 1..20: mol_type = other DNA
gttcaccagtgtctcaacaa

SEQ ID NO: 959 - DNA, 20 bp, synthetic construct
  source 1..20: mol_type = other DNA
acagggaaatcacagaggtt

SEQ ID NO: 960 - DNA, 20 bp, synthetic construct
  source 1..20: mol_type = other DNA
gcagactgaaagctatcagg

SEQ ID NO: 961 - DNA, 20 bp, synthetic construct
  source 1..20: mol_type = other DNA
ccccattggaagtacagagg

SEQ ID NO: 962 - DNA, 20 bp, synthetic construct
  source 1..20: mol_type = other DNA
tctcccataggctgattcca

SEQ ID NO: 963 - DNA, 20 bp, synthetic construct
  source 1..20: mol_type = other DNA
cacacagaatcctagagcta

SEQ ID NO: 964 - DNA, 20 bp, synthetic construct
  source 1..20: mol_type = other DNA
caaggatgcccttttatctc

SEQ ID NO: 965 - DNA, 20 bp, synthetic construct
  source 1..20: mol_type = other DNA
gatgcaaagcacgaggttct

SEQ ID NO: 966 - DNA, 20 bp, synthetic construct
  source 1..20: mol_type = other DNA
gatctggtaaatttctgcac

SEQ ID NO: 967 - DNA, 20 bp, synthetic construct
  source 1..20: mol_type = other DNA
gtttccatacctggttcttt

SEQ ID NO: 968 - DNA, 20 bp, synthetic construct
  source 1..20: mol_type = other DNA
ttgagcttagatatggacaa

SEQ ID NO: 969 - DNA, 20 bp, synthetic construct
  source 1..20: mol_type = other DNA
ttgccctggaagttattgat

SEQ ID NO: 970 - DNA, 20 bp, synthetic construct
  source 1..20: mol_type = other DNA
ccattgaaatcattattctg

SEQ ID NO: 971 - DNA, 20 bp, synthetic construct
  source 1..20: mol_type = other DNA
ctcgcttctctcttgtcata

SEQ ID NO: 972 - DNA, 20 bp, synthetic construct
  source 1..20: mol_type = other DNA
cagagtgtgaaatgccaatc

SEQ ID NO: 973 - DNA, 20 bp, synthetic construct
  source 1..20: mol_type = other DNA
gaagaaggtagacagggtcc

SEQ ID NO: 974 - DNA, 20 bp, synthetic construct
  source 1..20: mol_type = other DNA
cataccctttcaattgaggc

SEQ ID NO: 975 - DNA, 20 bp, synthetic construct
  source 1..20: mol_type = other DNA
gaggattatctctgaccccc

SEQ ID NO: 976 - DNA, 20 bp, synthetic construct
  source 1..20: mol_type = other DNA
ttattcaaacaggtgggccc

SEQ ID NO: 977 - DNA, 20 bp, synthetic construct
  source 1..20: mol_type = other DNA
attgattcaggggtgttttc

SEQ ID NO: 978 - DNA, 20 bp, synthetic construct
  source 1..20: mol_type = other DNA
ggtaatggtaatggtagttt

SEQ ID NO: 979 - DNA, 20 bp, synthetic construct
  source 1..20: mol_type = other DNA
caagcgatctgagagttact

SEQ ID NO: 980 - DNA, 20 bp, synthetic construct
  source 1..20: mol_type = other DNA
gagaatctgagaattacttt

SEQ ID NO: 981 - DNA, 20 bp, synthetic construct
  source 1..20: mol_type = other DNA
tcaccaaaagagctcctgat

SEQ ID NO: 982 - DNA, 20 bp, synthetic construct
  source 1..20: mol_type = other DNA
tttctctccacgtgtgtgtg

SEQ ID NO: 983 - DNA, 20 bp, synthetic construct
  source 1..20: mol_type = other DNA
atcacccattagctggctcc

SEQ ID NO: 984 - DNA, 20 bp, synthetic construct
  source 1..20: mol_type = other DNA
taaattagaattattatagt

SEQ ID NO: 985 - DNA, 20 bp, synthetic construct
  source 1..20: mol_type = other DNA
ctgtgtggaaactggcaata

SEQ ID NO: 986 - DNA, 20 bp, synthetic construct
  source 1..20: mol_type = other DNA
gacggtattgttttgcggct

SEQ ID NO: 987 - DNA, 20 bp, synthetic construct
  source 1..20: mol_type = other DNA
cccagaatgttttataagct

SEQ ID NO: 988 - DNA, 20 bp, synthetic construct
  source 1..20: mol_type = other DNA
cttcatgtgttaatattcta

SEQ ID NO: 989 - DNA, 20 bp, synthetic construct
  source 1..20: mol_type = other DNA
taacgtgctgaaggacccaa

SEQ ID NO: 990 - DNA, 20 bp, synthetic construct
  source 1..20: mol_type = other DNA
gagtcctctatgtcccaggt

SEQ ID NO: 991 - DNA, 20 bp, synthetic construct
  source 1..20: mol_type = other DNA
tccacatgtggatgcccaat

SEQ ID NO: 992 - DNA, 20 bp, synthetic construct
  source 1..20: mol_type = other DNA
gcttgaggttgagattgagt

SEQ ID NO: 993 - DNA, 20 bp, synthetic construct
  source 1..20: mol_type = other DNA
gatccactgtgagttttgtc

SEQ ID NO: 994 - DNA, 20 bp, synthetic construct
  source 1..20: mol_type = other DNA
gccttacattttccattcag

SEQ ID NO: 995 - DNA, 20 bp, synthetic construct
  source 1..20: mol_type = other DNA
gattctctccctatgtcttc

SEQ ID NO: 996 - DNA, 20 bp, synthetic construct
  source 1..20: mol_type = other DNA
catgctgttgaactctttgc

SEQ ID NO: 997 - DNA, 20 bp, synthetic construct
  source 1..20: mol_type = other DNA
gatcaagttcaatttaccat

SEQ ID NO: 998 - DNA, 20 bp, synthetic construct
  source 1..20: mol_type = other DNA
tatgtttttcacaaagcaaa

SEQ ID NO: 999 - DNA, 20 bp, synthetic construct
  source 1..20: mol_type = other DNA
tgcttgtcttttcagtctcc

SEQ ID NO: 1000 - DNA, 20 bp, synthetic construct
  source 1..20: mol_type = other DNA
ttattggatatgtaacttgc

SEQ ID NO: 1001 - DNA, 20 bp, synthetic construct
  source 1..20: mol_type = other DNA
gttctacatatatattctgg

SEQ ID NO: 1002 - DNA, 20 bp, synthetic construct
  source 1..20: mol_type = other DNA
tacttgaattgtttattttc

SEQ ID NO: 1003 - DNA, 20 bp, synthetic construct
  source 1..20: mol_type = other DNA
gagcttgcctgacccctatt

SEQ ID NO: 1004 - DNA, 20 bp, synthetic construct
  source 1..20: mol_type = other DNA
gtcttgaacagtcctccagc

SEQ ID NO: 1005 - DNA, 20 bp, synthetic construct
  source 1..20: mol_type = other DNA
tttggtaggtatagagtctc

SEQ ID NO: 1006 - DNA, 20 bp, synthetic construct
  source 1..20: mol_type = other DNA
aaatagctaggagtataggt

SEQ ID NO: 1007 - DNA, 20 bp, synthetic construct
  source 1..20: mol_type = other DNA
aggcggtttcacaatagtgt

SEQ ID NO: 1008 - DNA, 20 bp, synthetic construct
  source 1..20: mol_type = other DNA
ttcactgtctgtcataattt

SEQ ID NO: 1009 - DNA, 20 bp, synthetic construct
  source 1..20: mol_type = other DNA
atggattcggttaagttaga

SEQ ID NO: 1010 - DNA, 20 bp, synthetic construct
  source 1..20: mol_type = other DNA
ccataaactataaactaagt

SEQ ID NO: 1011 - DNA, 20 bp, synthetic construct
  source 1..20: mol_type = other DNA
ggggtgttaccgtctttgtt

SEQ ID NO: 1012 - DNA, 20 bp, synthetic construct
  source 1..20: mol_type = other DNA
gcaggaaggaggtcactttg

SEQ ID NO: 1013 - DNA, 20 bp, synthetic construct
  source 1..20: mol_type = other DNA
caccaccagatcaataaact

SEQ ID NO: 1014 - DNA, 20 bp, synthetic construct
  source 1..20: mol_type = other DNA
gtactgcacttgatggatca

SEQ ID NO: 1015 - DNA, 20 bp, synthetic construct
  source 1..20: mol_type = other DNA
ctaaaatcagtgcttggggt

SEQ ID NO: 1016 - DNA, 20 bp, synthetic construct
  source 1..20: mol_type = other DNA
atatggtttaggagttatgc

SEQ ID NO: 1017 - DNA, 20 bp, synthetic construct
  source 1..20: mol_type = other DNA
ggtcagagacttgggtttga

SEQ ID NO: 1018 - DNA, 20 bp, synthetic construct
  source 1..20: mol_type = other DNA
gatggtcagagacttgggtt

SEQ ID NO: 1019 - DNA, 20 bp, synthetic construct
  source 1..20: mol_type = other DNA
ggtaagtgatggtcagagac

SEQ ID NO: 1020 - DNA, 20 bp, synthetic construct
  source 1..20: mol_type = other DNA
gcagcttttagaaatggtca

SEQ ID NO: 1021 - DNA, 20 bp, synthetic construct
  source 1..20: mol_type = other DNA
gtttaatctgaggatcctgt

SEQ ID NO: 1022 - DNA, 20 bp, synthetic construct
  source 1..20: mol_type = other DNA
tggctaacttgctgtggagt

SEQ ID NO: 1023 - DNA, 20 bp, synthetic construct
  source 1..20: mol_type = other DNA
ggtggagcttggcataaagt

SEQ ID NO: 1024 - DNA, 20 bp, synthetic construct
  source 1..20: mol_type = other DNA
ggccaggcaataaaagagtc

SEQ ID NO: 1025 - DNA, 20 bp, synthetic construct
  source 1..20: mol_type = other DNA
tcacagcagccggtaccgca

SEQ ID NO: 1026 - DNA, 20 bp, synthetic construct
  source 1..20: mol_type = other DNA
ggcaattagaggtagaaaag

SEQ ID NO: 1027 - DNA, 20 bp, synthetic construct
  source 1..20: mol_type = other DNA
gtggaaagactggcaattag

SEQ ID NO: 1028 - DNA, 20 bp, synthetic construct
  source 1..20: mol_type = other DNA
aaagggagctccaggactga

SEQ ID NO: 1029 - DNA, 20 bp, synthetic construct
  source 1..20: mol_type = other DNA
gtcaggtgctgttagaatca

SEQ ID NO: 1030 - DNA, 20 bp, synthetic construct
  source 1..20: mol_type = other DNA
gactgtcaggtgctgttaga

SEQ ID NO: 1031 - DNA, 20 bp, synthetic construct
  source 1..20: mol_type = other DNA
gtgatctgaataggctgcta

SEQ ID NO: 1032 - DNA, 20 bp, synthetic construct
  source 1..20: mol_type = other DNA
gatggccccttagtgatctg

SEQ ID NO: 1033 - DNA, 20 bp, synthetic construct
  source 1..20: mol_type = other DNA
gtctggacctctcctatggg

SEQ ID NO: 1034 - DNA, 20 bp, synthetic construct
  source 1..20: mol_type = other DNA
ctcctacccttcctacctta

SEQ ID NO: 1035 - DNA, 20 bp, synthetic construct
  source 1..20: mol_type = other DNA
tctgtaatcagatttcaccg

SEQ ID NO: 1036 - DNA, 20 bp, synthetic construct
  source 1..20: mol_type = other DNA
agacagctgcctctaattcc

SEQ ID NO: 1037 - DNA, 20 bp, synthetic construct
  source 1..20: mol_type = other DNA
tagtgccacgctctgcttta

SEQ ID NO: 1038 - DNA, 20 bp, synthetic construct
  source 1..20: mol_type = other DNA
atattctgcagtaaggcctc

SEQ ID NO: 1039 - DNA, 20 bp, synthetic construct
  source 1..20: mol_type = other DNA
gggtttgatttaggaatcat

SEQ ID NO: 1040 - DNA, 20 bp, synthetic construct
  source 1..20: mol_type = other DNA
tttcggtaagtgatggtcag

SEQ ID NO: 1041 - DNA, 20 bp, synthetic construct
  source 1..20: mol_type = other DNA
tggtcacaacttcatggttc

SEQ ID NO: 1042 - DNA, 20 bp, synthetic construct
  source 1..20: mol_type = other DNA
atgctgtttaatctgaggat

SEQ ID NO: 1043 - DNA, 20 bp, synthetic construct
  source 1..20: mol_type = other DNA
ttcaagggcatagaatcgtc

SEQ ID NO: 1044 - DNA, 20 bp, synthetic construct
  source 1..20: mol_type = other DNA
cttagtttttaggtggcccc

SEQ ID NO: 1045 - DNA, 20 bp, synthetic construct
  source 1..20: mol_type = other DNA
gggtgatattacctttgctc

SEQ ID NO: 1046 - DNA, 20 bp, synthetic construct
  source 1..20: mol_type = other DNA
accaggggactttgataagg

SEQ ID NO: 1047 - DNA, 20 bp, synthetic construct
  source 1..20: mol_type = other DNA
atgctcctacaccctgccct

SEQ ID NO: 1048 - DNA, 20 bp, synthetic construct
  source 1..20: mol_type = other DNA
agcggtggagcttggcataa

SEQ ID NO: 1049 - DNA, 20 bp, synthetic construct
  source 1..20: mol_type = other DNA
ccaggcaataaaagagtcag

SEQ ID NO: 1050 - DNA, 20 bp, synthetic construct
  source 1..20: mol_type = other DNA
acatgcgtgtgacagtataa

SEQ ID NO: 1051 - DNA, 20 bp, synthetic construct
  source 1..20: mol_type = other DNA
atcctcaatgagtacttgtc

SEQ ID NO: 1052 - DNA, 20 bp, synthetic construct
  source 1..20: mol_type = other DNA
ttcacagcagccggtaccgc

SEQ ID NO: 1053 - DNA, 20 bp, synthetic construct
  source 1..20: mol_type = other DNA
ctgagtgagttacttctact

SEQ ID NO: 1054 - DNA, 20 bp, synthetic construct
  source 1..20: mol_type = other DNA
tgtgtttttgcagtgccaat

SEQ ID NO: 1055 - DNA, 20 bp, synthetic construct
  source 1..20: mol_type = other DNA
aatagtactactcaggactg

SEQ ID NO: 1056 - DNA, 20 bp, synthetic construct
  source 1..20: mol_type = other DNA
aagactggcaattagaggta

SEQ ID NO: 1057 - DNA, 20 bp, synthetic construct
  source 1..20: mol_type = other DNA
agggcacttctttctatttg

SEQ ID NO: 1058 - DNA, 20 bp, synthetic construct
  source 1..20: mol_type = other DNA
ctatcatcttgcacccccaa

SEQ ID NO: 1059 - DNA, 20 bp, synthetic construct
  source 1..20: mol_type = other DNA
ggactgagatatttttacta

SEQ ID NO: 1060 - DNA, 20 bp, synthetic construct
  source 1..20: mol_type = other DNA
gaagacttatgtgcaaggca

SEQ ID NO: 1061 - DNA, 20 bp, synthetic construct
  source 1..20: mol_type = other DNA
caggtgctgttagaatcaat

SEQ ID NO: 1062 - DNA, 20 bp, synthetic construct
  source 1..20: mol_type = other DNA
gcatatggtatccccacttc

SEQ ID NO: 1063 - DNA, 20 bp, synthetic construct
  source 1..20: mol_type = other DNA
tctgaataggctgctagggg

SEQ ID NO: 1064 - DNA, 20 bp, synthetic construct
  source 1..20: mol_type = other DNA
tgaagttgcagatggcccct

SEQ ID NO: 1065 - DNA, 20 bp, synthetic construct
  source 1..20: mol_type = other DNA
ctgatttctaactgaggata

SEQ ID NO: 1066 - DNA, 20 bp, synthetic construct
  source 1..20: mol_type = other DNA
gtgcagctcggtatctgata

SEQ ID NO: 1067 - DNA, 20 bp, synthetic construct
  source 1..20: mol_type = other DNA
gatgatgtcatcttccctct

SEQ ID NO: 1068 - DNA, 20 bp, synthetic construct
  source 1..20: mol_type = other DNA
accacctttactgaactgtg

SEQ ID NO: 1069 - DNA, 20 bp, synthetic construct
  source 1..20: mol_type = other DNA
tcctatgggcttgtattatg

SEQ ID NO: 1070 - DNA, 20 bp, synthetic construct
  source 1..20: mol_type = other DNA
cttcttcgaagccagacaac

SEQ ID NO: 1071 - DNA, 20 bp, synthetic construct
  source 1..20: mol_type = other DNA
acccttcctaccttagaggg

SEQ ID NO: 1072 - DNA, 20 bp, synthetic construct
  source 1..20: mol_type = other DNA
aattgaccagaagaggcagc

SEQ ID NO: 1073 - DNA, 383 bp, Mus musculus
  source 1..383: mol_type = genomic DNA
agagatagagtagggcagggtgcaggggcacttgaactttaagcctaactccagggttgtttttgaaagaaagaatcctaactctttcagagcccagcctcttcgtttttcataatgtatttatgctgtcacttgcttggataaaaatcagttgacaagtacccattgagcatgtaatatatgcaaggcactgccatgctggggctgctgtgaaggtacactgaaataactcattcagaaaatggaaattgacagtacaaaaccccagatggagctatgacagccctgtgtattcctgttgatttcactttcctacttagcactcccatccattccacaccaagaaaaaaaataataataaataaataaataatgggatggat

SEQ ID NO: 1074 - DNA, 808 bp, Mus musculus
  source 1..808: mol_type = genomic DNA
tcccaccccttagaagttcagaggtcaacctgtgcataaaggagatttattagcacaccaaaggctatcaggcattaagcaaacaaggaaatcacaaaggtttaaagttcatctaacagctaagccactaacagacacttccatggccaggagattggaggccctgtgagaactttggcgttgtctcctgttgacaagtgcttagtataaaagcacggtctgtataagaatttcactcttggagggcaggcaggtcacatgtccttgaacaccctcaaaggccattgcttggtgaatgacccctttcccaattctagttcttgacaatcctgtagtcatctttctggttgacactaacacctacttgtgaactctttttcctctgctctgggttgtctcggatcaatatctgccacacaacaaaggtgtttccttgctttaagctattggtttggtgcatcttttcttaaggttgttccactgctaactactgtgctgtgtgtaaaatgggacctagacacagcttgaagatagattttatgaaaactttttgtatccaatatgaaagtttgcatagaatttggtcaaggcttgcttattggatttagatattcaccatcactgattttaatccttcacagttcttcagtggagctcagctggaaagtttcttcgagggaatcaagtagttcatggatatttagtcaccctggatttttacacagcgtactgccataaaggaacattcaggtcaggtcacatgggaatcactataaagagaactgactcaaaggtgttggagccagtt

SEQ ID NO: 1075 - DNA, 989 bp, Mus musculus
  source 1..989: mol_type = genomic DNA
ctggccatggaagtgtctgttagtggcttagctgttagatgaactttaaacctttgtgatttccttgtttgcttaatgcctgatagcctttggtgtgctaataaatctcctttatgcacaggttgacctctgaacttctaaggggtgggataggaaccagcctatgggaaaggagatggctctagaattctcagtgggatcatgaacaaacaaaaggtatcctggaagagaattggcccttttcatctcgcctgcctcagatacaaacttggatcactgaccgtacttatttgcctttttccaatctcattaattgtgccgtgcatttcataatcttatctgctttaccagatcacaaaataaaaagcatctggaaacagttgagatttttctatcactttctaaacacagaaaaattcatagctatgctcaaaatcaagaactggcagggtaagtgataaagggagattgacttcagtggtaatgtgctctactagaaatattagaattaaatcccttgatcagcatttcacattgttcagaattttggtcttccaatgtttcagttgaaagcgtatggtgagaaagggggtggaaaaaaatctttactgatttcacagttttccctaaggcttgcctatttaaataaatattttgcaatgacgtagaaagaaaacatccctgagtcaatatttttagttaaagtcccattaccatctgttaattgaacagtaattttcagattttctataaatcatgagctacttggataacatatttatacatgaaaagaaaaatatagccagatgataggtaatttggtttttcactatcttgattcaatttcatttcctattgccagttctcatgaacattcaagctccattccagctgcttacgaagcactgccagatactaagaagtactaaattctccagcatgtggttttgacctaggacacagaatttgaacaaattacatttatttat

SEQ ID NO: 1076 - DNA, 594 bp, Mus musculus
  source 1..594: mol_type = genomic DNA
ctaaaaatattgactcagggatgttttctttctacgtcattgcaaaatatttatttaaataggcaagccttagggaaaactgtgaaatcagtaaagatttttttccaccccctttctcaccatacgctttcaactgaaacattggaagaccaaaattctgaacaatgtgaaatgctgatcaagggatttaattctaatatttctagtagagcacattaccactgaagtcaatctccctttatcacttaccctgccagttcttgattttgagcatagctatgaatttttctgtgtttagaaagtgatagaaaaatctcaactgtttccagatgctttttattttgtgatctggtaaagcagataagattatgaaatgcacggcacaattaatgagattggaaaaaggcaaataagtacggtcagtgatccaagtttgtatctgaggcaggcgagatgaaaagggccaattctcttccaggataccttttgtttgttcatgatcccactgagaattctagagccatctcctttcccataggctggttcctatcccaccccttagaagttcagaggtcaacctgtgcataaaggaga

SEQ ID NO: 1077 - DNA, 1620 bp, Homo sapiens
  source 1..1620: mol_type = genomic DNA
cacggtgaaatctgattacagaaggactgagaaatgaaagctttgctggaattagaggcagctgtctcaaataagagtaaagcagagcgtggcactacattctgcagaaagaggccttactgcagaatatagttttatgcatcaccatgattcctaaatcaaacccaagtctctgaccatcacttaccgaaaatttcgaaccatgaagttgtgaccatttctaaaagctgcattgtttaacaggatcctcagattaaacagcatcttcttttaattgtgtaaaggacgattctatgcccttgaaaactccacagcaagttagccagcgggggccacctaaaaactaagaaatgtgttcagttgcagtgagggagcaaaggtaatatcacccttcagcttccttatcaaagtcccctggttagagatactgcagggcagggtgtaggagcatttgaactttatgccaagctccaccgctgtgtattttttttggaagaaatcctgactcttttattgcctggcctcctcatttttcataatatatttatactgtcacacgcatgtacaaaatcaattgacaagtactcattgaggatataatatatgcaaggcactgcggtaccggctgctgtgaaggtgagtagaagtaactcactcagaaaatgcaaattggcactgcaaaaacacagatgtagatgtgacagtcctgagtagtactatttccatttatttcacttttctacctctaattgccagtctttccacaaaaacaaatagaaagaagtgccctggaagctattttgggggtgcaagatgatagagaaggtatagtaaaaatatctcagtcctggagctccctttgtgccttgcacataagtcttcaataaagaaataattgctggccgggcacggtggctcacgcctgtaatcccagcactttgggaggctgaggcgggcggatcacttgagcccaggagttcgagaccagcctggccaacatggtgaatccccgtctccactaaaaatacaaaaattagctagacgtggtggtgcacacctgtaatcccagctatttgggaggctgaggcatgagagtcgcttgaacccgggagacagaggttgcagtgagcccagattgcaccactgtactccagcctgggtgacagagagagacactgtctcaaaaaaaagaaagaaagaaagaaaaagaaaagaaatagttgcctattgattctaacagcacctgacagtcagtacaagtgggaagtggggataccatatgctcttcccctagcagcctattcagatcactaaggggccatctgcaacttcacatttattcatttatcctcagttagaaatcaggtgattcaaagtcaattctgtatcagataccgagctgcactgaagtttcagagagggaagatgacatcatctttgcagagttcacagttcagtaaaggtggtatgatttataaacaaatatccataatacaagcccataggagaggtccagacaaagttgtctggcttcgaagaagcgggagccctctaaggtaggaagggtaggaggaaggagaggaggggcaggggtagggctgcctcttctggtcaatttat

SEQ ID NO: 1078 - DNA, 700 bp, Mus musculus
  source 1..700: mol_type = genomic DNA
gccatgtgatttctcaacagcatgttccaaaatgtgcacaagagtttatgtaagccagcaaggtcaaatctctgaacatagtccacactggaacagaaggaaaggcatgtagacagctcttggacagtgaaacatggtgggaagcagggatctttctacagtcttaggacaagttggggtgacttgtatggctcttggtcttcactggaagacaagctacaacaaagaattggcccaacattctgaggctttgcttggttttgcctagcctgcatgaggagtcagaaactgaagggaagcccgaggaccagaacagagccatagcaggtgaatatcaaggagaaggagaagcaatgactgttgctccatctcgttccctctcccactaaggctgctttcacttcaagcagaacagacaaaccagaaaattgcaggaatcaaagttcctttcaggaataaatctcttggccactgtgatgttttgaatgagaatggcctccagatgcccttttgtttgaataattagtccctggttagtagagctgtttggggaggattaggatgtgtggccttgctggaaaaggtgtgtcaccggggttggctttgaggtttcaaaagcctgtactattgccagccggttctctctctctctctctctctctctctctctctctctctctctctctctctctcttccctc

SEQ ID NO: 1079 - DNA, 20 bp, synthetic construct
  source 1..20: mol_type = other DNA
ggcggagacagagagaaggt

SEQ ID NO: 1080 - DNA, 20 bp, synthetic construct
  source 1..20: mol_type = other DNA
cagagggcggagacagagag

SEQ ID NO: 1081 - DNA, 20 bp, synthetic construct
  source 1..20: mol_type = other DNA
aaaacagagggcggagacag

SEQ ID NO: 1082 - DNA, 20 bp, synthetic construct
  source 1..20: mol_type = other DNA
acaaaacagagggcggagac

SEQ ID NO: 1083 - DNA, 20 bp, synthetic construct
  source 1..20: mol_type = other DNA
gagggcttggcagggagatc

SEQ ID NO: 1084 - DNA, 20 bp, synthetic construct
  source 1..20: mol_type = other DNA
aagttaaagagggcttggca

SEQ ID NO: 1085 - DNA, 20 bp, synthetic construct
  source 1..20: mol_type = other DNA
gaacacaaagttaaagaggg

SEQ ID NO: 1086 - DNA, 20 bp, synthetic construct
  source 1..20: mol_type = other DNA
gaggaacacaaagttaaaga

SEQ ID NO: 1087 - DNA, 20 bp, synthetic construct
  source 1..20: mol_type = other DNA
gggaggaacacaaagttaaa

SEQ ID NO: 1088 - skipped

SEQ ID NO: 1089 - DNA, 20 bp, synthetic construct
  source 1..20: mol_type = other DNA
ctttgggaggaacacaaagt

SEQ ID NO: 1090 - DNA, 20 bp, synthetic construct
  source 1..20: mol_type = other DNA
gggctttgggaggaacacaa

SEQ ID NO: 1091 - DNA, 20 bp, synthetic construct
  source 1..20: mol_type = other DNA
gggggctttgggaggaacac

SEQ ID NO: 1092 - DNA, 20 bp, synthetic construct
  source 1..20: mol_type = other DNA
gaccaggtttggtaataggg

SEQ ID NO: 1093 - DNA, 20 bp, synthetic construct
  source 1..20: mol_type = other DNA
caagaacttggaccaggttt

SEQ ID NO: 1094 - DNA, 22 bp, synthetic construct
  source 1..22: mol_type = other DNA
gcggagcagggagattgaacag

SEQ ID NO: 1095 - DNA, 24 bp, synthetic construct
  source 1..24: mol_type = other DNA
agcggagcagggagattgaacaga

SEQ ID NO: 1096 - DNA, 22 bp, synthetic construct
  source 1..22: mol_type = other DNA
tttgggaggaacacaaagttaa

SEQ ID NO: 1097 - DNA, 24 bp, synthetic construct
  source 1..24: mol_type = other DNA
ctttgggaggaacacaaagttaaa

SEQ ID NO: 1098 - DNA, 23 bp, synthetic construct
  source 1..23: mol_type = other DNA
ttaatacagctctggagtggggt

SEQ ID NO: 1099 - DNA, 20 bp, synthetic construct
  source 1..20: mol_type = other DNA
tatgaagtaagaaaggggta